SEQUENCE LISTING (13 sequences)

SEQ ID NO: 1 - DNA, 30 bp, Artificial Sequence
  Description of Artificial Sequence Synthetic primer
tttttttttttttttttttttttttttttt

SEQ ID NO: 2 - DNA, 50 bp, Artificial Sequence
  Description of Artificial Sequence Synthetic primer
tttttttttttttttttttttttttttttttttttttttttttttttttt

SEQ ID NO: 3 - DNA, 22 bp, Artificial Sequence
  Description of Artificial Sequence Synthetic primer
gaagctggtatctccaagaatg

SEQ ID NO: 4 - DNA, 26 bp, Artificial Sequence
  Description of Artificial Sequence Synthetic primer
ctgctttgatgacacccaccgcaact

SEQ ID NO: 5 - DNA, 20 bp, Artificial Sequence
  Description of Artificial Sequence Synthetic primer
tttttttttttttttttttt

SEQ ID NO: 6 - DNA, 25 bp, Artificial Sequence
  Description of Artificial Sequence Synthetic primer
ttttttttttttttttttttttttt

SEQ ID NO: 7 - DNA, 37 bp, Artificial Sequence
  Description of Artificial Sequence Synthetic oligonucleotide
uuuuuuugcaggugacaggttttttcctgtcacctgc

SEQ ID NO: 8 - DNA, 20 bp, Artificial Sequence
  Description of Artificial Sequence Synthetic primer
tttttttttttttttttttt

SEQ ID NO: 9 - DNA, 20 bp, Artificial Sequence
  Description of Artificial Sequence Synthetic primer
tttttttttttttttttttt

SEQ ID NO: 10 - DNA, 22 bp, Artificial Sequence
  Description of Artificial Sequence Synthetic primer
tututututututututututt

SEQ ID NO: 11 - DNA, 25 bp, Artificial Sequence
  Description of Artificial Sequence Synthetic primer
ttttttttttttttttttttttttt

SEQ ID NO: 12 - DNA, 30 bp, Artificial Sequence; identical to SEQ ID NO: 1
  Description of Artificial Sequence Synthetic primer

SEQ ID NO: 13 - DNA, 30 bp, Artificial Sequence; identical to SEQ ID NO: 1
  Description of Artificial Sequence Synthetic primer

(19) United States
(12) Patent Application Publication
(10) Pub. No.: US 2016/0264950 A1
(43) Pub. Date: Sep. 15, 2016

(54) REVERSIBLY INACTIVATED THERMOSTABLE REVERSE TRANSCRIPTASES, COMPOSITIONS AND METHODS FOR USE

(71) Applicant: LIFE TECHNOLOGIES CORPORATION, Carlsbad, CA (US)

(72) Inventors: Jeffrey Rogers, San Diego, CA (US); Bianca Lam, Encinitas, CA (US); Joanna Guo, San Diego, CA (US); Lushen Li, San Diego, CA (US)

(21) Appl. No.: 14/835,609

(22) Filed: Aug. 25, 2015

Related U.S. Application Data
(60) Provisional application No. 62/041,584, filed on Aug. 25, 2014.

(51) Int. Cl. C12N 9/12; C12P 19/34

(52) U.S. Cl. CPC C12N 9/1276; C12P 19/34; C12Y 207/07049

(57) ABSTRACT

The present invention provides reversibly inactivated reverse transcriptase enzymes, particularly those having increased thermostability and/or thermoreactivity and compositions, methods and kits that include such enzymes, for the reverse transcription of nucleic acid molecules. Also provided are compositions and methods for the reactivation of reversibly inactivated reverse transcriptases. More particularly, the present invention relates to compositions and methods that can increase the speed of reactivation and stabilize reactivation of chemically modified reverse transcriptase enzymes prior to, or as part of a reverse transcription reaction. As compared to existing compositions and methods, the reversibly inactivated reverse transcriptase enzymes of the present invention provide for a significant reduction in non-specific reverse transcription from template nucleic acid molecules.

### SEQUENCE LISTING

The instant application contains a Sequence Listing which has been submitted electronically in ASCII format and is hereby incorporated by reference in its entirety. Said ASCII copy, created on Oct. 22, 2015, is named LT00967_SL.txt and is 5,550 bytes in size.

### FIELD OF THE INVENTION

The present invention provides reversibly inactivated reverse transcriptase enzymes, particularly those having increased thermostability and/or thermoreactivity and compositions, methods and kits that include such enzymes, for the reverse transcription of nucleic acid molecules. Also provided are compositions and methods for the reactivation of reversibly inactivated reverse transcriptases.

### BACKGROUND

A common technique used to study gene expression in living cells is to produce a DNA copy (complementary DNA or “cDNA”) of the cellular complement of RNA. This technique provides a means to study RNA from living cells which avoids the direct analysis of inherently unstable RNA. As a common first step in cDNA synthesis, the RNA molecules from an organism are isolated from an extract of cells or tissues of the organism. After mRNA isolation, using methods such as affinity chromatography utilizing a short sequence of deoxy-thymine nucleotides (oligo dT), oligonucleotide sequences are annealed to the isolated mRNA molecules and enzymes with reverse transcriptase activity can be utilized to produce cDNA copies of the RNA sequence, utilizing the RNA/DNA primer as a template. Thus, reverse transcription of mRNA is a key step in many forms of gene expression analyses. Generally, mRNA is reverse transcribed into cDNA for subsequent analysis by primer extension, polymerase chain reaction, and/or sequencing.

The specificity of reverse transcription depends on the specificity of primer hybridization to a target RNA sequence. The reverse transcription of RNA templates requires a primer sequence which is annealed to an RNA template in order for DNA synthesis to be initiated from the 3′ OH of the primer. Primers may be selected to be complementary to, or substantially complementary to, sequences occurring at the 3′ end of each strand of the target nucleic acid sequence. At temperatures used in a typical reverse transcriptase reaction, the primers may hybridize too many non-target sequences as well as the intended target sequence. Additionally, reverse transcription reaction mixtures are typically assembled at room temperature, well below the temperature needed to ensure specific primer hybridization. Under such less stringent conditions, primers may bind non-specifically to partially complementary RNA sequences (or even to other primers) and initiate the synthesis of undesired extension products, which can be reverse transcribed along with the correct target sequence, resulting in the production non-specific cDNA. Non-specific cDNA extension products can compete with the specific cDNA reverse transcriptase products in later applications. For example, the presence of non-specific cDNA extension products can significantly decrease the efficiency of the detection of specific cDNA products in RT-PCR. Thus, in these instances it would be highly advantageous to be able to reverse transcribe RNA templates at temperatures which preclude the formation of non-specific primer template complexes (e.g., at temperatures above 50° C.). In such circumstances, the reduction of non-specific reverse transcriptase activity would result in greater specificity of cDNA synthesis. Currently, there are no, or very few, reliable and easy to use reagents and/or methods for improving the specificity of reverse transcription. Therefore, there is a need for reverse transcriptase enzymes that can be activated at elevated temperatures that inhibit the formation of non-specific primer/templates.

Several methods exist to address the problem of non-specific amplification products that arise from non-specific extension by thermostable DNA polymerases during a polymerase chain reaction (PCR). In the case of PCR, non-specific products are caused by the extension of misprimed oligonucleotides during the reaction set-up or the initial heating phase of a PCR reaction. Accordingly, essential components such as the oligonucleotide primers, nucleotide triphosphates, magnesium ions, or thermostable nucleic acid polymerases can be sequestered for release at higher temperatures, thereby reducing the probability of having non-specific hybridization or the extension of misprimed oligonucleotides. These techniques are referred to as “manual hot-start PCR” methods. Another method for reducing formation of extension products from misprimed oligonucleotides during a PCR reaction set-up entails the use of a reversible chemically modified thermostable DNA polymerase that becomes active only after incubation at an elevated temperature, thus preventing the production of non-specific DNA synthesis during reaction set-up and the initial heating phase of PCR. See, for example, U.S. Pat. Nos. 5,677,152 and 5,773,258 which describe a method for the amplification of a target nucleic acid using a thermostable DNA-dependent DNA polymerase reversibly inactivated using dicarboxylic acid anhydride compounds.

However DNA polymerase enzymes (i.e., DNA-dependent polymerases) and reverse transcriptase enzymes (i.e., RNA-dependent polymerases) are quite different both structurally and functionally. For example, reverse transcriptase enzymes can generate cDNA from an RNA template, a process termed reverse transcription, while DNA polymerases generate cDNA from a DNA template only. Reverse transcriptase activity is often associated with retroviruses which comprise three activities: (a) RNA-dependent DNA polymerase activity, (b) ribonuclease H activity, and (c) DNA-dependent DNA polymerase activity. As such, retroviral reverse transcriptases are able to convert single-stranded genomic RNA into double-stranded cDNA.

Because reverse transcription reaction assembly is typically performed on ice or at room temperature, a number of events can lead to non-specific RNA interactions which, in turn, generate non-specific priming and unwanted cDNA generation. For example, RNA self-hybridizes, RNA hybridizes to other RNA, and the reverse transcription primer (oligo(dT) of various lengths or any other primer) can non-specifically hybridize to non-mRNAs. Because typical reverse transcriptases have activity at room temperature and below, all of these non-specific interactions may result in non-specific priming events and subsequent non-specific cDNA generation.

Moreover, approximately 80% of total RNA is composed of ribosomal RNA and polyadenlyated RNA (mRNA) only comprises 1-3% of total RNA. This poses a significant problem for applications such as those used for generating cDNA libraries and for RNA sequencing (RNA-Seq) because it is not desirable or efficient to convert ribosomal RNA (rRNA) to a complementary DNA (cDNA). Currently, to get around this problem of ribosomal cDNA being non-specifically synthesized, researchers utilize selection methods (such as oligo (dT)-based magnetic beads to isolate mRNA) or ribosomal RNA depletion (such as rRNA probes-based magnetic beads to pull out ribosomal RNAs) prior to reverse transcription. These isolation and depletion methods are not very thorough, meaning some unwanted ribosomal RNAs are still present, and they create bias where some desired mRNAs are lost.

The inability to easily and reliably control non-specific reverse transcription resulting from mismatched primer sequences involving a non-thermostable reverse transcriptase (such as a retroviral reverse transcriptase) is a common problem in the field. In many instances it is desirable to initiate reverse transcription reactions at temperatures or under conditions, above which, the formation of non-specific primer complexes is inhibited. Thus, in many instances, it is desirable to reverse transcribe RNA at temperatures above 37° C. However, nonthermostable reverse transcriptases generally lose activity when incubated at temperatures much above 37° C. (e.g., at 50+° C.).

Modified thermally stable and/or thermally reactive reverse transcriptases would potentially solve these problems by allowing researchers to skip isolation and depletion methods because these enzymes would not be active until the reaction temperature is raised to temperatures that destroy most non-specific nucleic acid hybridization. Consequently, hot start reverse transcriptases that can withstand elevated temperatures (e.g., >50° C.) would enable specific conversion of mRNA to cDNA and prevent non-specific conversion of rRNA into ribosomal cDNA.

### SUMMARY

The present invention provides reversibly inactivated reverse transcriptase enzymes, particularly those having increased thermostability and/or thermoreactivity, for use in hot start reverse transcription reactions which can provide a simple and economical solution to the problem of non-specific reverse transcription. The invention further provides methods for generating such reversibly inactivated reverse transcriptases, methods and reagents for the reactivation of reversibly inactivated reverse transcriptases, and methods for using reversibly inactivated reverse transcriptases. In some embodiments, the disclosed methods use chemically modified reverse transcriptase enzymes which can be reactivated by incubation of the reverse transcription reaction mixture at an elevated temperature in a thermally-dependent manner. Thus, non-specific reverse transcription is greatly reduced because the reaction mixture does not support primer extension until the temperature of the reaction mixture has been elevated to a temperature which improves primer hybridization specificity and/or minimizes non-specific primer hybridization.

Traditionally, reverse transcription reactions are performed at a pH >8, namely about pH 8.3 and at temperatures between 37° C. to 50° C. For example, reverse transcriptase suppliers commonly provide reaction buffers at pH 8.3 and recommend using a reaction temperature between 37° C.-50° C. This is likely because thermostable and/or thermoreactive reverse transcriptase enzymes, in particular retroviral reverse transcriptase enzymes, able to withstand exposure to a lower pH and/or increased temperatures have not been identified until relatively recently. See, for example, U.S. patent applications Ser. Nos. 14/603,256; 13/921,989; and U.S. Pat. Nos. 8,580,548 and 8,835,148. Surprisingly, the present inventors have discovered that the use of reaction buffers with a pH <8 can accelerate reactivation of chemically modified thermostable/thermoreactive reverse transcriptases to 10 minutes or less under activating conditions. Moreover, the present inventors have discovered that the use of stabilizing components, such as saccharides and/or polyalcohols enables reactivation of chemically modified reverse transcriptases to occur at higher temperatures than previously utilized (e.g., at elevated temperatures of 55° C. to 60° C.). The present inventors have also discovered that the use of protein additives, such as bovine serum albumin (BSA) and apotransferrin, enhances activity of chemically modified thermostable/thermoreactive reverse transcriptases during reactivation at alkaline pH.

Accordingly, one aspect of the present invention provides modified reverse transcriptase enzymes having increased thermostability and/or thermoreactivity, in which, the modified reverse transcriptase enzymes are produced by the reaction of a mixture of a thermostable and/or thermoreactive reverse transcriptase enzyme which catalyzes a primer extension reaction and a modifier reagent. In some embodiments, the reaction between the reverse transcriptase enzyme and the modifier reagent is through a covalent chemical modification of the enzyme which results in reversible inactivation of reverse transcriptase enzyme activity. In some embodiments, the modified enzyme exhibits less than 50% (e.g., 30%, 20%, 10%, 5%, 1%) activity in an aqueous buffer under non-activating conditions while incubation of the same modified enzyme in an aqueous buffer under activating conditions results the enzyme having greater than 50% (e.g., 70%, 80%, 90%, 95%, 99%, 100%) enzyme activity.

In some embodiments, the instant invention provides thermostable and/or thermoreactive reverse transcriptase enzymes comprising a chemical modification, wherein the modified reverse transcriptase is thermostable and/or thermoreactive at a temperature exceeding about 42° C. (e.g., 50° C.) at a pH between 6.0 to 9.0, and wherein the chemical modification results in at least a 50% (e.g., 90%, 100%) decrease in reverse transcriptase activity as compared to a corresponding unmodified reverse transcriptase when activity is measured under non-activating conditions. In some embodiments, the non-activating conditions comprise a pH of at least 8.0 and/or a temperature less than about 37° C. In some embodiments, the decrease in reverse transcriptase activity of the modified reverse transcriptase provided herein is maintained for at least about 30 minutes under non-activating conditions.

In some embodiments, the modified reverse transcriptases provided herein, exhibit an increase in reverse transcriptase activity in a reaction mixture under activating conditions compared to the reverse transcriptase activity observed under non-activating conditions. In some embodiments, the activating conditions comprise a pH less than about 8.0 and/or a temperature greater than about 37° C. In some embodiments, the increase in reverse transcriptase activity is at least two-fold under activating conditions. In some embodiments, the increase in reverse transcriptase activity occurs in less than about 30 minutes under activating conditions.

In some embodiments, the thermostable and/or thermoreactive reverse transcriptases of the instant invention are mutant Moloney Murine Leukemia Virus (M-MLV) reverse transcriptases. In other embodiments, the chemically modified reverse transcriptase enzymes are enzymes having increased thermostability and/or thermoreactivity, such as those described in U.S. patent applications Ser. Nos. 14/603,256; 13/921,989; and U.S. Pat. Nos. 8,580,548 and 8,835,148, the disclosures of which are herein incorporated by reference in their entireties. In some embodiments, the enzymes are thermostable at a temperature above 37° C. (e.g., at 42° C., 53° C., 55° C., 57° C., 60° C.). In some embodiments, there is no significant increase in reverse transcriptase enzyme activity of the modified reverse transcriptase enzyme in at least about 30 minutes under non-activating conditions.

In some embodiments, the modifier reagent can be maleic anhydride; exo-cis-3,6-endoxo-Δ⁴-tetrahydropthalic anhydride; citraconic anhydride; 3,4,5,6-tetrahydrophthalic anhydride; cis-aconitic anhydride; and 2,3-dimethylmaleic anhydride. In some favorable aspects, the modifier reagent is 2,3-dimethylmaleic anhydride. In some embodiments, the modifier reagent is used at a ratio of about 50× to 800×:1× (modifier:enzyme) for modification of the reverse transcriptase. In some embodiments, the modifier reagent is used at a ratio of about 300× to 500×:1× (modifier:enzyme) for modification of the reverse transcriptase.

In yet other embodiments, the modifier reagent can be imide. In some embodiments the modifier reagent is used at a ratio of about 1400× to 1800× to 1× (modifier:enzyme) for modifications of the reverse transcriptase. In some embodiments the modifier reagent is used at a ratio of about 1600× to 1× (modifier:enzyme) for modifications of the reverse transcriptase.

In yet further embodiments, the modifiedx reverse transcriptase can have an inactivation that results in at least 50%, 60%, 70%, 80%, or 90% inactivity compared to a corresponding unmodified reverse transcriptase. In some exemplary aspects, the inactivation is essentially complete (i.e., 100%).

In other embodiments, the present invention provides a composition comprising a thermostable and/or thermoreactive reverse transcriptase enzyme comprising a chemical modification, wherein the modified reverse transcriptase is thermostable and/or thermoreactive at a temperature exceeding about 42° C. at a pH between 6.0 to 9.0, and wherein the chemical modification results in at least a 50% decrease in reverse transcriptase activity as compared to a corresponding unmodified reverse transcriptase when activity is measure under non-activating conditions. In some embodiments, the composition is at a pH of 8.0 or less.

In some embodiments, the disclosed compositions optionally comprise an RNA template and/or a primer. In some embodiments, the primer is an oligo(dT)ₙ primer, where “n” is a number equal to or between 20 to 30 (SEQ ID NO: 1).

In some embodiments, the disclosed compositions comprise at least one stabilizing component. In some embodiments the at least one stabilizing component is selected from the group consisting of glycerol, trehalose, lactose, maltose, galactose, glucose, sucrose, dimethyl sulfoxide (DMSO), polyethylene glycol, and sorbitol. In some embodiments, when the at least one stabilizing component is glycerol and/or trehalose, the glycerol concentration is between 1% and 15% and the trehalose concentration is between 1% and 18%.

In some embodiments, the disclosed compositions comprise at least one protein additive. In some embodiments, the at least one protein additive is unacetylated bovine serum albumin (BSA) and/or apotransferrin. In some embodiments, the concentration of the unacetylated bovine serum albumin (BSA) is about 500 ng/uL and/or the concentration of the apotransferrin is about 200 ng/uL.

In other embodiments, the present invention provides methods for the reactivation of a reversibly inactivated reverse transcriptase by subjecting the modified enzyme to an aqueous buffer having a pH less than 8.0 whereby exposure of the modified enzyme to a pH less than 8.0 enhances reactivation (e.g., increases the rate of reactivation) of the reversibly inactivated modified enzyme while under activating conditions.

In further embodiments, the present invention provides methods for the reactivation of a reversibly inactivated reverse transcriptase by inclusion of the modified enzyme in an aqueous buffer comprising an added component(s) for stabilization of the modified enzyme, such as a saccharide and/or polyalcohol or protein additive, under activating conditions. In some embodiments, the disclosed methods for reactivation of a reversibly inactivated reverse transcriptase in a reaction mixture comprise reactivating the reversibly inactivated reverse transcriptase at a pH of 8.3 or less (e.g., 6.9 to 8.0).

In some embodiments, the reversibly inactivated reverse transcriptase is an M-MLV reverse transcriptase. In some preferred embodiments, the M-MLV reverse transcriptase is a mutant M-MLV reverse transcriptase. In some embodiments, the reversibly inactivated reverse transcriptase is reversibly inactivated by a covalent chemical modification. In some embodiments, the covalent chemical modification comprises a lysine modified by an anhydride or an imide.

In some embodiments, the instant invention provides reaction mixtures used for reactivating the reversibly inactivated reverse transcriptase. In some embodiments, the reaction mixture used for reactivation comprises at least one saccharide and/or polyalcohol compound. In some embodiments, the at least one saccharide compound is trehalose and the at least one polyalcohol compound is glycerol or sorbitol. In some embodiments, the concentration of trehalose is about 0.1 M to 0.5 M. In some embodiments, the concentration of glycerol is about 1% to 15% and/or the concentration of sorbitol is about 1% to 18%. In some embodiments, the reaction mixture used for reactivation comprises at least one protein additive. In some embodiments, the at least one protein additive is bovine serum albumin (BSA) or apotransferrin. In some embodiments, the concentration of BSA is about 500 ng/uL and/or the concentration of apotransferrin is about 200 ng/uL.

In some embodiments, reactivating comprises increasing the temperature of the reaction mixture (e.g. to a temperature greater than about 37° C.; between 50° C. and 60° C.).

In other embodiments, the present invention provides methods for the reverse transcription of a target nucleic acid contained in a sample comprising the steps of: (a) contacting the sample with a reverse transcription reaction mixture containing a primer complementary to the target nucleic acid and a modified reverse transcriptase enzyme having increase thermostability and/or thermoreactivity. In some embodiments, the modified reverse transcriptase enzyme is produced by the reaction of a mixture of a reverse transcriptase enzyme which catalyzes a primer extension reaction and a modifier reagent, in which, the reaction results in a covalent chemical modification of the reverse transcriptase enzyme which results in inactivation of enzyme activity. In some other embodiments, incubation of the modified reverse transcriptase enzyme in an aqueous buffer under non-activating conditions results in no significant increase in enzyme activity over the course of at least 30 minutes, and incubation of the modified enzyme in an aqueous buffer under activating conditions results in an increase in enzyme activity in less than about 30 minutes. In some other embodiments, the methods further include a step (b) incubating the resulting mixture of step (a) under activating conditions for a time sufficient to reactivate said reverse transcriptase enzyme and allowing formation of primer extension products. In some embodiments, the activating conditions comprise incubation for a period of time at a temperature greater than 37° C. and/or at a pH less than about 8.0. In some embodiments, the activating conditions comprise addition of a stabilizing component, such as a saccharide and/or a polyalcohol. In some embodiments, the stabilizing component is glycerol and/or sorbitol.

In other embodiments, the present invention provides methods for the reverse transcription of a target nucleic acid contained in a sample comprising the steps of: (a) contacting the sample with a reverse transcription reaction mixture containing a primer and a reversibly inactivated thermostable and/or thermoreactive reverse transcriptase, wherein the reverse transcriptase is thermostable and/or thermoreactive at a temperature above 42° C. at a pH between 6.0 to 9.0, and wherein the reverse transcriptase exhibits essentially no reverse transcriptase activity in reaction mixture under non-activating conditions; and (b) incubating the resulting mixture of step (a) under activating conditions for an amount of time sufficient to reactivate the reversibly inactivated reverse transcriptase and allow formation of primer extension products. In some embodiments, the non-activating conditions comprise subjecting the reverse transcriptase to pH of at least 8.0 and/or to a temperature less than about 37° C. or less. In some embodiments, the activating conditions comprise incubation in an aqueous buffer having a pH less than about 8.0 and/or exposure to a temperature greater than about 37° C. In some embodiments, the amount of time sufficient to reactivate the reversibly inactivated reverse transcriptase is 30 minutes (e.g., 10 minutes) or less.

In other aspects of the above embodiments, non-activating conditions can comprise a pH greater than about 8.0 and a temperature less than about 37° C., and activating conditions can include subjecting the enzyme to a pH greater than 8.0 and/or to a temperature greater than about 37° C. In some embodiments the activating temperature may be 40° C. to 50° C., 50° C. to 60° C., or 60° C. to 65° C. In some embodiments, there is a significant increase in reverse transcriptase enzyme activity in less than about 30 minutes under activating conditions. In yet other embodiments, the increase in enzyme activity is at least two-fold (e.g., three-fold, four-fold, five-fold, six-fold, etc.), and the increase in enzyme activity occurs in less than about 30 minutes under activating conditions. In some embodiments, the increase in enzyme activity under activity conditions occurs in less than about 30 minutes, 15 minutes, 10 minutes, 5 minutes, or 1 minute.

In certain embodiments, a stabilizing component may be used to support enhanced stability of the compositions upon storage and/or reaction mixtures during synthesis. In even further embodiments, the stabilizing component can comprise a saccharide compound, such as trehalose, maltose, glucose, sucrose, lactose, xylobiose, agarobiose, cellobiose, levanbiose, quitobiose, 2-3-glucuronosylglucuronic acid, allose, altrose, galactose, gulose, idose, mannose, talose, sorbitol, levulose, xylitol and arabitol. However, the stabilizing component is not limited to these examples.

In certain embodiments, a protein additive may be used to support enhanced stability of the compositions upon storage and/or reaction mixtures during synthesis. In some further embodiments, the protein additive can comprise Taq mutant M6D, SpermineCassein, β-lactalnumi lactalbumin, α-lactalbumin, apotransferrin and/or acetylated BSA.

Additional embodiments provide methods for the reverse transcription of a target nucleic acid contained in a sample including the steps of: (a) contacting the sample with a reverse transcription reaction mixture containing a primer complementary to the target nucleic acid and a modified reverse transcriptase enzyme. In some embodiments, the modified reverse transcriptase enzyme is produced by a reaction of a mixture of a reverse transcriptase enzyme which catalyzes a primer extension reaction and a modifier reagent, in which, the reaction results in a covalent chemical modification of the reverse transcriptase enzyme which results in essentially complete inactivation of enzyme activity. In some other embodiments, incubation of the modified reverse transcriptase enzyme in an aqueous buffer at a pH >8.0 and/or at a temperature less than about 37° C. results in no significant increase in enzyme activity in less than about 60 minutes, and incubating the modified enzyme to an aqueous buffer, formulated to a pH ≦8.0 (at 25° C.) and/or to a temperature greater than about 37° C. results in at least a two-fold increase in enzyme activity in less than about 30 minutes. In some other embodiments, the methods further include a step (b) incubating the resulting mixture of step (a) at a temperature which is greater than about 37° C. for a time sufficient to reactivate the reverse transcriptase enzyme and allow formation of primer extension products. In some embodiments the incubation temperature may be 40° C. to 50° C., 50° C. to 60° C., or 60° C. to 65° C.

In other embodiments, methods are provided for strand specific reverse transcription of a target nucleic acid in a sample comprising sense and antisense transcription products comprising (a) contacting the sample with a reverse transcription reaction mixture containing a primer complementary to one of the sense or antisense transcription products and a modified reverse transcriptase enzyme. In some embodiments, the modified reverse transcriptase enzyme is produced by a reaction of a mixture of a reverse transcriptase enzyme which catalyzes a primer extension reaction and a modifier reagent, wherein the reaction results in a covalent chemical modification of the reverse transcriptase enzyme which results in inactivation of enzyme activity. In some embodiments, incubation of the modified reverse transcriptase enzyme in an aqueous buffer under non-activating conditions results in no significant increase in enzyme activity, and incubation of the modified enzyme in an aqueous buffer under activating conditions results in an increase in enzyme activity. In some embodiments, the methods further comprise step (b) incubating the resulting mixture of step (a) under activating conditions for a time sufficient to reactivate the reverse transcriptase enzyme and allow formation of primer extension products. In some embodiments, primer extension products are preferably those which are produced only from strand-specific hybridization of primer to template.

Further embodiments of the present invention provide kits for a reverse transcription reaction including a modified reverse transcriptase enzyme and/or compositions as described in the embodiments and aspects disclosed herein. In some embodiments, the kit comprises a modified reverse transcriptase enzyme as disclosed herein in a container and, optionally further comprises instructions for making a cDNA.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

### BRIEF DESCRIPTION OF THE DRAWINGS

The skilled artisan will understand that the drawings described below are for illustration purposes only. The drawings are not intended to limit the scope of the present teachings in any way.

FIG. 1 shows a depiction of an RNA221 fluorescent hairpin substrate (SEQ ID NO: 7) for use in an exemplary fluorescent activity assay for hot start reverse transcription.

FIGS. 2A, 2B, and 2C show the results of fluorescent activity assays using unmodified Maxima™ H Minus reverse transcriptase and chemically modified Maxima™ H Minus reverse transcriptase modified with varying concentrations of 2,3-dimethylmaleic anhydride (DMMA) as indicated (i.e., 50×, 100×, 200×, 400×, 600× and 800×). Enzyme activity for unmodified or modified reverse transcriptase was determined after preincubation for 10 minutes at (A) 25° C., (B) 50° C. and (C) 60° C.

FIG. 3 shows the results of radioactive activity assays performed using unmodified Maxima™ H Minus reverse transcriptase and chemically modified Maxima™ H Minus reverse transcriptase modified with a 400×:1× molar ratio of 2,3-dimethylmaleic anhydride (DMMA):enzyme or a 1600×:1× molar ratio of N-ethoxycarbonyl-2,3-dimethylmaleimide:enzyme.

FIGS. 4A through 4X show the results of fluorescent activity assays using chemically modified Maxima™ H Minus reverse transcriptase and a novel mutant MMLV (“Mut MMLV”) reverse transcriptase after preincubation at various temperatures as indicated (i.e., 25° C., 30° C., 37° C., 42° C., 50° C. and 60° C.) for varying lengths of time as indicated (i.e., 5 minutes, 10 minutes, 20 minutes, and 30 minutes) at pH 8.3 or 7.3. Modified reverse transcriptases were modified with 300× 2,3-dimethylmaleic anhydride (DMMA).

FIGS. 5A and 5B show the results of fluorescent activity assays using chemically modified Maxima™ H Minus reverse transcriptase and a novel mutant MMLV (“Mut MMLV”) reverse transcriptase after preincubation at various temperatures as indicated (i.e., 53° C., 53.5° C., 54.3° C., 55.7° C., 57.3° C., 58.6° C., 59.5° C. and 60° C.) for 10 minutes at pH 7.3 in the absence of any stabilizers.

FIGS. 6A through 6X and FIGS. 7A through 7X show the results of fluorescent activity assays using chemically modified Maxima™ H Minus reverse transcriptase (FIGS. 6A through 6X) and a novel mutant MMLV (“Mut MMLV”) reverse transcriptase (FIGS. 7A through 7X) after preincubation at various temperatures as indicated (i.e., 25° C., 30° C., 37° C., 42° C., 50° C. and 60° C.) for varying lengths of time as indicated (i.e., 5 minutes, 10 minutes, 20 minutes, and 30 minutes) at pH 8.3 or 7.3 in the presence of no additives or in the presence of 10% glycerol and 0.3M trehalose. Modified reverse transcriptases were modified with 300× 2,3-dimethylmaleic anhydride (DMMA).

FIGS. 8A through 8D shows the results of fluorescent activity assays using unmodified and chemically modified SuperScript™ III reverse transcriptase and wild type MMLV reverse transcriptase after preincubation at various temperatures as indicated (i.e., 25° C., 37° C., 50° C. and 60° C.) for 10 minutes at pH 8.3 or 7.3 in the presence of no additives or in the presence of 10% glycerol and 0.3M trehalose. Modified SuperScript™ III reverse transcriptase was modified with 200× 2,3-dimethylmaleic anhydride (DMMA) and wild type M-MLV reverse transcriptase was modified with 400× DMMA.

FIGS. 9A, 9B, and 9C show the results of fluorescent activity assays using unmodified Maxima™ H Minus reverse transcriptase and chemically modified Maxima™ H Minus reverse transcriptase modified with varying concentrations of 2,3-dimethylmaleic anhydride (DMMA) as indicated (i.e., 50×, 100×, 200×, 400×, 600× and 800×) at pH 7.3 in the presence of 10% glycerol and 0.3M trehalose. Enzyme activity for unmodified or modified reverse transcriptase was determined after preincubation for 10 minutes at (A) 25° C., (B) 50° C. and (C) 60° C.

FIGS. 10A through 10R and FIGS. 11A through 11R show the results of fluorescent activity assays for unmodified and chemically modified Maxima™ H Minus reverse transcriptase (FIGS. 10A through 10R) and a novel mutant MMLV (“Mut MMLV”) reverse transcriptase (FIGS. 11A through 11R) each having (a) no dialysis or (b) with dialysis following modification after preincubation at various temperatures as indicated (i.e., 25° C., 30° C., 37° C., 42° C., 50° C. and 60° C.) for varying lengths of time as indicated (i.e., 5 minutes, 10 minutes, 20 minutes, and 30 minutes) at pH 7.3 in the presence of 10% glycerol and 0.3M trehalose.

FIGS. 12A and 12B show the results of fluorescent activity assays using chemically modified Maxima™ H Minus reverse transcriptase and a novel mutant MMLV (“Mut MMLV”) reverse transcriptase after preincubation for 10 minutes at 60° C. and pH 7.3 in the presence of no additives or in the presence of various stabilizing additives, as indicated.

FIGS. 13A through 13U and FIGS. 14A through 14U show the results of fluorescent activity assays using chemically modified Maxima™ H Minus reverse transcriptase (FIGS. 13A through 13U) and a novel mutant MMLV (“Mut MMLV”) reverse transcriptase (FIGS. 14A through 14U) after preincubation for either 5 minutes or 10 minutes at various temperatures as indicated (i.e., 25° C., 50° C. and 60° C.) at varying pH (as indicated) in the presence of 10% glycerol and 0.3M trehalose.

FIG. 15 shows the products of reverse transcription reactions performed using an RNA ladder as the template at varying pH (e.g., 7.3 to 8.0) using chemically modified Maxima™ H Minus reverse transcriptase (modified with a 400×:1× molar ratio of 2,3-dimethylmaleic anhydride (DMMA):enzyme) in the presence of no protein additives (lanes, 2, 6, 10, 14, 18, 22, 26 and 30), in the presence of 500 ng/uL of bovine serum albumin (BSA) (lanes, 3, 7, 11, 15, 19, 23, 27 and 31), in the presence of 200 ng/uL of apotransferrin (lanes 4, 8, 12, 16, 20, 24, 28 and 32) or in the presence of both 500 ng/uL BSA and 200 ng/uL apotransferrin (lanes 5, 9, 13, 17, 21, 25, 29 and 33). Reverse transcription reactions using unmodified Maxima™ H Minus reverse transcriptase at pH 8.3 and with no protein additives are also shown (lane 1).

FIG. 16 depicts the results of various reverse transcription reactions performed using several modified (+) and unmodified (−) reverse transcriptases (thermostable RTs=Maxima™ H Minus and a mutant MMLV; non-thermostable RTs=SuperScript™ III and wild type MMLV) at different temperatures as indicated (i.e., 60° C., 50° C. and 37° C.) for 1 hour. The PCR products of these reverse transcription reactions were run on E-Gel® 48 Agarose Gels, 1% (Invitrogen™) and visualized by E-Gel® Imager System with UV Light Base (Invitrogen™)™) (SEQ ID NOS 8-10, 5, 6 and 11-13, respectively, in order of appearance).

FIGS. 17A and 17B are tables that quantify the fold increase (positive values) or fold reduction (negative values) in reverse transcription quantitative PCR reactions (RT-qPCR). Reverse transcription was performed using unmodified enzymes at 50° C. with pH 8.3 and 7.3 buffer, manual hot start using unmodified enzymes at 50° C-57° C. with pH 8.3 and 7.3 buffer, and actual hot start using modified Maxima™ H Minus at 50° C-57° C. with pH 7.3 buffer.

### DETAILED DESCRIPTION

Provided herein are reversibly inactivated (“hot start”) reverse transcriptase enzymes, compositions and kits comprising the same, and methods for reactivation and use of such enzymes. In particular, reversibly inactivated reverse transcriptase enzymes that are thermostable and/or thermoreactive are provided. Also provided are compositions and methods for enhancing the reactivation rate and stabilization of chemically modified reverse transcriptase enzymes under activating conditions involving elevated temperatures and/or conditions having a pH less than about 8.0. Also provided are mechanisms for lowering the temperature at which reactivation of chemically-modified reverse transcriptases can occur, which can be beneficial in situations where the use of thermostable and/or thermoreactive enzymes is not an option.

The present invention further provides compositions and methods for reverse transcribing a nucleic acid molecule using a primer-based reverse transcription reaction which provides a simple and economical solution to the problem of non-specific reverse transcription. The compositions and methods disclosed herein use reversibly inactivated reverse transcriptase enzymes which can be reactivated by subjecting the reverse transcription reaction mixture to an elevated temperature and/or by lowering the pH of the reaction. Non-specific reverse transcription is greatly reduced because the reaction mixture does not support primer extension until the temperature of the reaction mixture has been elevated to a temperature which improves primer hybridization specificity (or minimizes non-specificity). Reduced non-specific reverse transcription may also allow for the selective transcription of either the sense or antisense transcript from a biological sample containing both transcripts.

Specifically, the present disclosure relates to reversibly inactivated reverse transcriptase enzymes which are, for example, produced by a reaction between a reverse transcriptase enzyme and a modifier reagent. The modification reactions disclosed herein result in a significantly reduced activity (e.g., reduced by at least 50%), and preferably essentially complete inactivity in reverse transcriptase enzyme activity. In some preferred embodiments, such inactivation is maintained at low temperature and/or at a pH greater than 8.0 (i.e., non-activating conditions) and is reversed when the modified reverse transcriptase is exposed to an elevated temperature and/or a pH less than about 8.0 (i.e., activating conditions). As discussed in greater detail herein, the present inventors have generated modified thermostable and/or thermoreactive reverse transcriptase enzymes through the reaction of a thermostable and/or thermoreactive reverse transcriptase enzyme and a modifier reagent.

In some embodiments, the reactions disclosed herein result in essentially complete inactivation of enzyme activity at ambient temperatures, such as those used to set-up reverse transcription reactions. In some other embodiments, activity is restored upon exposure to increased temperatures and/or a decreased pH which aids in inhibiting the formation of mismatched or non-specific primer/template complexes.

I. Definitions

For the purposes of interpreting this specification, the following definitions will apply, and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The term “hybridization” refers generally to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions”. Stable duplexes of substantially complementary sequences can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs, following the guidance provided by the art (see, e.g., Sambrook et al., 1989, supra). Generally, stringent hybridization conditions are selected to be about 5° C. lower than the thermal melting point (Tm) for the specific sequence at a defined ionic strength and pH. The Tm is the temperature (under defined ionic strength and pH) at which 50% of the base pairs have dissociated. Relaxing the stringency of the hybridization conditions will allow sequence mismatches to be tolerated; the degree of mismatch tolerated can be controlled by suitable adjustment of the hybridization conditions.

The term “primer” refers generally to an oligonucleotide, whether natural or synthetic (which may comprise nucleotide analogs or modified nucleotides, such as phosphorothioates), capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature.

A primer is preferably a single-stranded oligodeoxyribonucleotide, although oligonucleotide analogues, such as “peptide nucleic acids”, can act as primers and are encompassed within the meaning of the term “primer” as used herein. The appropriate length of a primer depends on the intended use of the primer, but typically ranges from 6 to 50 nucleotides. Short primer molecules generally require cooler temperatures to form sufficiently stable hybrid complexes with the template. A primer need not reflect the exact sequence of the template nucleic acid, but must be sufficiently complementary to hybridize with the template.

The term “primer extension” as used herein refers to both to the synthesis of DNA resulting from the polymerization of individual nucleoside triphosphates using a primer as a point of initiation, and to the joining of additional oligonucleotides to the primer to extend the primer. Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning of the amplified product. The region of the primer which is sufficiently complementary to the template to hybridize is referred to herein as the hybridizing region. The terms “target region” and “target nucleic acid” refers to a region or subsequence of a nucleic acid which is to be reverse transcribed.

The primer hybridization site can be referred to as the target region for primer hybridization. As used herein, an oligonucleotide primer is “specific” for a target sequence if the number of mismatches present between the oligonucleotide and the target sequence is less than the number of mismatches present between the oligonucleotide and non-target sequences which may be present in the sample. Hybridization conditions can be chosen under which stable duplexes are formed only if the number of mismatches present is no more than the number of mismatches present between the oligonucleotide and the target sequence. Under such conditions, the oligonucleotide can form a stable duplex only with a target sequence. Thus, the use of target-specific primers under suitably stringent reverse transcription conditions enables the specific reverse transcription of those target sequences which contain the target primer binding sites. The use of sequence-specific reverse transcription conditions enables the specific reverse transcription of those target sequences which contain the exactly complementary primer binding sites.

The term “antisense strand” refers to the strand of a double stranded DNA molecule which is transcribed into mRNA during transcription. The term “sense strand” refers to the strand of a double stranded molecule which is not transcribed into mRNA during transcription.

The term “non-specific reverse transcription” refers generally to the reverse transcription of nucleic acid sequences other than the target sequence which results from primers hybridizing to sequences other than the target sequence and non-target sequences self-hybridizing or hybridizing to other non-target sequences, and then serving as substrates for primer extension. The hybridization of a primer to a non-target sequence and non-target sequence hybridization to itself or other non-target sequences are referred to as “non-specific hybridization”, and can occur during the lower temperature, reduced stringency pre-reaction conditions.

The term “reverse transcriptase enzyme” refers generally to an enzyme that has RNA-dependent DNA polymerase activity, namely an enzyme which can utilize an RNA template to incorporate dNTP starting at the 3′OH of an annealed primer sequence. Although retroviral reverse transcriptase enzymes are commonly appreciated by those skilled in the art, it is to be understood that reverse transcriptase enzymes may also be isolated from non-retroviral sources. Example reverse transcriptase enzymes that can be isolated from non-retroviral sources include mobile genetic elements such as the LTR and non-LRT retrotransposons, among others. The term “reverse transcriptase” can also refer to telomerase enzymes which use RNA to template DNA synthesis at the ends of chromosomes to form telomeres.

A reverse transcriptase enzyme may also have the property of thermostability and/or thermoreactivity. The term “thermostability” or “thermostable” refers generally to the ability to withstand exposure to elevated temperatures, but not necessarily show activity at such elevated temperatures. The term “thermoreactivity” or “thermoreactive” refers generally to the ability of a reverse transcriptase to exhibit enzyme activity at elevated temperatures.

The thermostable and/or thermoreactive reverse transcriptase enzymes contemplated by the current invention can withstand the high temperature incubation used to remove the modifier groups, typically greater than 40° C., preferably greater than about 50° C., without suffering an irreversible loss of activity. In some embodiments, modified reverse transcriptase enzymes usable in the methods of the present invention include thermostable and/or thermoreactive reverse transcriptase enzymes, such as retroviral reverse transcriptase enzymes, as well as thermostable and/or thermoreactive DNA polymerases with substantial reverse transcriptase activity.

For the purposes of this disclosure, a thermostable and/or thermoreactive reverse transcriptase retains a greater percentage or amount of activity after a heat treatment of at least 50° C. than is retained by a non-thermostable or non-thermoreactive reverse transcriptase enzyme, after an identical treatment. Thus, a reverse transcriptase having increased/enhanced thermostability may be defined as a reverse transcriptase having any increase in thermostability, preferably from about 1.2 to about 10,000 fold, from about 1.5 to about 10,000 fold, from about 2 to about 5,000 fold, or from about 2 to about 2000 fold (preferably greater than about 5 fold, more preferably greater than about 10 fold, still more preferably greater than about 50 fold, still more preferably greater than about 100 fold, still more preferably greater than about 500 fold, and most preferably greater than about 1000 fold) retention of activity after a heat treatment greater than 50° C. sufficient to cause a reduction in the activity of a reverse transcriptase that is not thermostable. In some embodiments, the reverse transcriptase of the invention is a mutated reverse transcriptase that can be compared to a corresponding un-mutated or wild type reverse transcriptase to determine the relative enhancement or increase in thermostability.

For example, after a heat treatment at 60° C. for 5 minutes, a thermostable reverse transcriptase may retain approximately 90% of the activity present before the heat treatment, whereas a reverse transcriptase that is wild type for thermostability may retain 10% of its original activity. Likewise, after a heat treatment at 60° C. for 15 minutes, a thermostable reverse transcriptase may retain approximately 80% of its original activity, whereas a reverse transcriptase that is wild type for thermostability may have no measurable activity. Similarly, after a heat treatment at 60° C. for 15 minutes, a thermostable reverse transcriptase may retain approximately 50%, approximately 55%, approximately 60%, approximately 65%, approximately 70%, approximately 75%, approximately 80%, approximately 85%, approximately 90%, or approximately 95% of its original activity, whereas a reverse transcriptase that is wild type for thermostability may have no measurable activity or may retain 20%, 15%, 10%, or none of its original activity. In the first instance (i.e., after heat treatment at 60° Cfor 5 minutes), the thermostable reverse transcriptase would be said to be 9-fold more thermostable than the wild type reverse transcriptase (90% compared to 10%). Examples of conditions which may be used to measure thermostability of an enzyme such as reverse transcriptases are well-known in the art and can be readily determined by persons of ordinary skill in the art.

For example, thermostability of a reverse transcriptase can be determined by comparing the residual activity of a reverse transcriptase that has been subjected to a heat treatment, e.g., incubated at a temperature greater than 50° C. for a given period of time, for example, five minutes, to a control sample of the same reverse transcriptase that has been incubated at room temperature for the same length of time as the heat treatment. Typically the residual activity may be measured by following the incorporation of a radiolabeled deoxyribonucleotide into an oligodeoxyribonucleotide primer using a complementary oligoribonucleotide template. For example, the ability of the reverse transcriptase to incorporate [α-32P]-dGTP into an oligo-dG primer using a poly(riboC) template may be assayed to determine the residual activity of the reverse transcriptase. Other methods for measuring residual activity are known by those of skill in the art, such as by incorporation of unlabeled nucleotides into a fluorescently-labeled primer. See, for example, Nikiforov, T. T., Anal Biochem., 2011, 412(2): 229-36, which is hereby incorporated by reference.

In another aspect, thermostable reverse transcriptases of the invention may include any reverse transcriptase which is inactivated at a higher temperature compared to the corresponding wild type, un-mutated reverse transcriptase. Preferably, the inactivation temperature for the thermostable reverse transcriptases of the invention is from about 2° C. to about 50° C. (e.g., about 2° C., about 4° C., about 6° C., about 8° C., about 10° C., about 12° C., about 14° C., about 16° C., about 18° C., about 19° C., about 20° C., about 21° C., about 22° C., about 23° C., about 24° C., about 26° C., about 28° C., about 30° C., about 32° C., about 34° C., about 36° C., about 38° C., about 40° C., about 42° C., about 44° C., about 46° C., about 48° C., or about 50° C.) higher than the inactivation temperature for the corresponding wild type, un-mutated reverse transcriptase. More preferably, the inactivation temperature for the reverse transcriptases of the invention is from about 5° C. to about 50° C., from about 5° C. to about 40° C., from about 5° C. to about 30° C., or from about 5° C. to about 25° C. greater than the inactivation temperature for the corresponding wild type, un-mutated reverse transcriptase, when compared under the same conditions. In some embodiments, mutant reverse transcriptases of the invention possess reverse transcriptase activity after at least one minute (e.g., 1 minute, 2 minutes, 5 minutes, 10 minutes, 15 minutes, 30 minutes, etc.) at an elevated temperature (e.g., 50° C., 55° C., 60° C., 65° C.) that is at least 10% (e.g., 10%, 25%, 50%, 75%, 100%, 150%, 200%, 300%, etc.) of the reverse transcriptase activity of wild type reverse transcriptase after 5 minutes at a lower temperature (e.g., 50° C., 45° C., 42° C., 40° C., 37° C.).

The difference in inactivation temperature for a thermostable and/or thermoreactive reverse transcriptase of the invention compared to a non-thermostable or non-thermoreactive reverse transcriptase (e.g., a corresponding wild type or un-mutated reverse transcriptase) can be determined by treating samples of such reverse transcriptases at different temperatures for a defined time period and then measuring residual reverse transcriptase activity, if any, after the samples have been heat treated. Determination of the difference or delta in the inactivation temperature between the test reverse transcriptase compared to a control reverse transcriptase is determined by comparing the difference in temperature at which each reverse transcriptase is inactivated (i.e., no residual reverse transcriptase activity is measurable in the particular assay used). As will be recognized, any number of reverse transcriptase assays may be used to determine the different or delta of inactivation temperatures for any reverse transcriptases tested.

In another aspect, thermostability of a reverse transcriptase of the invention is determined by measuring the half-life of the reverse transcriptase activity of a reverse transcriptase of interest. Such half-life may be compared to a control or wild type reverse transcriptase to determine the difference (or delta) in half-life. Half-life of the reverse transcriptases of the invention are preferably determined at elevated temperatures (e.g., greater than 37° C.) and preferably at temperatures ranging from 40° C. to 80° C., more preferably at temperatures ranging from 45° C. to 75° C., 50° C. to 70° C., 55° C. to 65° C., and 58° C. to 62° C. Preferred half-lives of the reverse transcriptases of the invention may range from 4 minutes to 10 hours, 4 minutes to 7.5 hours, 4 minutes to 5 hours, 4 minutes to 2.5 hours, or 4 minutes to 2 hours, depending upon the temperature used. For example, the reverse transcriptase activity of the reverse transcriptases of the invention may have a half-life of at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 20 minute, at least 25 minutes, at least 30 minutes, at least 40 minutes, at least 50 minutes, at least 60 minutes, at least 70 minutes, at least 80 minutes, at least 90 minutes, at least 100 minutes, at least 115 minutes, at least 125 minutes, at least 150 minutes, at least 175 minutes, at least 200 minutes, at least 225 minutes, at least 250 minutes, at least 275 minutes, at least 300 minutes, at least 400 minutes, at least 500 minutes at temperatures of 48° C., 50° C., 52° C., 54° C., 56° C., 58° C., 60° C., 62° C., 64° C., 66° C., 68° C., and/or 70° C.

The term “reversibly inactivated”, as used herein, refers generally to an enzyme which has been inactivated by reaction with a compound which results in the covalent modification (also referred to as chemical modification) of the enzyme, wherein the modifier compound is removable under appropriate conditions. The reaction which results in the removal of the modifier compound need not be the reverse of the modification reaction. As long as there is a reaction which results in removal of the modifier compound and restoration of enzyme function, the enzyme is considered to be reversibly inactivated.

The term “reaction mixture” refers to a solution containing reagents necessary to carry out a given reaction.

A “reverse transcription reaction mixture”, refers generally to a solution containing reagents necessary to carry out a reverse transcription reaction, and typically contains oligonucleotide primers and a reverse transcriptase enzyme in a suitable buffer. A reaction mixture is referred to as complete if it contains all reagents necessary to enable the reaction, and incomplete if it contains only a subset of the necessary reagents.

It will be understood by one skilled in the art that reaction components are routinely stored as separate solutions, each containing a subset of the total components, for reasons of convenience, storage stability, and to allow for independent adjustment of the concentrations of the components depending on the application, and, furthermore, that reaction components are combined prior to the reaction to create a complete reaction mixture.

The term “non-activating conditions” refers generally to conditions, for instance of pH and/or temperature, under which the activity of a modified reverse transcriptase enzyme as described herein has substantially reduced or undetectable activity.

The term “activating conditions” refers generally to conditions, for instance of pH and/or temperature, under which the activity of a modified reverse transcriptase as described herein has increased activity as compared to the activity exhibited by the same enzyme under non-activating conditions.

The term “reverse transcriptase activity” or “activity” generally refers to any activity used by the reverse transcriptase to make DNA (e.g., a cDNA) from an RNA template, otherwise known as RNA-dependent DNA polymerase activity.

The activity of an reverse transcriptase is “substantially reduced” if a modified form of the enzyme has an activity which is reduced by at least 50%, 60%, 70%, 80%, 90% or more (and percentages in between) as compared to the unmodified enzyme.

The term “no substantial increase” in reverse transcriptase activity refers generally to no more than a 0%, 5%, 10%, 20%, 30%, 40%, or 50% (and percentages in between) increase in reverse transcriptase activity upon incubation for a particular amount of time under non-activating conditions. In exemplary embodiments, “no substantial increase” in activity refers to undetectable activity upon incubation for a particular amount of time under non-activating conditions.

The term “essentially complete inactivation” of enzyme activity refers generally to a level of activity of a modified enzyme which is at least 20% or less of the unmodified enzyme under non-activating conditions. In exemplary embodiments, “essentially complete inactivation” refers to undetectable activity under non-activating conditions.

The term “hot start” (when used to describe an enzyme) refers generally to a modified enzyme whereby enzyme activity is inhibited under non-activating conditions, but becomes active upon exposure to activating conditions. Modification of the enzyme can be a chemical modification, but is not limited to this type of modification. Inactivation under non-activating conditions prevents the formation of non-specific products during the reaction set up process resulting in improved specificity.

II. Introduction

Enzymes, such as reverse transcriptases typically have suboptimal activity at low temperatures. However, this minor activity can still result in nonspecific priming and self-priming when reactions are set up on ice, at room temperature, and the few seconds for the reaction to heat up to the appropriate incubation temperature. As such, reverse transcription reactions are often performed by assembling reaction mixtures in the absence of enzyme and pre-equilibrating the mix to an elevated temperature before the addition of enzyme (i.e., manual hot start). To circumvent this problem and the complex reaction set-up procedures, the current invention provides thermostable and/or thermoreactive reverse transcriptase enzyme comprising a chemical modification, wherein the modified reverse transcriptase is thermostable and/or thermoreactive at a temperature exceeding about 42° C., and wherein the chemical modification results in at least a 50% decrease in reverse transcriptase activity as compared to a corresponding unmodified reverse transcriptase when activity is measured under non-activating conditions. In particular, thermostable and/or thermoreactive reverse transcriptase enzymes can be chemically modified to inactivate their activity at room temperature, which is about 25° C. For example, at a basic pH (pH 8.0-9.0), 2,3-dimethylmaleic anhydride (2,3-DMMA) participates in an acylation reaction with the a-amino group of lysines within a protein sequence and can be used to inactivate a thermostable/thermoreactive reverse transcriptase as disclosed herein.

In some embodiments, the reversibly inactivated reverse transcriptase enzymes of the present invention are produced by a reaction between the enzyme and a modifier reagent, which results in a reversible modification of the enzyme, which leads to a substantial reduction or non-detectable reverse transcriptase activity under non-activating conditions. In some embodiments, the modification consists of a chemical covalent attachment of a modifier group to the enzyme protein. The modifier compound can be chosen such that the modification is reversible by incubation at an elevated temperature and/or by lowering the pH of the reverse transcription reaction buffer. The modifier is also chosen for compatibility with the integrity of RNA. Suitable enzymes and modifier groups are described herein.

In some embodiments, the modified enzymes according to the present invention can exhibit thermostability and/or thermoreactivity at temperatures above 37° C. (e.g., 42° C., 45° C., 50° C., 53° C., 57° C., 60° C., 65° C., 70° C., etc.). Thus, the presently disclosed reversibly inactivated enzymes show advantages over other reverse transcriptase enzymes, e.g., reverse transcriptase enzymes that do not exhibit thermostability at elevated temperatures above 42° C. This is at least because non-thermostable/thermoreactive enzymes typically are not able to withstand the increased temperature incubation which reactivates the enzyme, which helps to insure reaction specificity, thereby preventing their effective use in hot start reactions.

III. Reverse Transcriptase Enzymes

Reverse transcriptases for use in the compositions, methods and kits of the invention include any thermostable and/or thermoreactive reverse transcriptase or polypeptide having reverse transcriptase activity that has been reversibly inactivated using the reactions and methods disclosed herein.

Reverse transcriptase enzymes suitable for modification and subsequent use according to the present invention are well known in the art and can be derived from a number of sources. Three prototypical forms of retroviral reverse transcriptase have been studied thoroughly, and are discussed below for exemplary purposes.

Moloney Murine Leukemia Virus (M-MLV) reverse transcriptase contains a single subunit of 78 kDa with RNA-dependent DNA polymerase and RNase H activity. This enzyme has been cloned and expressed in a fully active form in E. coli (reviewed in Prasad, V. R., Reverse Transcriptase, Cold Spring Harbor, N.Y.: Cold Spring Harbor Laboratory Press, p. 135 (1993)).

Human Immunodeficiency Virus (HIV) reverse transcriptase is a heterodimer of p66 and p51 subunits in which the smaller subunit is derived from the larger subunit by proteolytic cleavage. The p66 subunit has both a RNA-dependent DNA polymerase and an RNase H domain, while the p51 subunit has only a DNA polymerase domain. Active HIV p66/p51 reverse transcriptase has also been cloned and expressed successfully in a number of expression hosts, including E. coli (reviewed in Le Grice, S. F. J., Reverse Transcriptase, Cold Spring Harbor, N.Y.: Cold Spring Harbor Laboratory press, p. 163 (1993)). Within the HIV p66/p51 heterodimer, the 51-kD subunit is catalytically inactive, and the 66-kD subunit has both DNA polymerase and RNase H activity (Le Grice, S. F. J., el al., EMBO Journal 10:3905 (1991); Hostomsky, Z., et al., J. Virol. 66:3179 (1992)).

Members of the Avian Sarcoma-Leukosis Virus (ASLV) reverse transcriptase family are also a heterodimers of two subunits, alpha (approximately 62 kDa) and beta (approximately 94 kDa), in which the alpha subunit is derived from the beta subunit by proteolytic cleavage (reviewed in Prasad, V. R., Reverse Transcriptase, Cold Spring Harbor, N.Y.: Cold Spring Harbor Laboratory Press (1993), p. 135). Members of this family include, but are not limited to, Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase, among others.

ASLV reverse transcriptase can exist in two additional catalytically active structural forms, αβ and α (Hizi, A. and Joklik, W. K., J. Biol. Chem. 252: 2281 (1977)).

Sedimentation analysis suggests the presence of alpha/beta and beta/beta are dimers and that the a form exists in an equilibrium between monomeric and dimeric forms (Grandgenett, D. P., et al., Proc. Nat. Acad. Sci. USA 70:230 (1973); Hizi, A. and Joklik, W. K., J. Biol. Chem. 252:2281 (1977); and Soltis, D. A. and Skalka, A. M., Proc. Nat. Acad. Sci. USA 85:3372 (1988)). The ASLV alpha/beta and beta/beta reverse transcriptases are the only known examples of retroviral reverse transcriptase that include three different activities in the same protein complex: DNA polymerase, RNase H, and DNA endonuclease (integrase) activities (reviewed in Skalka, A. M., Reverse Transcriptase, Cold Spring Harbor, N.Y.: Cold Spring Harbor Laboratory Press (1993), p. 193). The a form lacks the integrase domain and activity.

Various forms of the individual subunits of ASLV reverse transcriptase have been cloned and expressed. These include a 98-kDa precursor polypeptide that is normally processed proteolytically to beta and a 4 kDa polypeptide removed from the beta carboxy end (Alexander, F., et al., J. Virol. 61:534 (1987) and Anderson, D. et al., Focus 17:53 (1995)), and the mature beta subunit (Weis, J. H. and Salstrom, J. S., U.S. Pat. No. 4,663,290 (1987); and Soltis, D. A. and Skalka, A. M., Proc. Nat. Acad. Sci. USA 85:3372 (1988)). (See also Werner S. and Wohrl B. M., Eur. J. Biochem. 267:4740-4744 (2000); Werner S. and Wohrl B. M., J. Virol. 74:3245-3252 (2000); Werner S. and Wohrl B. M., J. Biol. Chem. 274:26329-26336 (1999).) Heterodimeric RSV alpha/beta reverse transcriptase has also been purified from E. coli cells expressing a cloned RSV beta gene (Chernov, A. P., et al., Biomed. Sci. 2:49 (1991)).

Although retroviral reverse transcriptase enzymes may be isolated from retroviral sources such as those describe above, it is appreciated that reverse transcriptase enzymes may also be isolated from a large number of mobile genetic elements which are not of retroviral origin. Such mobile genetic elements are resident in the genomes of higher order species and play a functional role in life cycle of these mobile genetic elements. Mobile genetic elements are known to encode genes for reverse transcriptase enzymes (reviewed in Howard M Temin, Reverse Transcription in the Eukaryotic Genome: Retroviruses. Pararetroviruses, Retrotransposons, and Retrotranscripts, Mol. Biol. Evol. 2(6):455-468). These elements include, but are not limited, to retrotransposons. Retrotransposons include the non-LTR and LTR mobile genetic elements LINES (such as L1) and SINES (such as SVA elements), and Au elements, among others. (Reviewed by Cordaux and Batzer, Nature Reviews, October 2009, volume 10, pp 691-703.)

Certain DNA polymerase enzymes possess the ability to use RNA as a template, and as such, have substantial reverse transcriptase activity. Therefore, thermostable DNA polymerase enzymes with substantial reverse transcriptase activity may be used in the practice of the present invention. Examples of thermostable DNA polymerase enzymes that possess substantial reverse transcriptase activity include thermostable DNA polymerases isolated from thermophilic eubacteria or archaebacteria comprising species of the genera: Thermus, Thermotoga, Thermococcus, Pyrodictium, Pyrococcus, and Thermosipho, among others. Representative species from which thermostable DNA polymerases possessing substantial reverse transcriptase activity have been derived include: Thermus aquaticus, Thermus thermophilus, Thermotoga maritima, Pyrodictium occultum, Pyrodictium abyssi, and Thermosipho africanus, among others.

In some embodiments, the thermostable and/or thermoreactive reverse transcriptases are mutant or derivative reverse transcriptases that exhibit increased thermostability and/or thermoreactivity compared to their wild type counterparts. In some embodiments, the mutant reverse transcriptases are thermostable at temperatures between 50° C. to 65° C. (e.g. 50° C., 52° C., 55° C., 58° C., 60° C., and 62° C.). For example, in some exemplary embodiments, reverse transcriptases of the present invention are mutant M-MLV reverse transcriptases that exhibit increased reverse transcriptases activity at a reaction temperature of at least 50° C. (e.g., 50° C., 55° C., 60° C., 65° C., 70° C., and 75° C.) when compared to wild type M-MLV. In some embodiments, they are thermostable for at least 1 minute (e.g., 1 minute, 5 minutes, 15 minutes, 60 minutes, 120 minutes, etc.) at a temperature between 50° C. to 65° C. (e.g., 55° C., 60° C., etc.). In some embodiments, the mutant reverse transcriptases are thermoreactive at temperatures between 50° C. to 65° C. (e.g. 50° C., 52° C., 55° C., 58° C., 60° C., and 62° C.). In some embodiments, the mutant reverse transcriptase are thermoreactive for at least 1 minute (e.g., 1 minute, 5 minutes, 15 minutes, 60 minutes, 120 minutes, etc.) at temperatures between 50° C. to 65° C. (e.g., 55° C., 60° C., etc.). In some embodiments, the mutant reverse transcriptases retain at least 10% (e.g., 10%, 20%, 30%, 40%, 50%, 60%, 70%, 80%, 90%, and 100%) reverse transcriptase activity after heating to at least 50° C. (e.g., 50° C., 55° C., 60° C., 62° C., 65° C., etc.) for at least 1 minute (e.g., 1 minute, 2 minutes, 5 minutes, 10 minutes, 15 minutes, etc.). In some embodiments, the reverse transcriptases retain at least 10% (e.g., 10%, 20%, 30%, 40%, 50%, 60%, 70%, 80%, 90%, and 100%) reverse transcriptase activity after heating to at least 60° C. (e.g., 60° C., 62° C., 65° C., etc.) for at least 1 minute (e.g., 1 minute, 2 minutes, 5 minutes, 10 minutes, 15 minutes, etc.). In some embodiments, the reverse transcriptases retain at least 50% (e.g., 50%, 60%, 70%, 80%, 90%, and 100%) reverse transcriptase activity after heating to at least 50° C. (e.g., 50° C., 55° C., 60° C., 62° C., 65° C., etc.) for at least 1 minute (e.g., 1 minute, 2 minutes, 5 minutes, 10 minutes, 15 minutes, etc.). In some embodiments, the reverse transcriptases retain at least 10% (e.g., 10%, 20%, 30%, 40%, 50%, 60%, 70%, 80%, 90%, and 100%) reverse transcriptase activity after heating to at least 50° C. (e.g., 50° C., 55° C., 60° C., 62° C., 65° C.,) for at least 5 minutes (e.g., 5 minutes, 10 minutes, 15 minutes, 30 minutes, etc.).

The methods of the present invention are not limited to the use of the enzymes exemplified above. Any enzyme described in the literature with reverse transcription activity can be potentially modified as described herein to produce a reversibly inactivated enzyme suitable for use in the present methods. In general, any reverse transcriptase enzyme which can withstand reactivation incubation temperatures without becoming irreversibly inactivated is a candidate for use in the compositions and methods, as described herein, to produce a reverse transcription product. One skilled in the art would be able to optimize the modification reaction and reverse transcription reaction conditions for any given enzyme based on the guidance provided herein.

IV. Modifier Reagents

In exemplary embodiments of the invention, reversible inactivation of a reverse transcriptase enzyme comprises reversible blocking of lysine residues by chemical modification of the ε-amino group of lysine residues. Modification of the lysines in the active region of the protein results in inactivation of the protein. Additionally, modification of lysines outside the active region may contribute to the inactivation of the protein through steric interaction or conformational changes. A number of compounds have been described in the literature which react with amino groups in a reversible manner. For example, amino groups have been reversibly modified by trifluoracetylation (see Goldberger and Anfinsen, 1962, Biochemistry 1:410), amidination (see Hunter and Ludwig, 1962, J. Amer. Chem. Soc. 84:3491), maleylation (see Butler et al., 1967, Biochem. J. 103:78), acetoacetylation (see Marzotto et al., 1967, Biochem. Biophys. Res. Commun. 26:517; and Marzotto et al., 1968, Biochim. Biophys. Acta 154:450), tetrafluorosuccinylation (see Braunitzer et al., 1968, Hoppe-Seyler's Z. Physiol. Chem. 349:265), and citraconylation (see Dixon and Perham, 1968, Biochem. J. 109:312-314; and Habeeb and Atassi, 1970, Biochemistry 9(25):4939-4944).

Exemplary reagents for the chemical modification of the ε-amino group of lysine residues are dicarboxylic acid anhydrides, of the general formula:

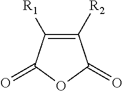

where R1 and R2 are hydrogen or organic radicals, which may be linked, or of the general formula:

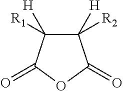

where R1 and R2 are organic radicals, which may be linked, and the hydrogens are cis. The organic radical may be directly attached to the ring by a carbon-carbon bond or through a carbon-heteroatom bond, such as a carbon-oxygen, carbon-nitrogen, or carbon-sulphur bond.

The organic radicals may also be linked to each other to form a ring structure as in, for example, 3,4,5,6-tetrahydrophthalic anhydride. Dicarboxylic acid anhydrides react with the amino groups of proteins to give the corresponding acylated products.

The reversibility of modifications by the above dicarboxylic acid anhydrides is believed to be enhanced by the presence of either the cis-carbon-carbon double bond or the cis hydrogens, which maintains the terminal carboxyl group of the acylated residues in a spatial orientation suitable for interaction with the amide group, and subsequent deacylation. (See Palacian et al., 1990, Mol. Cell. Biochem. 97:101-111 for descriptions of plausible mechanisms for both the acylation and deacylation reactions.)

Other substituents may similarly limit rotation about the 2,3 bond of the acyl moiety in the acylated product, and such compounds are expected to function in the methods of the present invention. Examples of the exemplary reagents include maleic anhydride; substituted maleic anhydrides such as citraconic anhydride, cis-aconitic anhydride, and 2,3-dimethylmaleic anhydride; exo-cis-3,6-endoxo-Δ⁴-tetrahydropthalic anhydride; and 3,4,5,6-tetrahydrophthalic anhydride. These reagents are commercially available from, for example, Aldrich Chemical Co. (Milwaukee, Wis.), Sigma Chemical Co. (St. Louis, Mo.), or Spectrum Chemical Mfg. Corp. (Gardena, Calif.). Modifications of reverse transcriptase enzymes using the substituted maleic anhydride reagent 2,3-dimethylmaleic anhydride (DMAA) are described herein.

The relative stabilities of the amino groups acylated using the above reagents decreases in the following order: maleic anhydride; exo-cis-3,6-endoxo-Δ⁴-tetrahydropthalic anhydride; citraconic anhydride; 3,4,5,6-tetrahydrophthalic anhydride; cis-aconitic anhydride; and 2,3-dimethylmaleic anhydride (see Palacian et al., supra).

In yet other embodiments, the modifier reagent is an imide of the general formula:

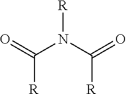

where any of the R groups can be hydrogen atoms, alkyl groups, aryl groups, or any combination thereof. In a more preferred embodiment, the imide is a cyclic imide of dicarboxylic acids, saturated or unsaturated, cyclic or acyclic.

Such imide modifier reagents can include, but are not limited to 2,3-dimethylmaleimide, N-ethoxycarbonyl-2,3-dimethylmaleimide, N-iso-butoxycarbonyl-2,3-dimethlmaleimide, N-ethoxycarbonyl-3,4,5,6-tetrahydrophtalimide, N-ethoxycarbonyl-1,2,3,6-tetrahydrophtalimide, N-ethoxycarbonyl-maleimide and N-ethoxycarbonyl-succinimide. See, for example, the imides as disclosed in U.S. application Ser. No. 11/649,819, the disclosure of which is incorporated herein by reference in its entirety.

Optimal activation incubation conditions for reverse transcriptase enzymes modified with a particular reagent can be determined empirically as described in the Examples. The methods of the present invention are not limited to the exemplified modifier compounds or to the modification of the protein by chemical modification of lysine residues. Any of the compounds described in the literature that react with proteins to cause the reversible loss of all, or nearly all, or a substantial amount of the reverse transcriptase activity, wherein the modification is reversible by incubation at an elevated temperature in the reverse transcription reaction buffer, is suitable for preparation of a reversibly inactivated reverse transcriptase enzyme. For example, those methods and reagents described in U.S. Pat. Nos. 6,183,998; 6,479,264; and 8,618,253 are also considered, the disclosures of which are herein incorporated by reference in their entireties. As new compounds which reversibly modify proteins become available, these too will be suitable for use in the present methods. Thus, compounds for the preparation of the modified reverse transcriptase enzymes of the present invention include compounds which satisfy the following properties: (1) reaction with a reverse transcriptase enzyme which catalyzes primer extension results in a significant inactivation of the enzyme; (2) incubation of the resulting modified enzyme in an aqueous alkaline buffer at a temperature at or below about room temperature (25° C.) results in no significant increase in reverse transcriptase activity in less than about 60 minutes; and (3) incubation of the resulting modified reverse transcriptase enzyme in a reverse transcription reaction buffer, formulated to less than about pH 8.0 and/or at an elevated temperature greater than about 37° C. (e.g., 40° C. to 50° C., 50° C. to 60° C. or 60° C. to 70° C.) results in at least a two-fold increase in reverse transcriptase activity in less than about 60 minutes. The suitability of a particular modifier compound can be empirically determined following the guidance provided herein. Experimental procedures for measuring the above properties, the degree of attenuation of enzyme activity resulting from modification of the protein and the degree of recovery of enzyme activity following incubation at elevated temperatures in a reverse transcription reaction mixture, are further described in the Examples.

The chemical modification of lysine residues in proteins is based on the ability of the ε-amino group of this residue to react as a nucleophile. The unprotonated amino group is the reactive form, which is favored at alkaline pH. In some embodiments, the modification reaction is carried out at pH 8.0 to 9.0 in an aqueous buffer at a temperature at or below room temperature (e.g., 25° C.). In other embodiments, the modification reaction is essentially complete following incubation for 1-2 hours. Suitable reaction conditions for chemical modification are known in the art and are described further in the Examples. Dicarboxylic acid anhydrides react easily with water to give the corresponding acids. Therefore, typically a large fraction of the reagent is hydrolyzed during modification of the protein amino groups. The rate of hydrolysis increases with pH. The increase in hydrolysis which occurs at pH greater than about 9 can result in suboptimal acylation of the protein.

In general, a molar excess of the modifier reagent relative to the enzyme protein is used in the acylation reaction. The optimal molar ratio of modifier reagent to enzyme depends on the reagent used and can be determined empirically as described further in the Examples. A molar ratio of modifier to enzyme in the reaction can be empirically selected that will result in either essentially complete inactivation or significant inactivation of the enzyme by following the guidance provided herein. In some embodiments, suitable molar ratios of modifier to enzyme for this purpose are provided in the Examples.

In some embodiments, the reverse transcriptase is chemically modified with the modifier reagent at a molar ratio of 100× to 5,000× (modifier to enzyme). In some embodiments, the molar ratio of modifier to enzyme is 200×-2000× to 1×. In some preferred embodiments the molar ratio of modifier to enzyme is about 300×-500× to 1× (e.g, 400×:1×) if the modifier is an anhydride. In some embodiments, the molar ratio of modifier to enzyme is about 1500× to 1800× to 1× (e.g., 1600×:1×) if the modifier is an imide.

As an example, in some embodiments, a commercially available thermostable reverse transcriptase, Maxima™ H Minus reverse transcriptase, is essentially completely inactivated (<5% of original activity) by reaction with a 200-fold or greater molar excess of 2,3-dimethylmaleic anhydride. The minimum molar ratio of modifier which results in essentially complete inactivation of the enzyme can be determined by carrying out inactivation reactions with a dilution series of modifier reagent, as described in the Examples. In the methods of the present invention, it is not necessary that the reverse transcriptase enzyme be completely inactivated, only that the reverse transcriptase enzyme be significantly inactivated (e.g., >50% inactive). A reduction in non-specific reverse transcription can be obtained using a significantly inactivated enzyme.

V. Reversibly Inactivated Reverse Transcriptase Enzymes

As used herein, an enzyme is considered to be significantly inactivated if the activity of the enzyme following reaction with the modifier is less than about 50% of the original activity. For example, a reverse transcriptase is considered to be inactivated if it exhibits less than about 50%, 40% 30%, 20%, 10%, 5%, 1%, 0.5%, or 0% of reverse transcriptase activity exhibited prior to modification (i.e., unmodified). A reverse transcriptase can also be considered to be inactivated if it is at least about 50%, 60%, 70%, 80%, 90%, 95%, 99%, 100% “dead” or inactive. In some preferred embodiments, a reverse transcriptase is considered to be essentially completely inactivated if it is more than 90% “dead” or inactive (e.g., >90%, 91%, 92%, 93%, 94%, 95%, 96%, 97%, 98%, 99%, or 100% inactive).

In some embodiments, “non-activating conditions” can comprise incubation at lower temperatures, e.g., ambient or room temperature. In some embodiments, modified enzymes are inactive at temperatures less than about 37° C., e.g., 35° C., 34° C., 32° C., 30° C., 28° C., 25° C., 22° C., 20° C., etc. In some embodiments, non-activating conditions can also comprise an alkaline pH, in particular a pH greater than 8.0 (e.g., 8.3).

In some embodiments, “activating conditions” can comprise incubation at elevated temperatures such as those above ambient or room temperature. In some embodiments, modified enzymes are reactivated at temperatures greater than about 25° C., e.g., 37° C., 42° C., 53° C., 55° C., 57° C., 60° C., 65° C. etc. In some other embodiments, activating conditions can further comprise incubation at pH lower than 8.3. In some embodiments, modified enzymes are reactivated at temperatures greater than about 25° C. and at a pH lower than about 8.3. In some embodiments, modified enzymes are reactivated at a pH less than about 8.3, e.g., 8.2, 8.0, 7.8, 7.5, 7.3, 7.1, 6.9, etc.

Another aspect of the reversibly inactivated reverse transcriptase enzymes of the present invention is their storage stability. In general, the compounds described herein are stable for extended periods of time (e.g., at least 1 week, 4 weeks, 8 weeks, etc.), which eliminates the need for preparation immediately prior to each use. In some embodiments, reverse transcriptase enzymes modified with reagents such as 2,3-dimethylmaleic anhydride are stored refrigerated or frozen. In some embodiments, reverse transcriptase enzymes modified with reagents such as 2,3-dimethylmaleic anhydride are lyophilized.

In some embodiments, the reversibly inactivated reverse transcriptase enzymes of the present invention are dialyzed following reaction with the modifier either before immediate use or before storage. In some embodiments, reverse transcriptase enzymes dialyzed following chemical modification exhibit an increase in reactivation rate. For example, in some embodiments, reverse transcriptase enzymes that are dialyzed following chemical modification are reactivated faster than those reverse transcriptase enzymes that are not dialyzed after preincubation for some period of time at the same temperature. In some embodiments, reverse transcriptase enzymes that are dialyzed following chemical modification are reactivated at least 2× faster (e.g., 2×, 3×, 4×, 5×, 6×, etc.) times faster than those reverse transcriptase enzymes that are not dialyzed after incubation for some period of time at the same temperature.

VI. Compositions and Reaction Mixtures Comprising Reversibly Inactivated Reverse Transcriptase Enzymes

The present teachings provide compositions comprising a variety of components in various combinations. In some embodiments of the present invention, the compositions are formulated by admixing one or more of the reversibly inactivated reverse transcriptases described herein in a in a buffered salt solution. One or more DNA polymerases and/or one or more nucleotides, and/or one or more primers may optionally be added to make the compositions of the invention. These compositions can be used in the present methods to produce, analyze, quantitate and otherwise manipulate nucleic acid molecules (e.g., using reverse transcription or one-step (coupled) RT-PCR procedures).

In addition to the enzyme components, the present compositions can comprise one or more stabilizing components to the compositions and/or synthesis reaction mixtures may also be advantageous, to support enhanced stability of the compositions upon storage and/or reaction mixtures during synthesis. In particular, in some embodiments, the disclosed compositions comprising reversibly inactivated reverse transcriptases involve the use of additives, such as saccharide or polyalcohol compounds, in cDNA synthesis reaction mixtures. As disclosed herein, these compounds, including reactions mixtures comprising such compounds, can be employed to enhance reactivation of thermostable reversibly inactivated enzymes.

In some embodiments, the concentration of the saccharide compound additive is between about 0.05 M to about 1.0 M. For example, the saccharide compound can be used at a concentration of about 0.05. 0.1, 0.2, 0.3, 0.4, 0.5, 0.6, 0.7, 0.8, 0.9 and 1.0 M. In some embodiments, the concentration of the polyalcohol compound additive is a between about 1% to about 40%. For example, the polyalcohol compound can be used at a concentration of about 1%, 5%, 10%, 15%, 18%, 20%, 25%, 30%, 35% and 40%.

Specific examples of saccharide compounds suitable for use herein include, but are not limited to, trehalose, maltose, glucose, sucrose, lactose, xylobiose, agarobiose, cellobiose, levanbiose, quitobiose, 2-3-glucuronosylglucuronic acid, allose, altrose, galactose, gulose, idose, mannose, talose, and levulose. Specific examples of polyalcohol compounds suitable for use herein include, but are not limited to methanol, ethylene glycol, glycerol, sorbitol, erythritol, threitol, arabitol, xylitol and ribitol.

in some embodiments, carbohydrates or sugars for inclusion in the compositions and/or synthesis reaction mixtures of the invention include, but are not limited to, sucrose, trehalose, glycerol, and the like. In some embodiments, trehalose is provided at concentrations ranging from 0.01M to 5M (e.g., 0.01 M, 0.05 M, 0.1 M, 0.5 M, 0.75 M, 1.0 M, 2.0 M, 3.0 M, 4.0 M or 5.0 M). In some embodiments, glycerol is provided at concentrations ranging from 5% to 60%. (e.g., 5%, 10%, 15%, 25%, 30%, 40%, 50%, 60%). Furthermore, such carbohydrates and/or sugars may be added to the storage buffers for the enzymes used in the production of the enzyme compositions and kits of the invention and may be provided in compositions that are either in liquid or dry form (e.g., lyophilized). Such carbohydrates and/or sugars are commercially available from a number of sources, including Sigma (St. Louis, Mo.).

Likewise, addition of one or more surfactants and/or detergents to the compositions and/or synthesis reaction mixtures may also be advantageous, to support enhanced stability of the compositions and/or reaction mixtures upon storage. Preferred such detergents for inclusion in the compositions and/or synthesis reaction mixtures of the invention include, but are not limited to Tween 20, Nonidet P 40 (NP-40), Brij58, CHAPS, Big CHAPS, CHAPS, and the like. Other surfactants or detergents, such as those described in pending U.S. application Ser. Nos. 13/492,575 and 61/895,876 (the disclosures of which are incorporated herein by reference in their entirety) may also be included in the compositions and/or synthesis reaction mixtures of the invention. Furthermore, such detergents may be added to the storage buffers for the enzymes used in the production of the enzyme compositions and kits of the invention. Examples of such detergents are commercially available from a number of sources, including Sigma (St. Louis, Mo.).

In some embodiments, one or more protein additives can also be added to the present compositions to assist in stabilizing the enzyme activity and/or overcoming the inhibition of RT reactions by a variety of compounds often found in samples used for nucleic acid preparation, isolation or purification. The addition of protein additives both individually or in combination, can increase tolerance to RT inhibitor contaminants. Thus, the present compositions can further comprise protein additives that work alone or in combination to increase tolerance to various inhibitors including, for example, ethanol, bile salts, humic acid, hematin, and heparin.

Such inhibitors can include, for example, heparin (blood); hematin (blood); EDTA (blood); citrate (blood); immunoglobin G (blood, serum); humic acid (soil, feces); lactoferrin (milk, saliva, other secretory fluids); urea (urine); plant polysaccharides (plants); melanin (skin, hair); myoglobin (tissue); and indigo dye (textiles). Such protein additives for use in stabilizing and/or overcoming RT inhibition can include proteins such as, but not limited to, albumin (e.g. bovine serum albumin (BSA), recombinant BSA and albumins derived from other species), α-lacalbumin, β-lactoblogulin, casein, apotransferrin, spermine, gelatin (e.g., human recombinant gelatin, fish gelatin and gelatins derived from other species), and DNA-binding proteins (e.g., phage T4 gene 32 (T4gP32)), or peptide or polypeptide variants, fragments or derivatives thereof. Other non-protein based PCR inhibitor blocking agents for use in the present teachings can include, for example, deferoxamine mesylate. Some preferred protein additives for stabilization and for use as PCR inhibitor blocking agents include bovine serum albumin (BSA), apotransferrin, fish gelatin, and T4gP32 proteins.

In some embodiments, protein additives are added to the present compositions to give a final concentration in a working solution of about 1 ng/μL to about 10,000 ng/μL, about 50 ng/μL to about 8000 ng/μL, about 100 ng/μL to about 6000 ng/μL, about 200 ng/μL, to about 5000 ng/μL or preferably about 500 ng/μL to about 3000 ng/μL. Protein additives can also be added as a percentage of the final concentration in a working solution, for example, from about 0.001% to about 15%, about 0.05% to about 10%, about 0.01% to about 5%, or preferably about 0.1% to about 1%.

Compositions of the invention may also contain one or more primers. in some embodiments, compositions of the invention comprise oligo(dT)ₙ primers. These primers are typically ˜20 to 30 bases in length, and anneal to the polyA tails of mRNA. in some embodiments, “n” is a number between 10 to 40, 15 to 35, or 20 to 39 (e.g., 25). By targeting the mRNA fraction, the complexity of the resultant cDNA population is dramatically reduced, since rRNA and tRNA species will not serve as templates in the reaction. The drawback of using oligo(dT) primers is that the resultant cDNA population will have a 3′ bias, thus compromising the effectiveness of PCR primers targeting the 5′ ends of transcipts. In addition, due to the 3′ bias, fragmented samples lacking a polyA tail will not be reverse transcribed.

In other embodiments, compositions of the invention comprise random primers. In some embodiments, the random primers are a random mixture of 4 bases of a specified oligo length. Random hexamer mixes, for example, can be used. Each of the random primers can anneal anywhere the complementary sequence exists within a given RNA molecule (including rRNA, tRNA, mRNA, and any fragments of these species). Reverse transcription using random primers overcomes concerns about RNA secondary structure, and RNA fragments, which are common headaches when using oligo(dT) primers.

In some other embodiments, compositions of the invention comprise locked nucleic acid (LNA) primers. The incorporation of LNA into oligonucleotide primers has been shown to increase template binding strength and specificity for DNA amplification. See, e.g., Ballantyne, K. N., et al., Genomics. March 2008; 91(3):301-5.doi: 10.1016/j.ygeno.2007.10.016. LNA primers bind to polyA sequences with a higher melting temperature (Tm) than those that do not comprise LNA.

In other embodiments, compositions of the invention comprise sequence-specific (or gene-specific) primers. Sequence specific primers typically offer the greatest specificity and have been shown to be the most consistent of the primer options for reverse transcription. However, they do not offer the flexibility of oligo(dT) and random primers, meaning that a new cDNA synthesis reaction must be performed for each gene to be studied. This can sometimes makes sequence-specific primers less than optimal for processing limiting tissue or cell samples. In some embodiments, a mixture of different types of primers (e.g., oligo, random, LNA and/or sequence-specific primers are used.

Reverse transcriptase reactions are typically performed at a pH greater than 8.0. The inventors have surprisingly discovered, however, that in some embodiments, the use of reaction buffers with a pH less than about 8.0 accelerates reactivation of modified reverse transcriptases. In some embodiments, reactivation occurs in 30 minutes, 20 minutes, or 10 minutes or less. Moreover, reactivation of reversibly inactivated non-thermostabile or non-thermoreactive reverse transcriptases, such as SuperScript™ III and wild type M-MLV is able to occur at lower temperatures when the pH of the reaction buffer is less than about 8.3, more preferably less than about 8.0. In some embodiments, reactivation using buffers having a pH less than 8.0 shortens reactivation time and allows these modified reverse transcriptase enzymes (whether they are thermostable and/or thermoreactive or not) to be reactivated at temperatures higher than their recommended reaction temperatures.

In some embodiments, the compositions disclosed herein are formulated to a pH less than about 8.3, more preferably to a pH less than about 8.0 (e.g., pH 8.0, pH 7.9, pH 7.8, pH 7.7, pH 7.6, pH 7.5, pH 7.4, pH 7.3, pH 7.2, pH 7.1, pH 7.0, pH 6.9, pH 6.8, etc.). In some embodiments, the use of reaction buffers having a pH less than 8.0 subsequently lowers the temperature at which reactivation of chemically-modified reverse transcriptases can occur. This can be beneficial in situations where the use of thermostable and/or thermoreactive enzymes is not an option.

The final pH of the compositions and/or reaction mixtures of the invention will generally be dependent on buffering agents present in compositions of the invention. The pH of compositions of the invention, and hence reaction mixtures of the invention, will vary with the particular use and the buffering agent present but are preferably from about pH 5.5 to about pH 8.5 (e.g., about pH 6.0, about pH 6.5, about pH 7.0, about pH 7.1, about pH 7.2, about pH 7.3, about pH 7.4, about pH 7.5, about H 7.6, about pH 7.7, about pH 7.8, about pH 7.9, about pH 8.0, about pH 8.1, about pH 8.2, about pH 8.3, about pH 8.4, about pH 8.5, from about pH 6.0 to about pH 8.5, from about pH 6.5 to about pH 8.5, from about pH 7.0 to about pH 8.5, from about pH 7.5 to about pH 8.5, from about pH 6.0 to about pH 8,0, from about pH 6.0 to about pH 7.7, from about p1-1 6.0 to about pH 7.5, from about pH 6.0 to about pH 7.0, from about pH 7.2 to about pH 7.7, from about pH 7.3 to about pH 7.7, from about pH 7.4 to about pH 7.6, from about pH 7.0 to about pH 7.4, from about pH 7.6 to about pH 8.0, from about pH 7.6 to about pH 8.5, from about pH 7.7 to about pH 8.5, from about pH 7.9 to about pH 8.5, from about pH 8.0 to about pH 8.5, from about pH 8.2 to about pH 8.5, from about pH 8.3 to about pH 8.5, from about pH 8.4 to about pH 8.5, etc.). In the most preferred embodiments, the compositions of the instant invention are at a pH less than about 8.0 e.g., 7.8. 7.6, 7.4, 7.2, 7.0, etc.)

The invention further includes compositions for reverse transcribing nucleic acid molecules, as well as reverse transcription methods employing such compositions and product nucleic acid molecules produced using such methods. In many instances, compositions of the invention may contain one or more of the following components: (1) one or more buffering agent (e.g., sodium phosphate, sodium acetate, 2-(N-morpholino)-ethanesulfonic acid (MES), tris-(hydroxymethyl)aminomethane (Tris), 3-(cyclohexylamino)-2-hydazroxy-1-propanesulfonic acid (CAPS), citrate, N-2-hydroxyethylpiperazine-N′-2-ethanesulfonic acid (HEPES), acetate, 3-(N-morpholino)propanesulfonic acid (MOPS), N-tris(hydroxymethyl)methyl-3-aminopropanesulfonio acid (TAPS), etc.), (2) one or more monovalent cationic salt (e.g., NaCl, KCl, etc.), (3) one or more divalent cationic salt (e.g., MnCl2, MgCl2, MgSO4, CaCl2, etc.), (4) one or more reducing agent (e.g., dithiothreitol, β-mercaptoethanol, etc.), (5) one or more ionic or non-ionic detergent (e.g., TRITON X100™, NONIDET P4™ sodium dodecyl sulphate, etc.), (6) one or more DNA polymerase inhibitor (e.g., Actinomycin D, etc.), (7) deoxynucleotides (e.g., dNTPs, such as dGTP, dATP, dCTP, dTTP, etc.), (8) RNA to be reverse transcribed and/or amplified, (9) one or more RNase inhibitor (e.g., RNASEOUT™, Invitrogen Corporation, Carlsbad, Calif., catalog number 10777-019 etc.), (10) a reverse transcriptase (e.g.reverse transcriptase of the invention, and/or (11) one or more diluent (e.g., water). Other components and/or constituents (e.g., DNA polymerases, RNA template, a label such as a radioactive label conjugated with the nucleotide base or a fluorescent label conjugated to the primer, and/or a terminating agent, such as a chain terminator base comprising a dideoxynucleotide) may also be present in compositions.

In some embodiments, such compositions can be formulated as concentrated stock solutions (e.g., 2×, 3×, 4×, 5×, 6×, 10×, etc.). In some embodiments, having the composition as a concentrated 5×) stock solution allows a greater amount of nucleic acid sample to be added (such as, for example, when the compositions are used for nucleic acid synthesis).

The present invention also provides kits or multi-container units comprising the enzymes disclosed herein, which may be provided with additional components used for practicing the present methods. A useful kit contains a reversibly-inactivated reverse transcriptase enzyme as disclosed herein and one or more reagents for carrying out a reverse transcription and optionally an amplification reaction, such as oligonucleotide primers, substrate nucleoside triphosphates, cofactors, and an appropriate buffer.

VII. Reactivation of Reversibly Inactivated Reverse Transcription Enzymes

The present invention further provides methods to enhance reactivation of reversibly inactivated thermostable enzymes. In certain embodiments of the present invention, methods are provided for reverse transcription of an RNA template using a reversibly inactivated thermostable and/or thermoreactive reverse transcriptase enzyme that can be reactivated at elevated temperatures (e.g., at temperatures above 50° C.). In some embodiments, modified reverse transcriptase enzymes of the present invention are substantially inactive at lower temperature and a pH>8.0 (‘inactivating conditions“) and do not support primer extension or the formation of extension products, non-specific or otherwise, prior to exposure to incubation at an increased temperature and/or decreased pH (“activating conditions”), which reactivates the reverse transcriptase enzyme and allows it to participate in nucleic acid synthesis. In other embodiments, the reverse transcriptase reaction is maintained at elevated temperatures, following an increased temperature preincubation that reactivates the enzyme, which helps to ensure reaction specificity. Under these activating conditions, only the heat-activated enzyme has substantial ability to catalyze the primer extension reaction. According to the methods presented herein, this modification can be reversed by a deacylation reaction that is favored at high temperatures (e.g., greater than 25° C., such as at 30° C., 42° C., 50° C., 55° C., 60° C. and 65° C.) and lower pH (e.g., pH <8.3, such as at pH 8.0, 7.9, 7.3, 7.0, and 6.9). Thus, reverse transcription products are formed only under conditions which enhance primer extension specificity.

In some embodiments, the methods of the present invention involve the use of a reaction mixture containing a reversibly inactivated reverse transcriptase enzyme and subjecting the reaction mixture to an elevated temperature incubation and/or reducing the pH (e.g., to less than 8.0) prior to, or as an integral part of, the reactivation reaction, such as during a reverse transcription reaction. The elevated temperature incubation results in deacylation of modified-amino groups and recovery of enzyme activity. The deacylation of the modified amino groups results from both the increase in temperature and a concomitant decrease in pH. Typically, reverse transcription reactions are carried out in a Tris-HCl buffer. In some embodiments, the buffer used for reverse transcription is formulated to an alkaline pH (e.g., pH 8.3) at room temperature. At room temperature, the reaction buffer conditions favor the acylated form of the amino group. Although the pH of the reaction buffer is adjusted to a pH greater than 8.0 (e.g., 8.3) at room temperature, the pH of a Tris-HCl reaction buffer decreases with increasing temperature. Thus, the pH of the reaction buffer is decreased at the elevated temperatures at which the reverse transcription is carried out and, in particular, at which the activating incubation is carried out. The decrease in pH of the reaction buffer favors deacylation of the amino groups. The change in pH which occurs resulting from the high temperature reaction conditions depends on the buffer used. The temperature dependence of pH for various buffers used in biological reactions is reported, for example, in Good et al., 1966, Biochemistry 5(2):467-477. For Tris buffers, the change in pKa, i.e., the pH at the midpoint of the buffering range, is related to the temperature as follows: ΔpKa/° C.=−0.031. For example, a Tris-HCl buffer assembled at 25° C. undergoes a drop in pKa of 2.17 when raised to 95° C. for the activating incubation. Although reverse transcription reactions are typically carried out in a Tris-HCl buffer, reverse transcription reactions may be carried out in buffers which exhibit a smaller or greater change of pH with temperature. Depending on the buffer used, a more or less stable modified enzyme may be desirable. For example, using a modifying reagent which results in a less stable modified enzyme allows for recovery of sufficient reverse transcriptase activity under smaller changes of buffer pH.

As disclosed herein, an empirical comparison of the relative stabilities of enzymes modified with various reagents can guide selection of a modified enzyme suitable for use in particular buffers. In some embodiments, reverse transcription enzymes may be stored in a buffer separate from other components required in to carry out reverse transcription until subsequent use in a reverse transcription reaction. In other embodiments, the reverse transcriptase enzyme may be stored in a composition comprising additional components required in the reverse transcription reaction (i.e., as part of a “master mix”). Such components can include, but are not limited to, for example, nucleotides (e.g., dCTP, dATP, dGTP, dTTP), cofactors, Mg²⁺, stabilizing additives (e.g., bovine serum albumin and/or glycerol), detergents and or surfactants (e.g., Tween-20 and/or NP-40), exosample nucleotides (e.g., dUTP), one or more polymerase enzymes (e.g., Taq polymerase), dyes, RNase inhibitors, other enzymes (e.g., uracil DNA glycosylase) and a buffer. Possible buffers contemplated for use in the instant invention such as those that decrease pH as temperature is increased can include, for example, carbonate (pK1), PIPES, ACES, MOPSO, Imidazole, BES, MOPS, TES, HEPES, DIPSO, TAPSO, TEA, HEPPSO, POPSO, tricine, glycylglycine, Tris, HEPPS, EPPS, Bicine, AMPD. Buffers available for use in the instant invention are readily apparent to those of skill in the art. See, for example, http://www.applichem.com/en/literature/brochures/biological-buffers.

In general, the length of incubation required to recover reverse transcriptase activity depends on the temperature and pH of the reaction mixture and on the stability of the acylated amino groups of the enzyme, which, in turn, depends on the modifier reagent used in the preparation of the modified enzyme. A wide range of incubation conditions are usable; optimal conditions can be determined empirically for each reaction. In general, incubation is carried out in the reverse transcription reaction buffer at a temperature greater than about 25° C. for between about 1 minute and about 30 minutes. Optimization of incubation conditions for the reactivation of reverse transcriptase enzymes or for reaction mixtures not specified herein can be determined by routine experimentation following the guidance provided herein.

Also disclosed herein are methods for the enhancement of the reactivation of thermostable reversibly inactivated enzymes. In some embodiments, reactivation methods comprise reactivating at least one thermostable reversibly inactivated enzyme in a buffer having a reduced pH. Such conditions can include, for example, buffers formulated to have a pH between 6.8 and 8.3 (e.g., pH 6.9, 7.2, 7.6, or 8.0).

In some other embodiments, methods for the enhancement of the reactivation of thermostable reversibly inactivated enzymes comprise reactivating at least one thermostable reversibly inactivated enzyme in the presence of at least one saccharide compound additive and/or at least one polyalcohol compound such as those described herein.

VIII. Methods for Use of Reversibly Inactivated Reverse Transcription Enzymes

The reverse transcriptase enzymes of the present invention may be used in reverse transcription reactions to produce cDNA molecules. Optionally, the reverse transcription reaction mixture may additionally contain components necessary for polymerase chain reaction (PCR). In some embodiments, the reverse transcription reaction mixture may be used in end point polymerase chain reaction (epPCR), quantitative polymerase chain reaction (qPCR) or real time polymerase chain reaction (rtPCR) methods. In some embodiments, the reverse transcription reaction mixture may be used in one-step reverse transcription polymerase chain reaction (RT-PCR) methods. Alternatively, the reverse transcription reaction mixture may additionally contain components necessary for RNA sequencing (“RNA-seq”). In some embodiments, the reverse transcription reaction mixture may be used in RNA-seq methods useful for transcriptome profiling.

In an exemplary embodiment, a reverse transcription reaction is carried out using a reversibly inactivated thermostable reverse transcriptase enzyme. The annealing temperature used in a reverse transcriptase reaction typically is about 42-50° C., and the reactivation incubation is carried out at a temperature equal to or higher than the annealing temperature. The reverse transcription reaction mixture preferably is incubated at about 37° C. to 65° C., 40° C. to 50° C., 50° C. to 60° C. or 60° C. to 65° C. for up to between 1 minutes and about 30 minutes to reactivate the reverse transcriptase enzyme. Suitable reaction incubation conditions for typical activation of modified reverse transcriptase are described in the Examples.

In an exemplary embodiment of the invention, the modified reverse transcription enzyme and initial activation conditions are chosen such that only a fraction of the recoverable reverse transcriptase activity is recovered during the initial incubation step.

Subsequently increasing the length of the incubation period will increase the recovery of the reverse transcriptase activity. It is known that an excess of reverse transcriptase enzymes contributes to a non-specific reverse transcription reaction. An advantage of the methods of the present invention is that the methods require no manipulation of the reaction mixture following the initial preparation of the reaction mixture. Thus, the methods are ideal for use in automated reverse transcription systems and with in-situ reverse transcription methods, wherein the addition of reagents after the initial denaturation step or the use of wax barriers is inconvenient or impractical.

In another exemplary embodiment, a sample comprising RNA template are contacted with primers (e.g., dTₙ primers, where n indicates the number of T nucleotides and is between 10 to 50 (SEQ ID NO: 2); preferably between 20 and 30) and heated to 65° C. for 5 minutes. In some embodiments, annealed RNA and primers are added to a buffer equilibrated to about pH<8.3 at room temperature and modified reverse transcriptase enzymes added at room temperature. In some embodiments, reverse transcriptase reactions are then incubated at a temperature greater than about 50° C. (e.g., 50° C. to 60° C., such as 50° C., 51° C., 52° C., 53° C., 54° C., 55° C., 56° C., 57° C., 58° C., 59° C., and 60° C.) for some period of time sufficient to allow reverse transcription to occur (e.g., 30 seconds, 1 minute, 5 minutes, 10 minutes, 30 minutes, 45 minutes, 60 minutes, etc.). In some embodiments, reverse transcription reactions are then stopped by heating at 70° C. for some period of time (e.g., 5 minutes, 10 minutes, 15 minutes, 20 minutes, 25 minutes, etc.).

The methods of the present invention are particularly suitable for the reduction of non-specific reverse transcription in a reverse transcription reaction. However, the invention is not restricted to any particular reverse transcription system. The reversibly-inactivated enzymes of the present invention can be used in any primer-based reverse transcription system which uses reverse transcriptase enzymes and relies on reaction temperature to achieve reverse transcription specificity.

The examples of the present invention presented below are provided only for illustrative purposes and not to limit the scope of the invention.

### EXAMPLES

### Example 1

### Modification of a Thermostable Reverse Transcriptase Enzymes with 2,3 Dimethyl Maleic Anhydride

This example describes the modification of a commercially available thermostable reverse transcriptase using 2,3-dimethylmaleic anhydride. This example generally illustrates a method for the modification of a reverse transcriptase enzyme that can be inactivated by manipulating the molar ratio of the modifier reagent to the reverse transcriptase enzyme to be modified.

Measurements were taken of the activity of the Maxima™ H Minus Reverse Transcriptase modified by 2,3-dimethymaleic anhydride (2,3-DMMA) to determine the molar ratio of modifier to enzyme required in the inactivation reaction to obtain complete inactivation of reverse transcriptase activity.

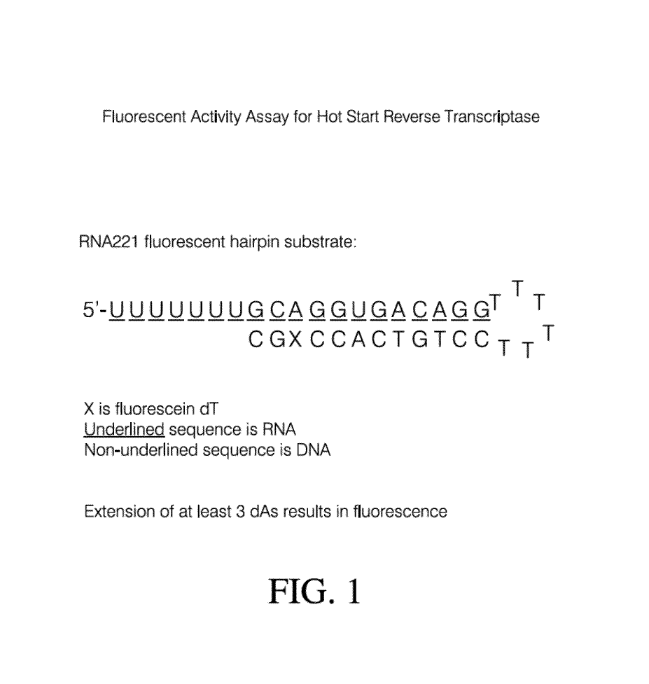

To determine the optimal molar concentration of 2,3-dimethylmaleic anhydride (ACROS Organics, Catalog #AC11639-0050) necessary to reversibly inactivate thermostable reverse transcriptases, Maxima™ H Minus Reverse Transcriptase (Thermo Scientific, Catalog#EP0751) was used as a model enzyme treated with different concentrations of 2,3-DMMA. Maxima™ H Minus was first dialyzed into modification buffer composed of 50 mM Tris-HCl pH 8.5, 30 mM NaCl, 1 mM DTT, and 0.1% Triton-X-1000 (Sigma-Aldrich, X100-100 mL). An initial 50 mg of 2,3-DMMA was dissolved in 1 mL dimethylformamide (DMF) (Fisher Scientific, Catalog #BP1160-500) to make a 50 mg/mL stock solution and serial dilutions corresponding to 50×-800×-fold final molar ratio concentration of 2,3-DMMA to enzyme were prepared in DMF. The various dilutions of 2,-3-DMMA in DMF was then added to Maxima™ H Minus while chilling on ice up to 2.5% of the total volume. The modification reactions were then incubated at 4° C. for one hour with stirring or rotation. At the end of one hour, the modified enzymes were frozen at −20° C. The assay for enzyme thermostability for chemically modified Maxima™ was performed using a fluorescent RNA221 RNA/DNA hairpin substrate (FIG. 1). Extension of 3 dATPs resulted in fluorescent readings.

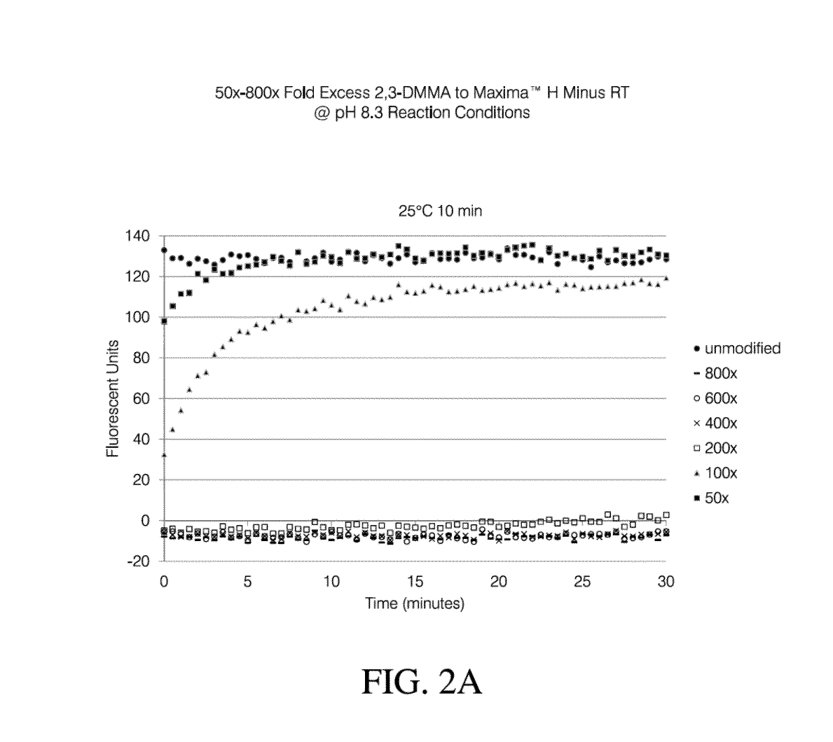

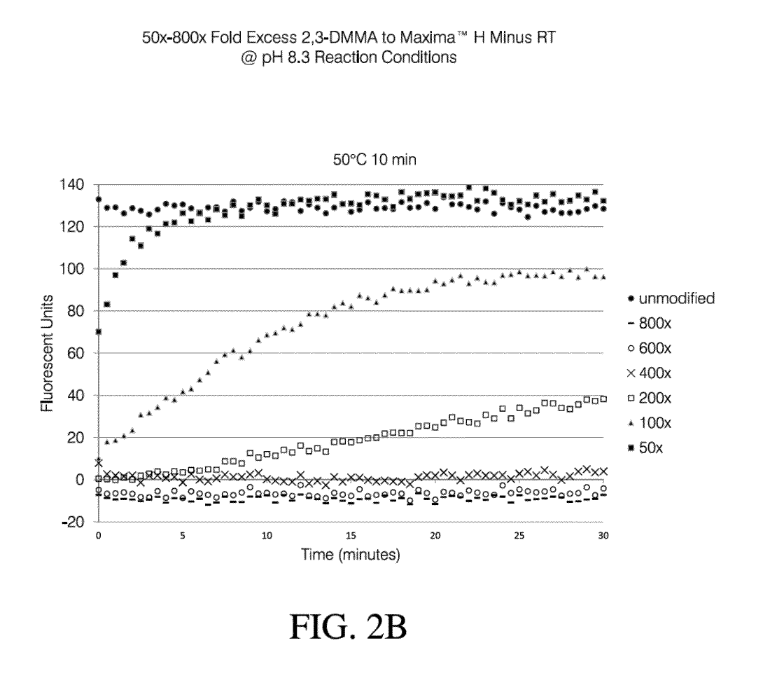

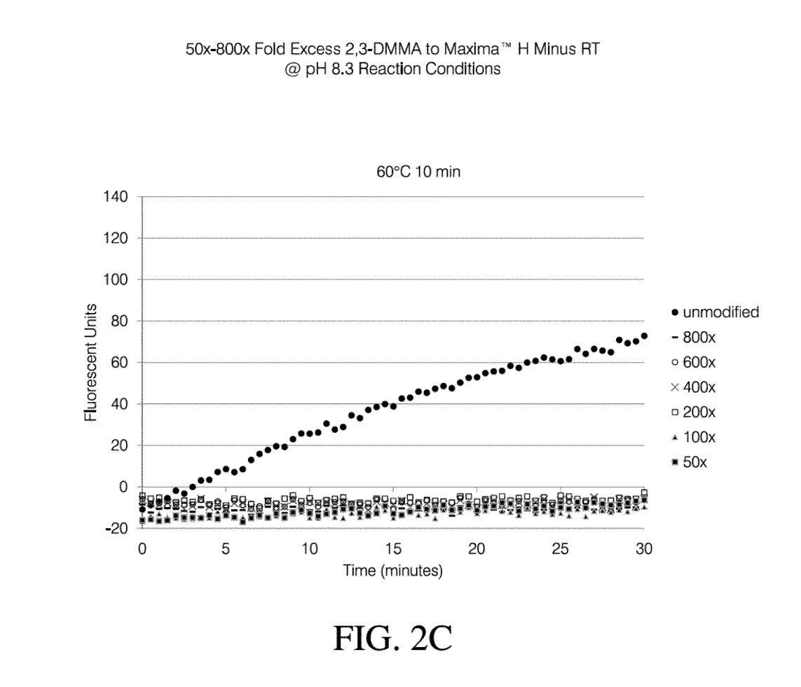

Approximately 30 ng/μL or 15 U/μL of Maxima™ per aliquot was assembled on ice with 50 mM Tris-HCl (pH 8.3 at room temperature), 75 mM KCl, 3 mM MgCl₂, 5 mM DTT, and 100 ng/μL calf thymus DNA (Invitrogen™, Catalog #15633-019). The enzyme mixture was then preincubated at 25° C., 50° C. or 60° C. for 10 minutes as indicated in FIGS. 2A, 2B, and 2C. Preincubation was stopped by incubating the mixtures on ice and then transferring to an assay plate containing 150 nM RNA221 substrate and 0.5 mM dATP final reaction concentrations. The assay plates were read every 30 seconds for 30 minutes using a Molecular Devices SpectraMAX Gemini EM plate reader with an excitation/emission of 490/520 nm. As shown in FIG. 2A, a minimum ratio of 50:1 2,3-DMMA:Enzyme reduced the initial reaction rate slightly by incubation at room temperature and pH 8.3. However at 60° C. for 10 minutes, the enzyme exhibits no residual activity due to apparent lack of thermostability at these conditions as indicated by a ˜50% reduction in the activity of unmodified Maxima™ H Minus (FIG. 2C). A range of 400-800:1 2,3-DMMA:Enzyme completely inactivates enzyme activity at low temperature (25° C.), a desirable characteristic in a hot start reaction. The enzyme was also not reactivated after preincubation at 50° C. for 10 minutes (FIG. 2B). At 60° C. for 10 minutes, the enzyme also shows no activity, presumable due to lack of thermostability as indicated by the drop in fluorescence of unmodified Maxima™ H Minus (FIG. 2C).

### Example 2

### Comparison of Reverse Transcriptases Modified With an Anhydride and an Imide

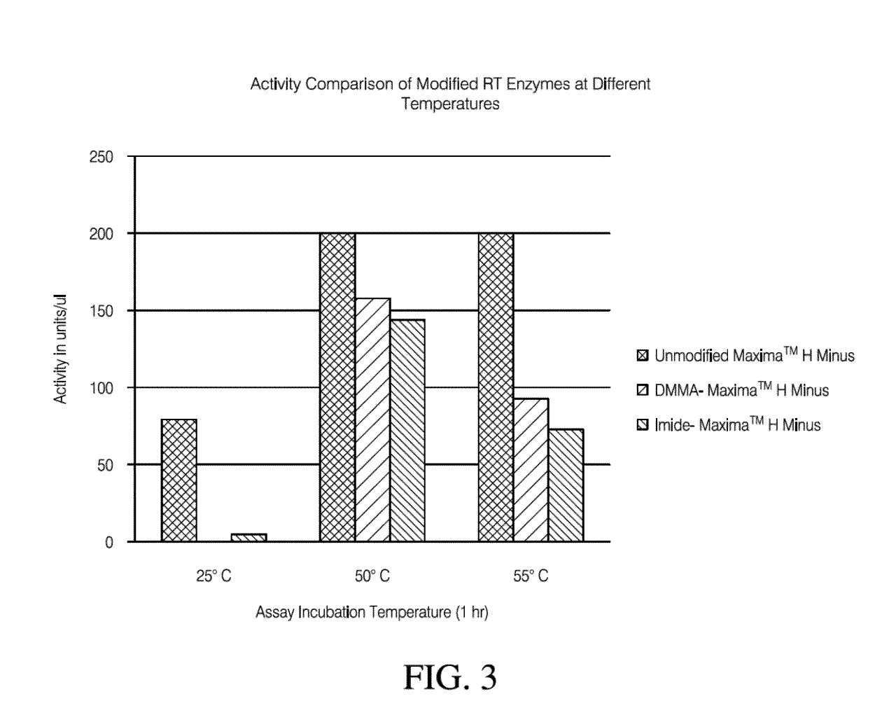

Maxima™ HMinus modified with either 2,3-DMMA (400× modifier: 1× reverse transcriptase in pH 8.5 Tris-based buffer) or N-ethoxycarbonyl-2,3-dimethylmaleimide (1600× modifier: 1× reverse transcriptase in pH 8.5 borate-based buffer) were compared in a radioactive activity assay based on [H3]-dTTP incorporation into poly(A)/oligo(dT) substrate. Unmodified Maxima™ H Minus enzyme at 200 u/μl was used as a calibration standard for activity. Modified enzymes were tested for activity at 25° C., 50° C. and 55° C. Results show that DMMA-modified enzyme exhibited lower unit activity at 25° C. and higher unit activity at 50° C. and 55° C. than imide-modified enzyme. As FIG. 3 illustrates, both modified enzymes were significantly less active at 25° C. than unmodified enzyme, and both modified enzymes could be reactivated at 50° C. and 55° C.

### Example 3

### Comparison of pH 8.3 and 7.3 for Reactivation of Modified Reverse Transcriptase Enzymes or Lower pH Reduced Time/Temp for Reactivation of Modified RTs

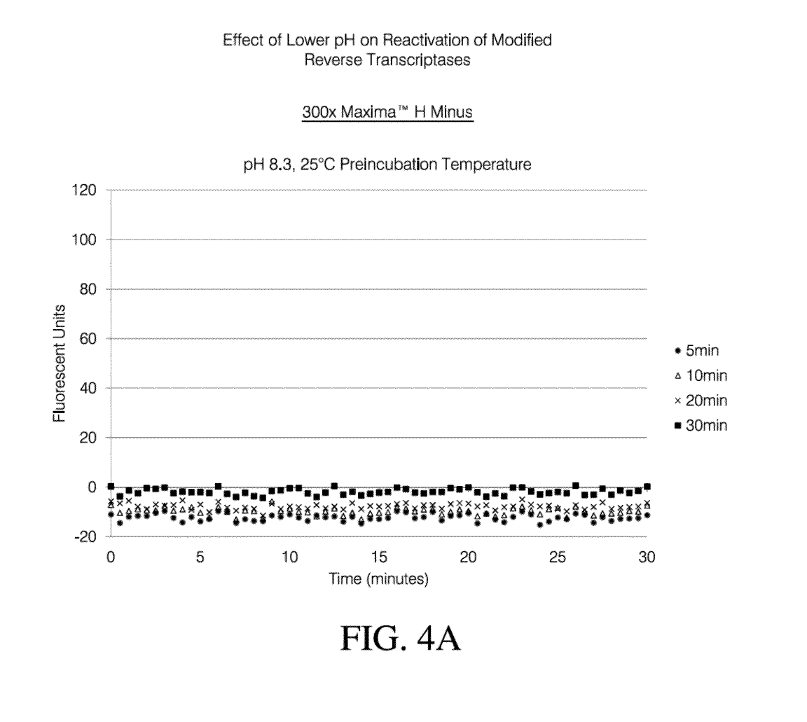

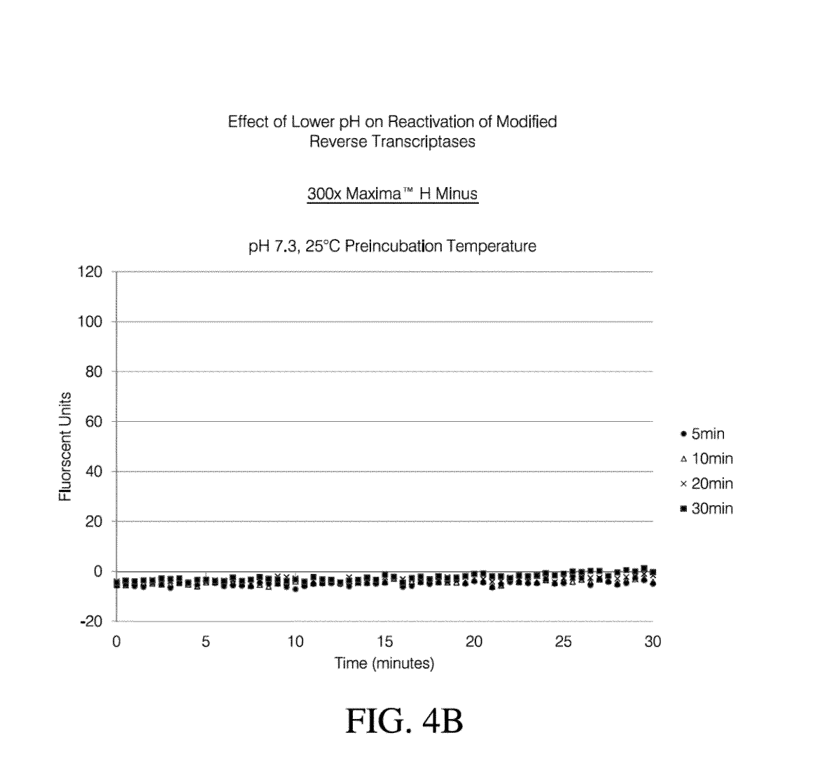

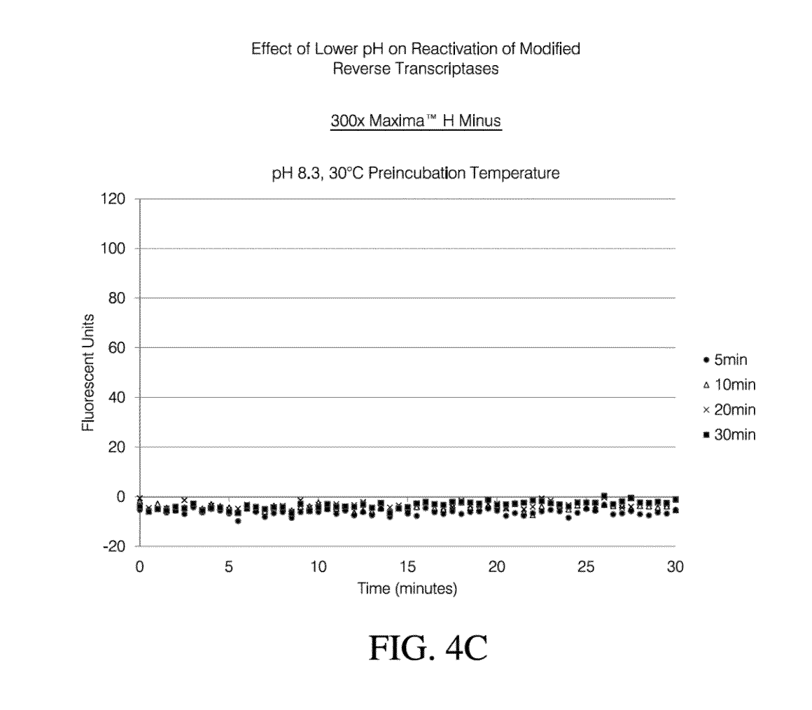

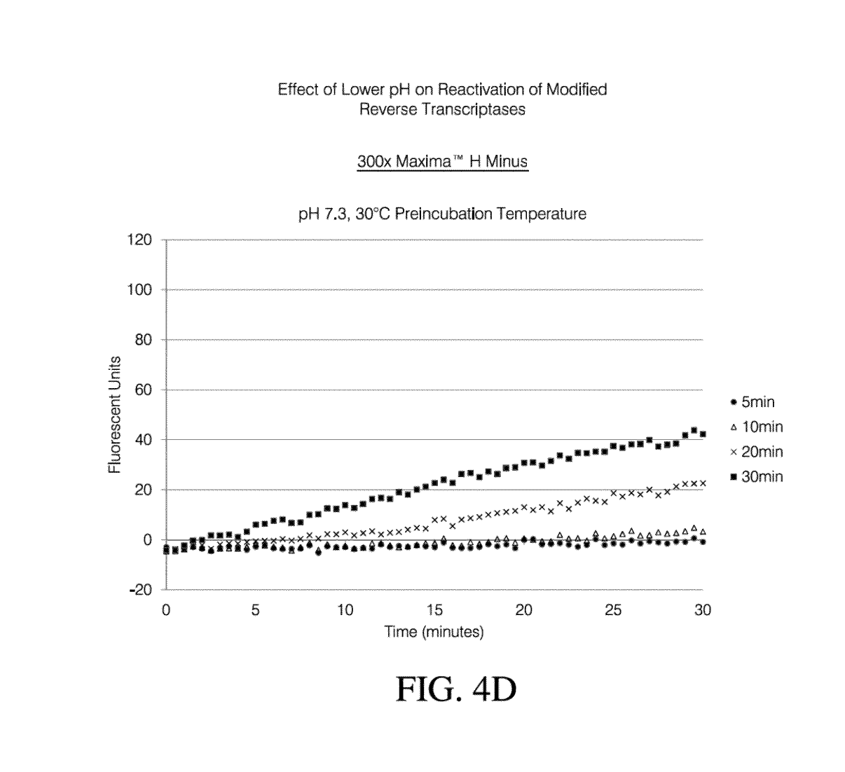

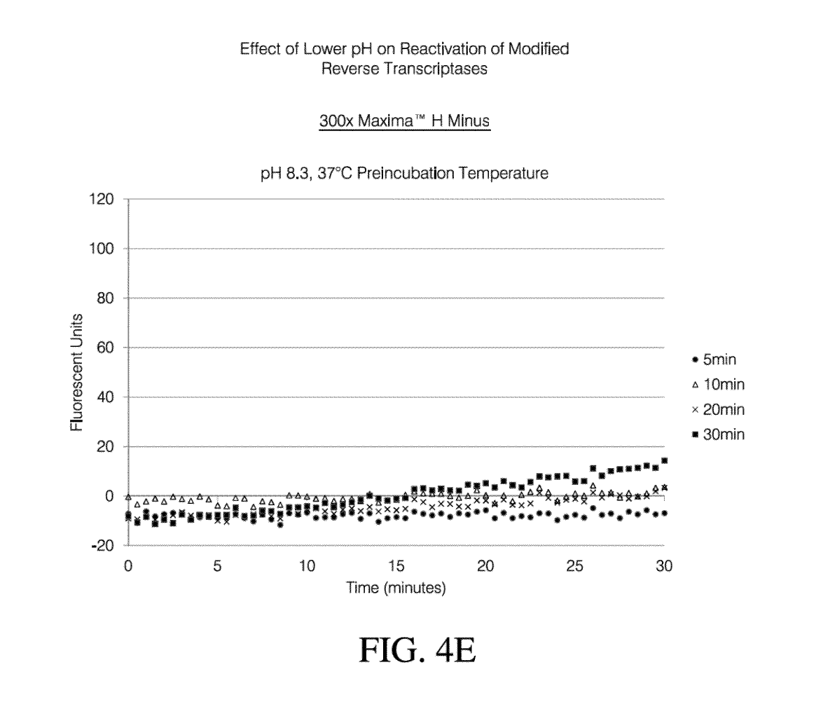

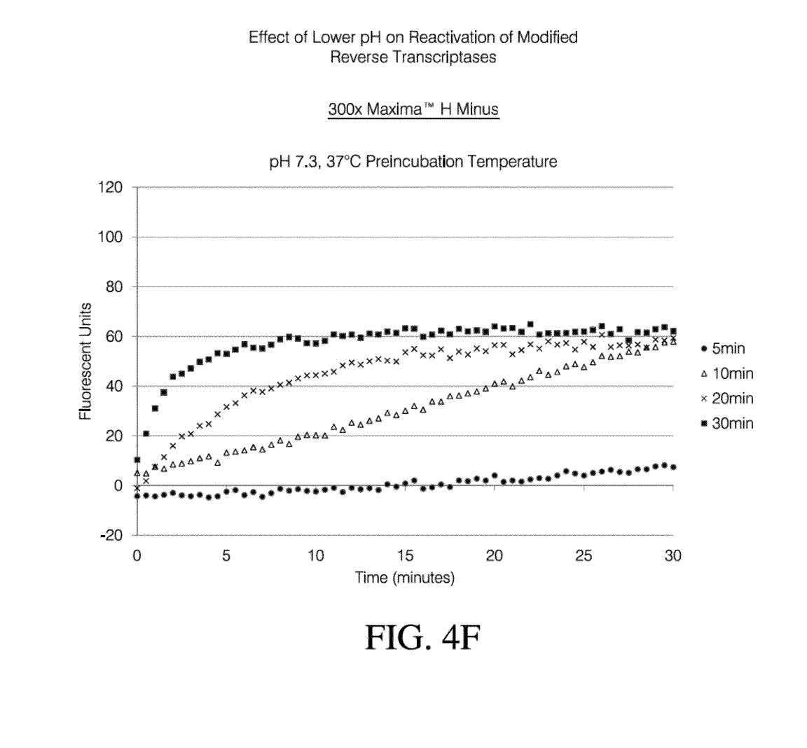

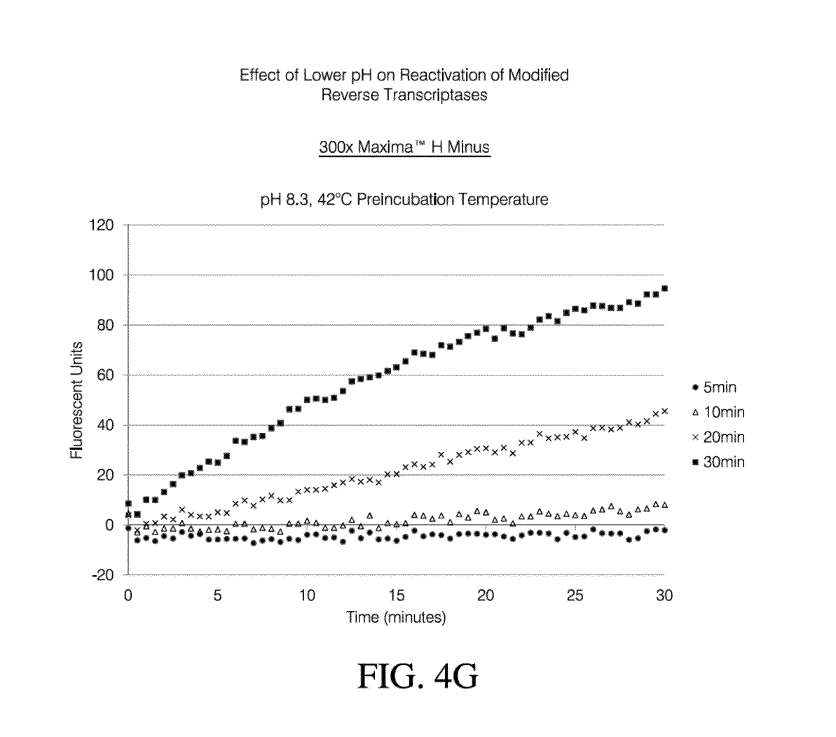

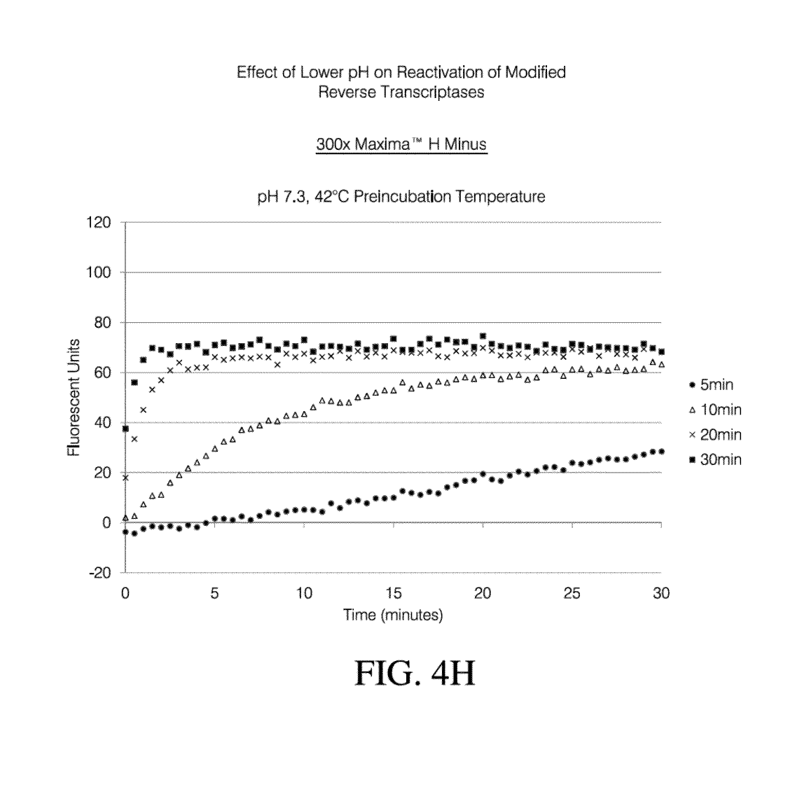

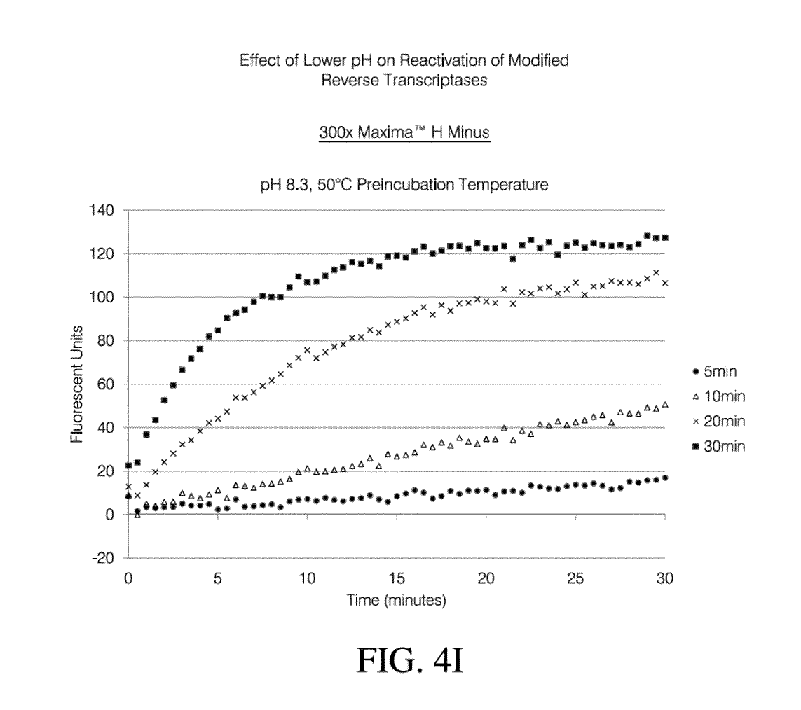

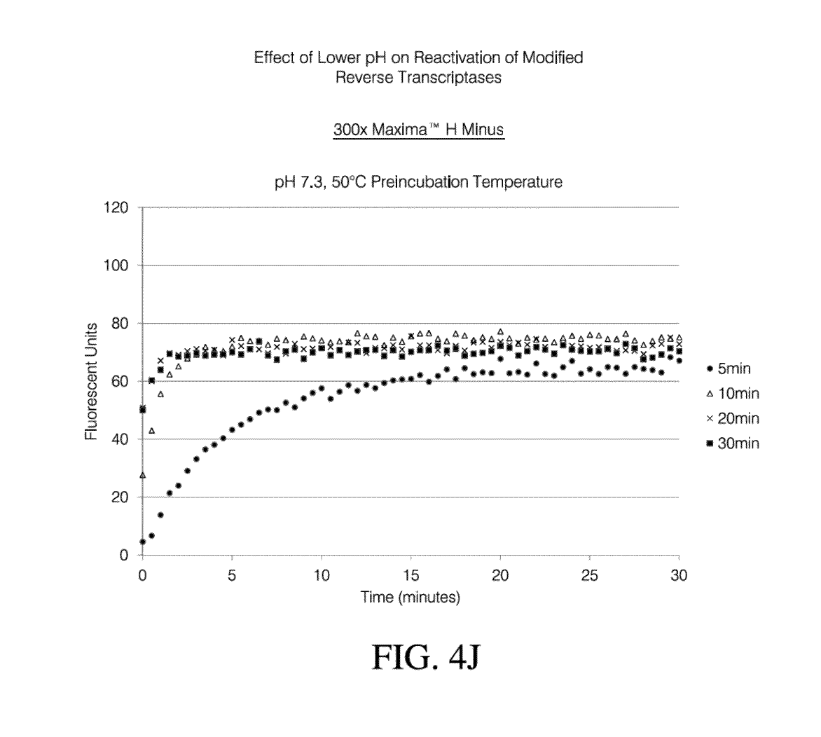

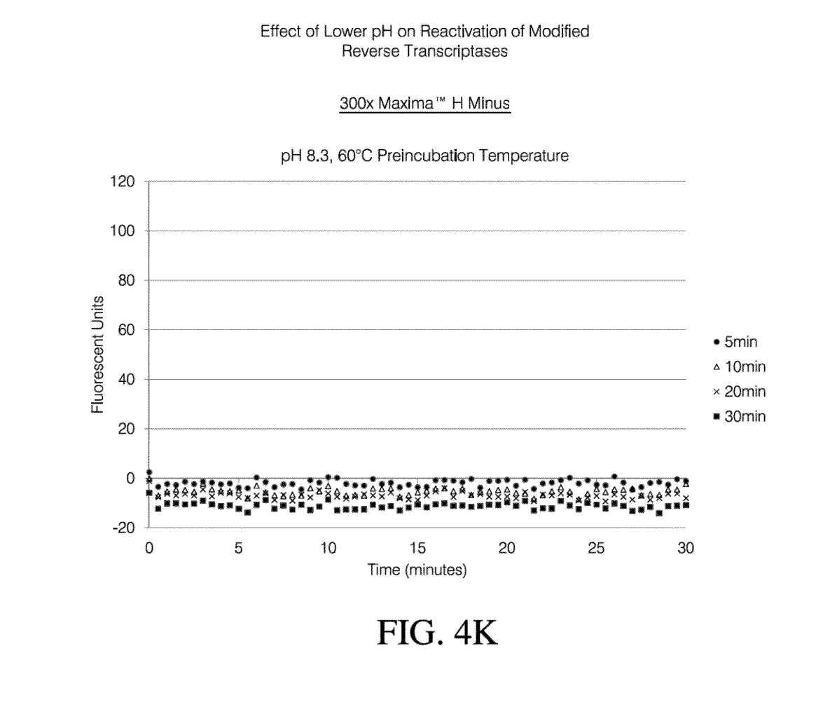

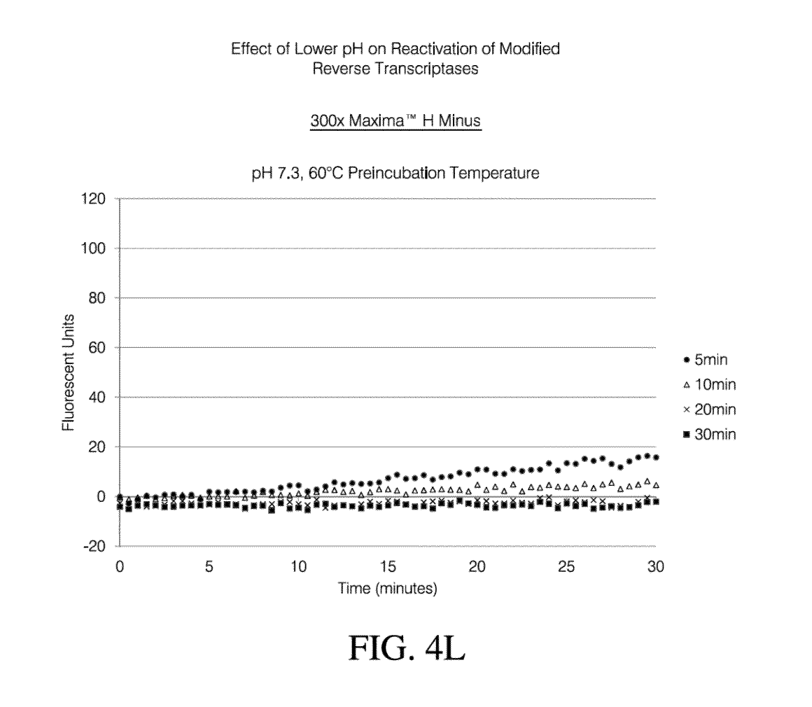

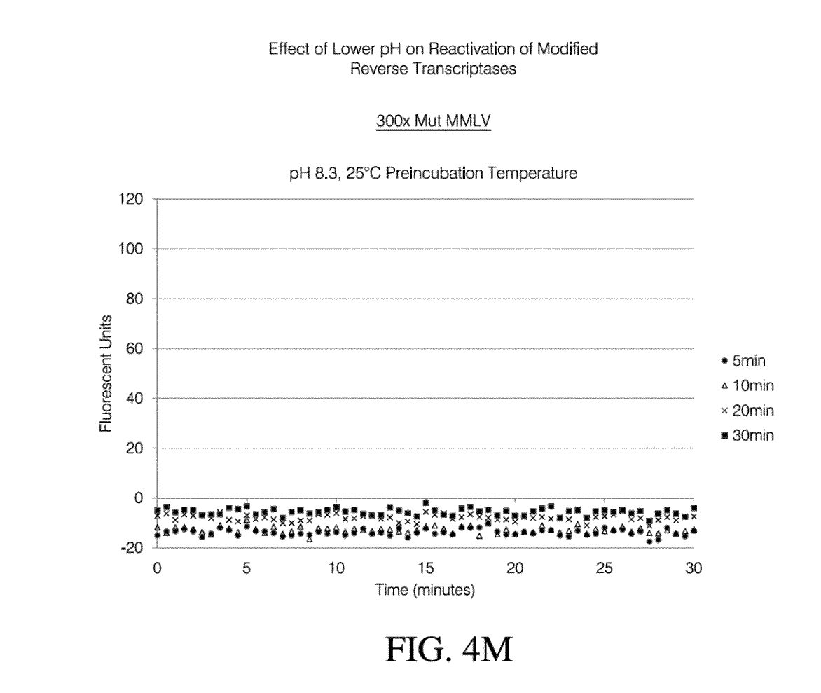

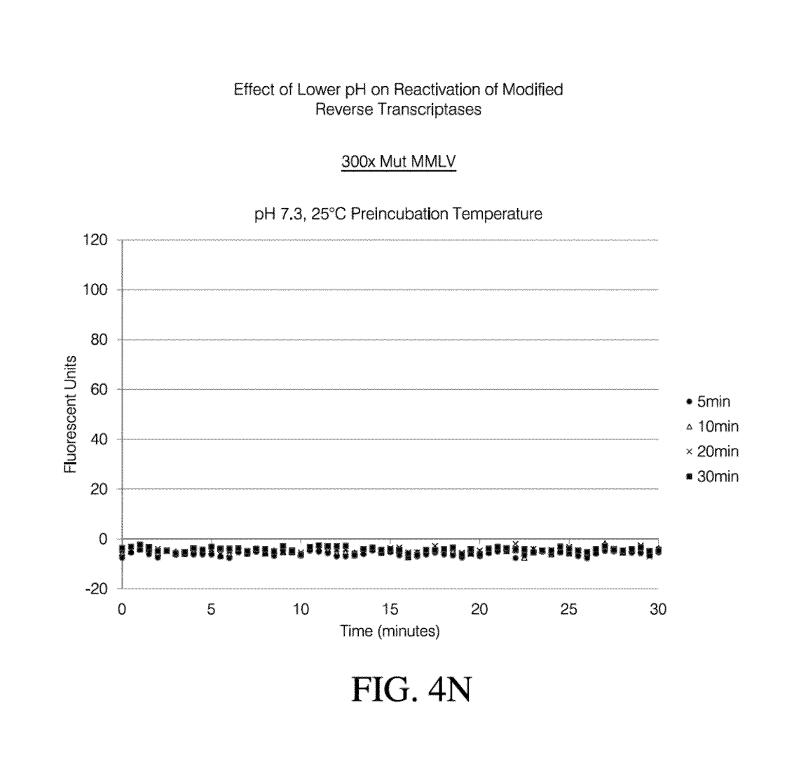

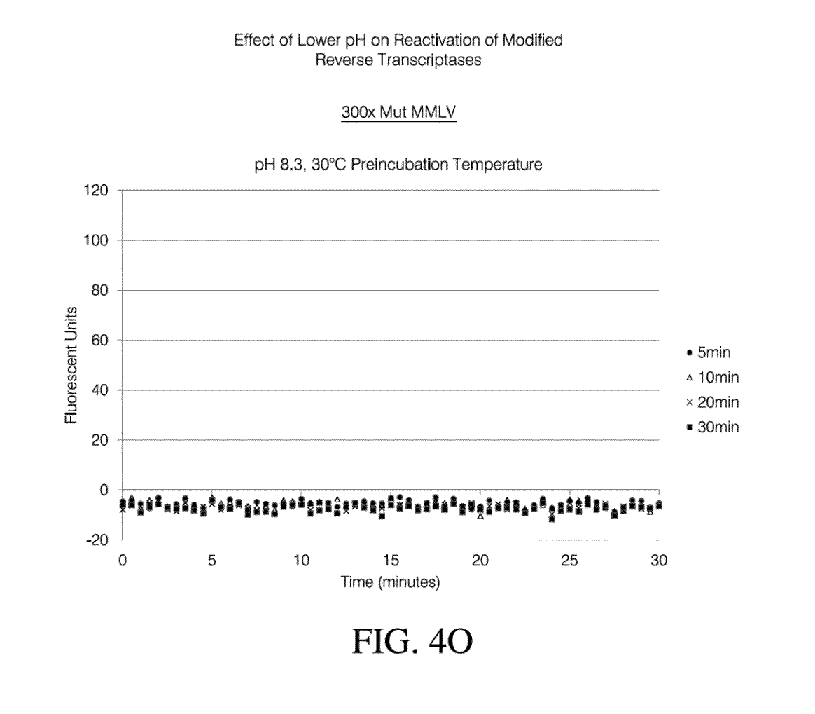

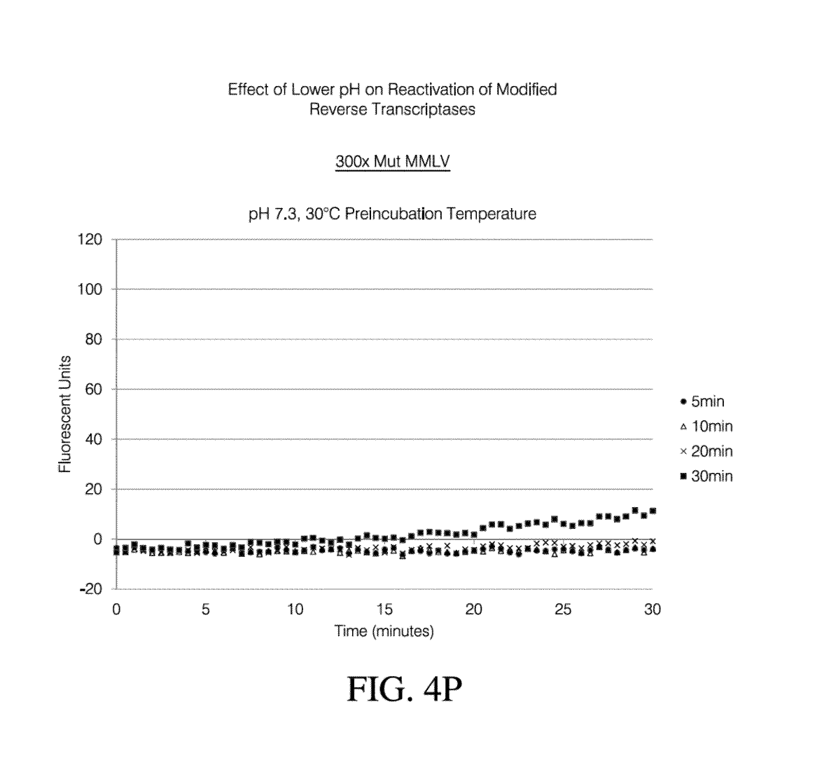

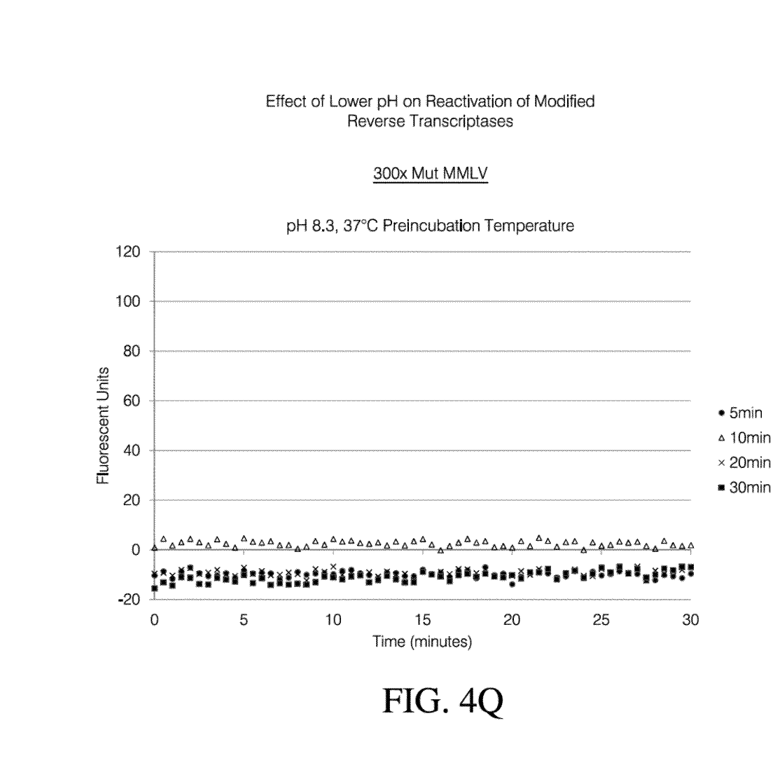

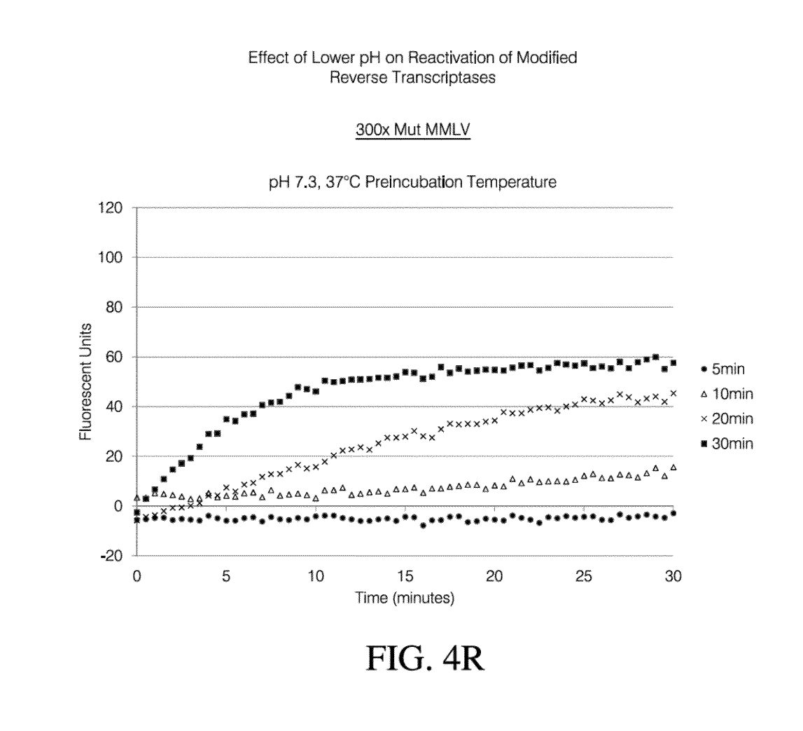

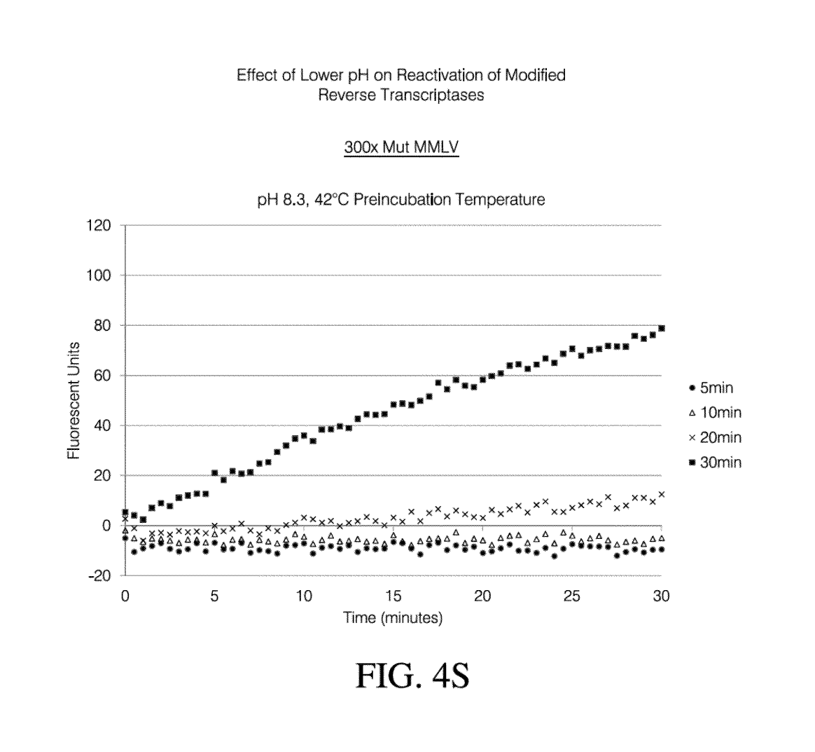

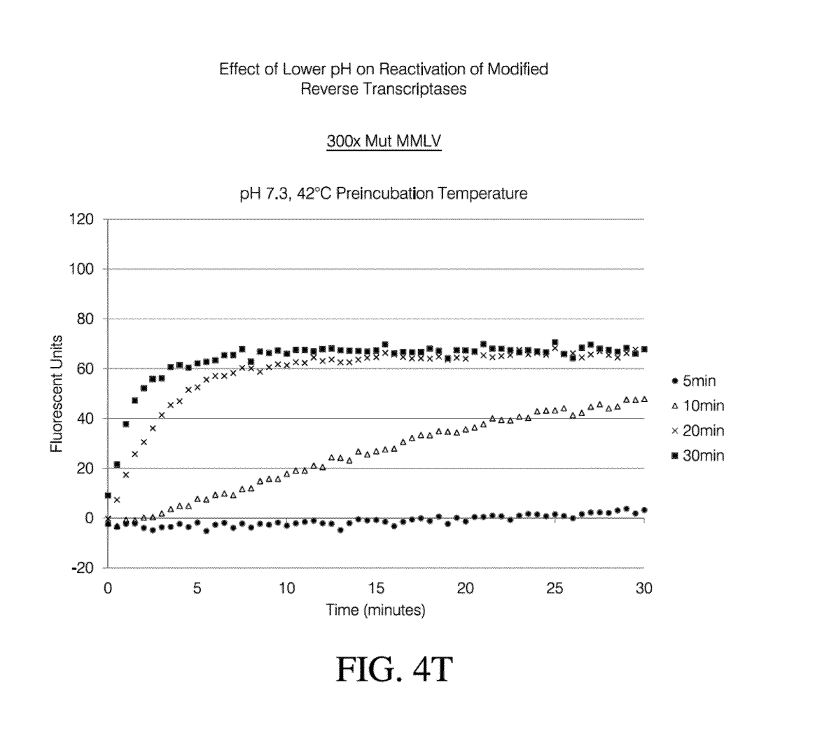

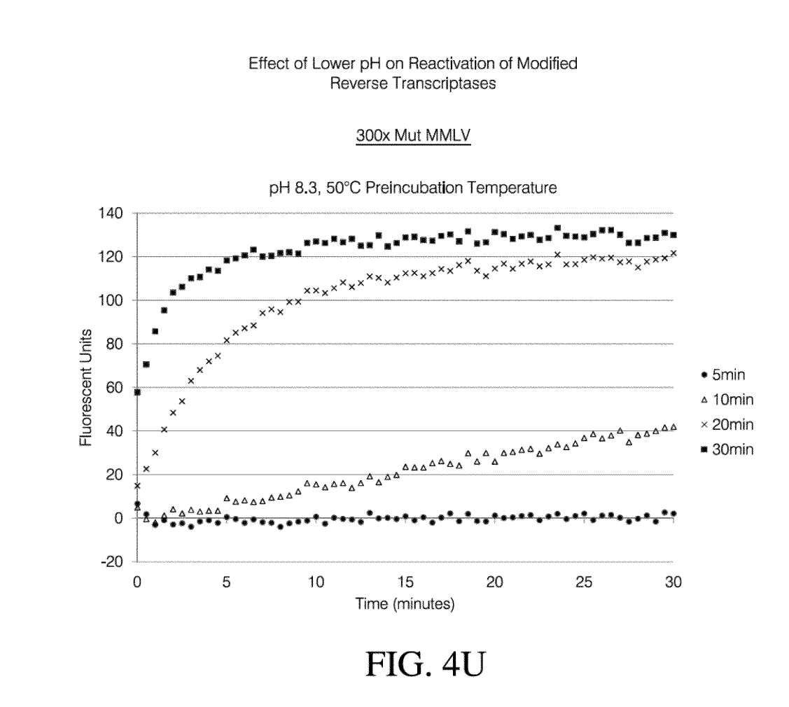

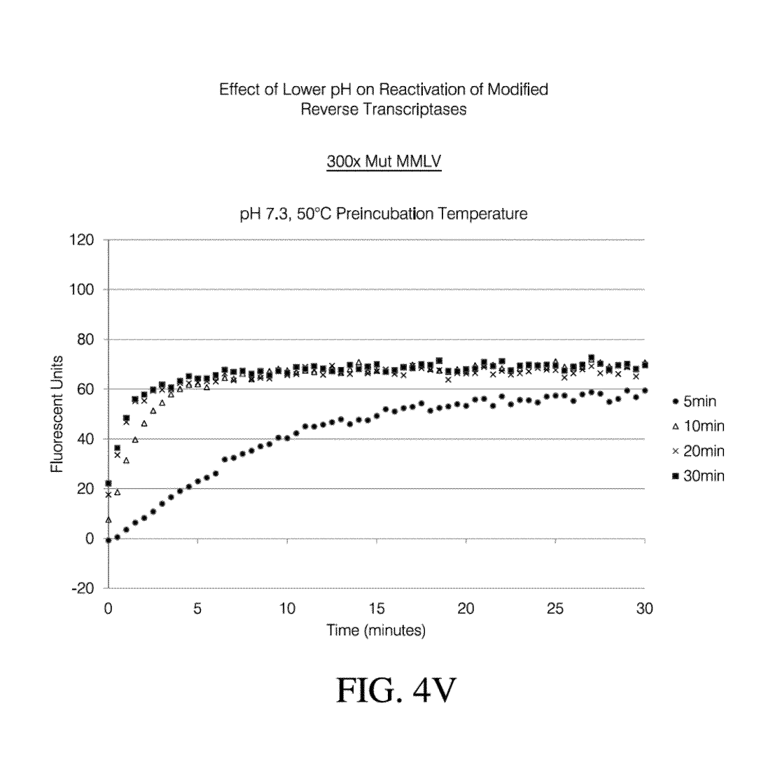

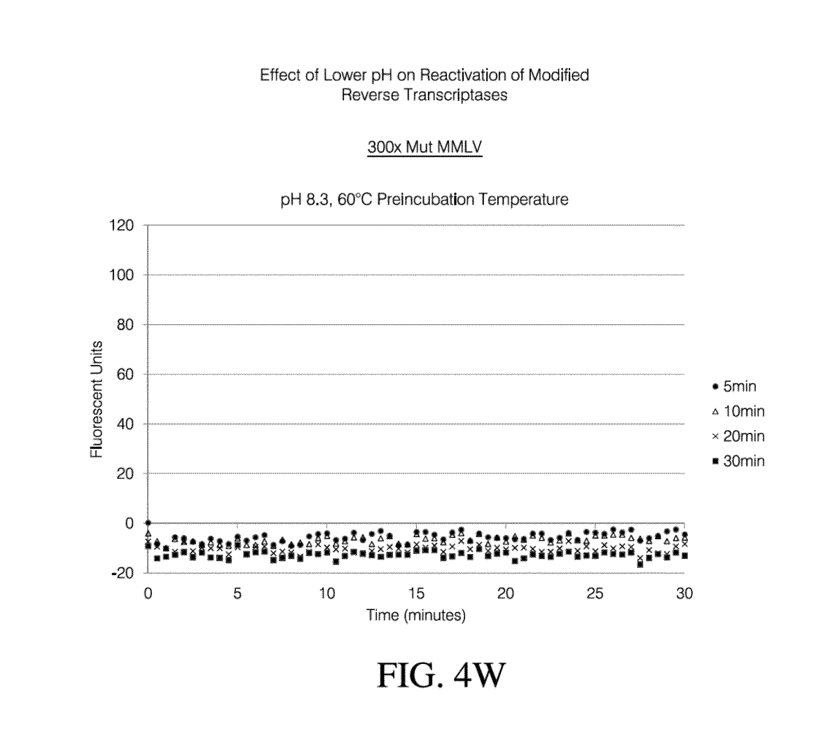

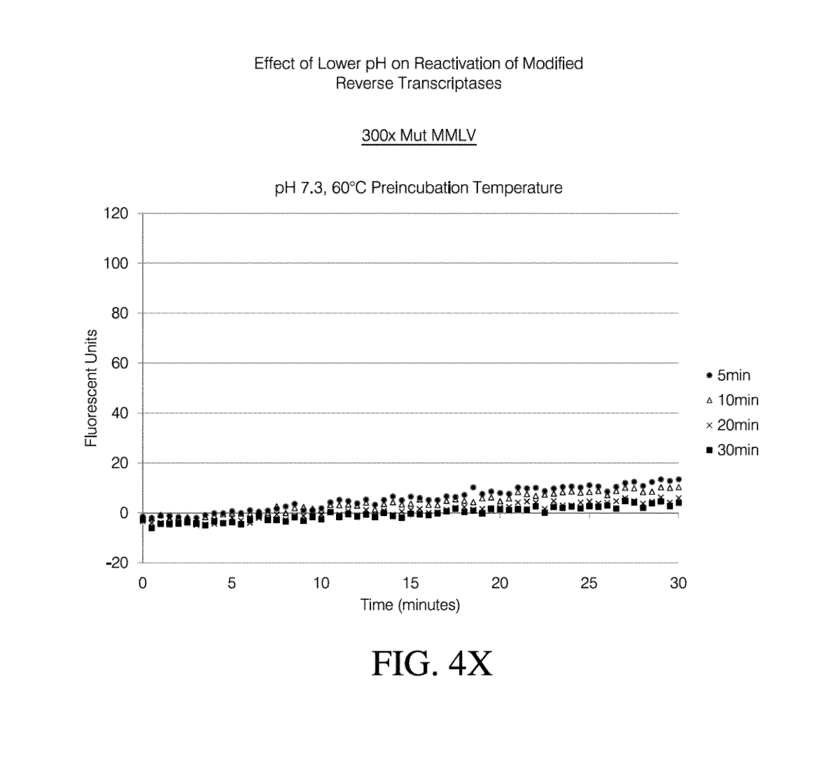

Reversion of chemical modification should occur more efficiently at lower pHs (Pintor-Toro J A, Vázquez D, Palacián E. Biochemistry. Jul. 24, 1979; 18(15):3219-23 and Dixon H B, Perham R N. Biochem J. September 1968; 109(2):312-4). To test whether this holds true for modified reverse transcriptases, a 300:1 DMMA:Enzyme preparation using a commercially available thermostable reverse transcriptase (Maxima™ H Minus) and a non-commercially available thermostable mutant MMLV reverse transcriptase (“Mut MMLV”) were tested utilizing the fluorescent assay described in Example 1, comparing reactions carried out at either pH 8.3 or pH 7.3. Briefly, chemical modification of Mut MMLV was performed exactly as Maxima™ H Minus, except 0.05% Tween-20 (Thermo Scientific, Catalog #PI-28320) was utilized as a detergent. According to FIGS. 4A through 4X, preincubation at 25° C. and pH 7.3 does not reactivate modified enzymes as desired for hot start enzymes. However, preincubation at temperatures between 30° C. to 60° C. at pH 7.3 resulted in faster reactivation than pH 8.3. Reactivation temperature for both Maxima™ H Minus and Mut MMLV enzymes was also lower at pH 7.3 compared to pH 8.3. However, maximum activity remains low for reactivation at 60° C.

### Example 4

### Determining the Highest Temperature Possible for Reactivation of Modified Reverse Transcriptase Enzymes

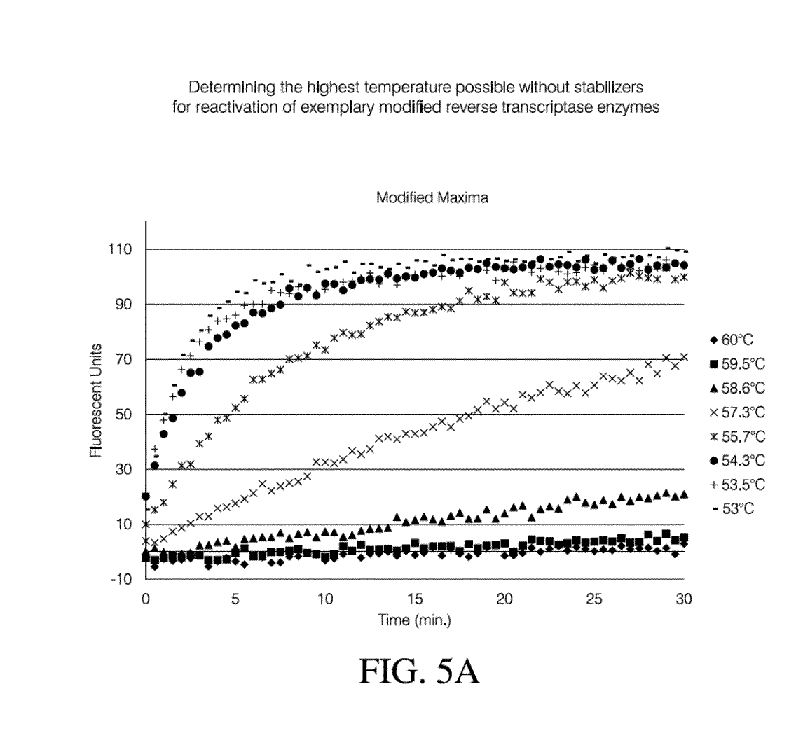

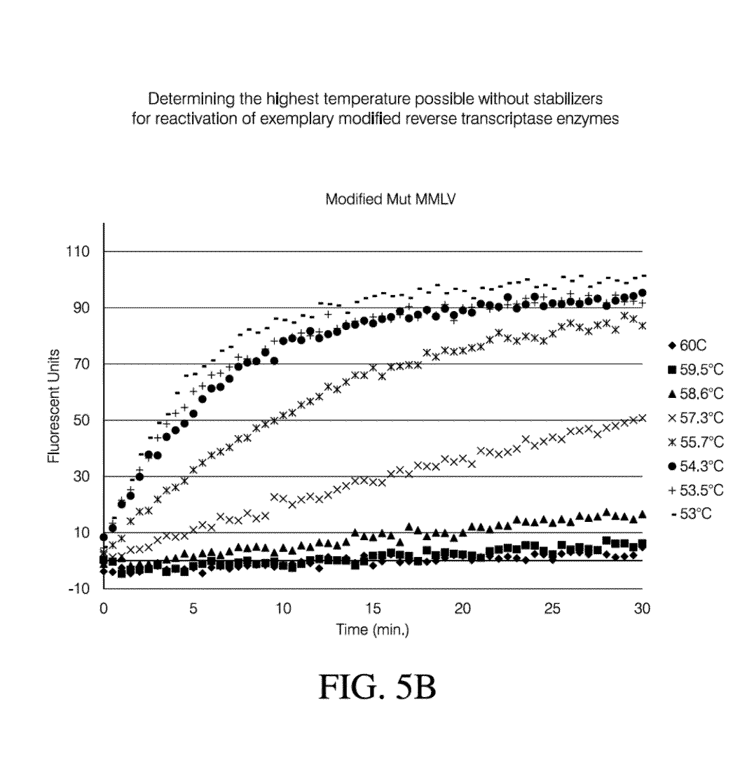

To determine the highest reactivation temperature possible at lower pH, modified enzymes were reactivated as described in Example 1, using incubation at pH 7.3 instead of 8.3, at temperatures ranging from 53° C. to 60° C. FIGS. 5A and 5B show that under conditions using a lower pH, modified enzyme instability starts at 55.7° C., where about 75% activity is retained. At 57.3° C., only about 25% activity remains. By 58.6° C., approximately 2% activity remains. In these assays, no activity was detected at 59.5° C. to 60° C.

### Example 5

### Addition of Stabilizing Components During Reactivation of Modified Reverse Transcriptase Enzymes

To determine whether reactivation of modified enzymes above 50° C. can be improved, experiments with modified enzymes were performed in the presence of stabilizers, such as 10% glycerol and 0.3 M trehalose. In the presence of these additives and at pH 7.3, reactivation occurs more efficiently and full activity is regained at 60° C.

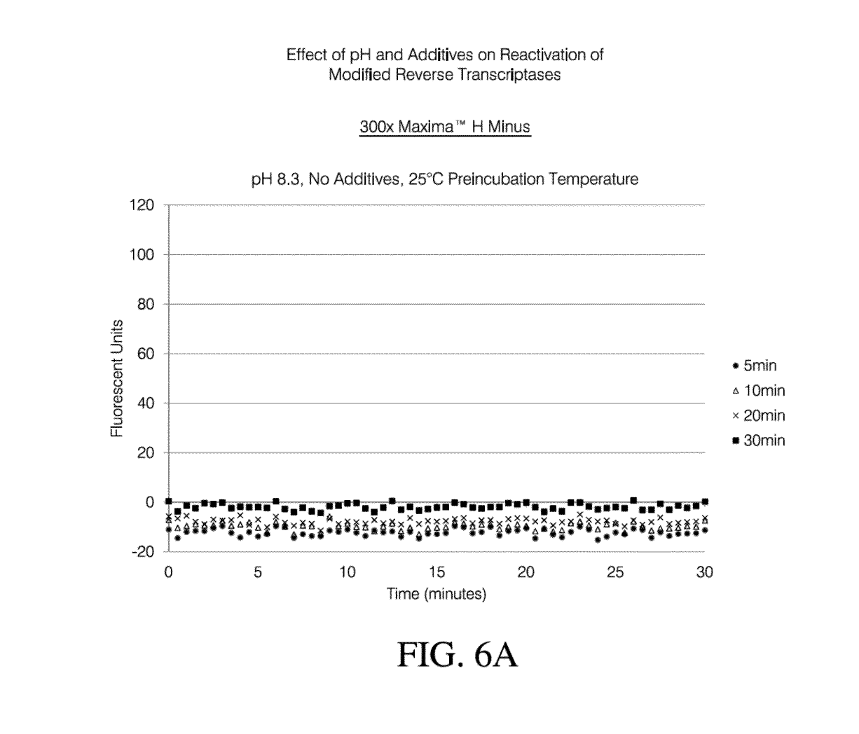

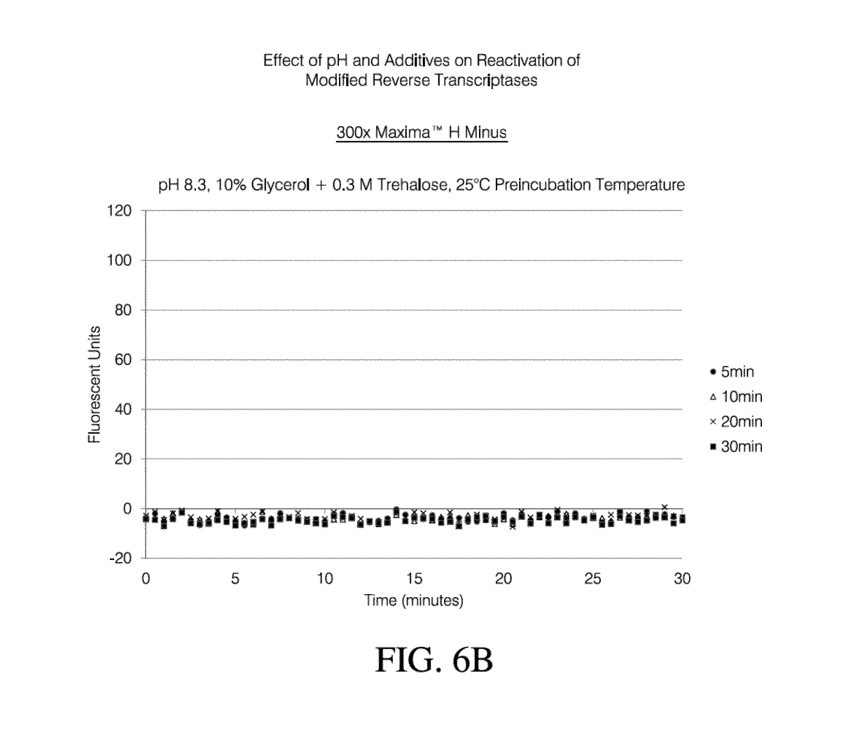

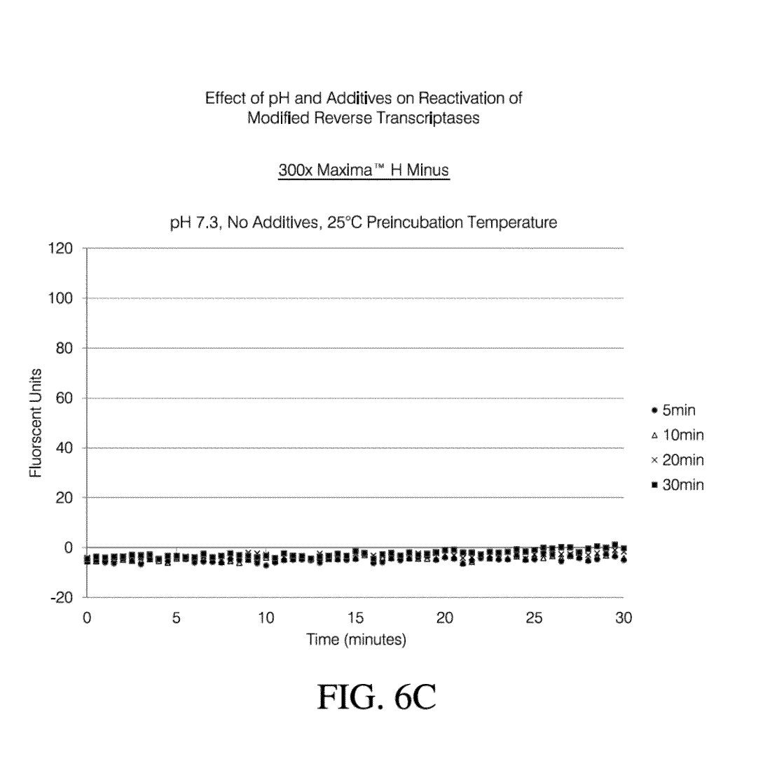

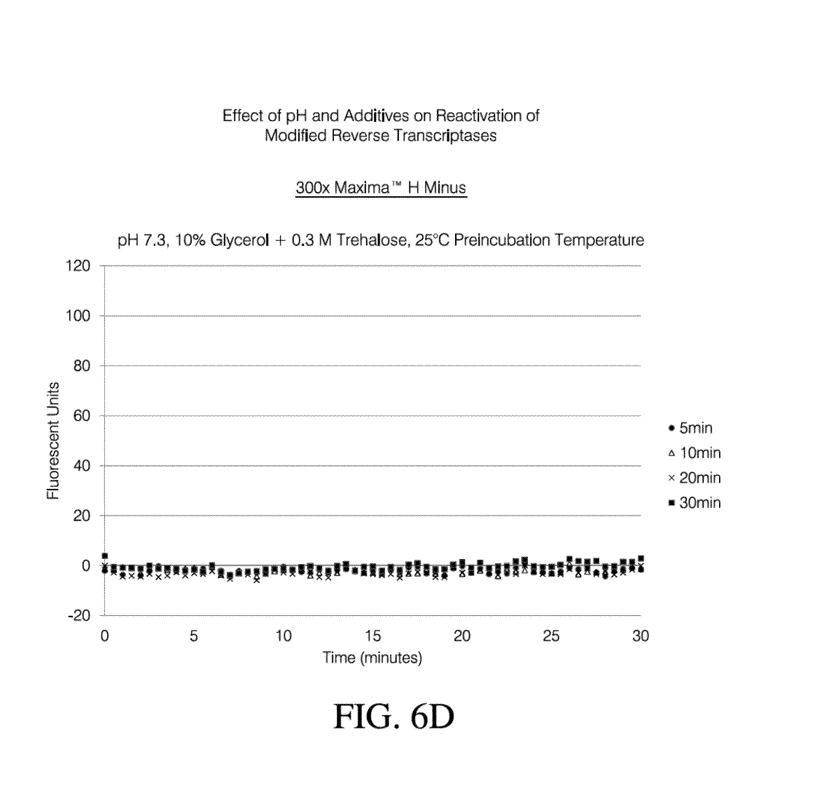

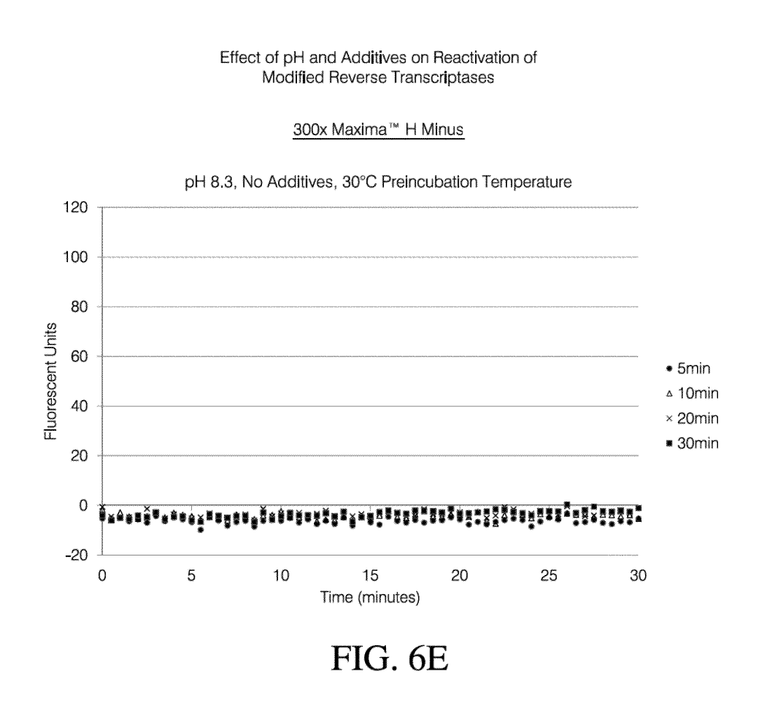

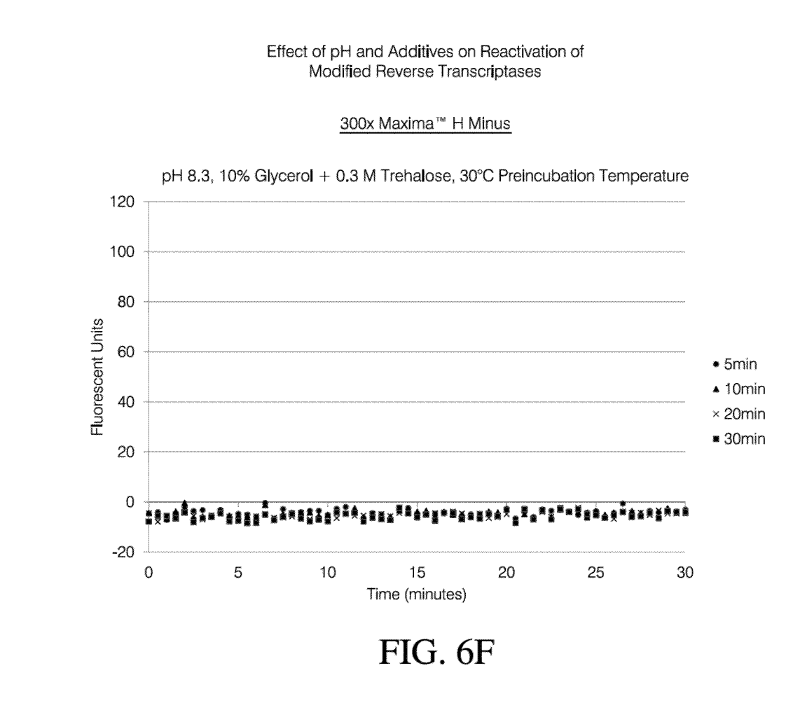

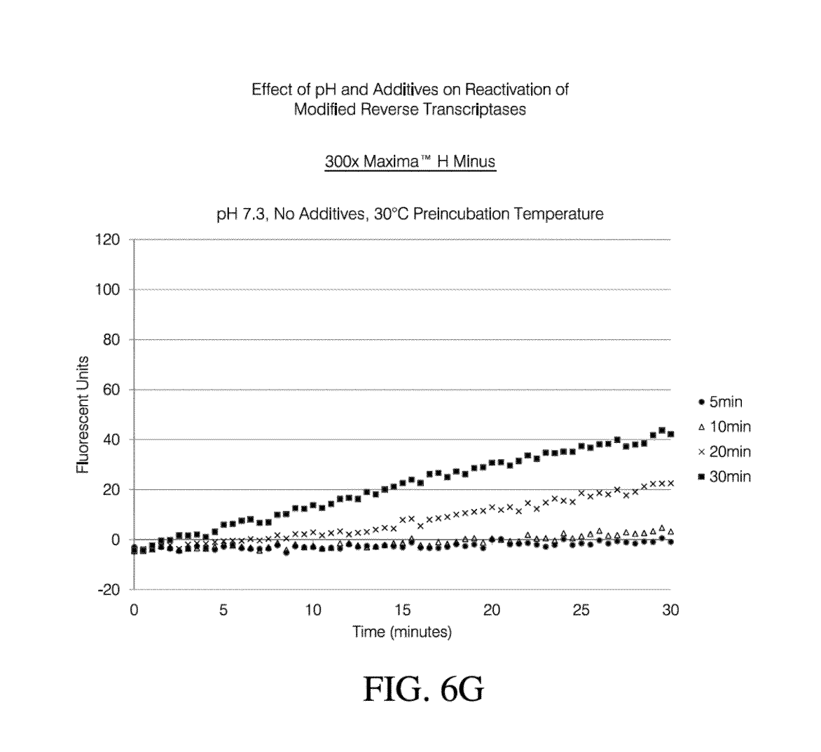

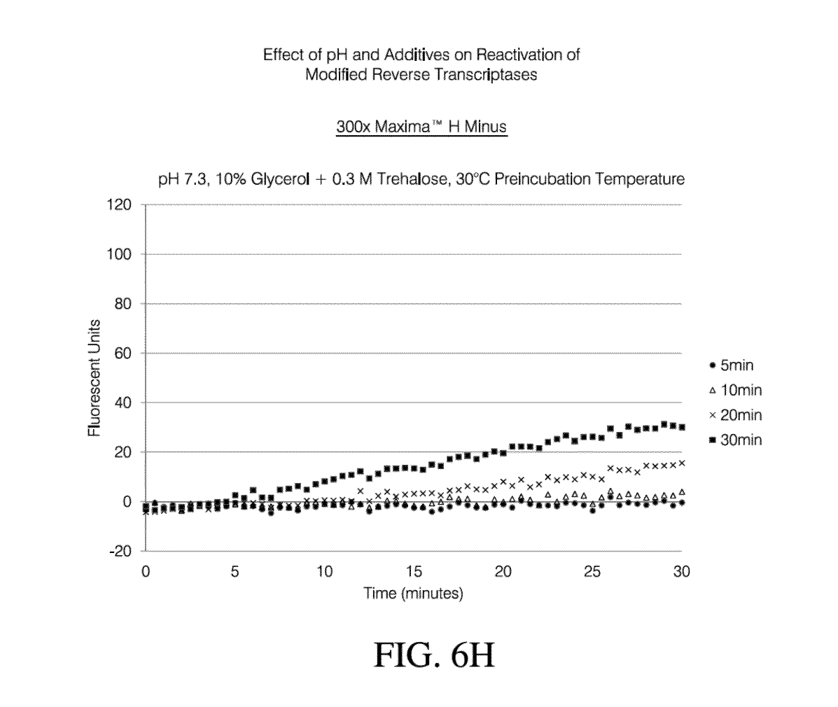

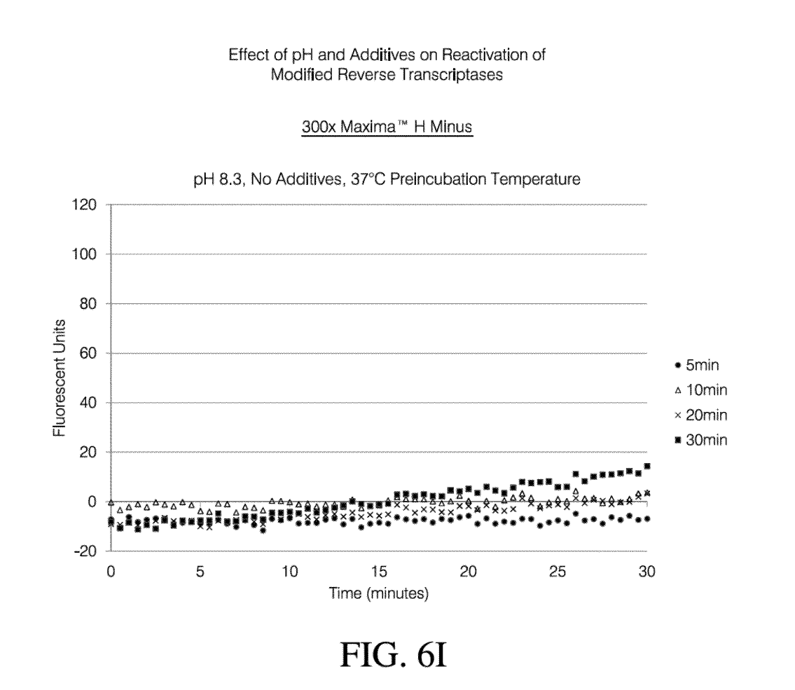

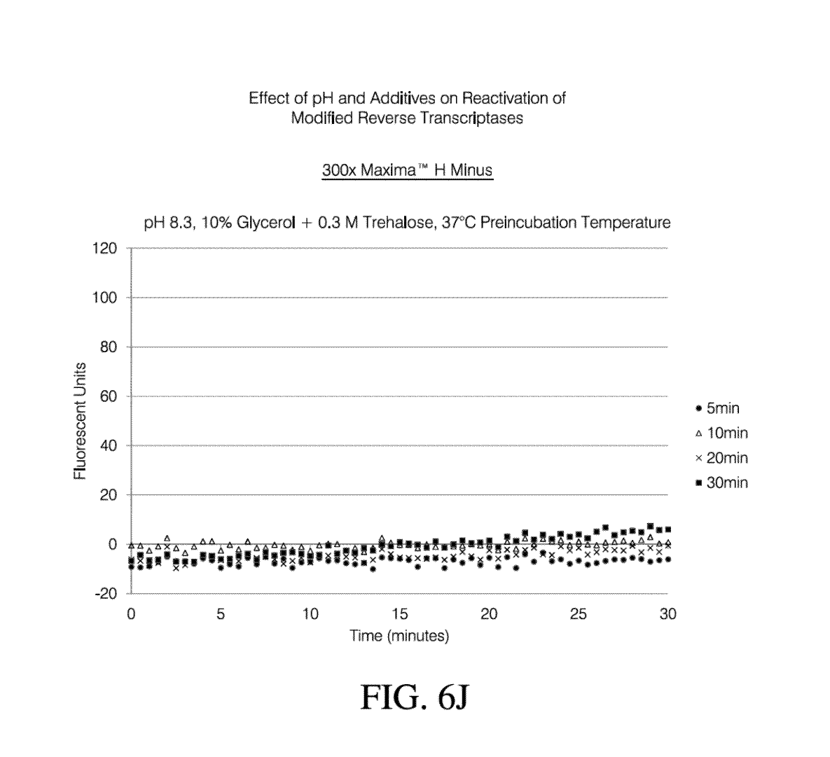

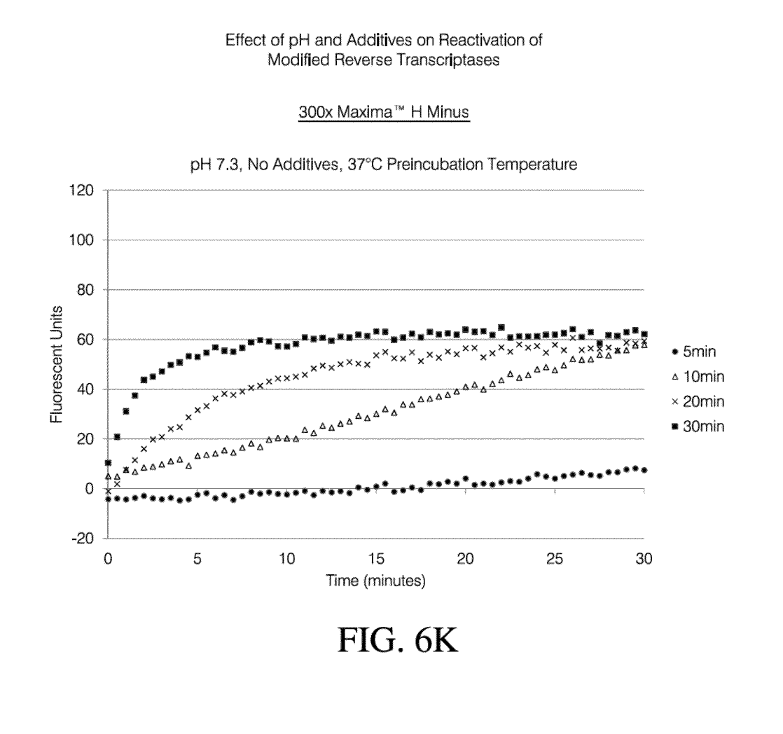

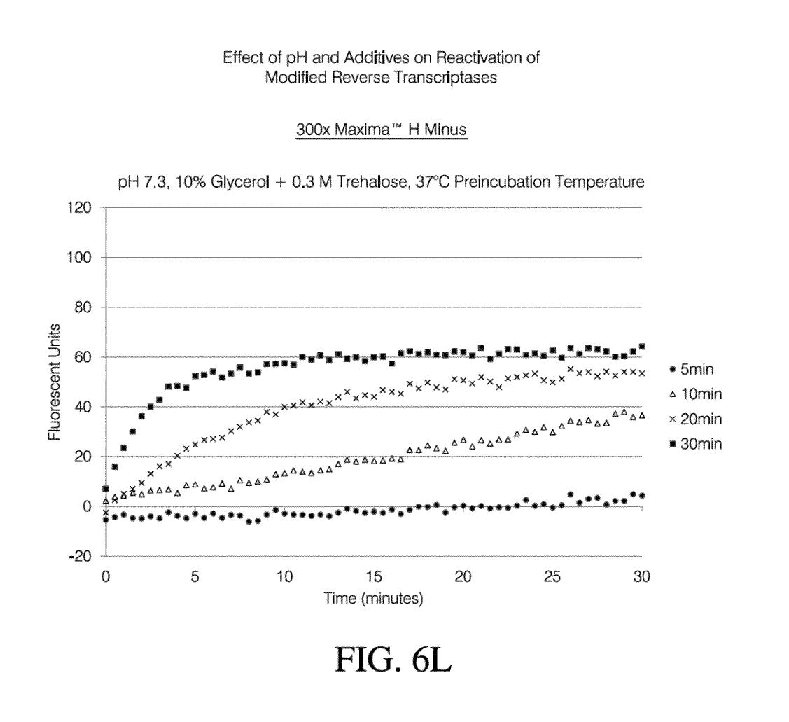

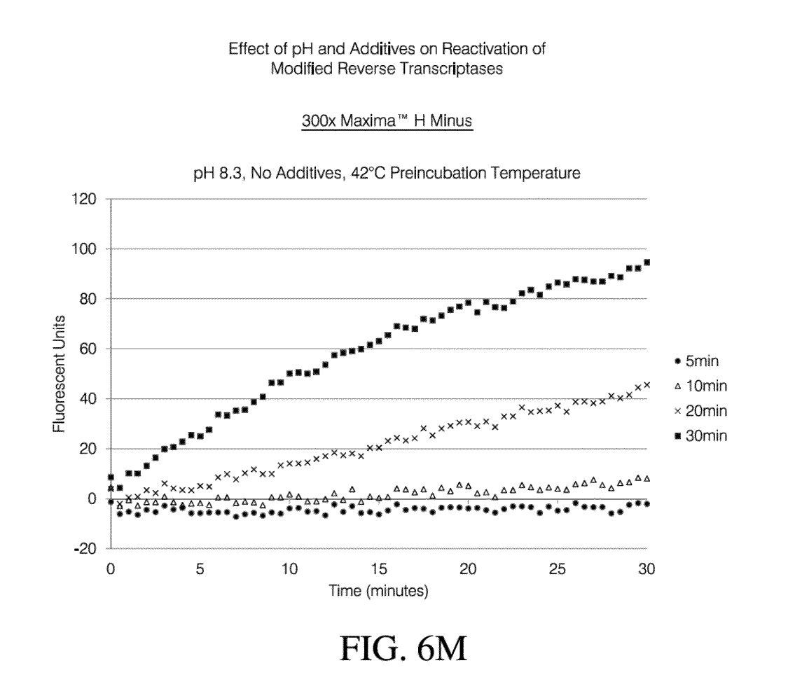

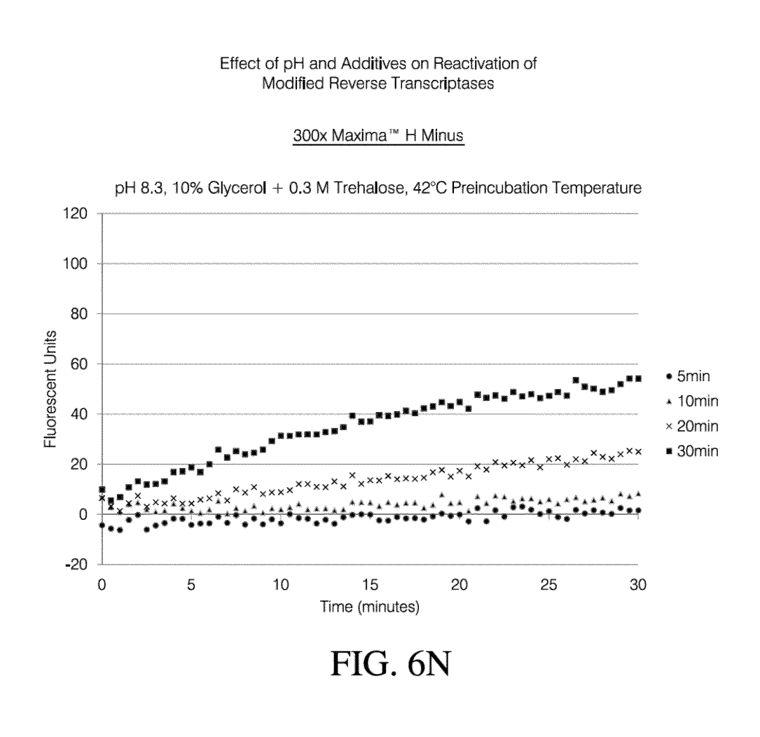

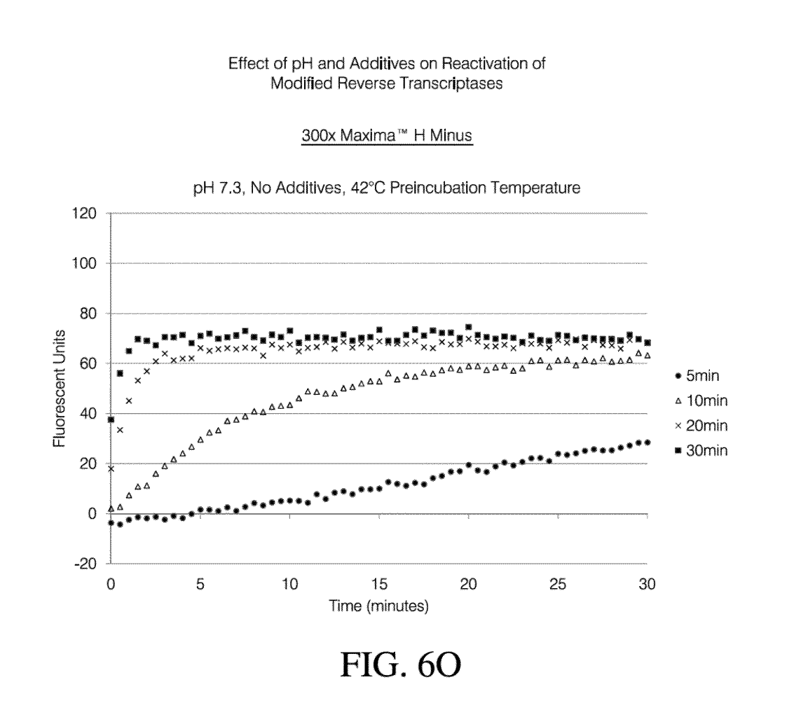

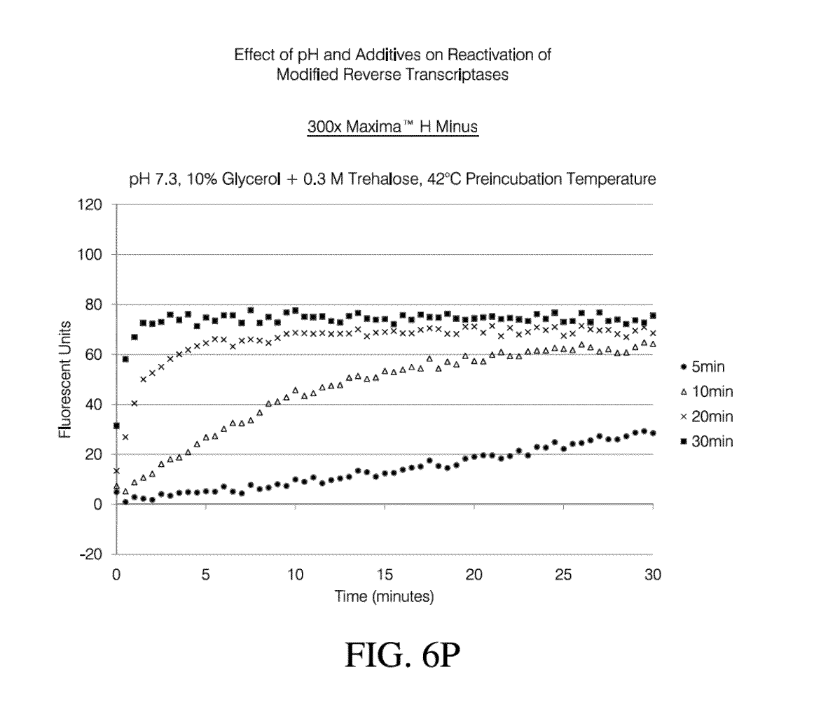

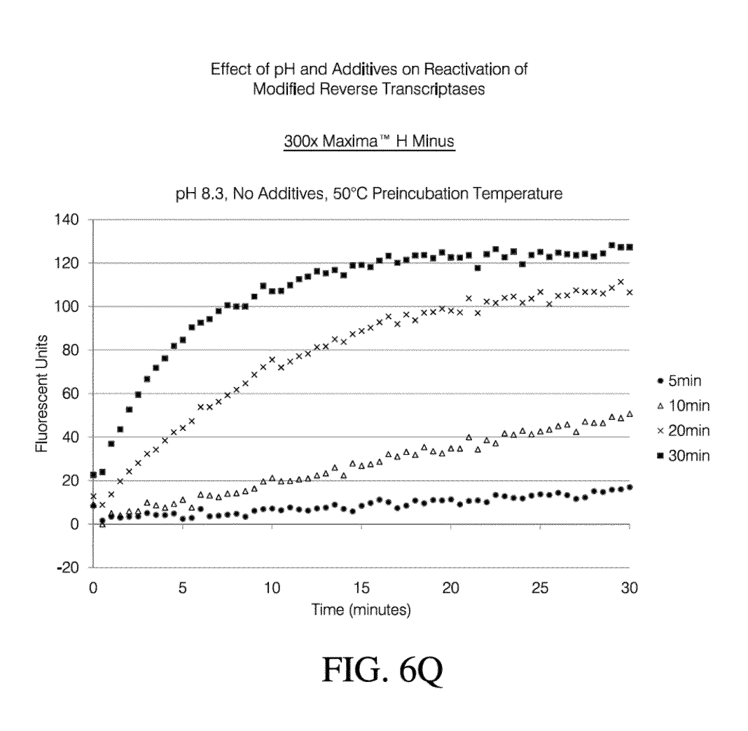

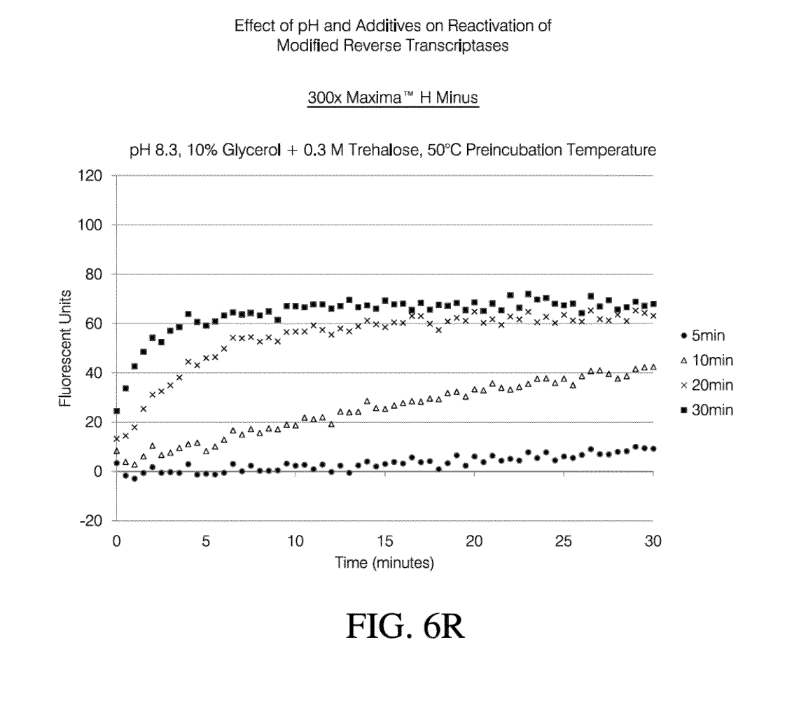

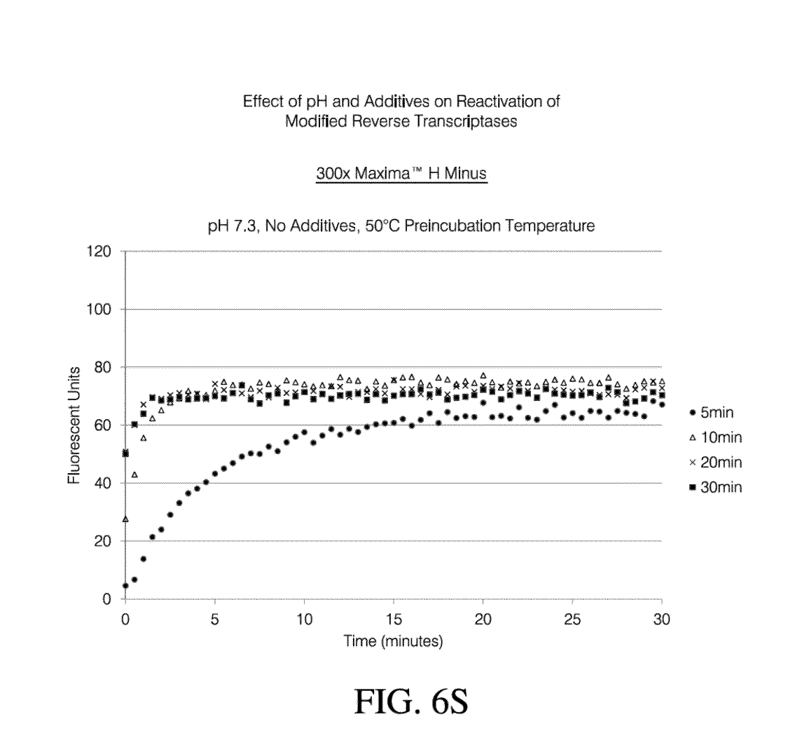

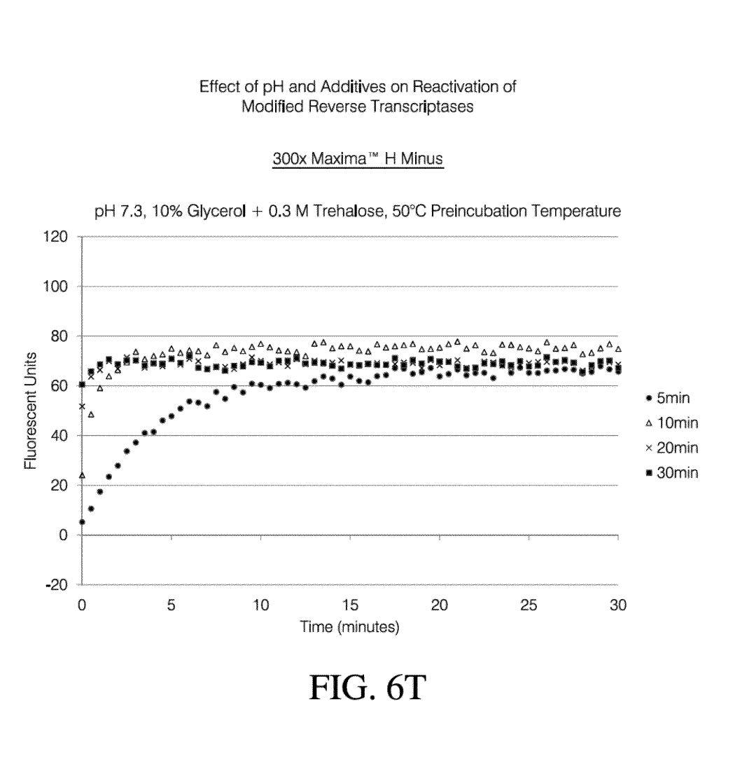

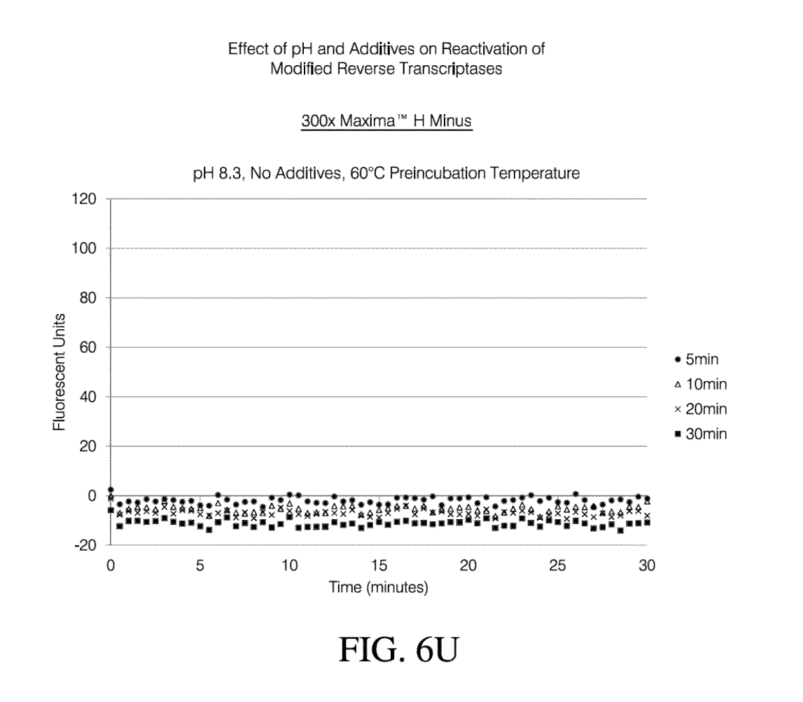

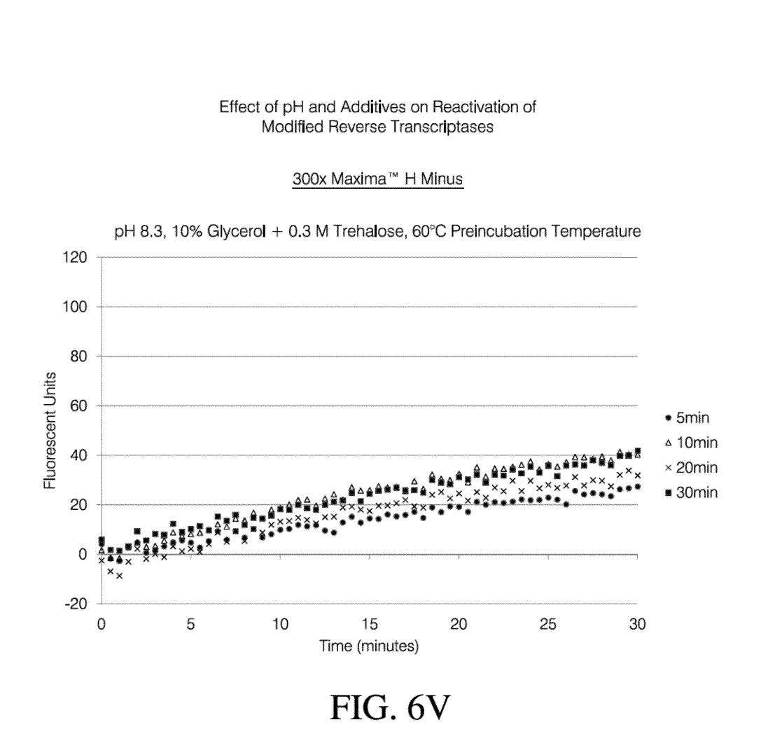

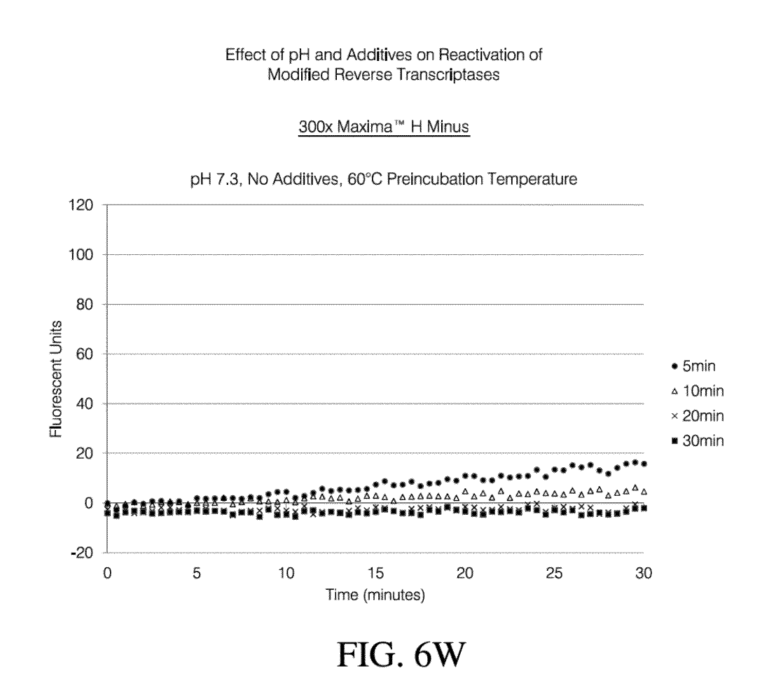

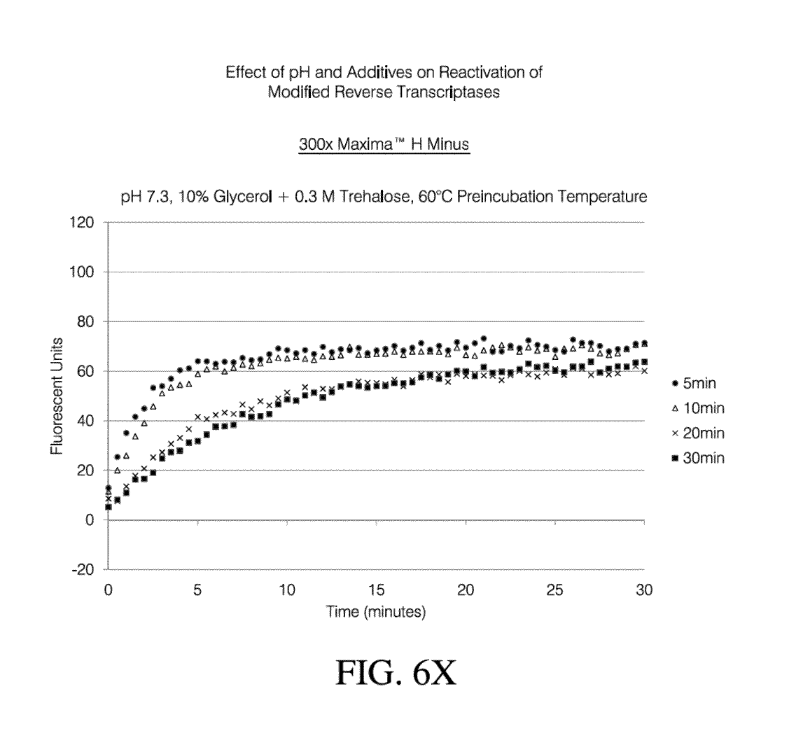

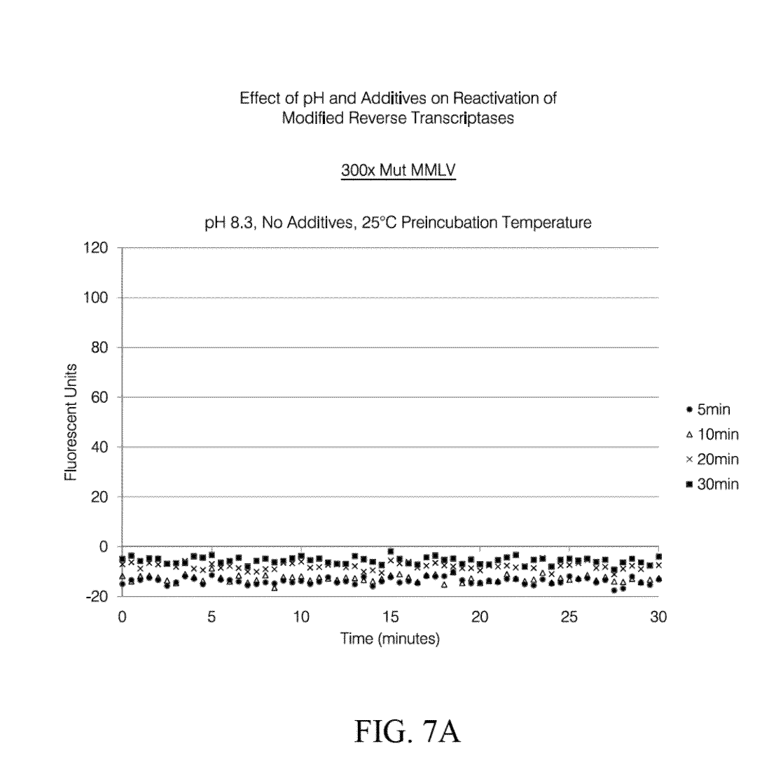

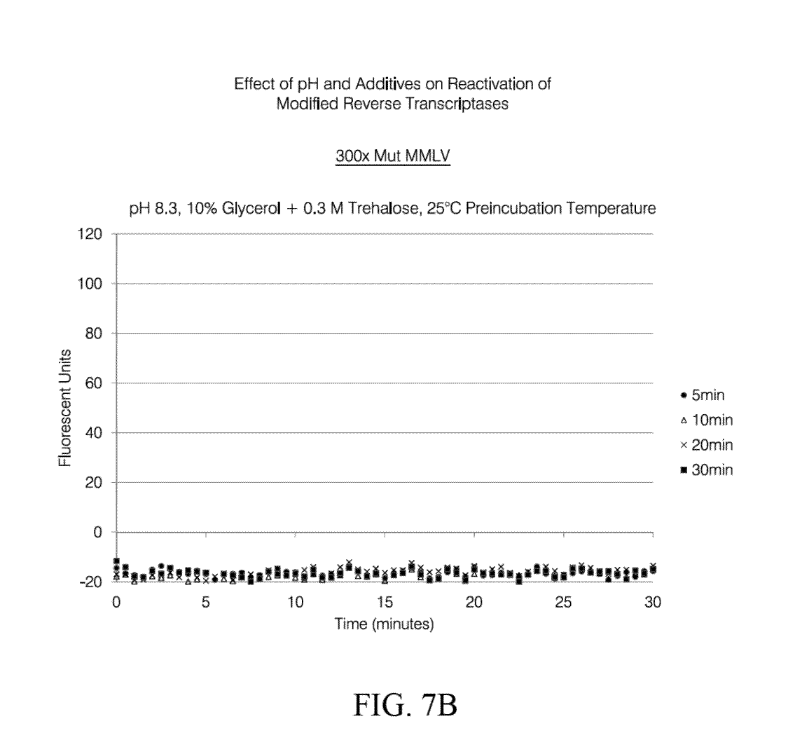

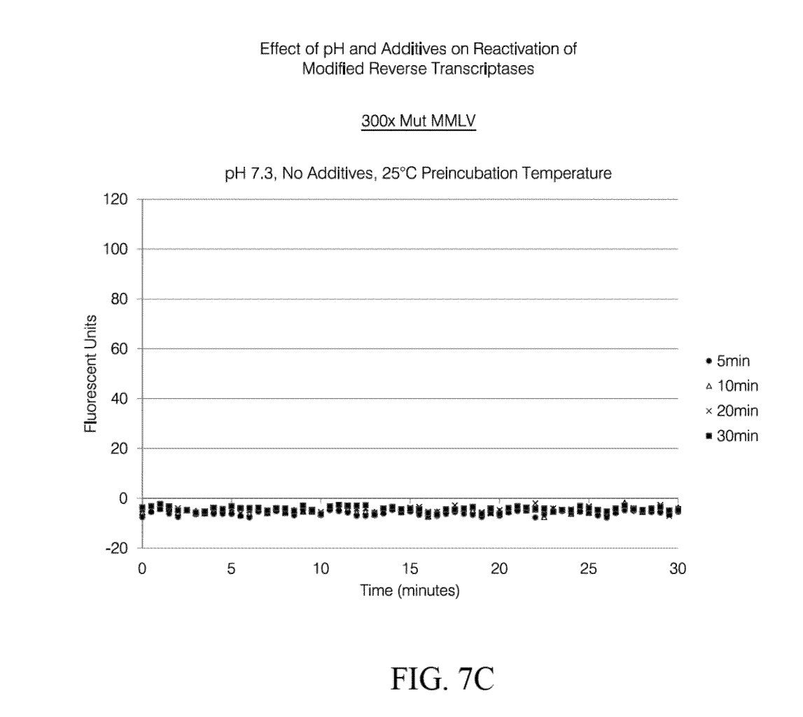

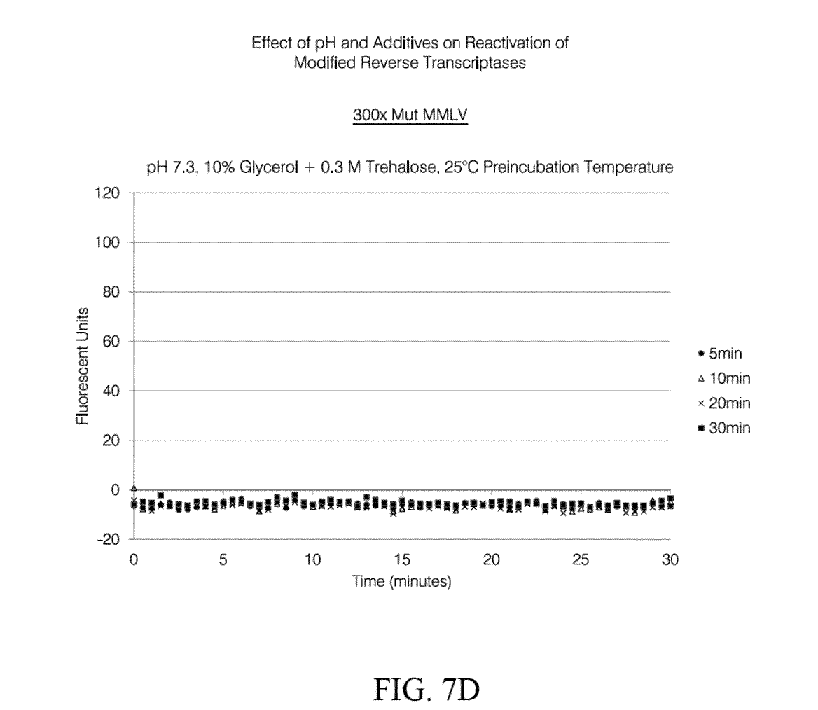

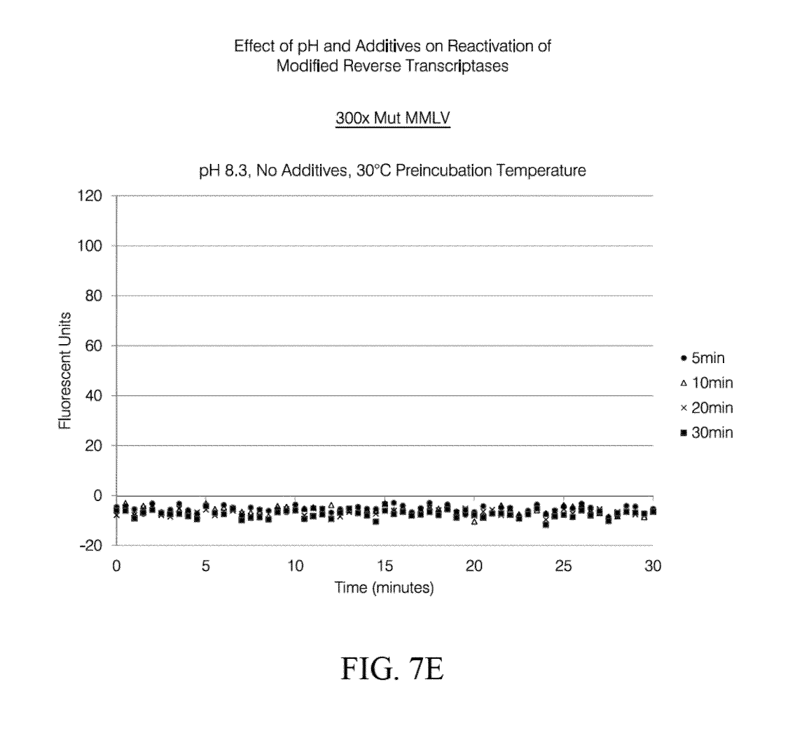

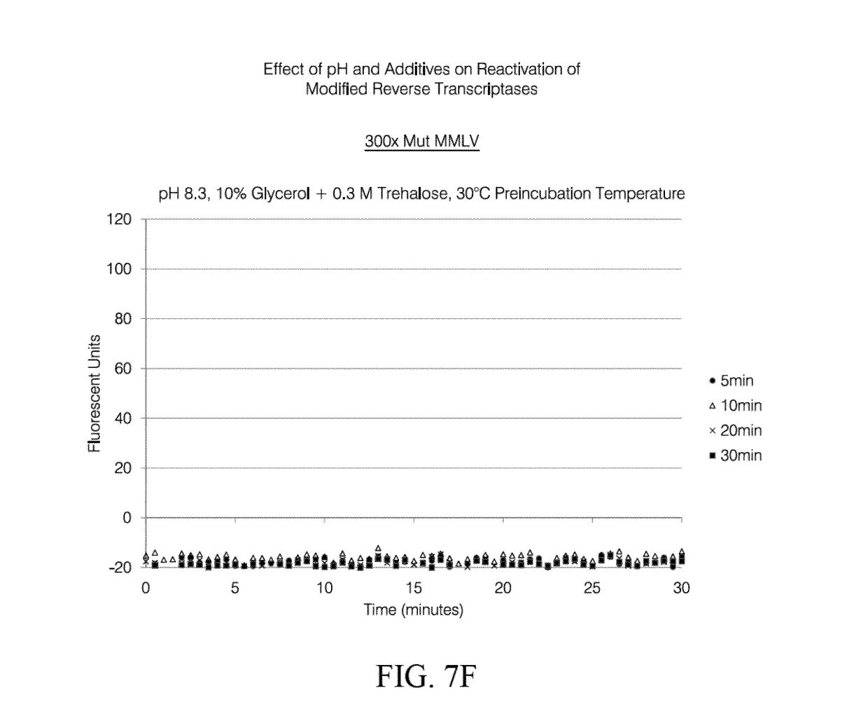

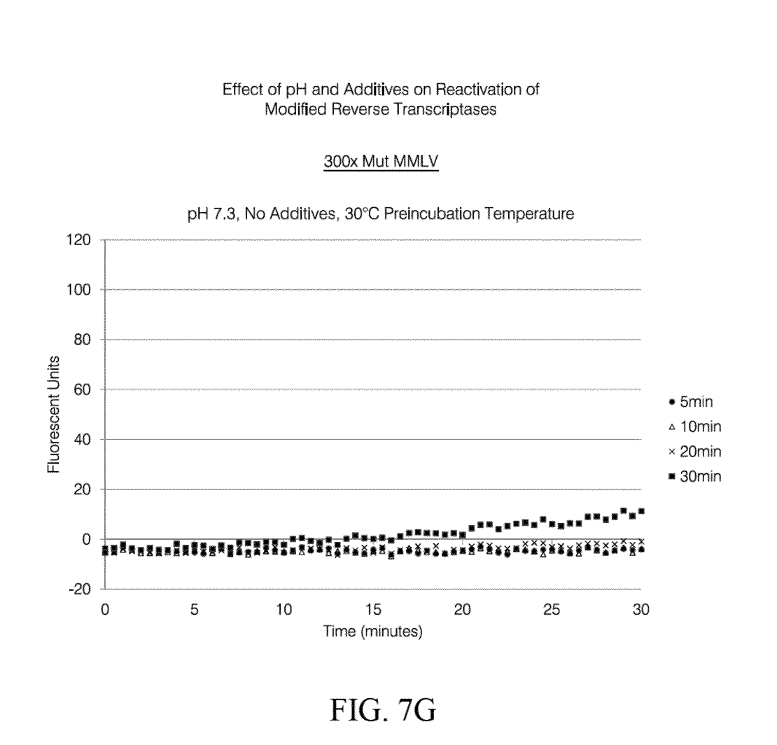

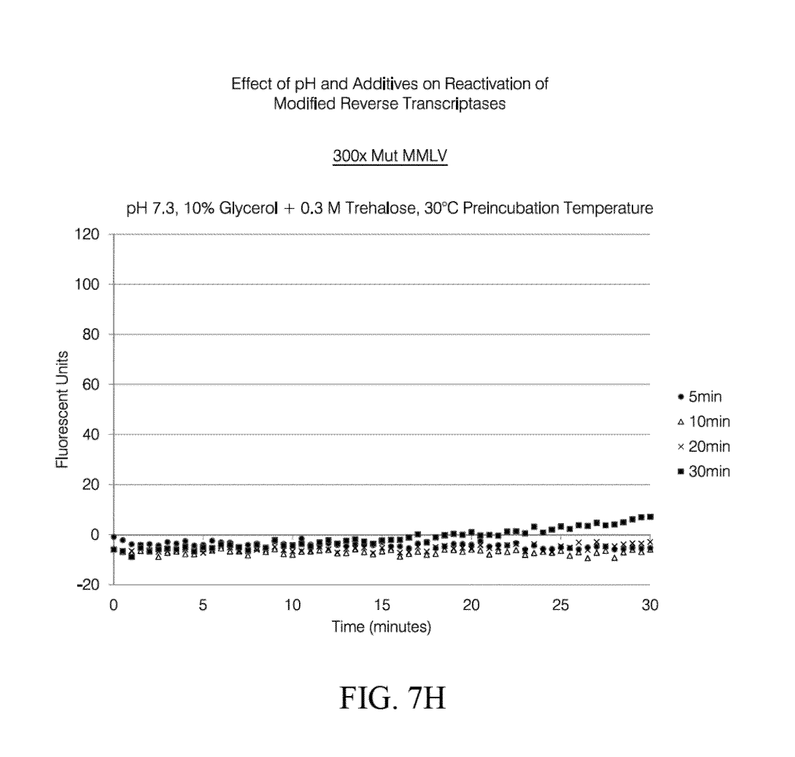

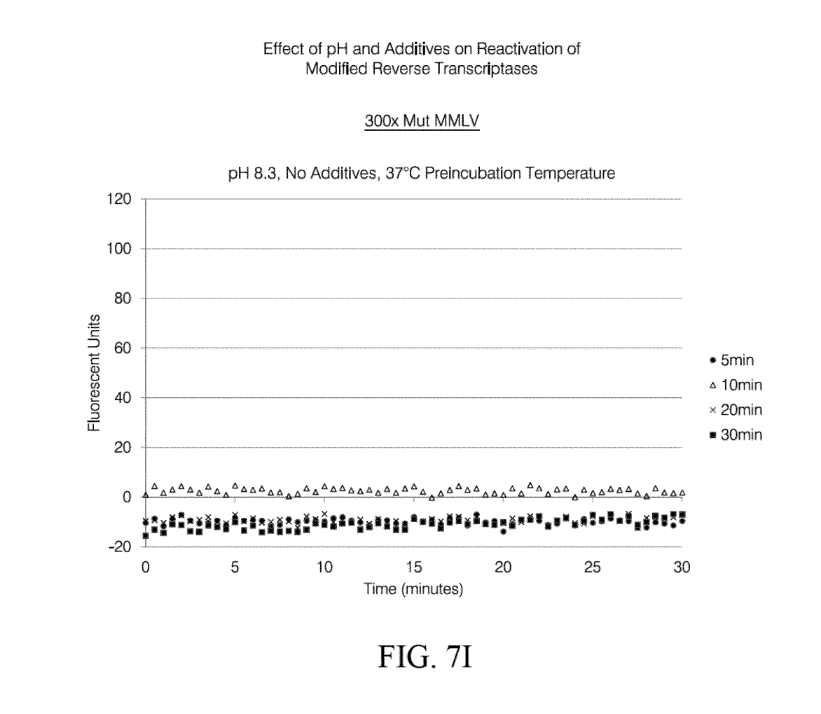

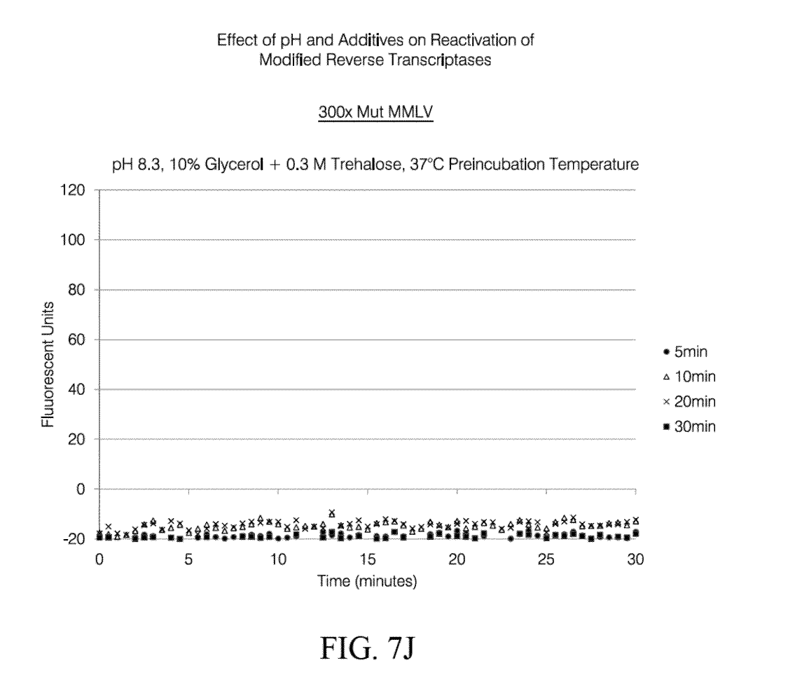

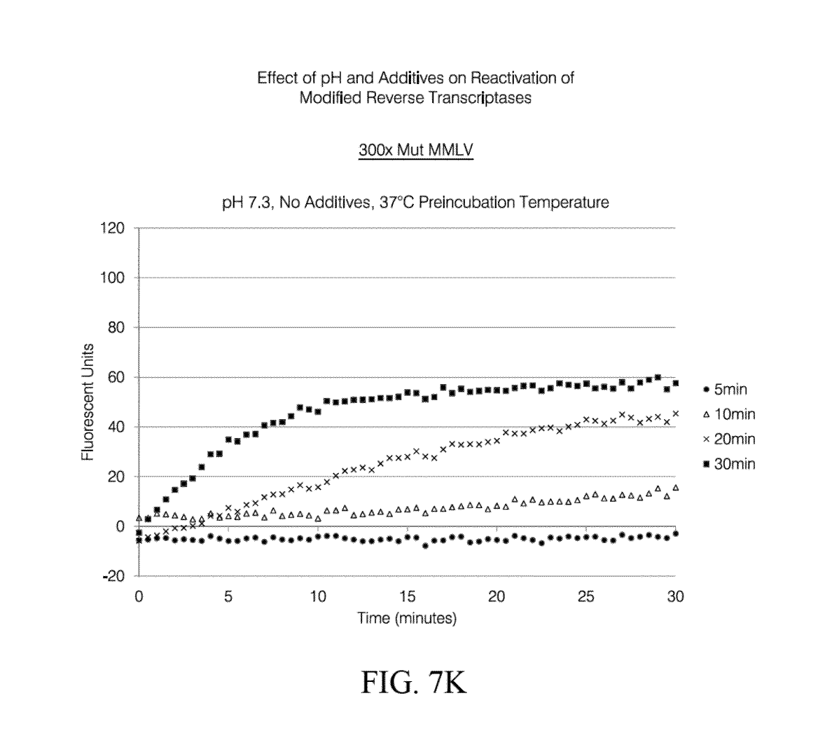

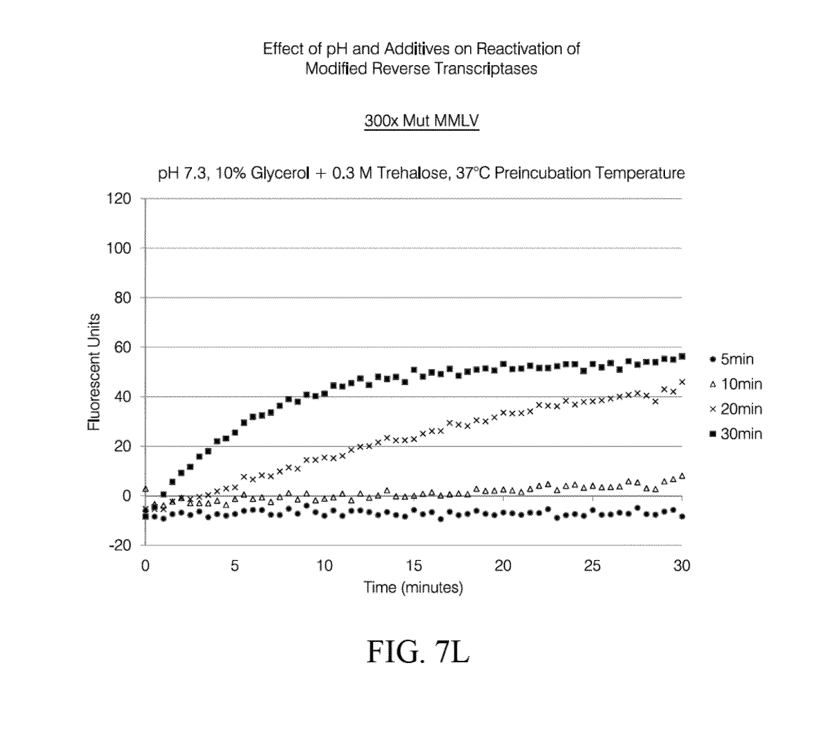

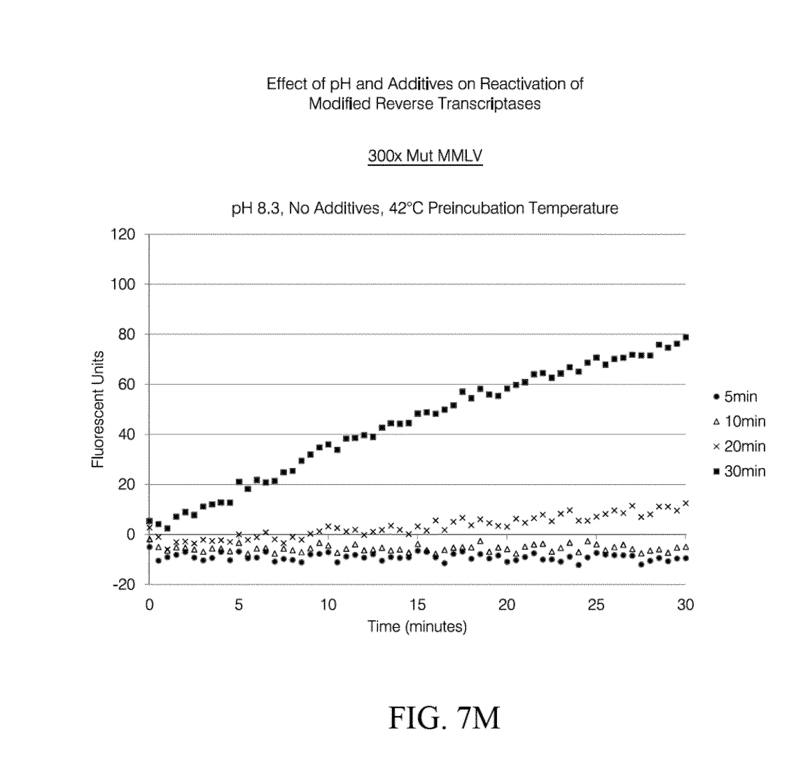

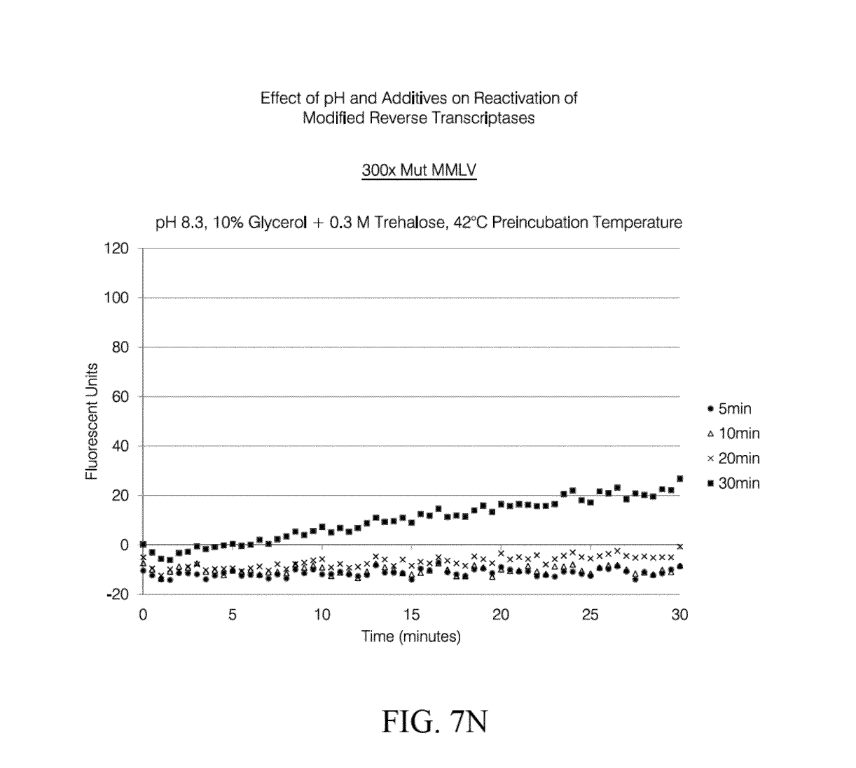

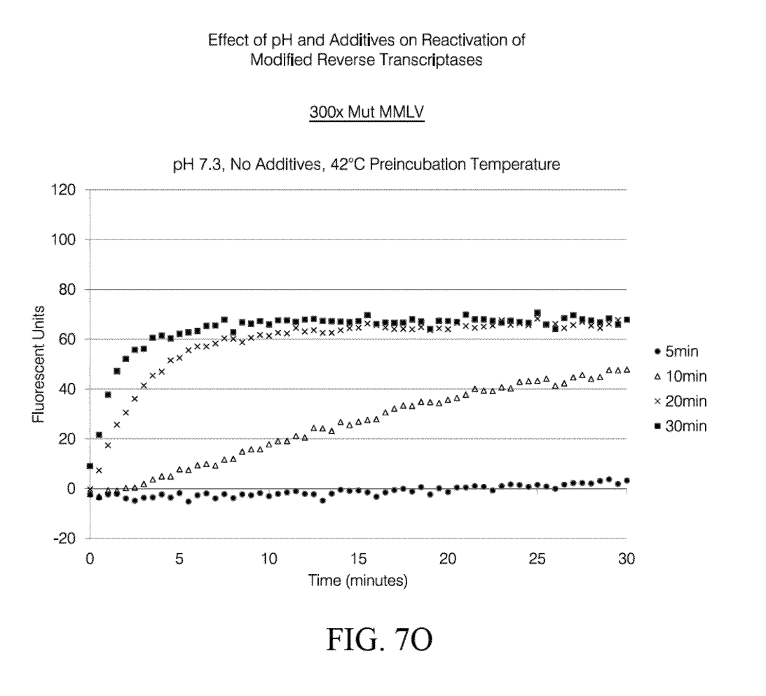

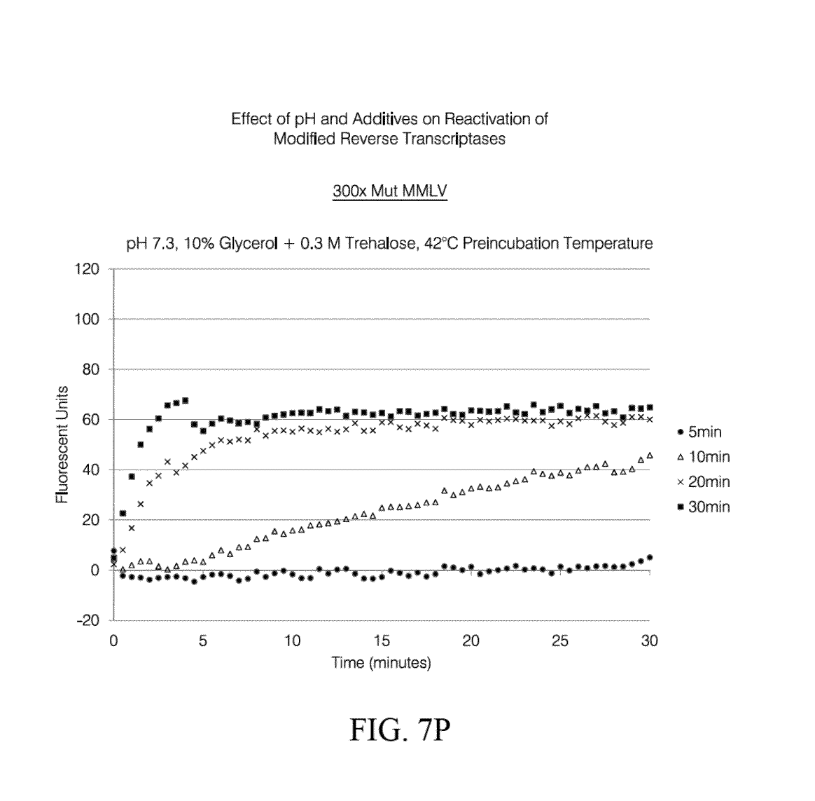

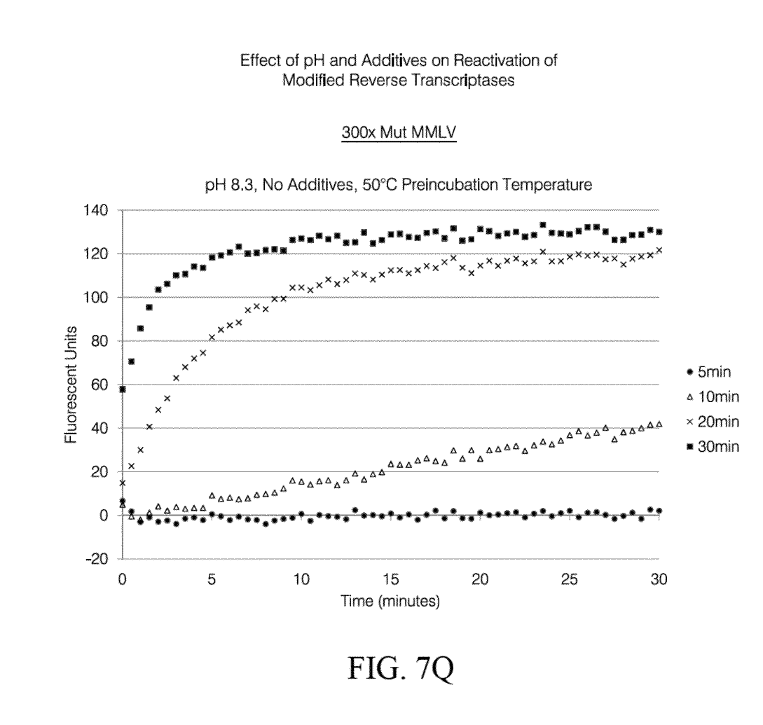

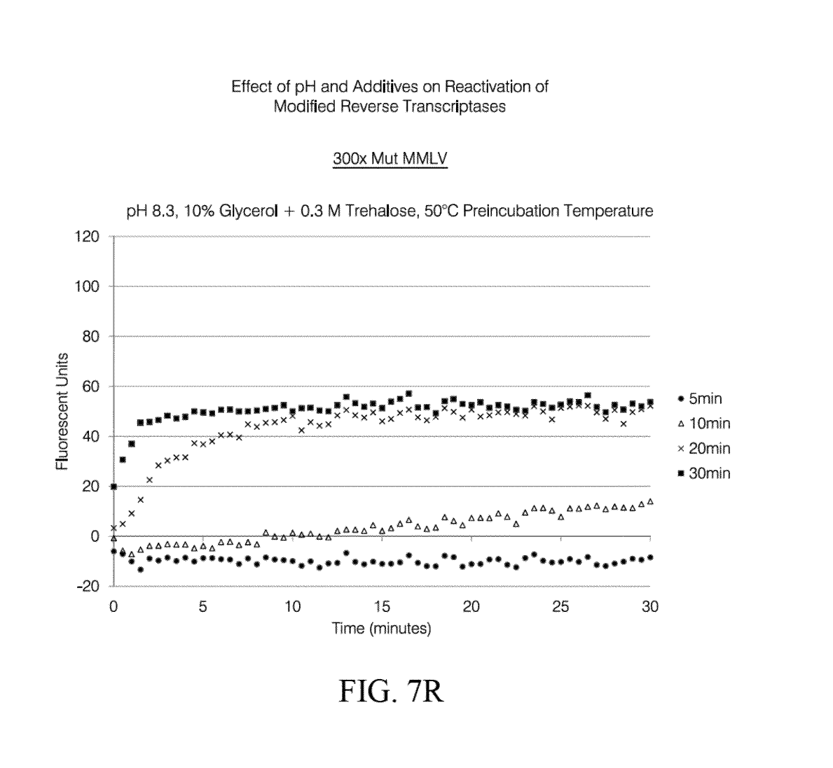

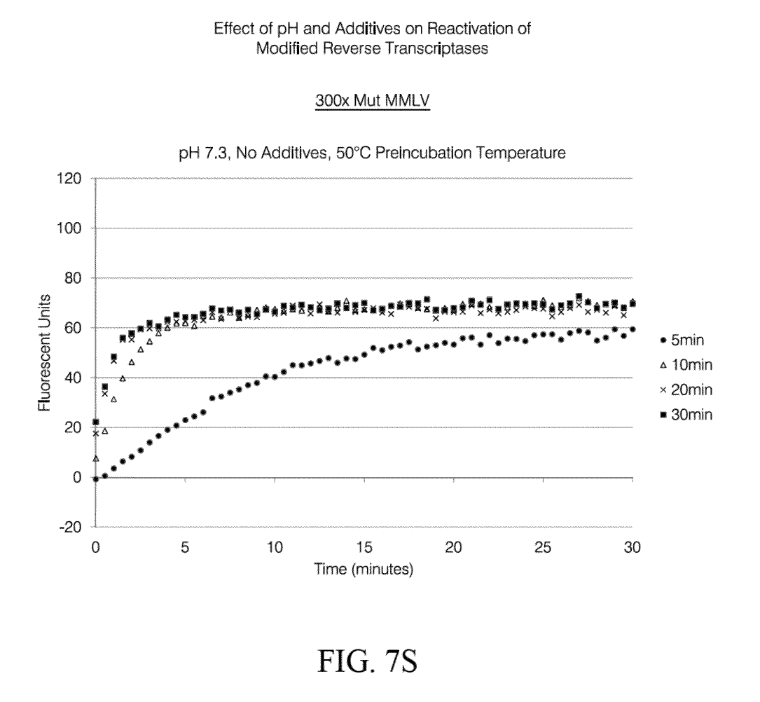

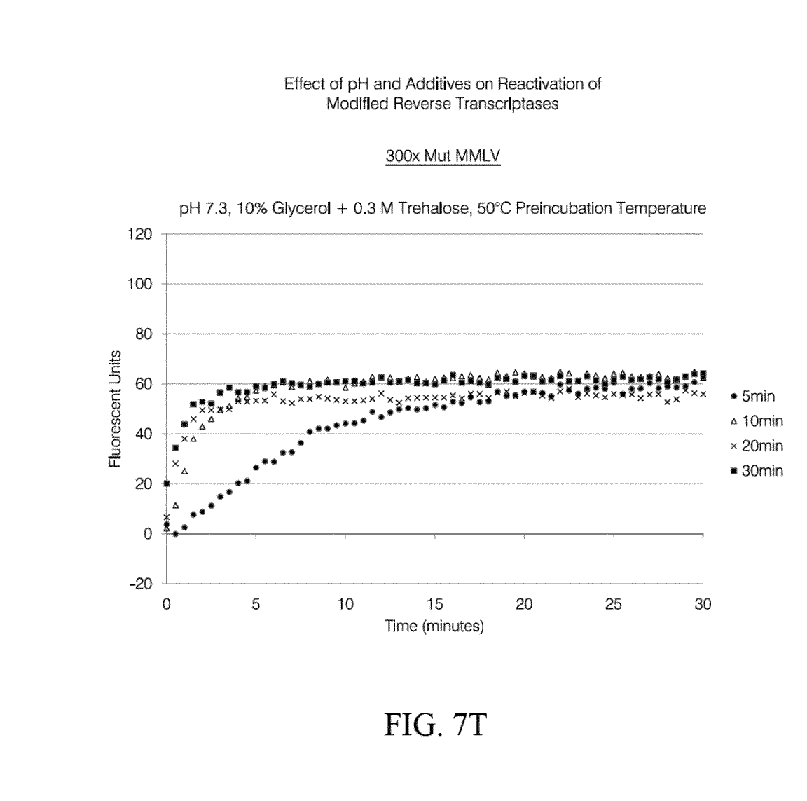

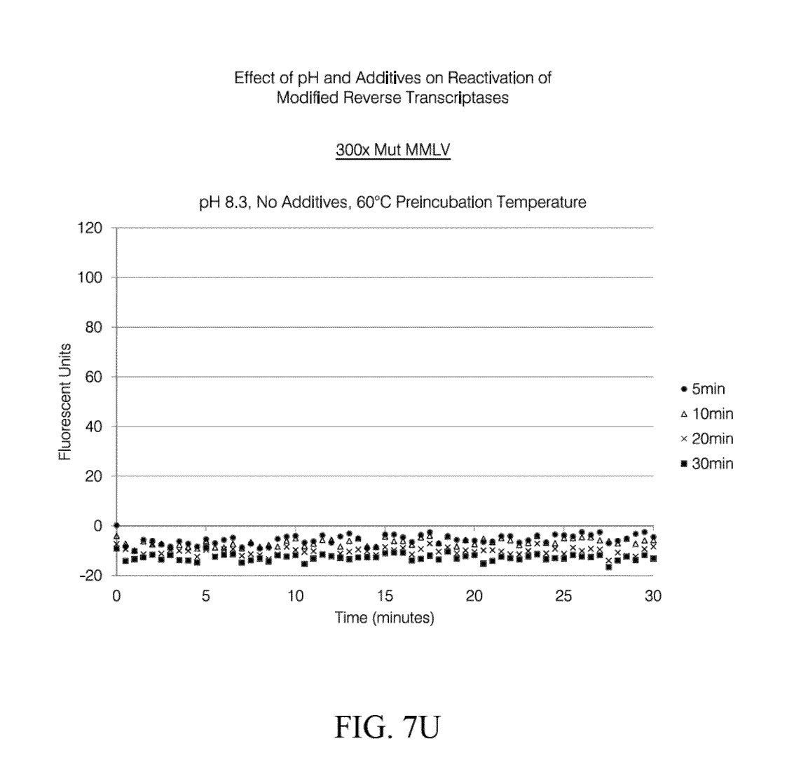

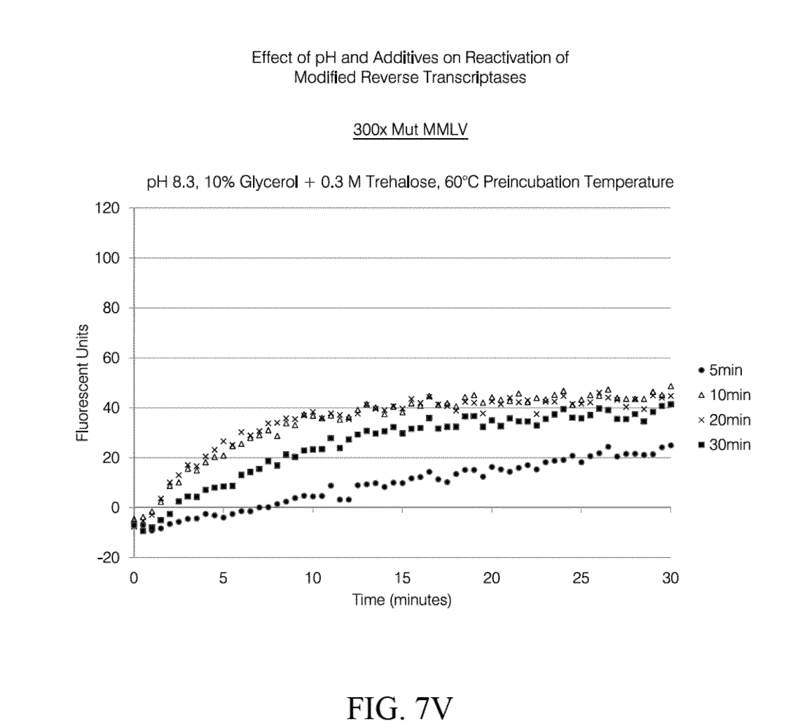

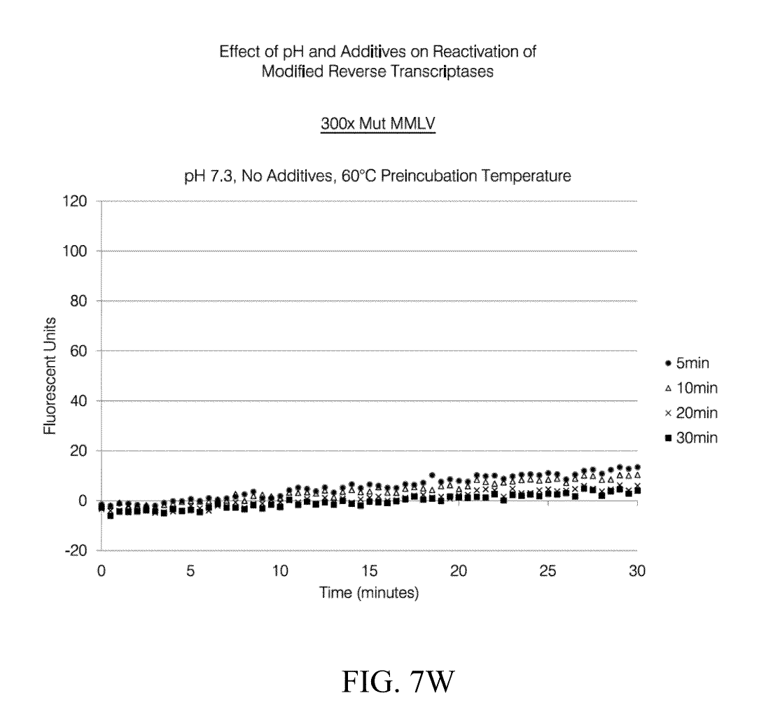

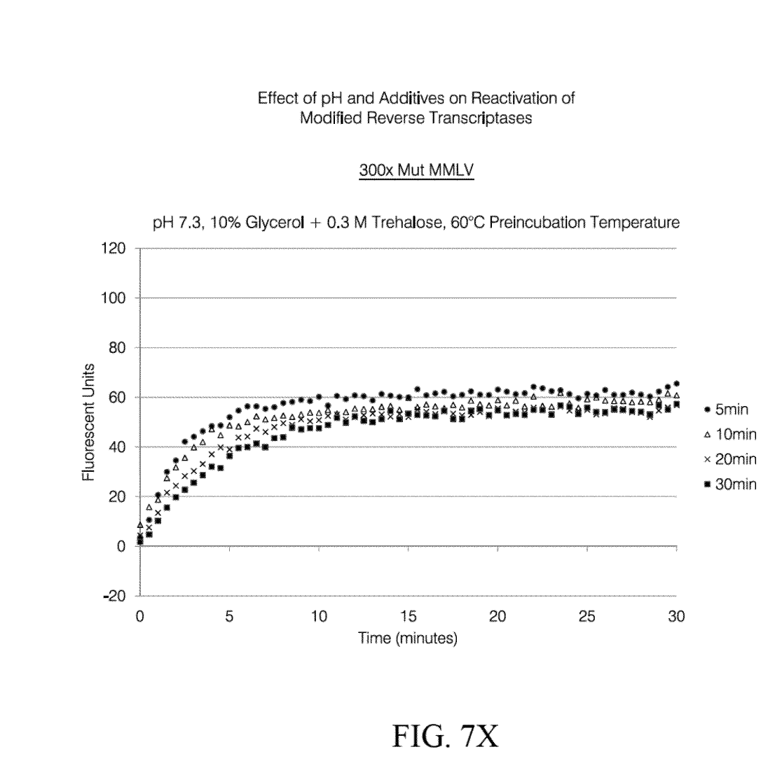

300:1 DMMA:Enzyme preparations at pH 8.3 and pH 7.3 with and without the addition of 10% glycerol and 0.3 M trehalose as stabilizers. Preincubation or reactivation temperatures include 25-60° C. for a duration of 5-30 minutes as indicated in FIGS. 6A through 6X and FIGS. 7A through 7X. The addition of these stabilizers resulted in measurable increases in fluorescent reading (enzyme activity) at 60° C. at both pH 8.3 and pH 7.3 conditions. At pH 7.3, the addition of stabilizers allows full reactivation of modified enzymes at 60° C.

### Example 6

### Modification and Reactivation of Non-Thermostable Reverse Transcriptases

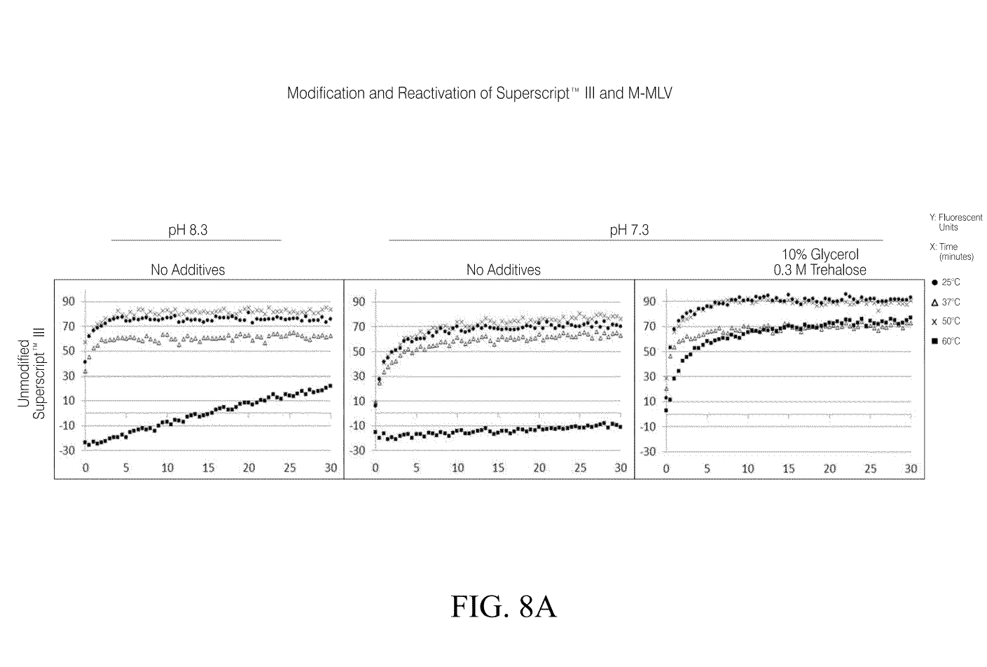

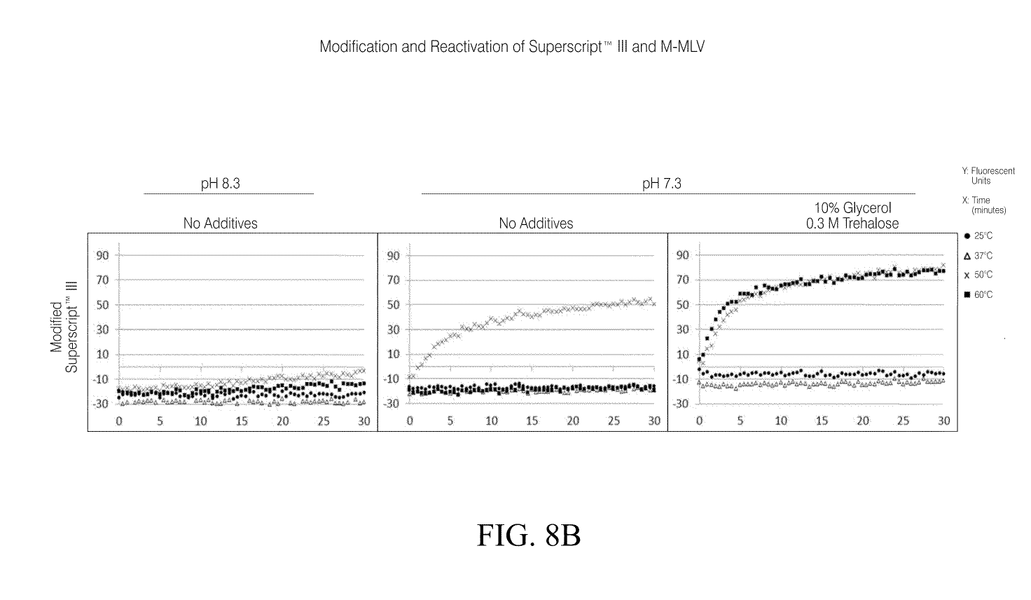

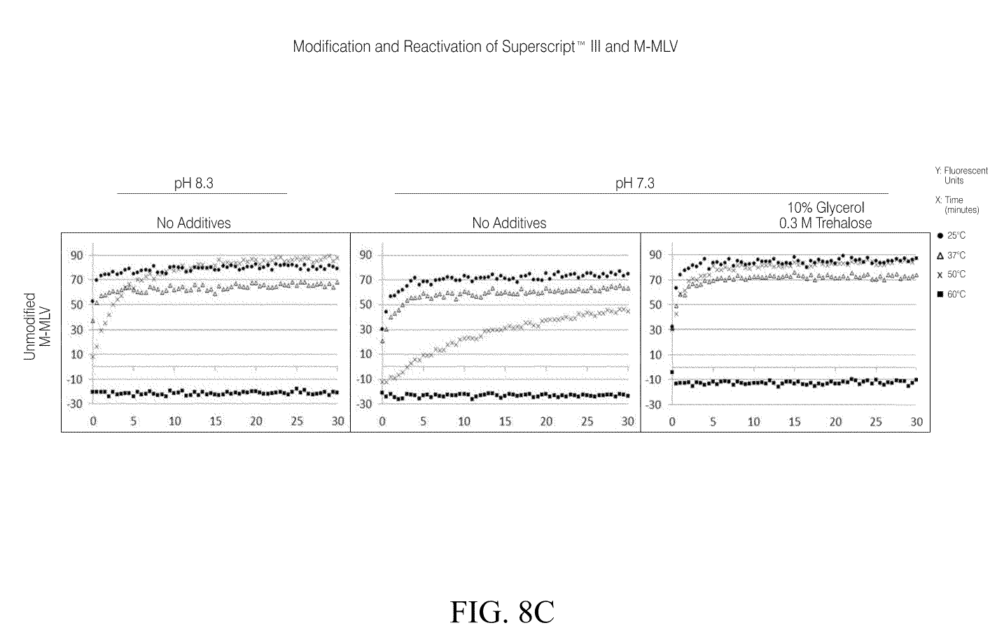

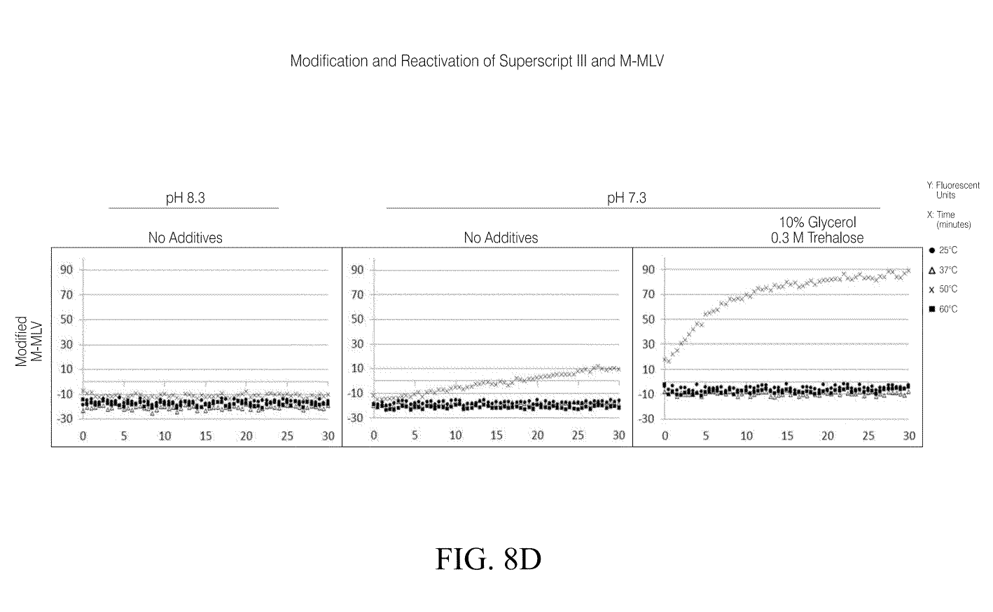

Based on improvements in reactivation of modified enzymes using pH 7.3 buffer and stabilizers, it is likely that reverse transcriptases that are less thermostable can also be hot started. To test this hypothesis, Superscript™ III (Life Technologies) and wild type M-MLV reverse transcriptases were chemically modified as described in Example 1 with the exception that 0.01% NP-40 (Thermo Scientific, Catalog #PI-28324) was used as the detergent and a 200:1 2,3-DMMA:Enzyme ratio was used for Superscript™ III and 400:1 2,3-DMMA:Enzyme ratio was used for M-MLV. Following modification, both modified enzymes were dialyzed into storage buffer composed of 20 mM Tris pH 8.5, 100 mM NaCl, 0.1 mM EDTA, 1 mM DTT, 0.01% NP-40, and 50% glycerol. Enzyme activity and reactivation were tested using the fluorescent assay described in Example 1. Changes include comparing pH 8.3, pH 7.3, and pH 7.3 buffer with the addition of additives (10% glycerol and 0.3 M Trehalose). Unmodified and modified enzymes were preincubated at designated temperatures for 10 minutes. As FIGS. 8A through 8D show, unmodified Superscript™ III was thermostable ≦50° C., but activity was significantly reduced after incubating at 60° C. at pH 8.3 and pH 7.3. The addition of 10% glycerol and 0.3 M trehalose stabilized activity at 60° C. Modified Superscript™ III did not result in reactivation at all temperatures tested when pH 8.3 buffer was used. However, modified Superscript™ III recovered activity after hot start activation at 50° C. with pH 7.3 buffer and at 60° C. when pH 7.3 buffer and stabilizers were utilized. Unmodified M-MLV shows no activity at 60° C. and reduced activity after preincubation at 50° C. at pH 8.3 and pH 7.3. The addition of stabilizers did recover unmodified M-MLV at 50° C., but not at 60° C. Modified M-MLV could not be reactivated using pH 8.3 buffer and showed low, but detectable activation using pH 7.3 buffer at 50° C. The addition of stabilizers to pH 7.3 buffer allowed modified M-MLV to be fully reactivated at 50° C., but not at 60° C. The absence of detectable activity at 25° C. for both modified enzymes is desirable for hot start properties. The challenges these enzymes have at ≧50° C. with or without stabilizers are due to their non-thermostable properties compared to Maxima™ H Minus and Mut MMLV. Nonetheless, reactivation using pH 7.3 buffer shortened preincubation time and the addition of stabilizers allowed both of these modified enzymes to be reactivated at temperatures higher than their recommended reaction temperatures, 50° C. for Superscript™ III and 37° C. for wild type M-MLV (Product Manual, Invitrogen™ Catalog #s 18080-04 and 28025-013, respectively).

### Example 7

### Molar Ratio Range of 2,3-DMMA to Thermostable Reverse Transcriptase for Chemical Modification

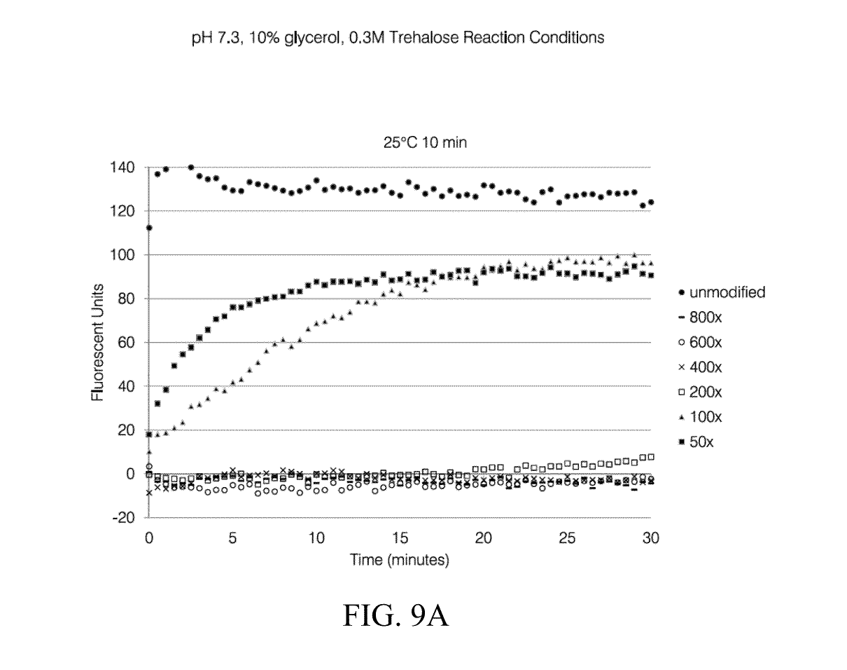

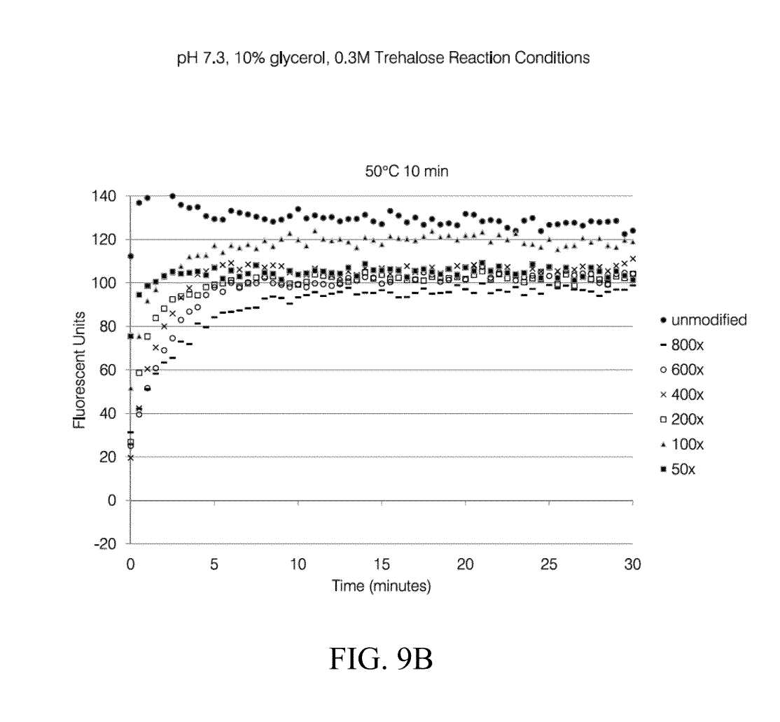

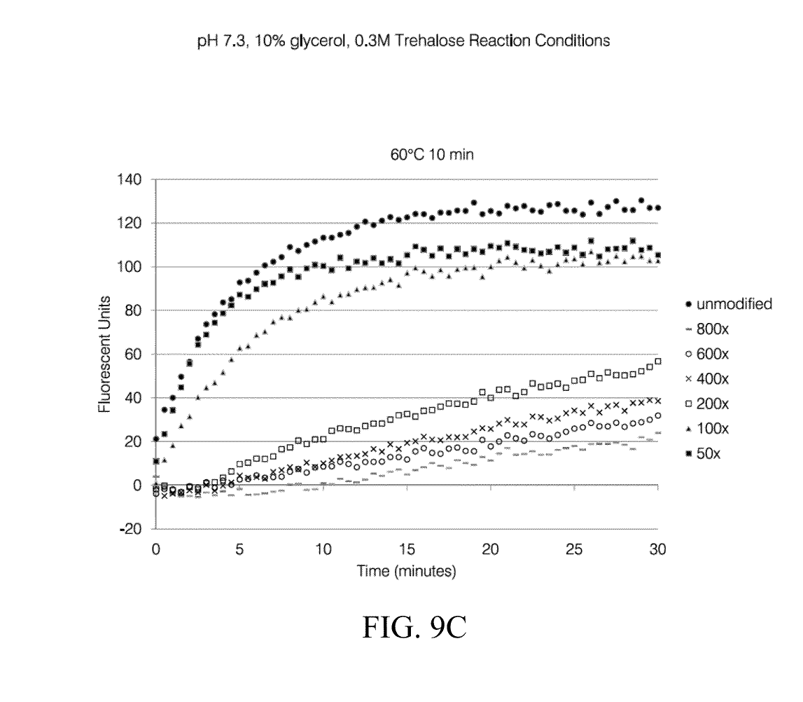

50:1-800:1 2,3-DMMA:enzyme ratios described in Example 1 were again tested using the fluorescent assay as described in Example 1, but at pH 7.3 and in the presence of 10% glycerol and 0.3 M trehalose. As shown in FIGS. 9A, 9B and 9C, under these activating conditions, enzymes modified using each of the various 2,3-DMMA concentrations were reactivated a both 50° C. and 60° C. contrary to assays run at pH 8.3 and without stabilizers, as demonstrated in FIGS. 2A, 2B, and 2C.

### Example 8

### Dialysis of Modified Reverse Transcriptase Enzymes Improves Reactivation

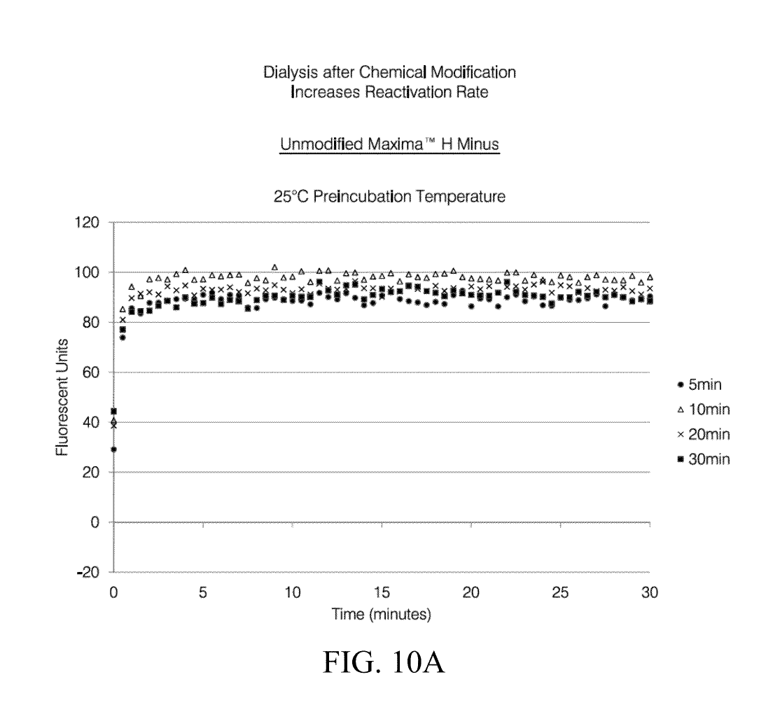

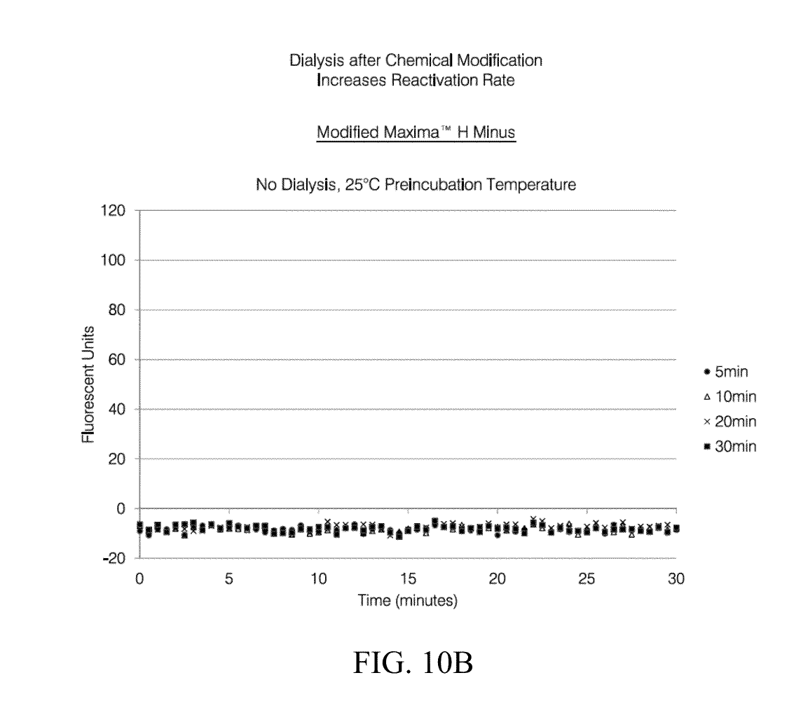

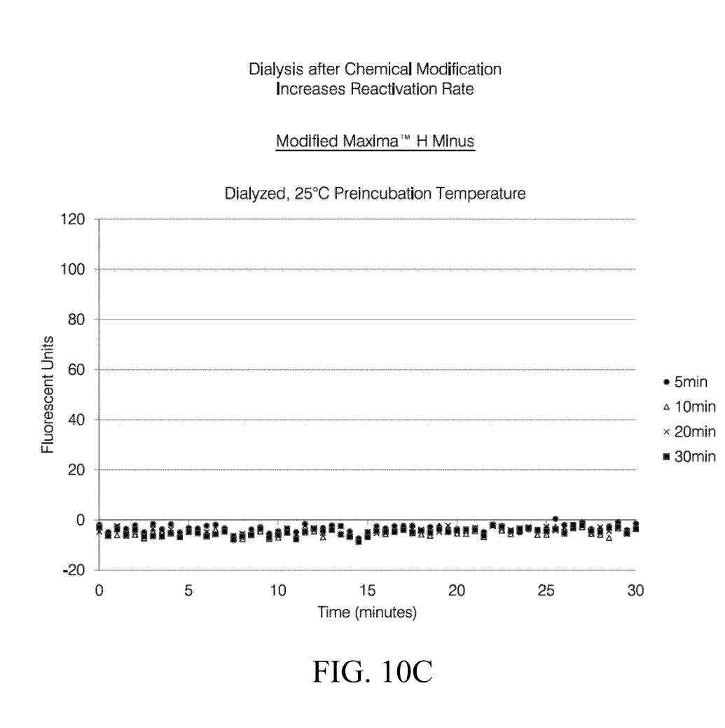

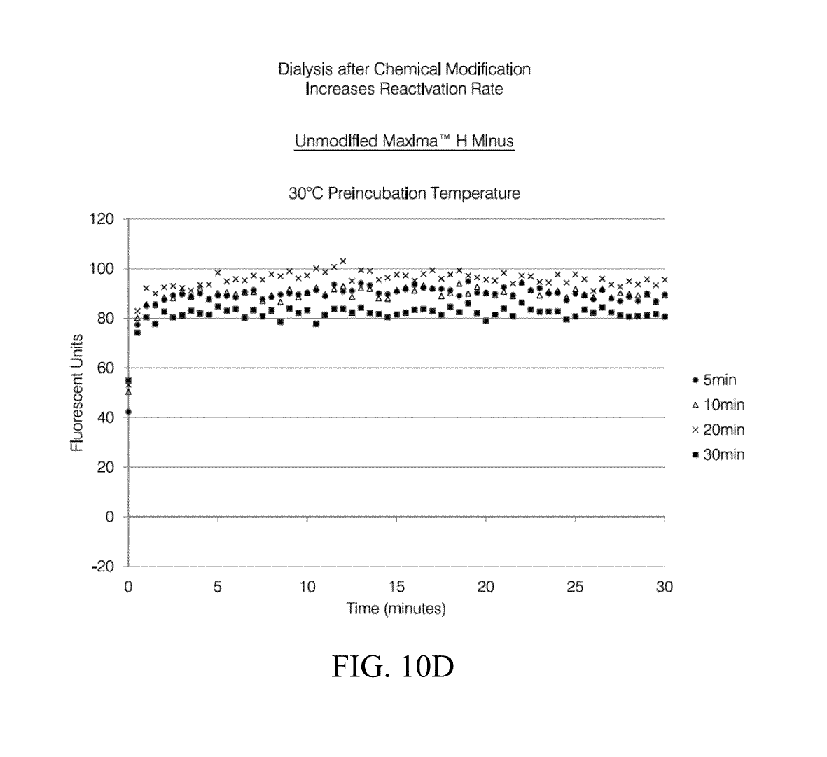

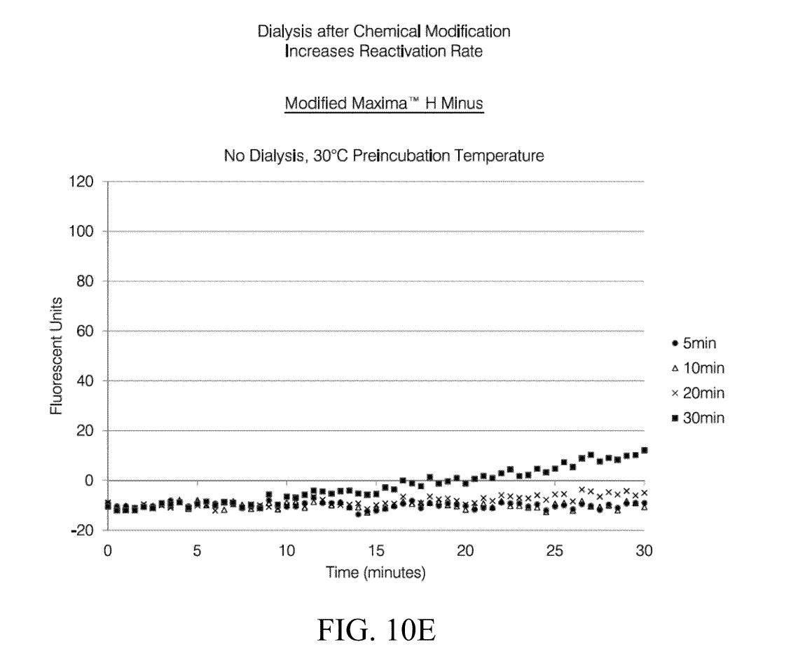

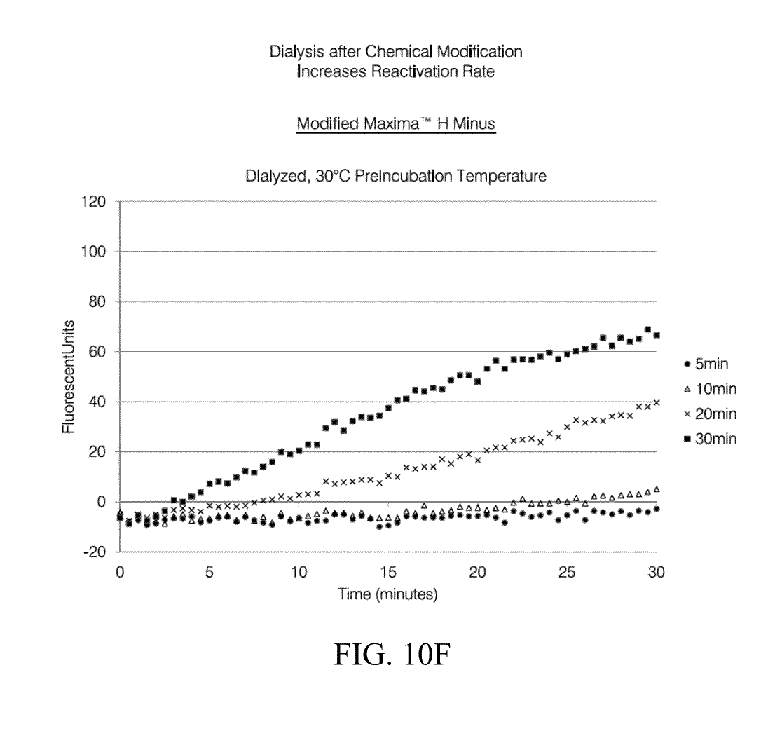

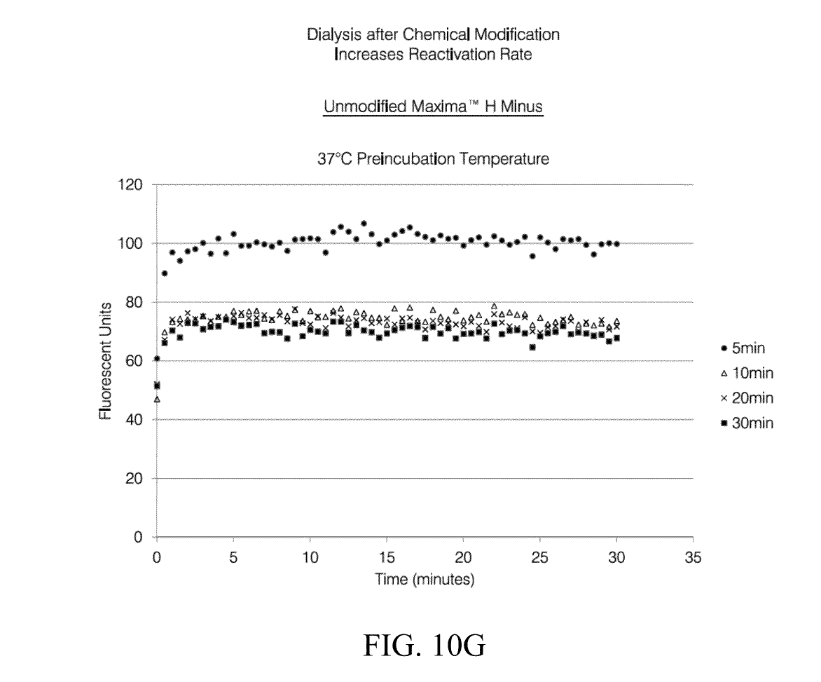

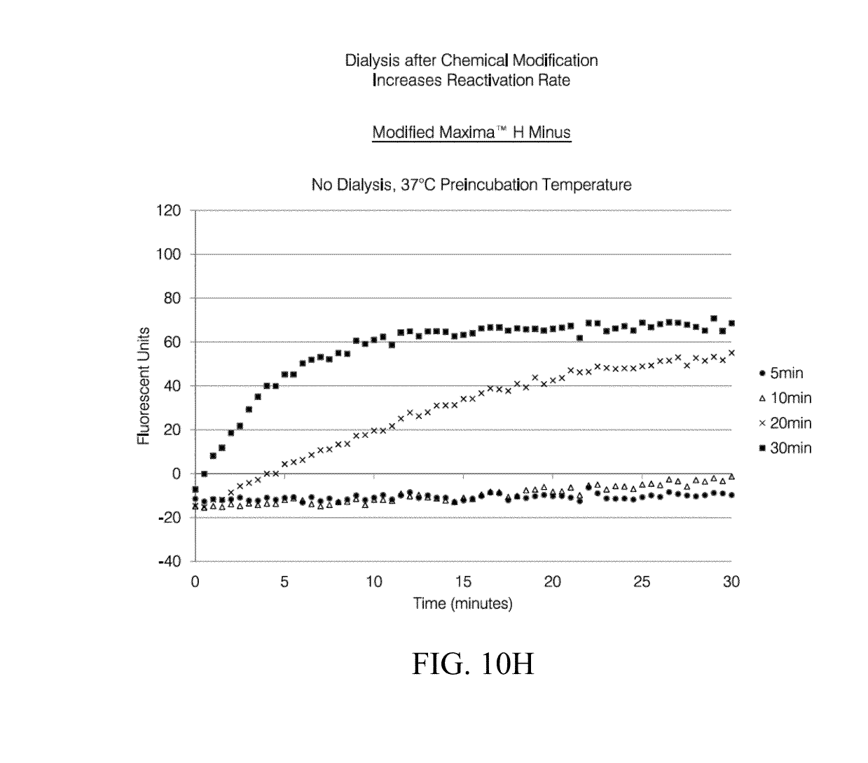

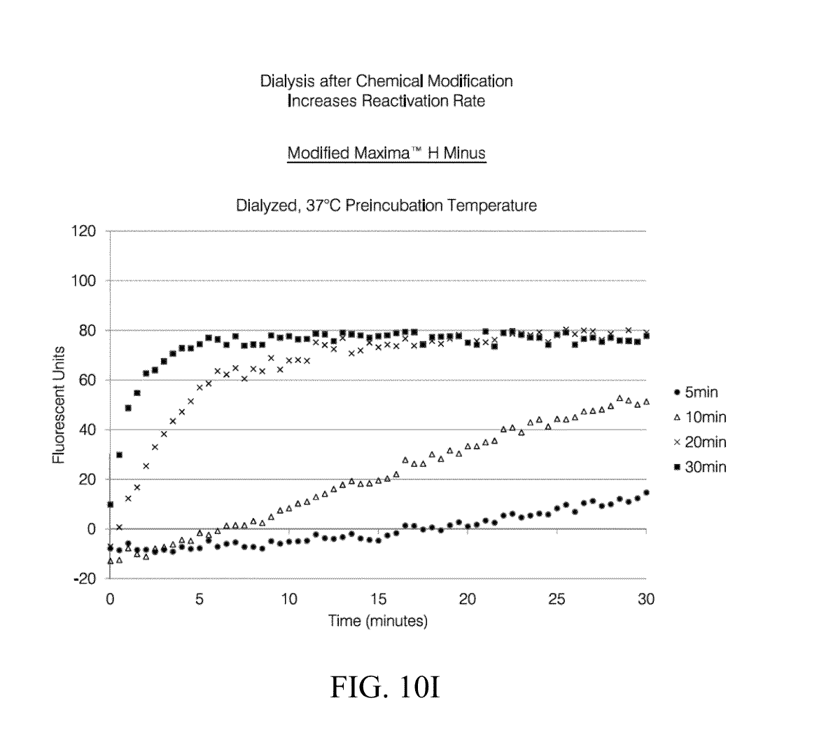

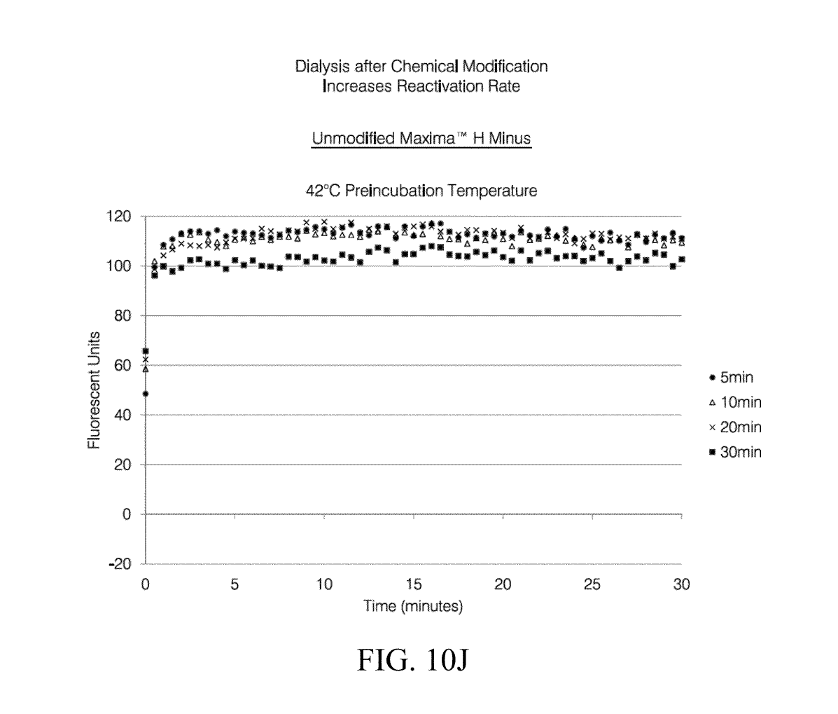

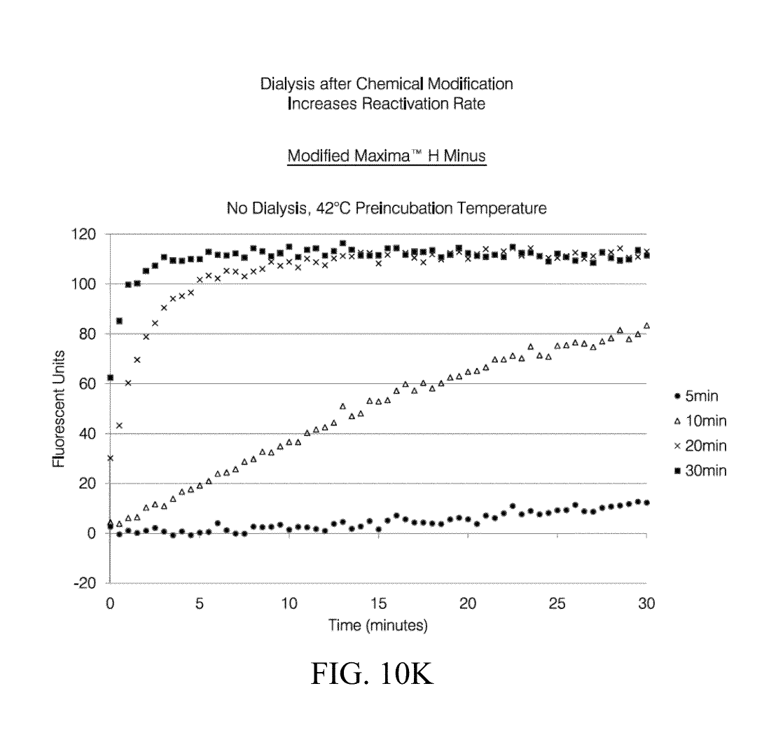

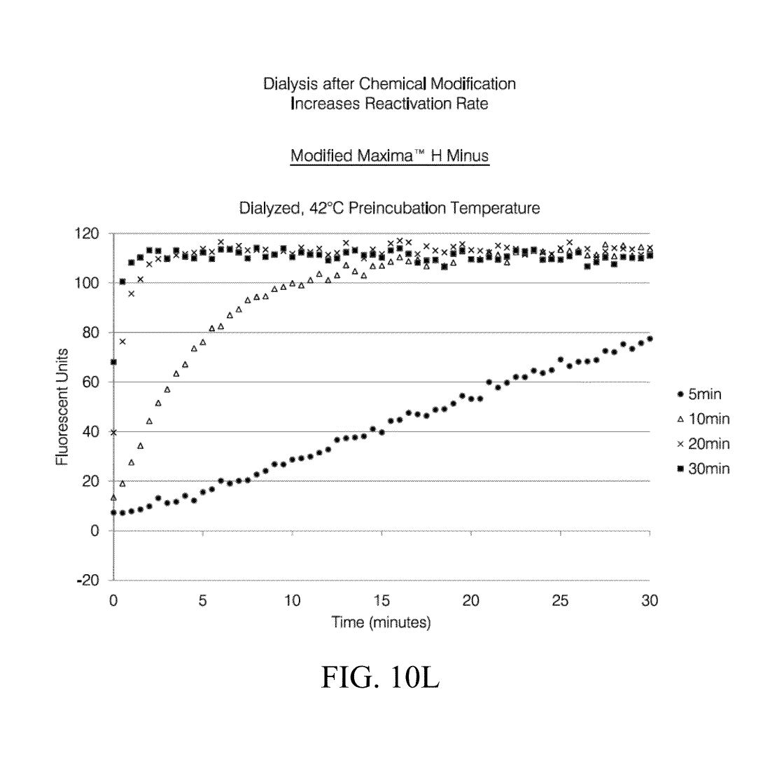

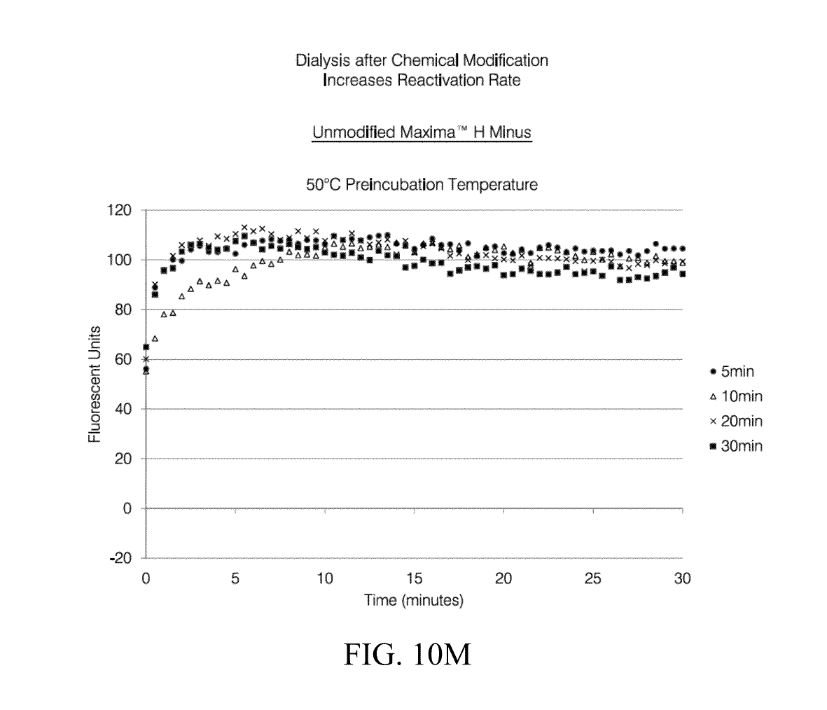

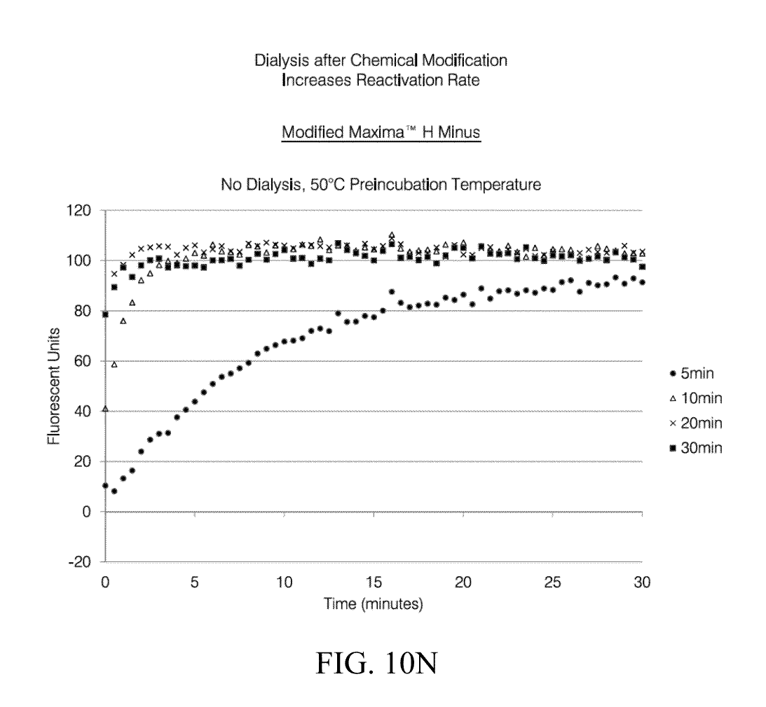

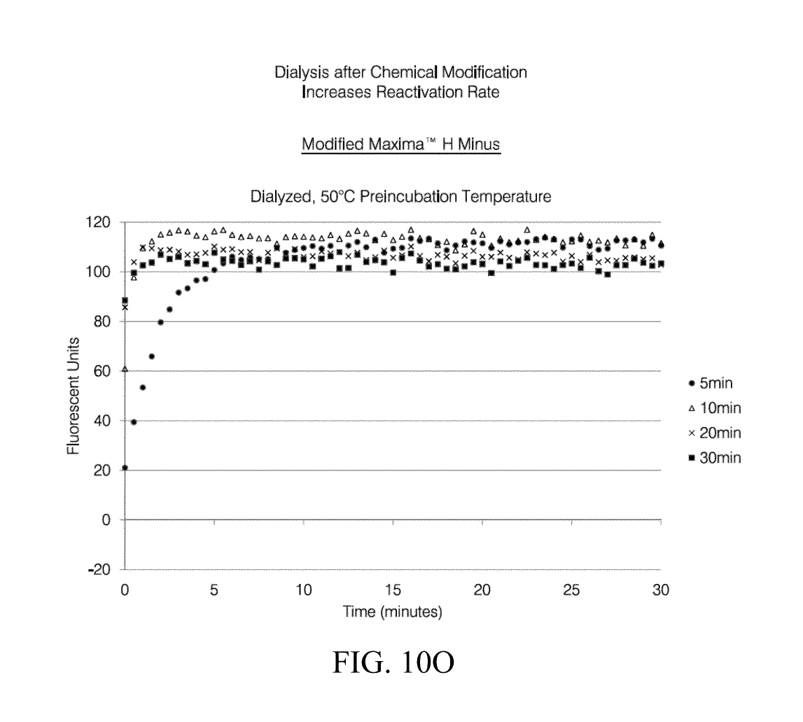

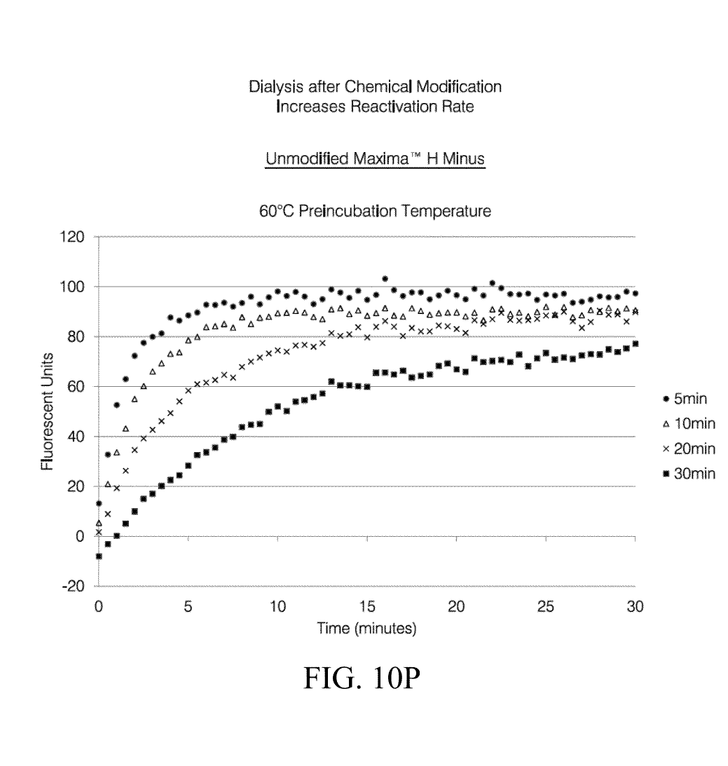

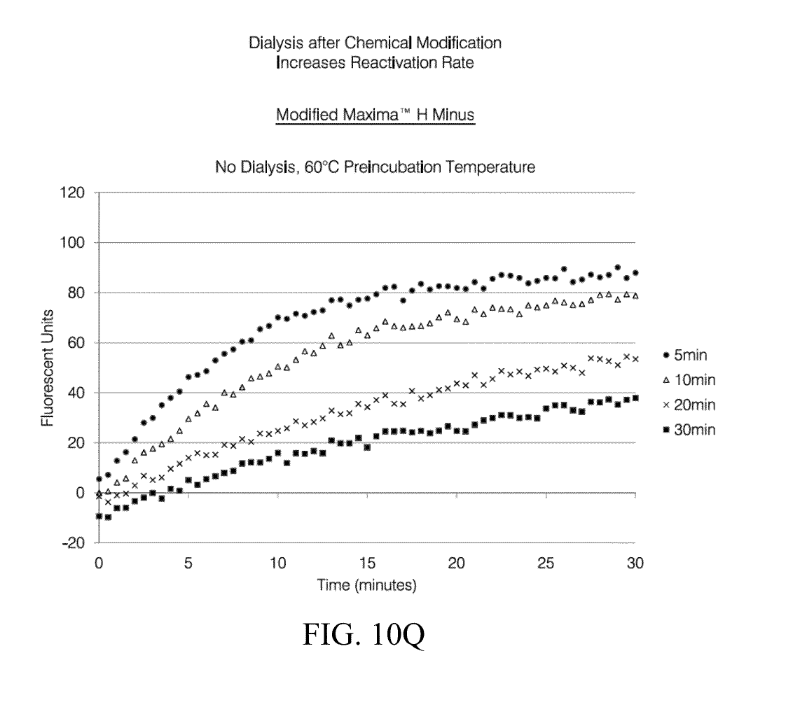

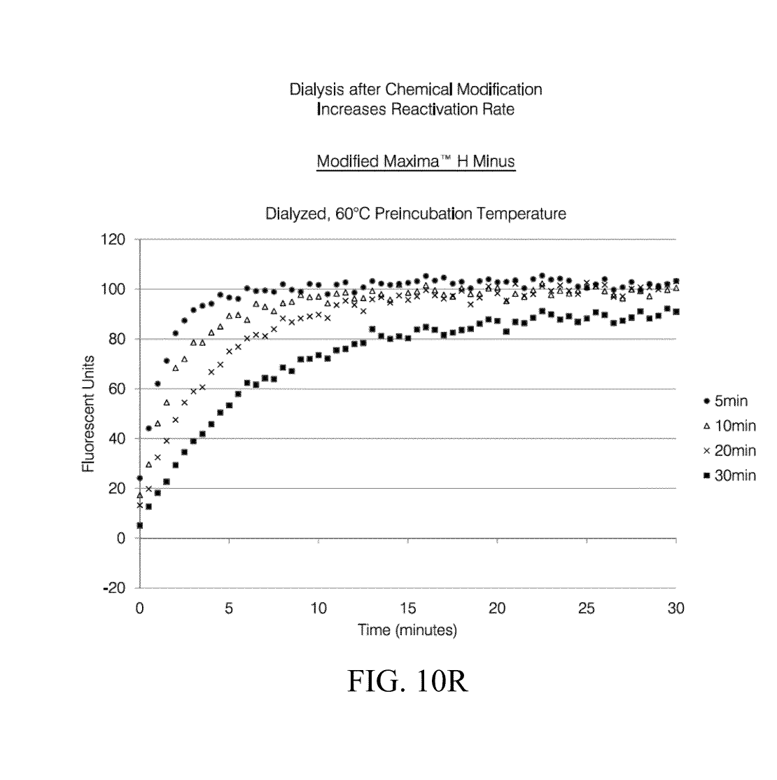

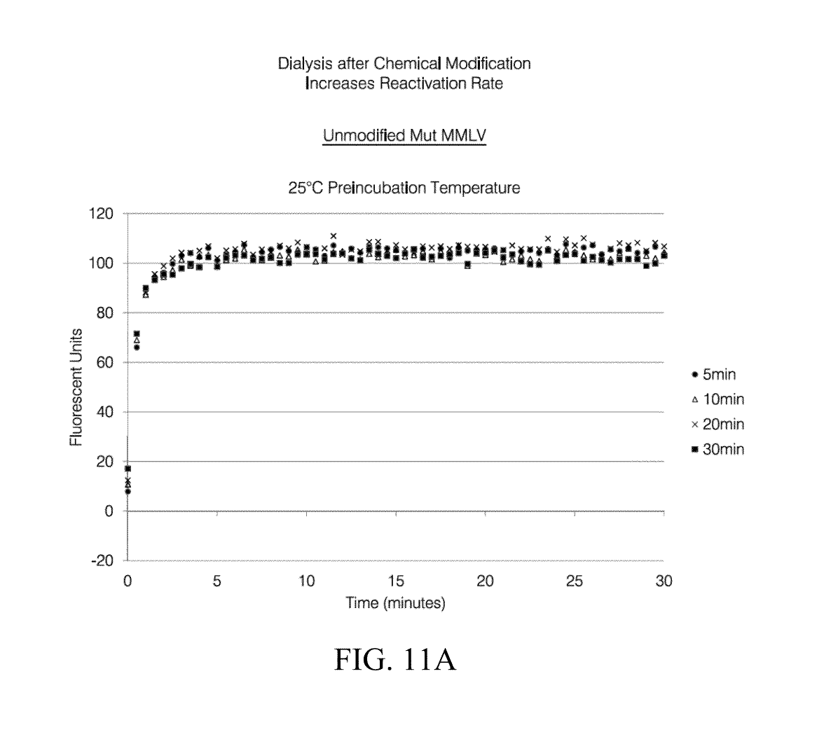

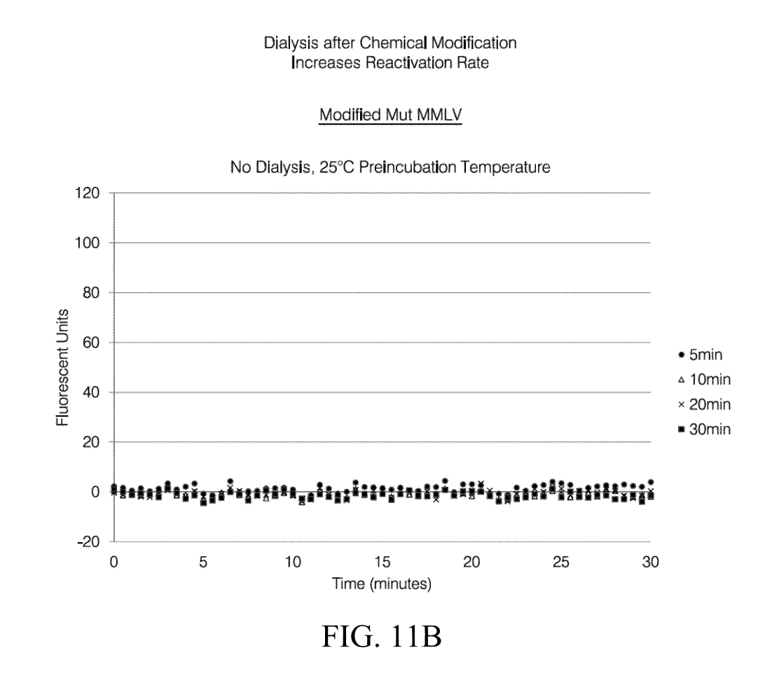

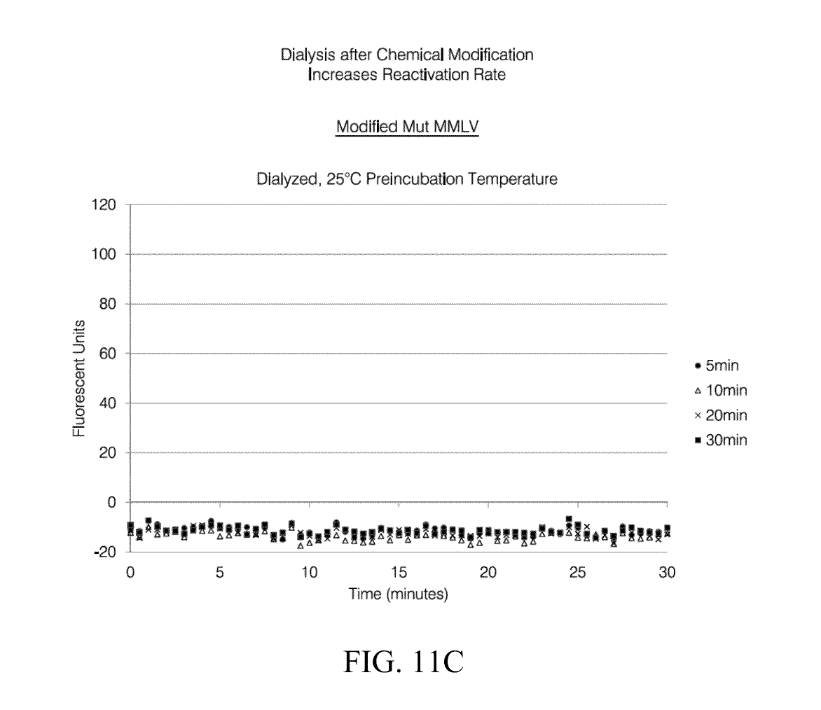

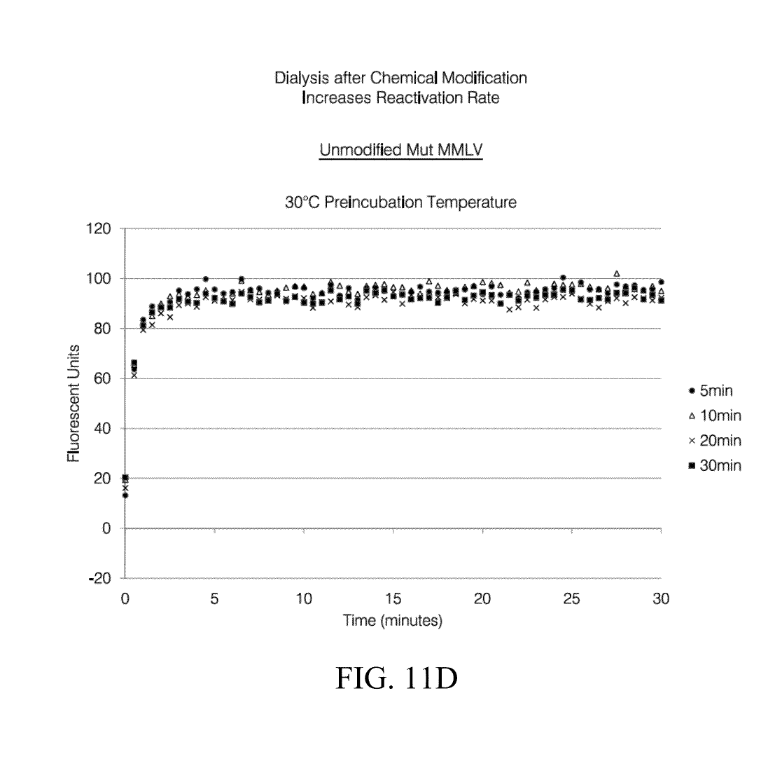

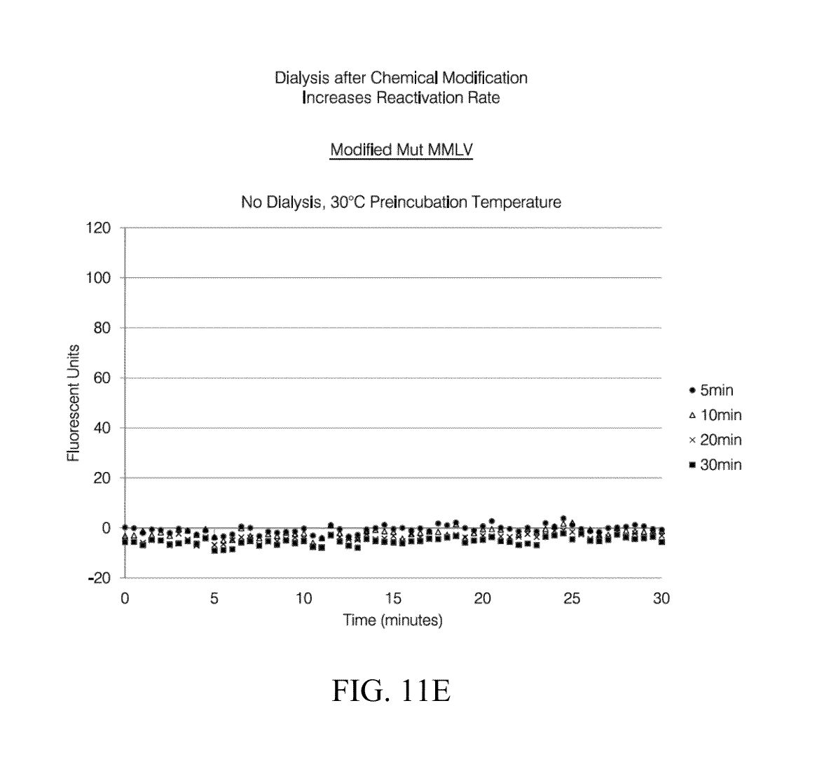

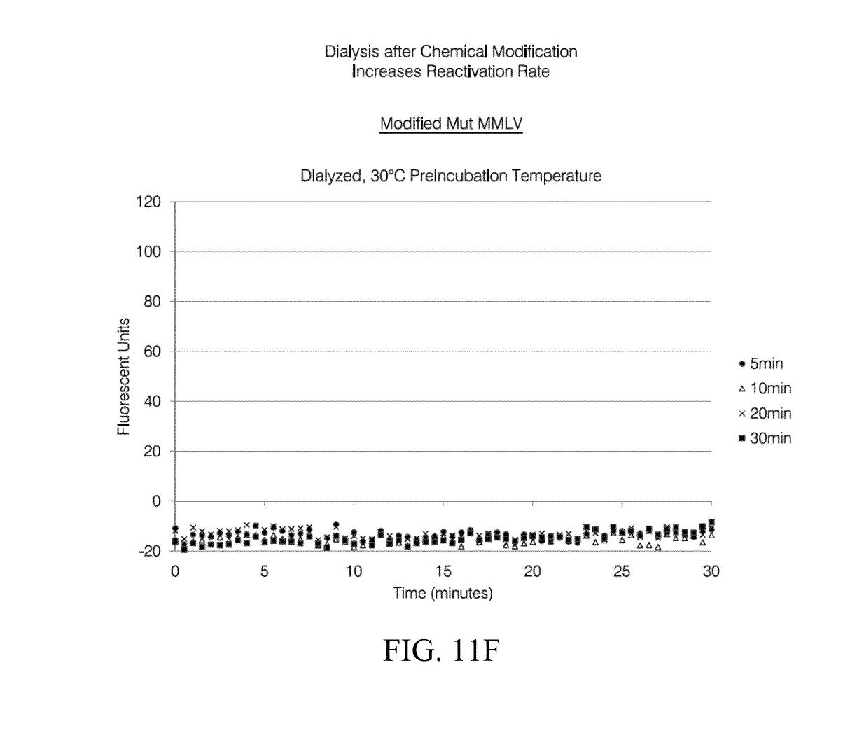

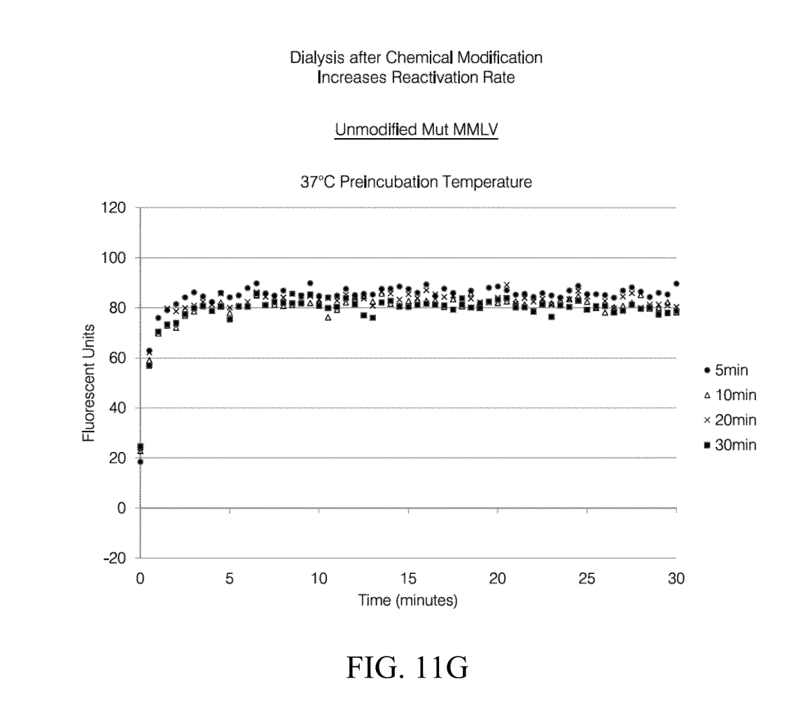

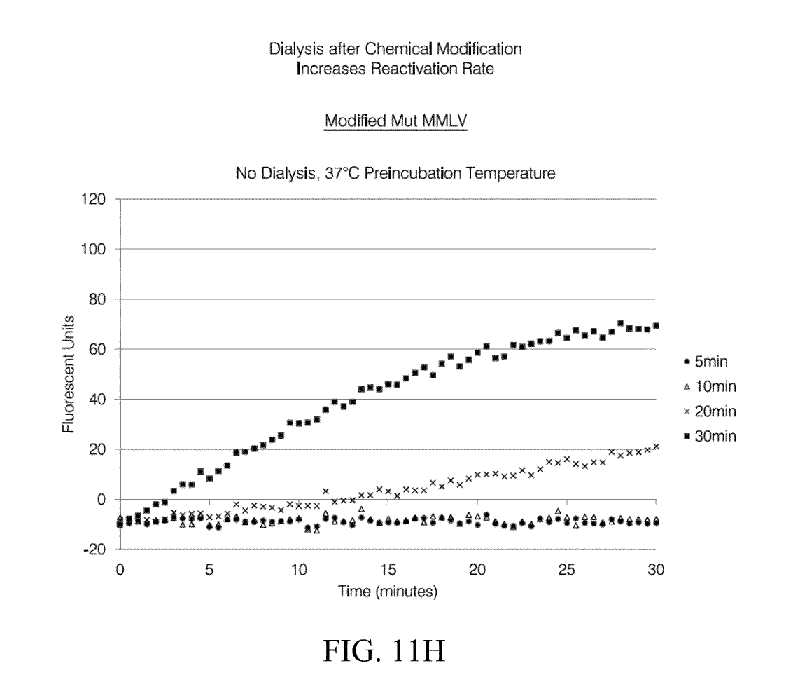

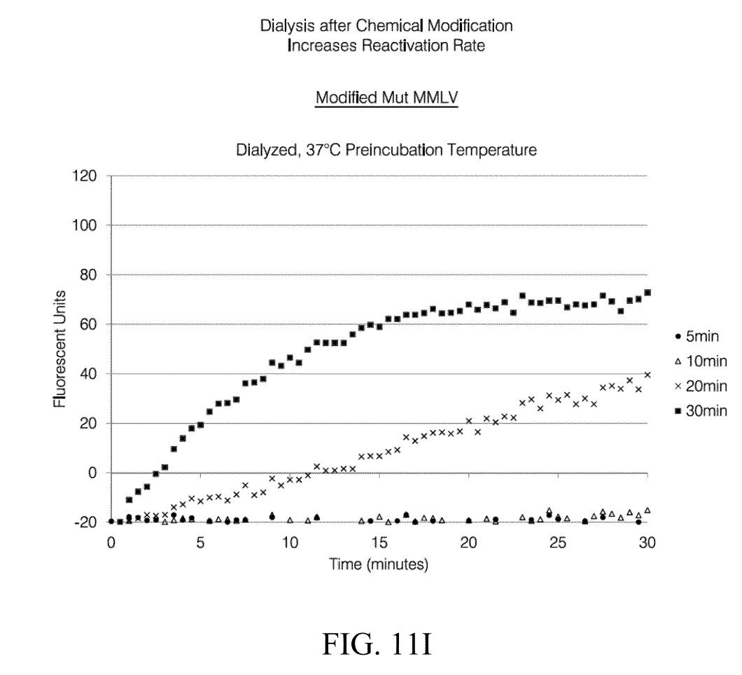

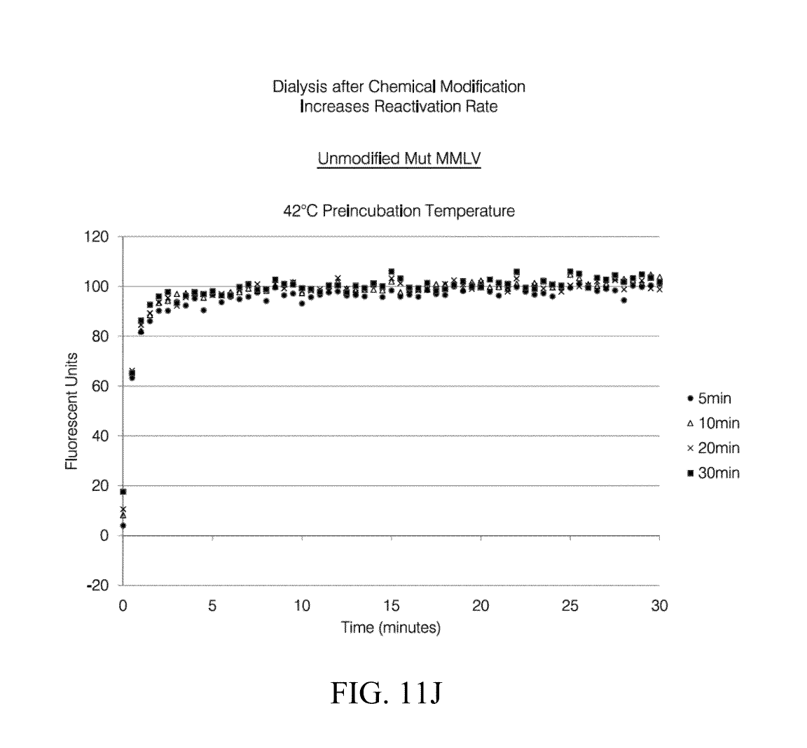

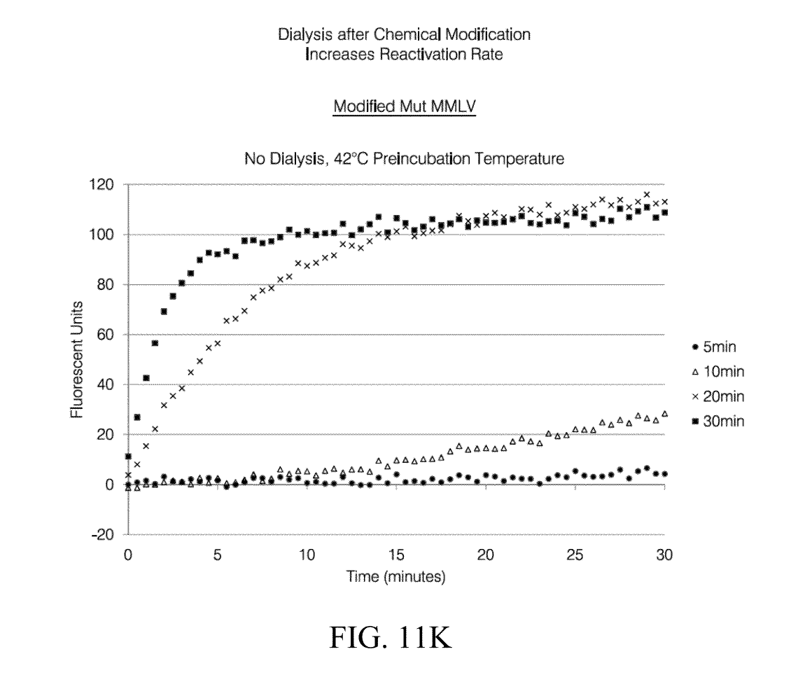

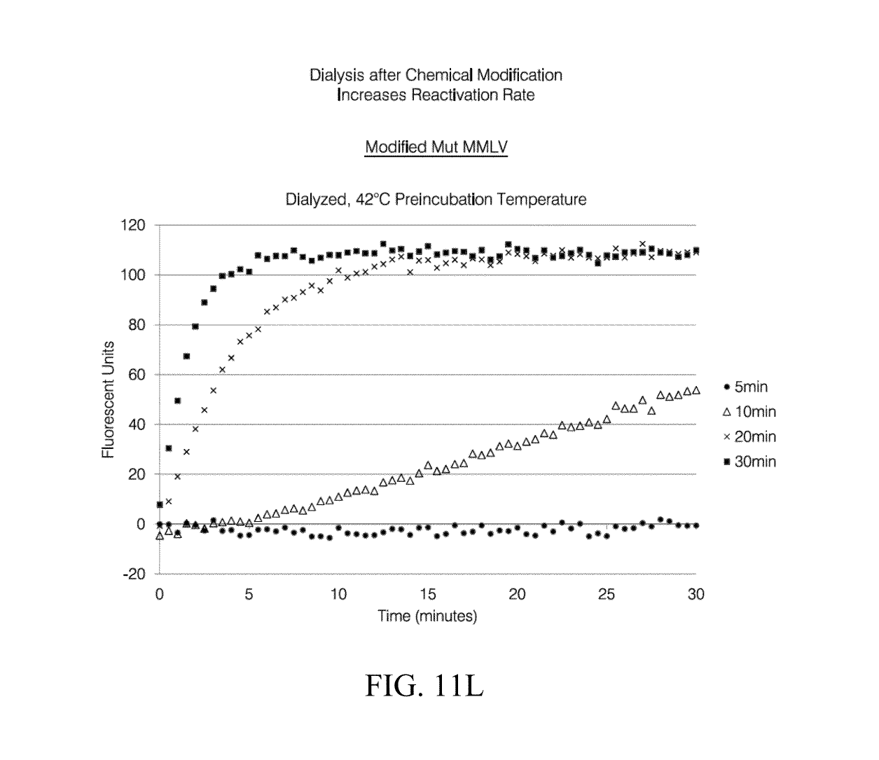

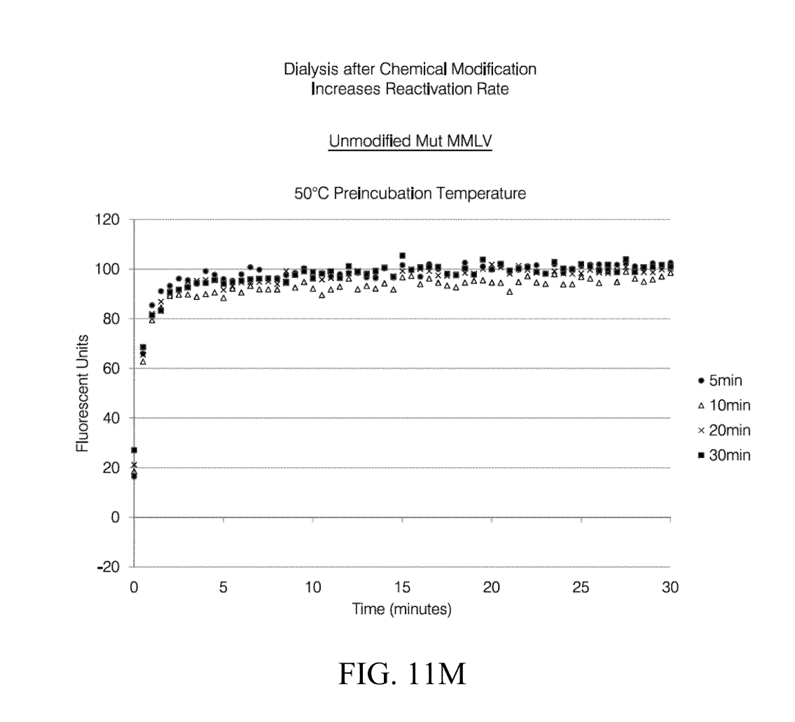

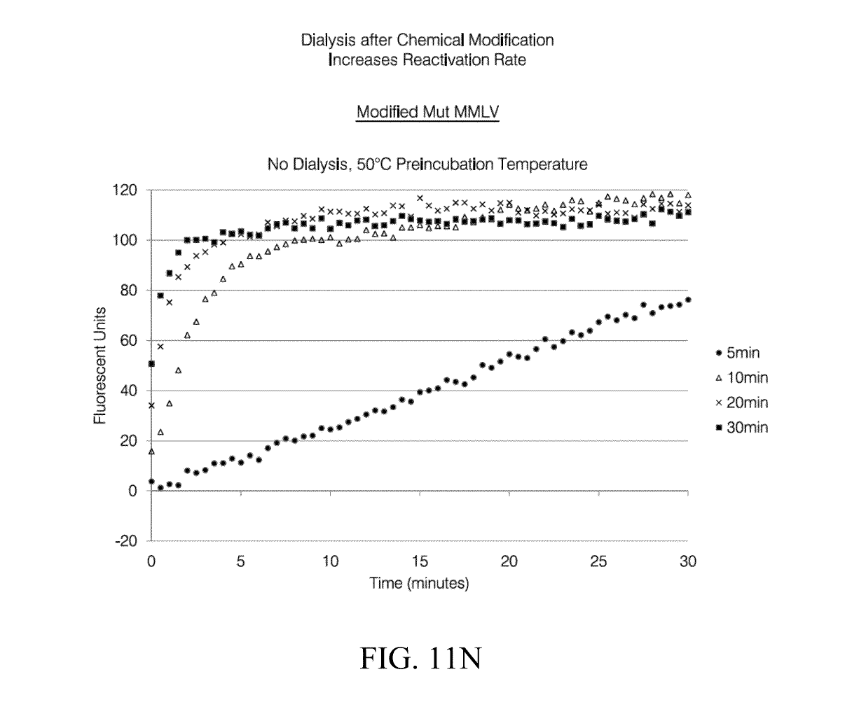

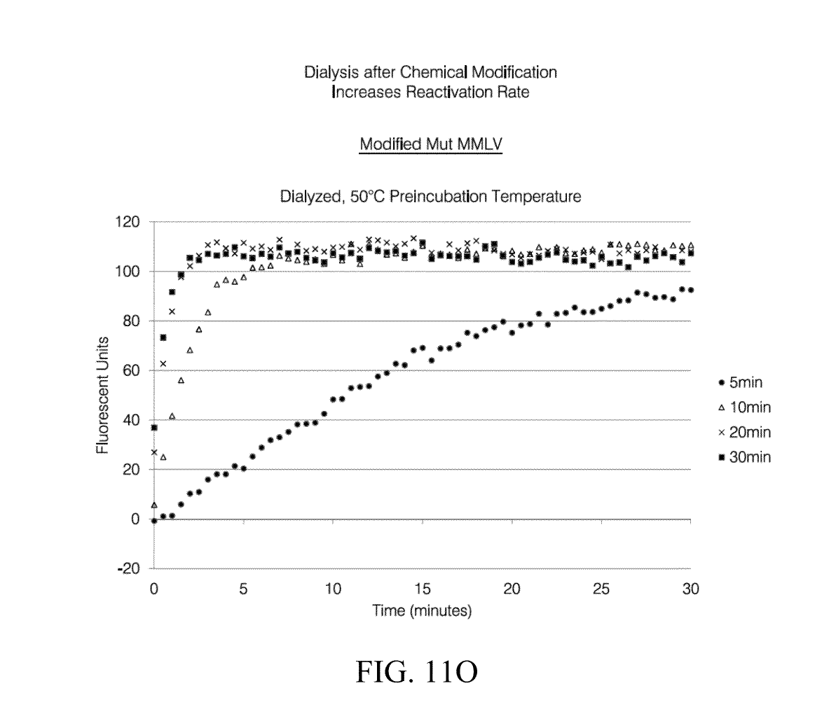

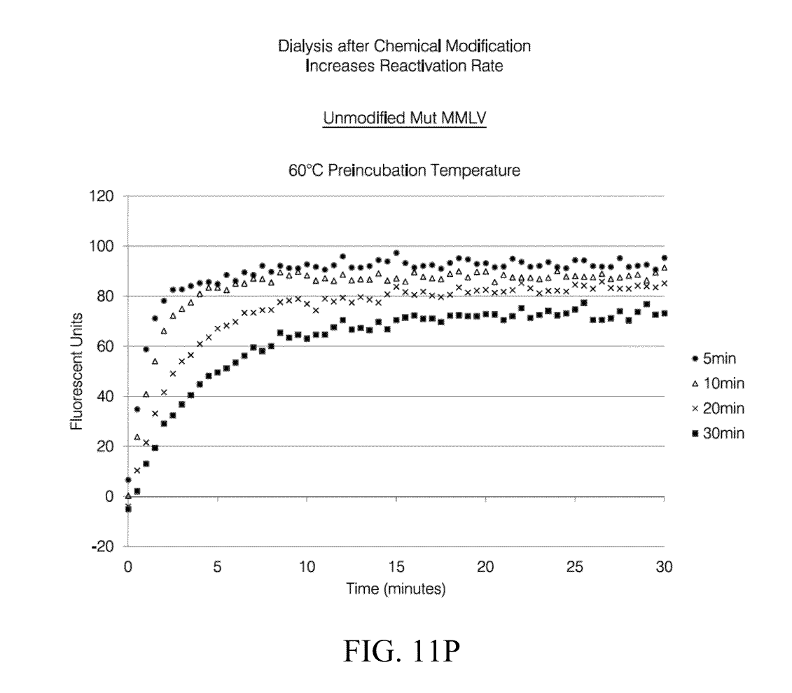

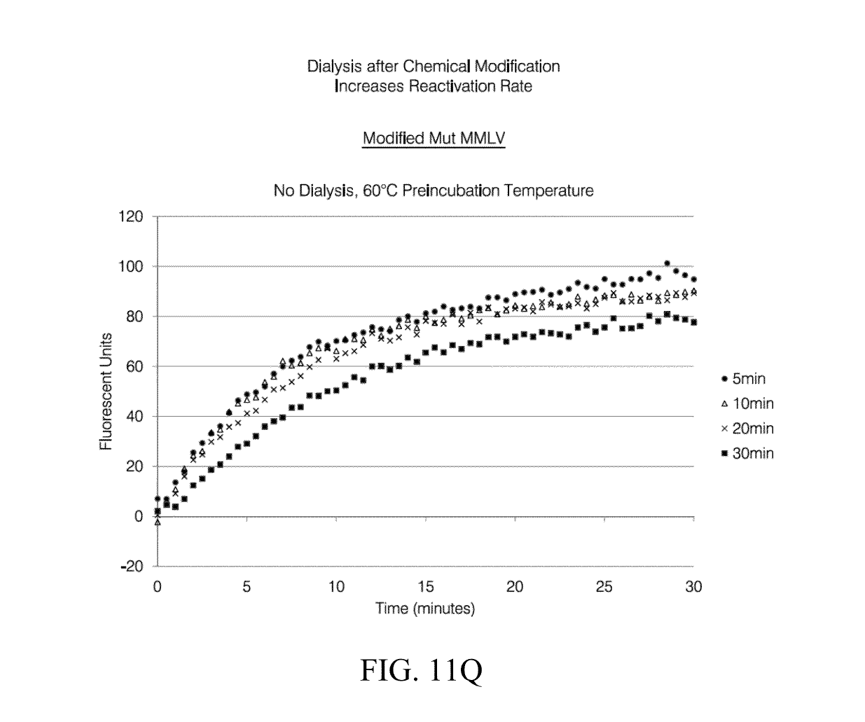

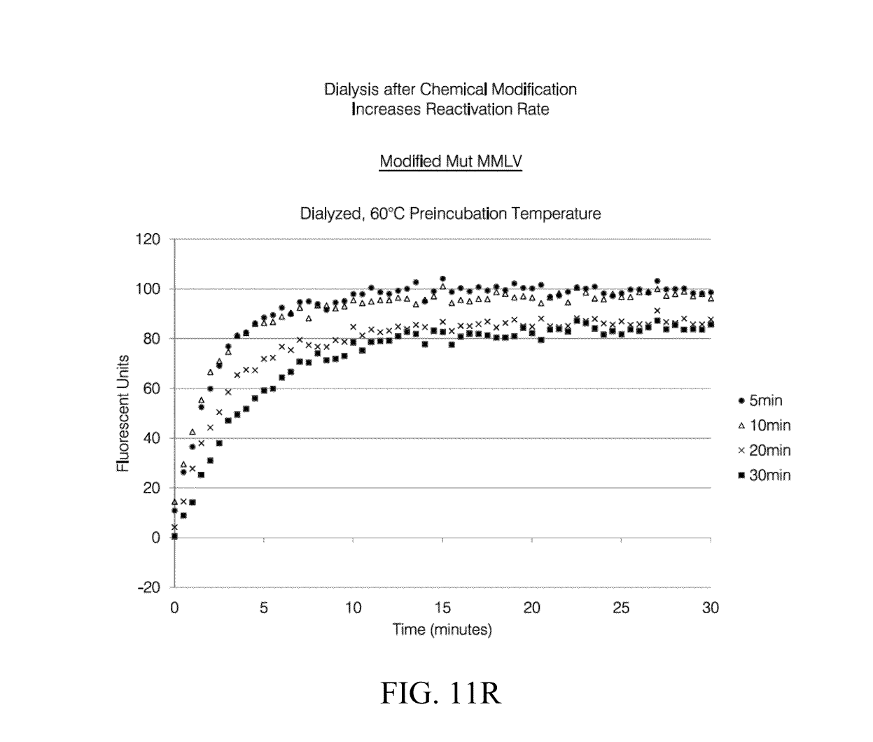

To prevent chemical modification from continuing when the hot start enzymes were not subsequently frozen, modified enzymes were immediately dialyzed overnight in a minimum of 200-fold excess volume:volume ratio of dialysis buffer:modified enzymes. The dialysis buffer for Maxima™ H Minus reverse transcriptase was composed of 50 mM Tris pH 8.5, 100 mM NaCl, 1 mM EDTA, 5 mM DTT, 0.1% Triton-X-100, and 50% glycerol. After modification, Mut MMLV was dialyzed into 20 mM Tris pH 8.5, 100 mM NaCl, 0.1 mM EDTA, 1 mM DTT, 0.05% Tween 20, and 50% glycerol. To compare whether dialysis affects the reactivation of reverse transcriptases, a 300:1 2,3-DMMA:enzyme modification was performed as described in Example 1 for Maxima™ H Minus and Example 2 for Mut MMLV. Half of the modified enzyme preparations were immediately frozen at −20° C. and the other half was immediately dialyzed overnight in dialysis buffer equilibrated to 4° C. Fluorescent assays were performed as described in Example 1, but at pH 7.3 and with 10% glycerol and 0.3 M trehalose. As shown in FIGS. 10A through 10R and FIGS. 11A through 11R, all reactivation temperatures ranging from 30° C. to 60° C. and for all preincubation times used, dialyzed modified enzymes resulted in much faster activation than enzymes subsequently frozen in modification buffer (composition listed in Example 1 for Maxima H Minus and Example 2 for Mut MMLV).

### Example 9

### Use of Other Stabilizing Components During Reactivation of Modified Reverse Transcriptases

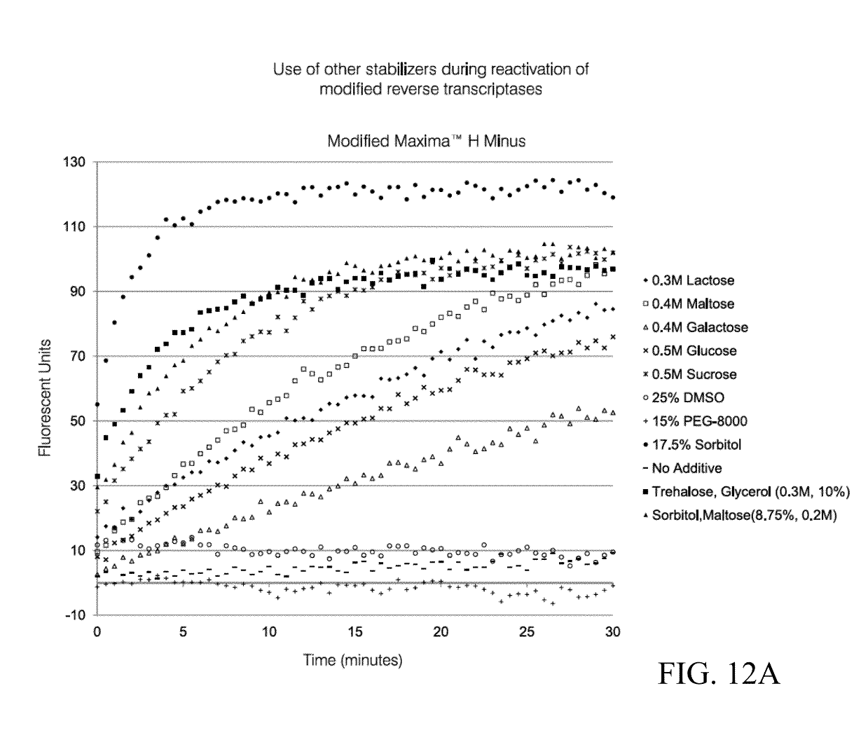

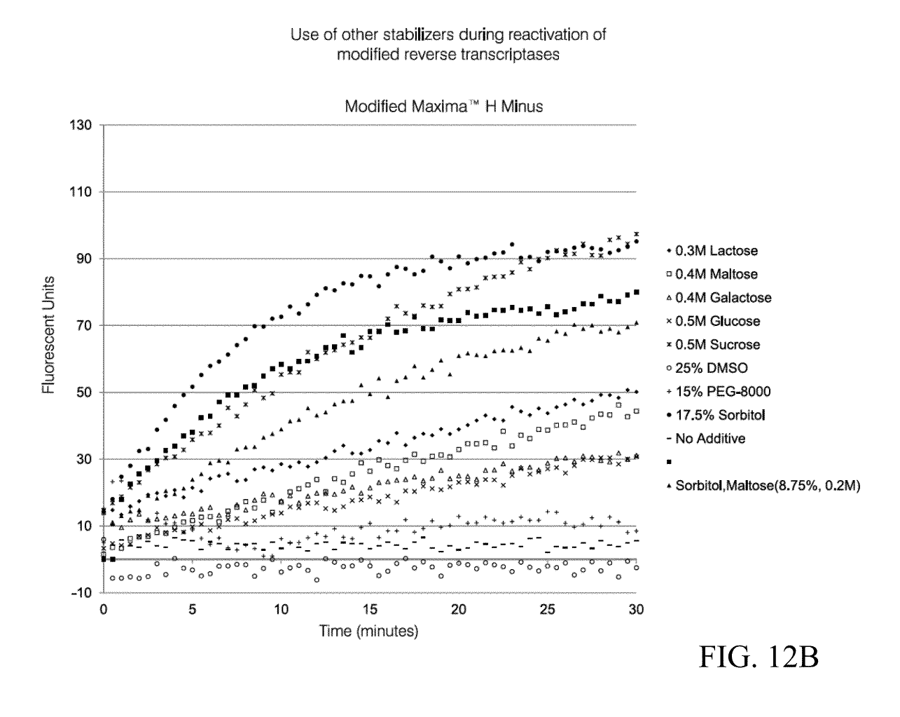

Additives other than glycerol and trehalose were tested to determine if they sustained reactivation of modified enzymes at temperatures higher than 50° C. Additives that were tested included lactose, maltose, galactose, glucose, sucrose, dimethyl sulfoxide (DMSO), polyethylene glycol (average molecular weight 8,000), and sorbitol. Experiments were performed as in Example 1, using a reaction buffer at pH 7.3 instead of 8.3. The modified enzyme mixtures were preincubated at 60° C. for 10 minutes prior to addition of substrate components and fluorescent readings. FIGS. 12A and 12B show that all additives tested stabilized modified enzymes with the exception of DMSO and polyethylene glycol.

### Example 10

### pH Influence on Reactivation of Modified RTs in the Presence of Stabilizers

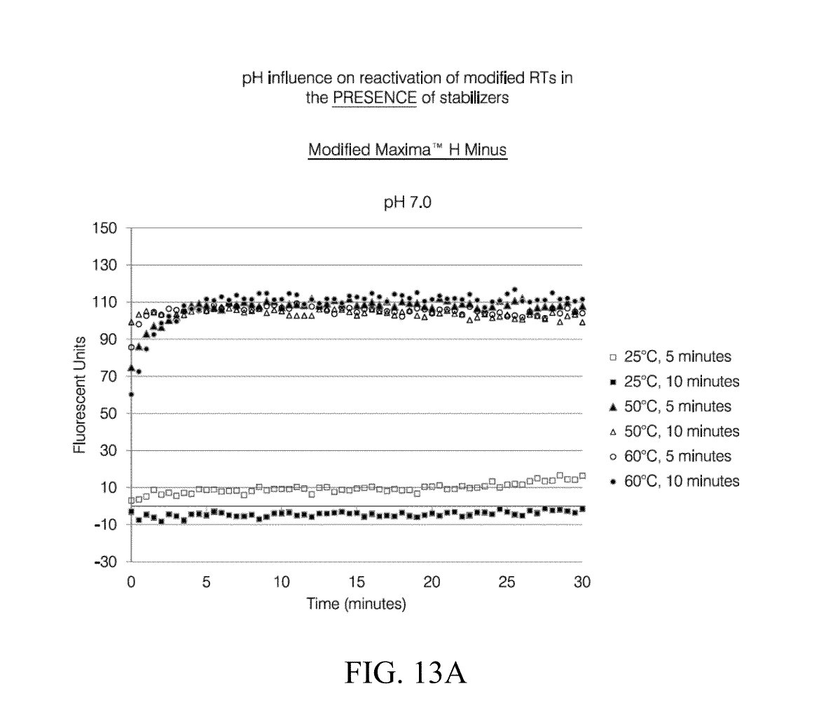

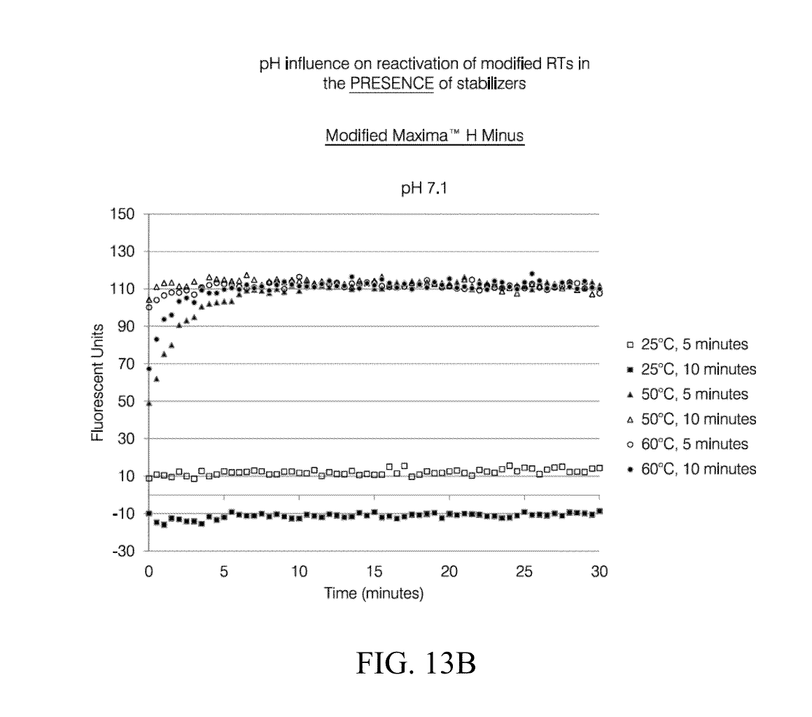

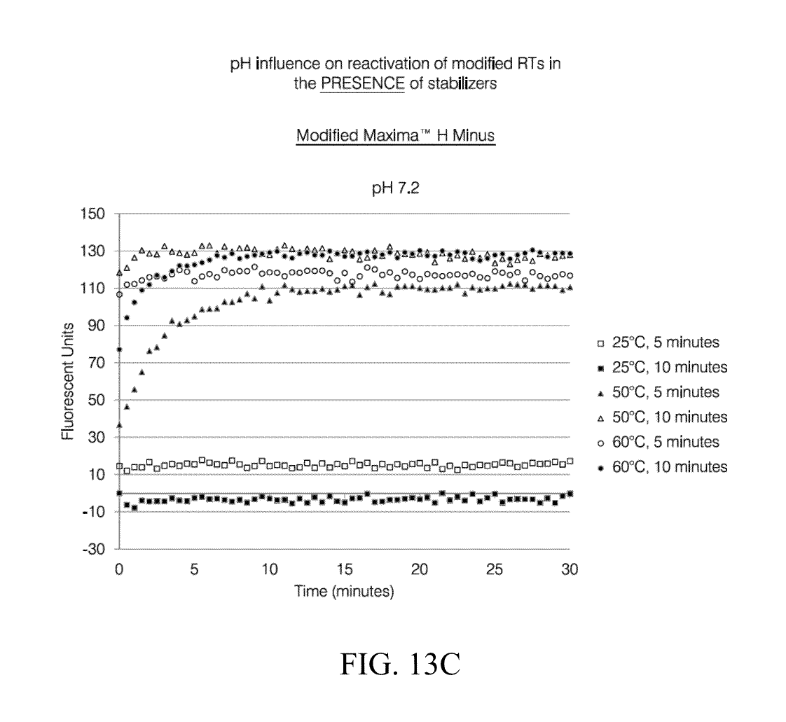

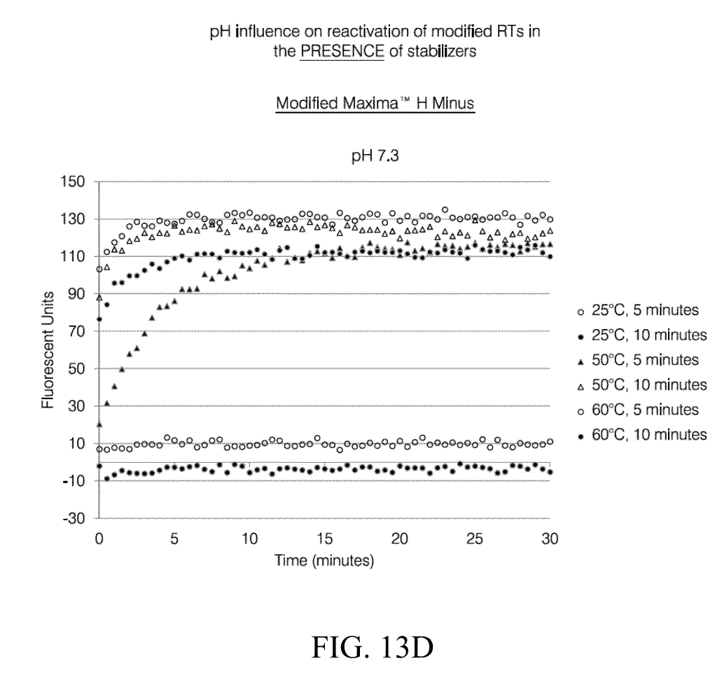

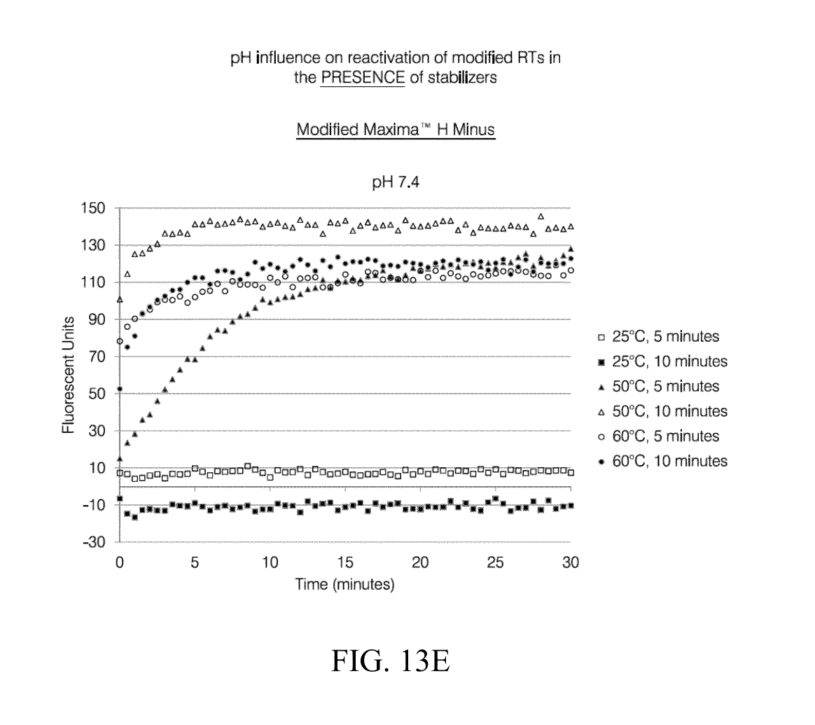

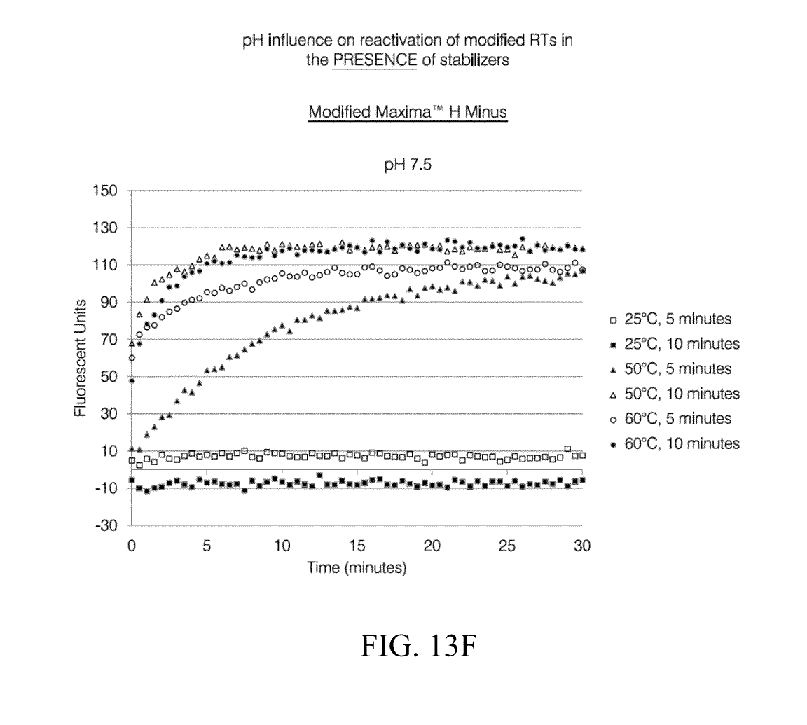

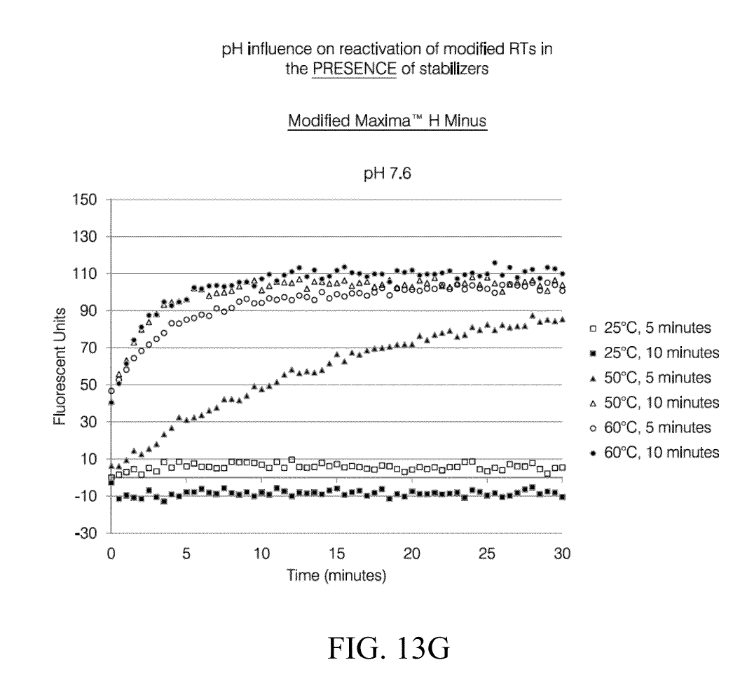

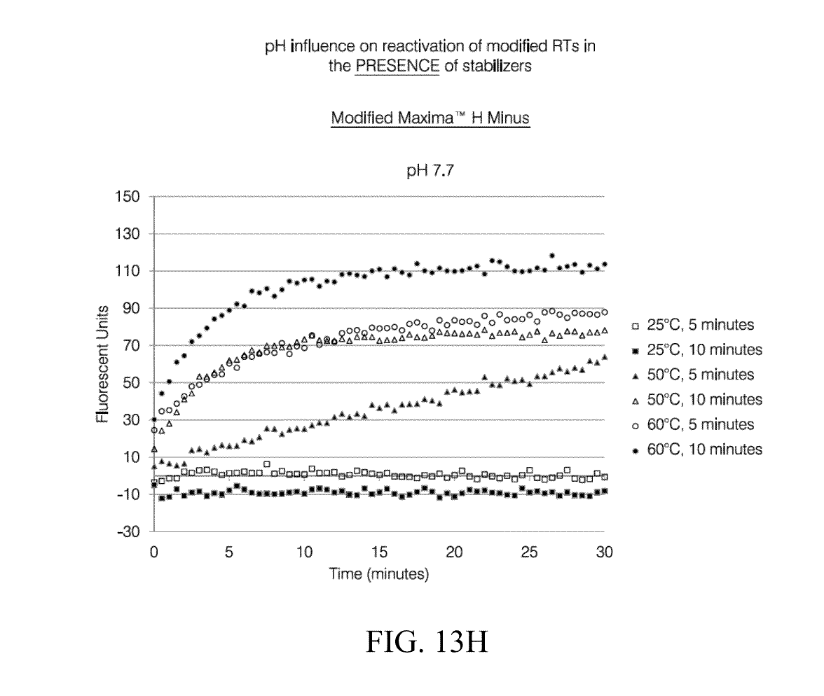

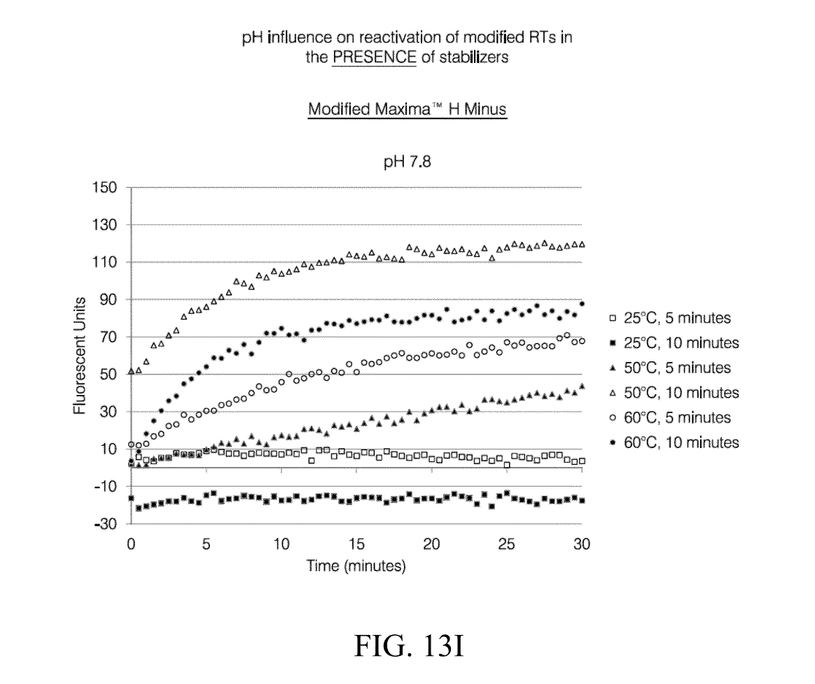

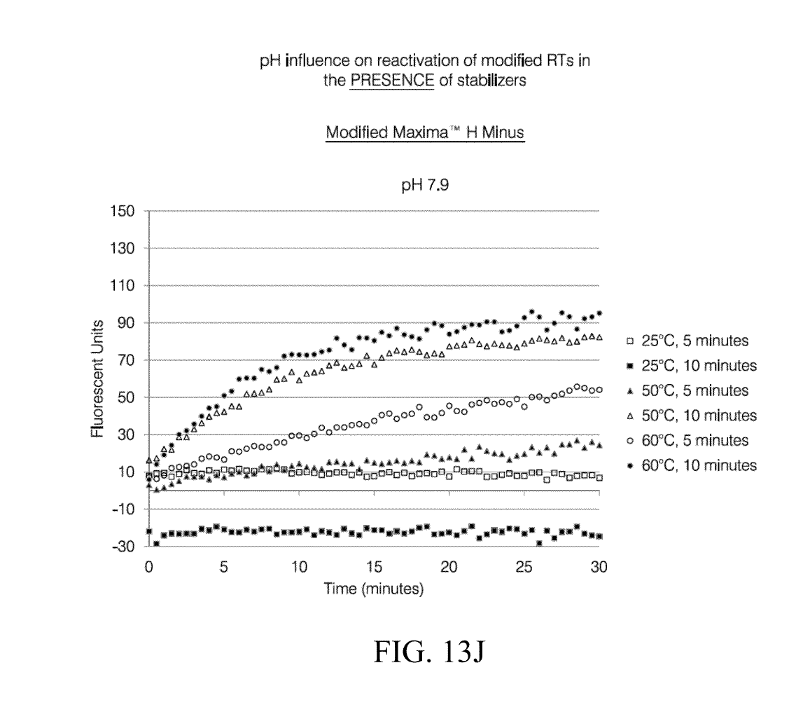

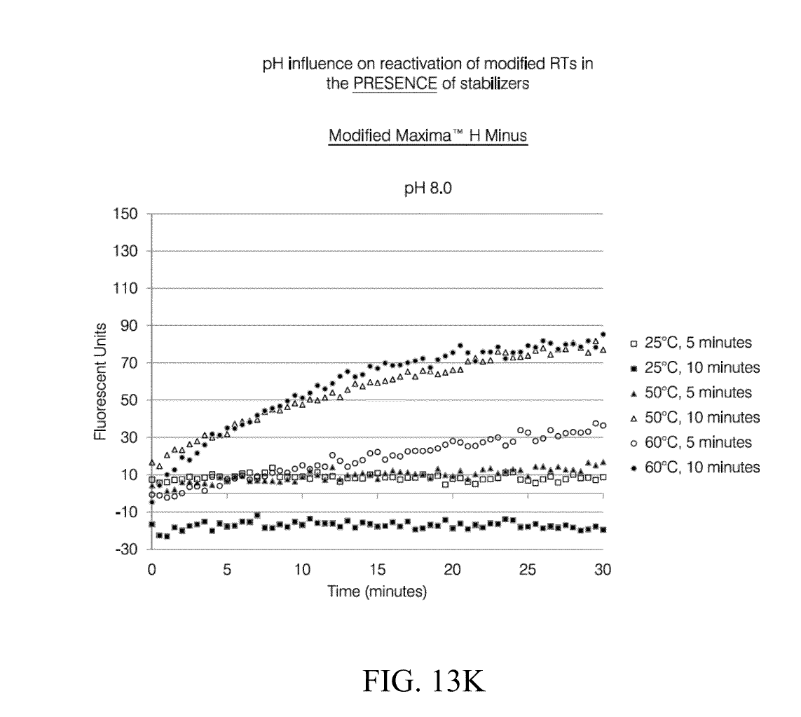

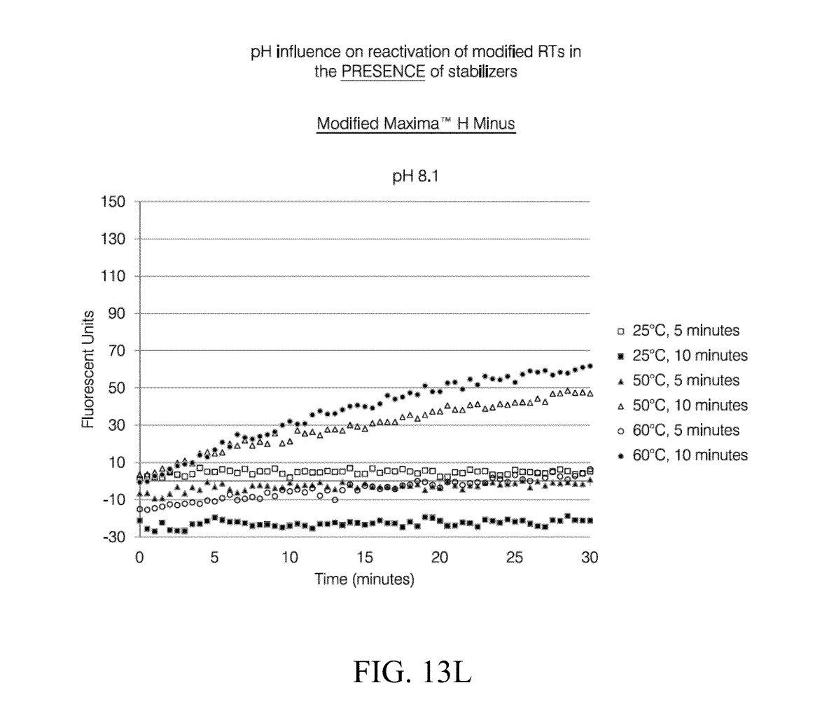

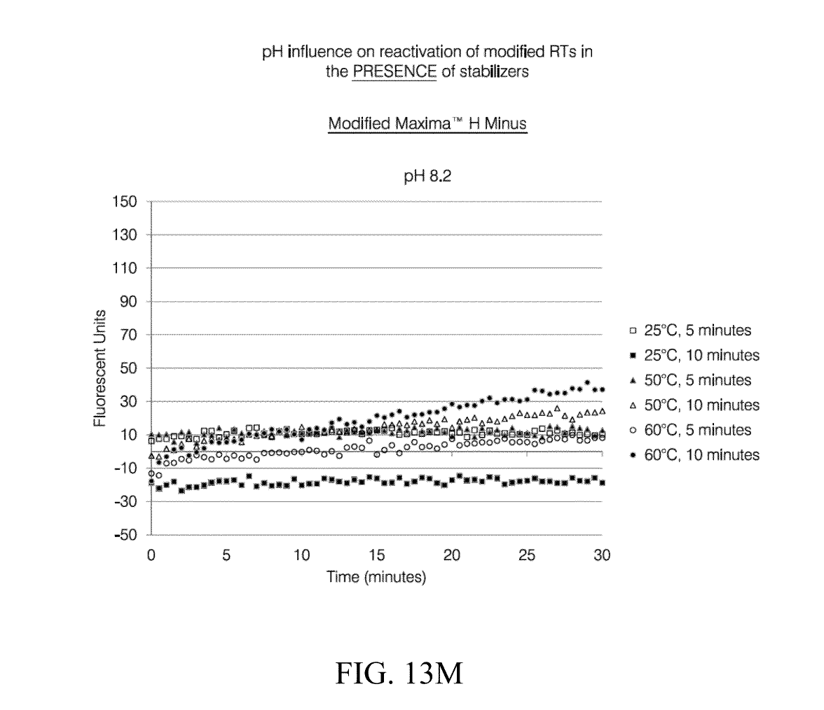

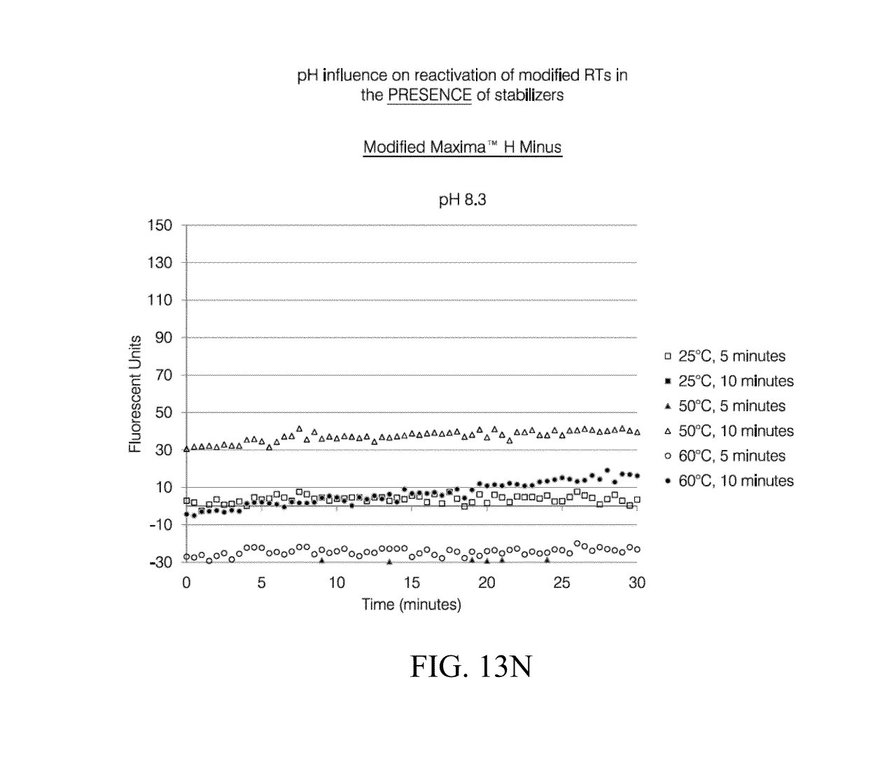

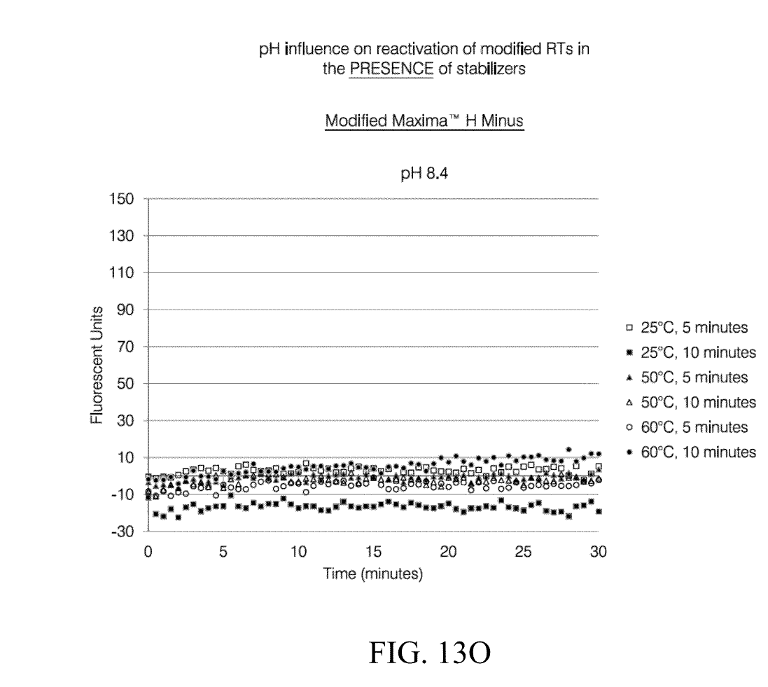

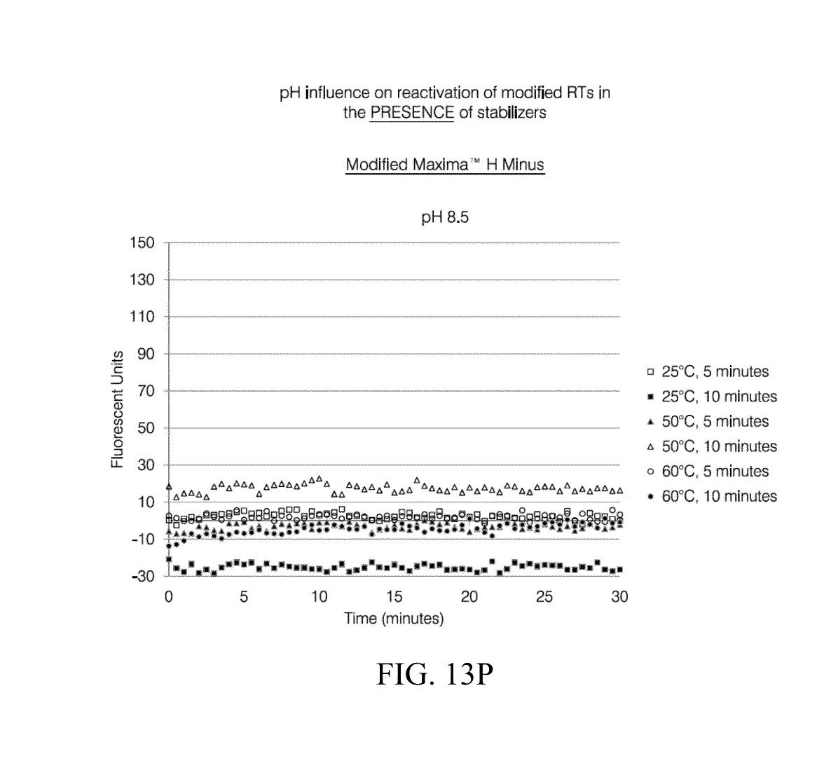

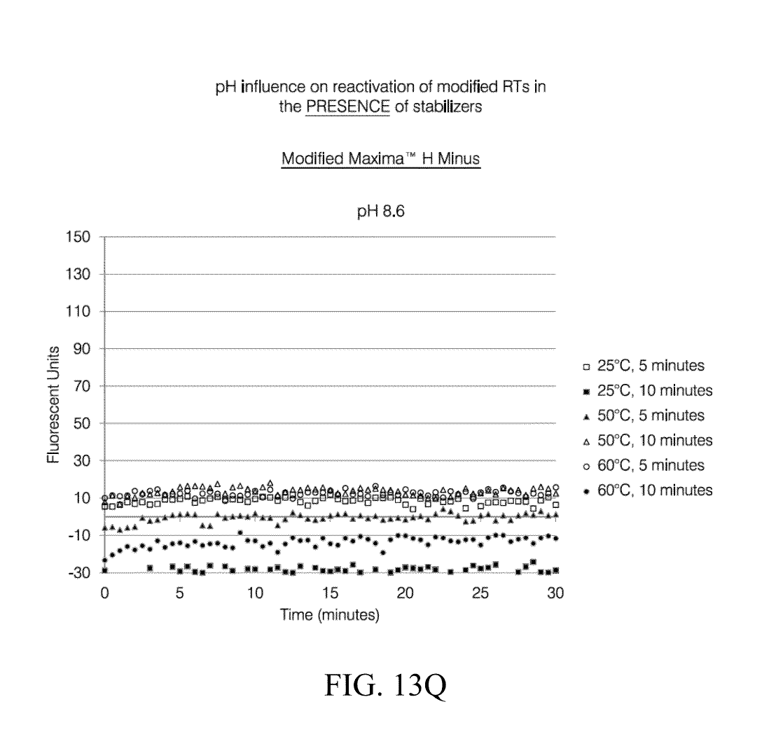

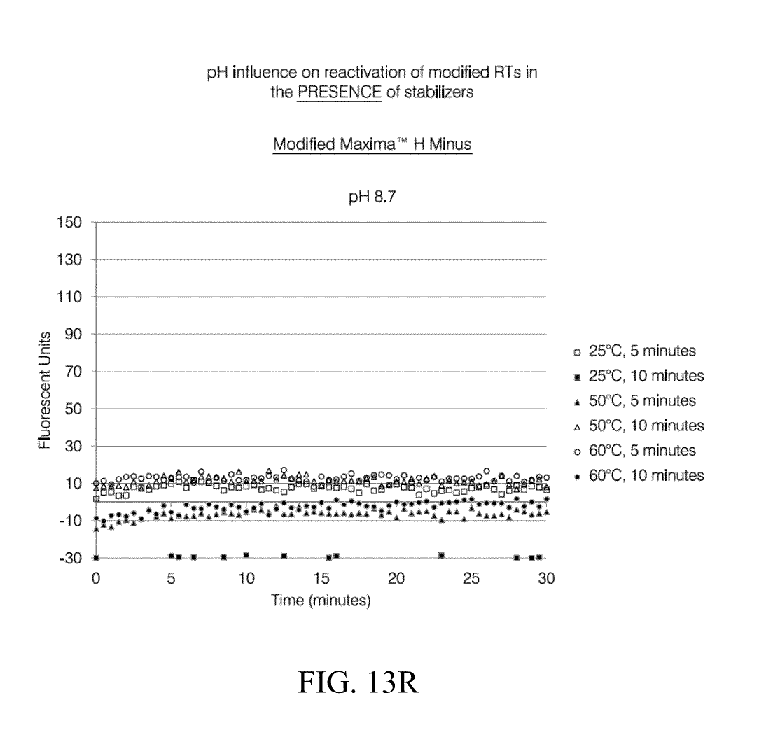

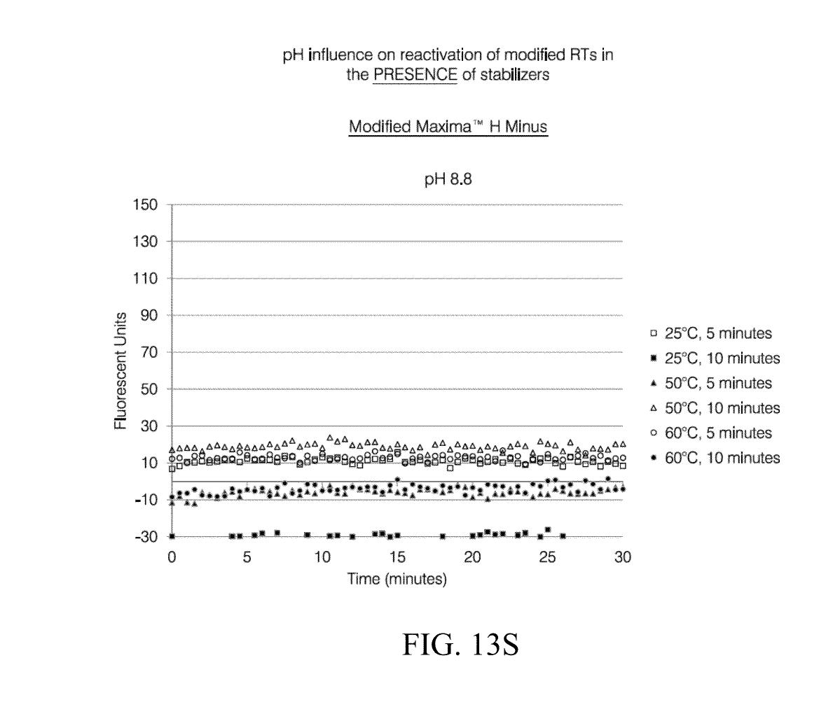

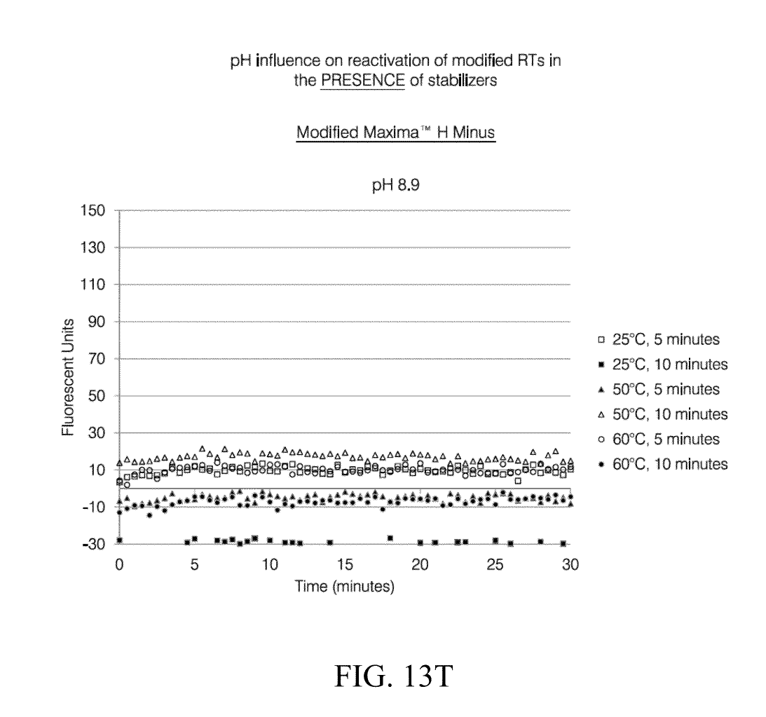

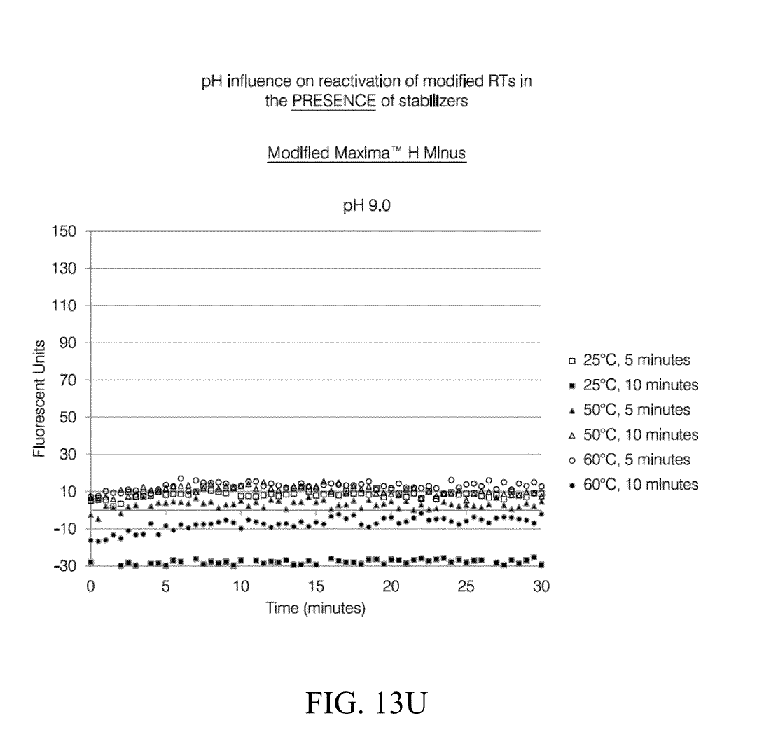

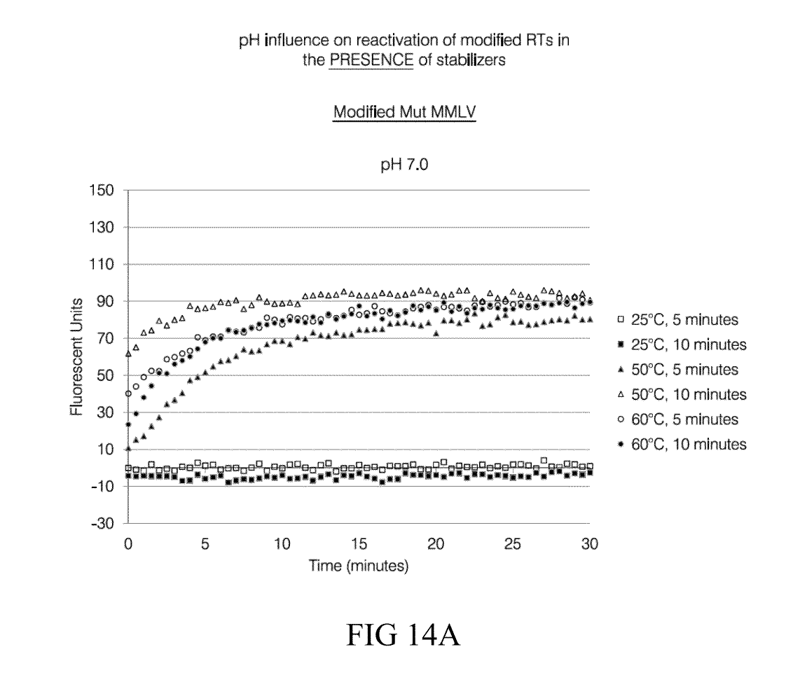

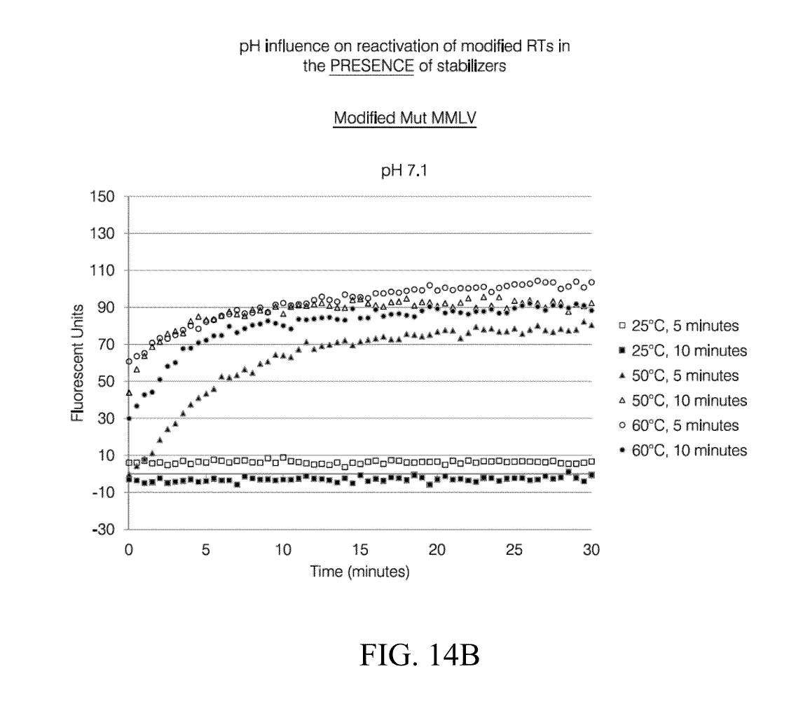

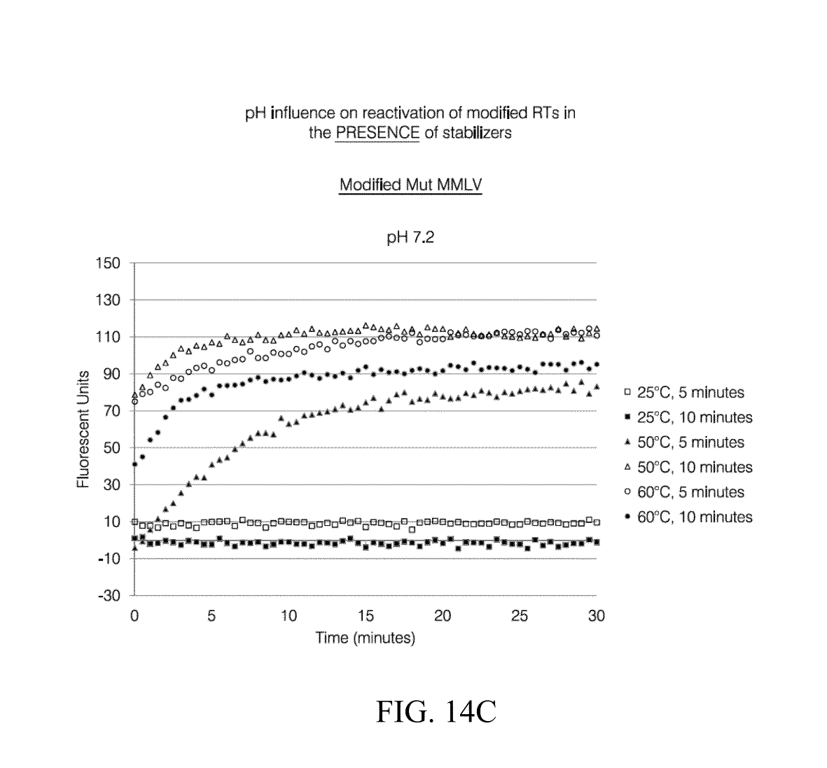

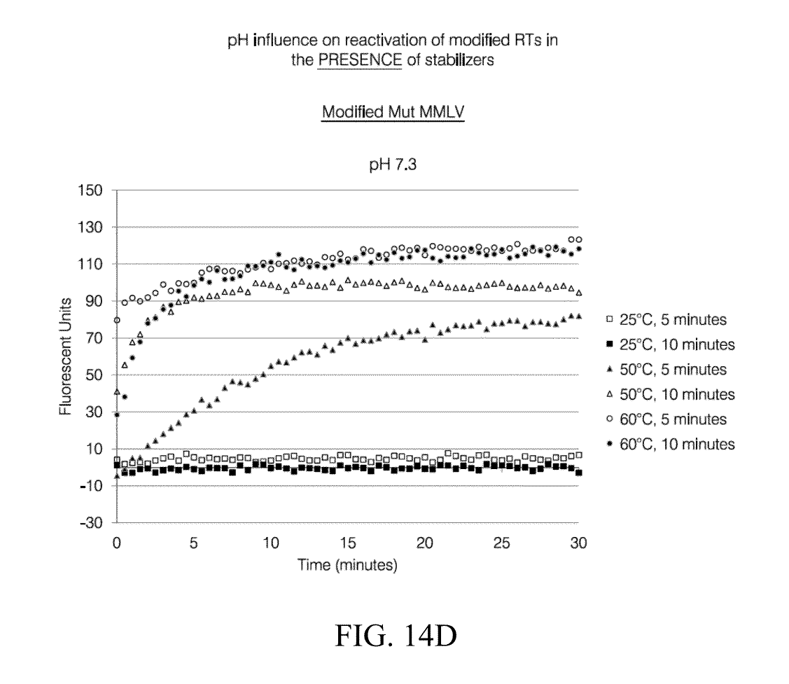

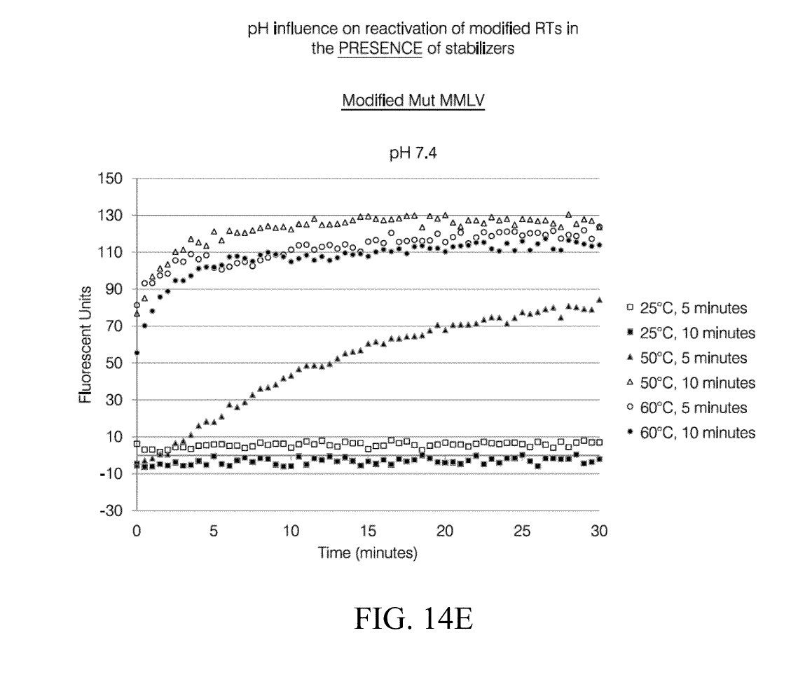

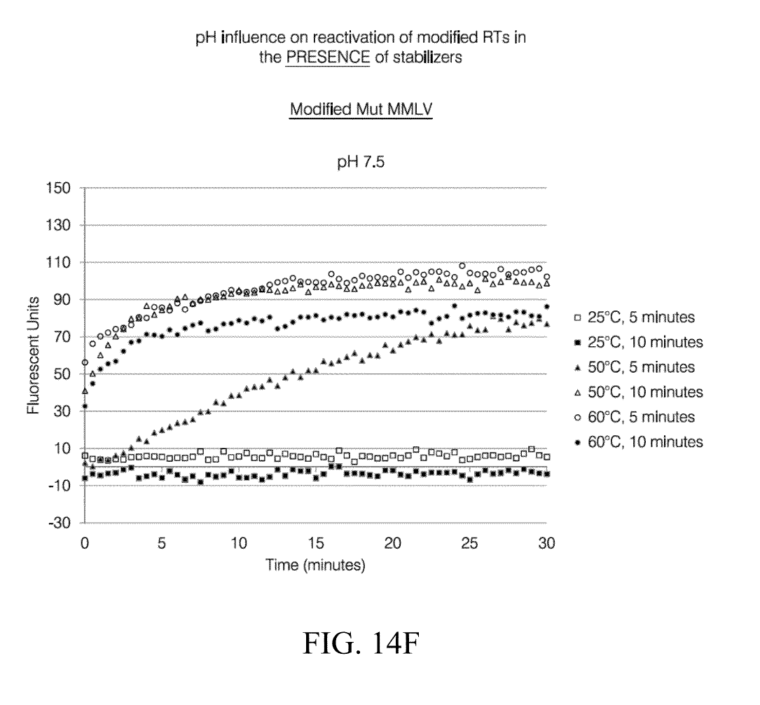

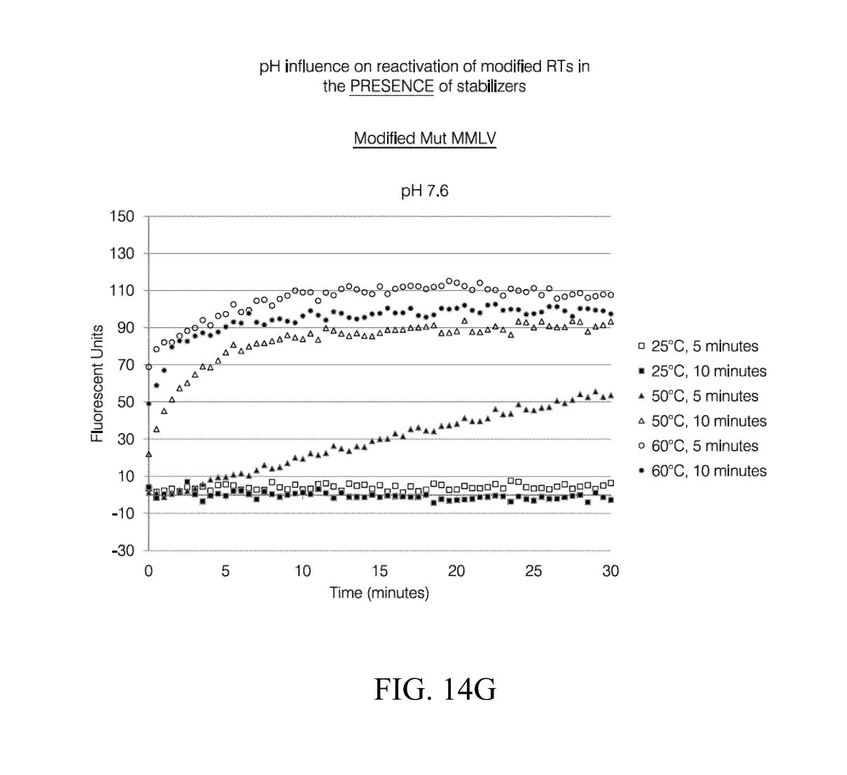

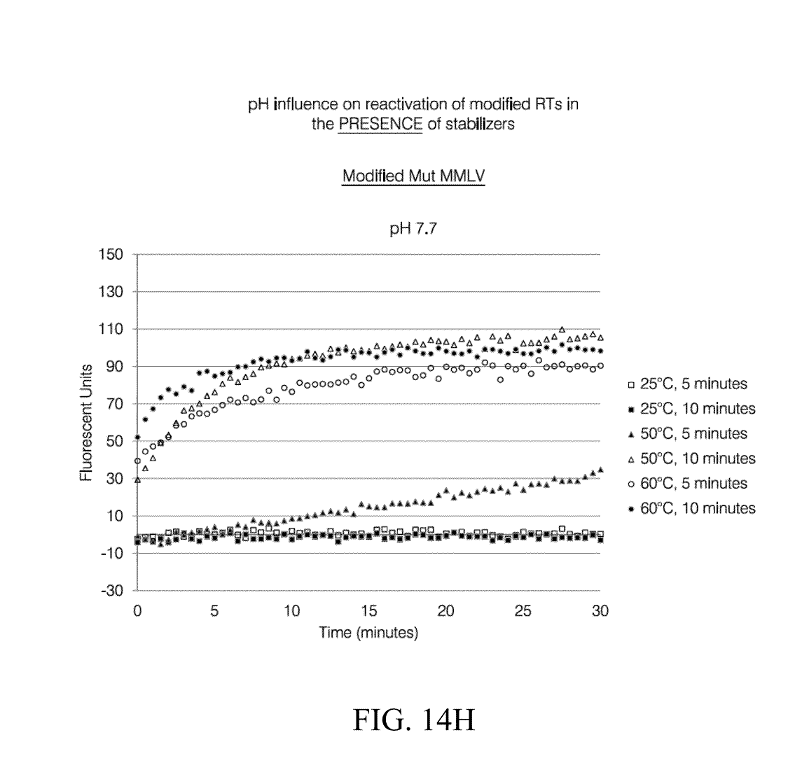

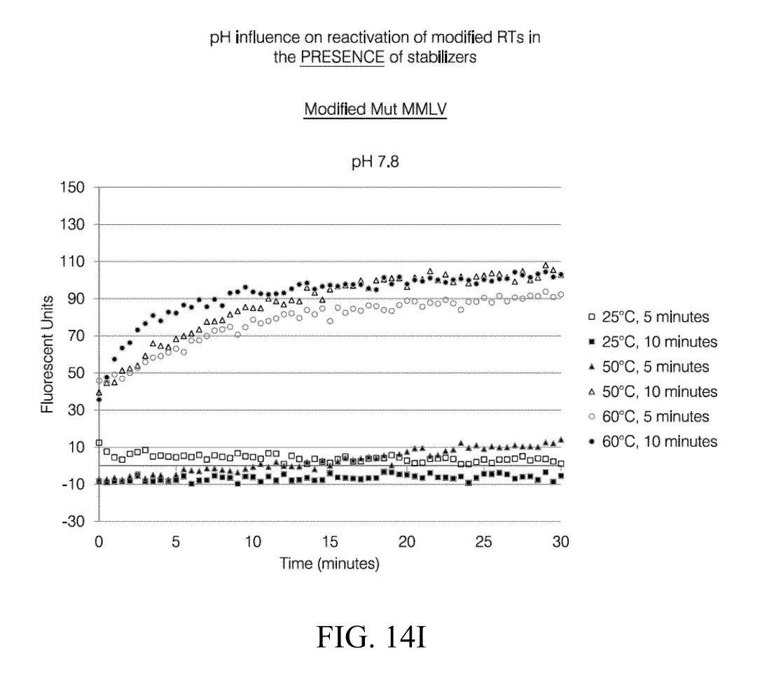

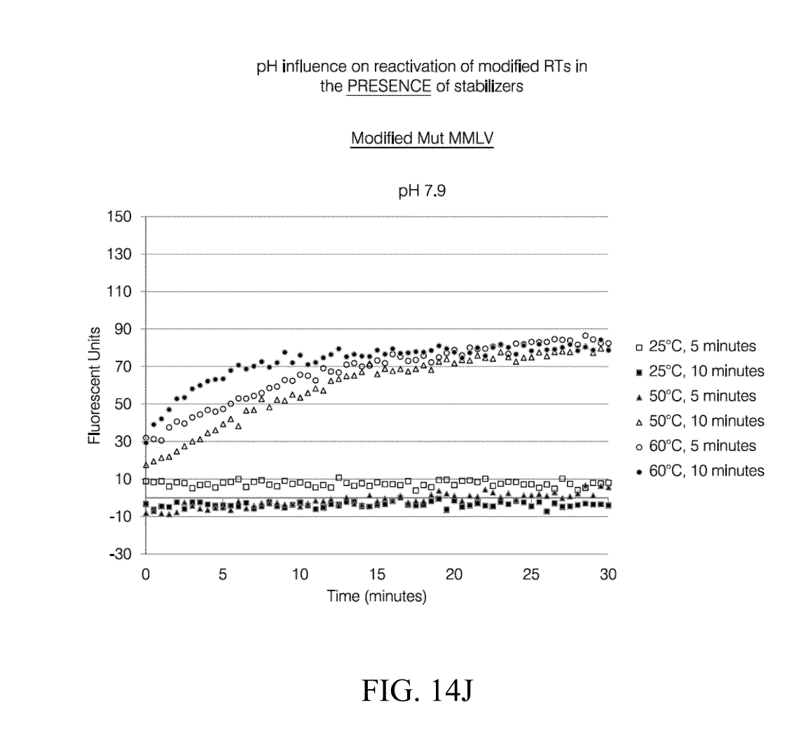

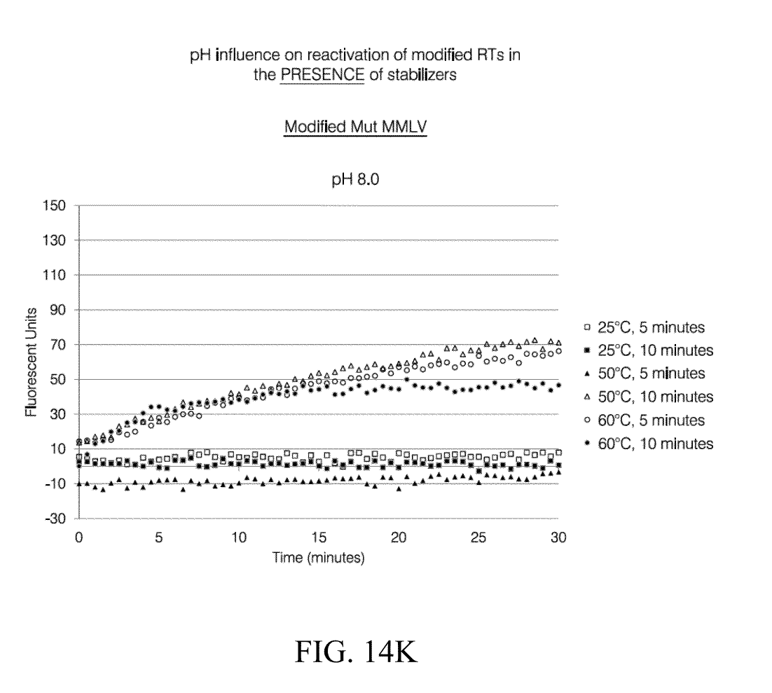

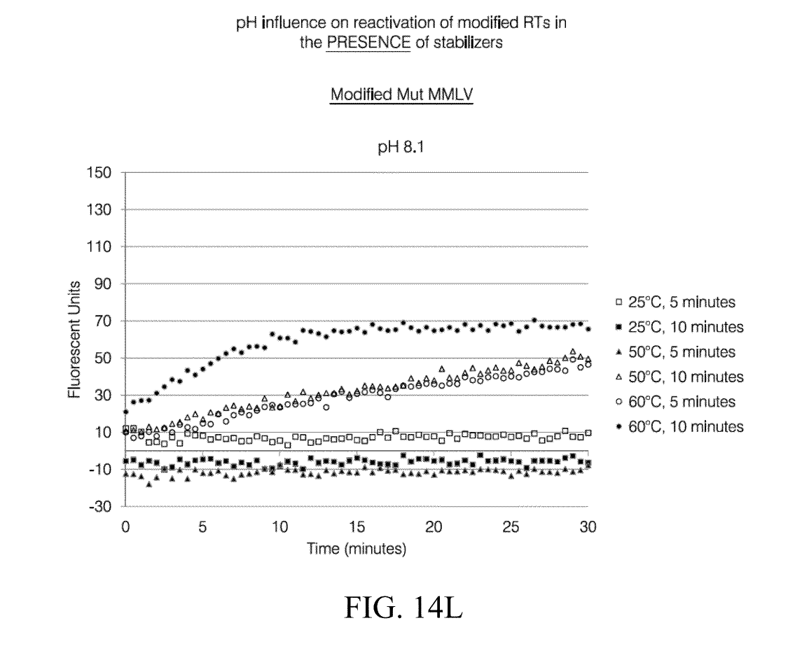

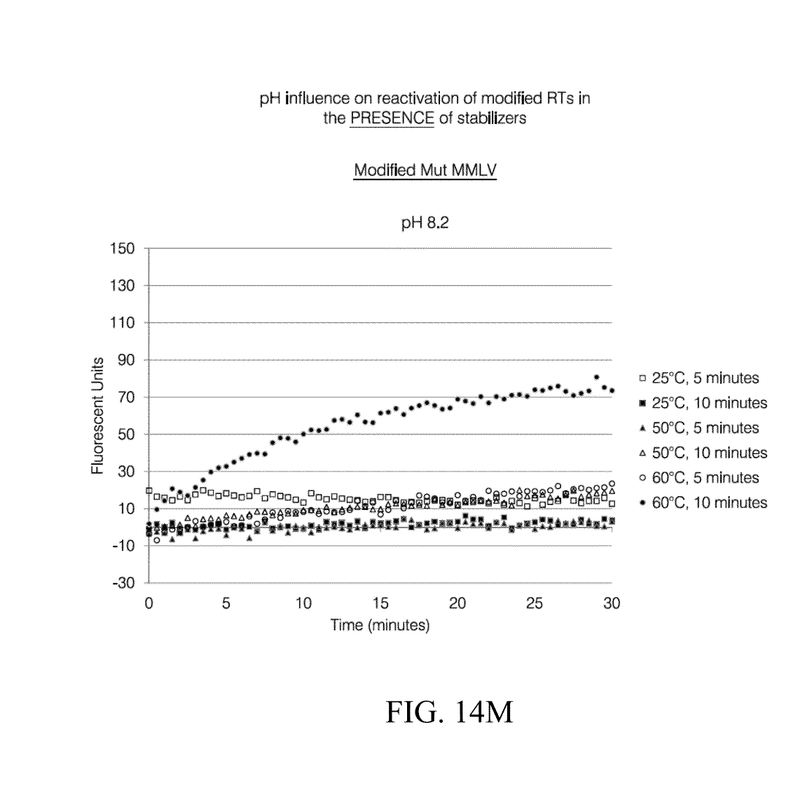

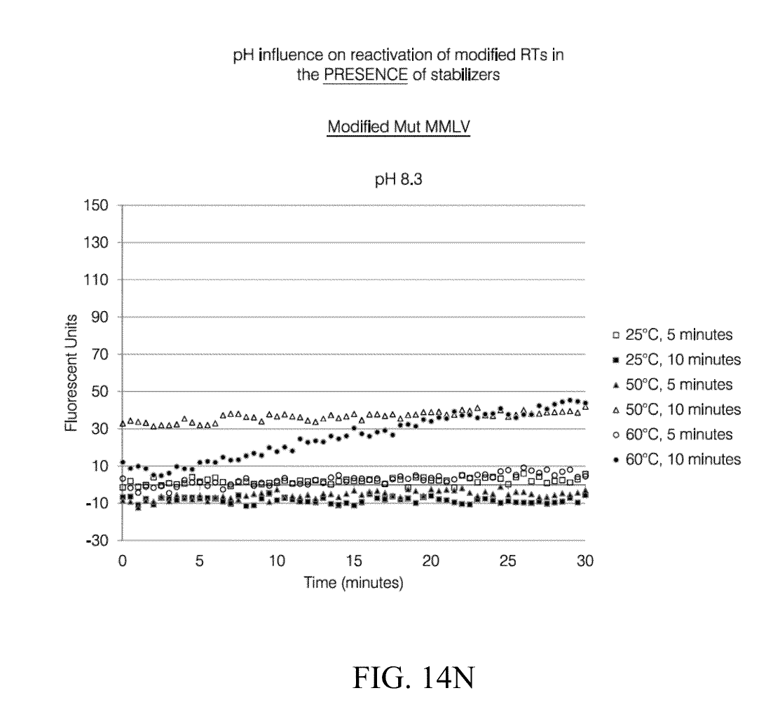

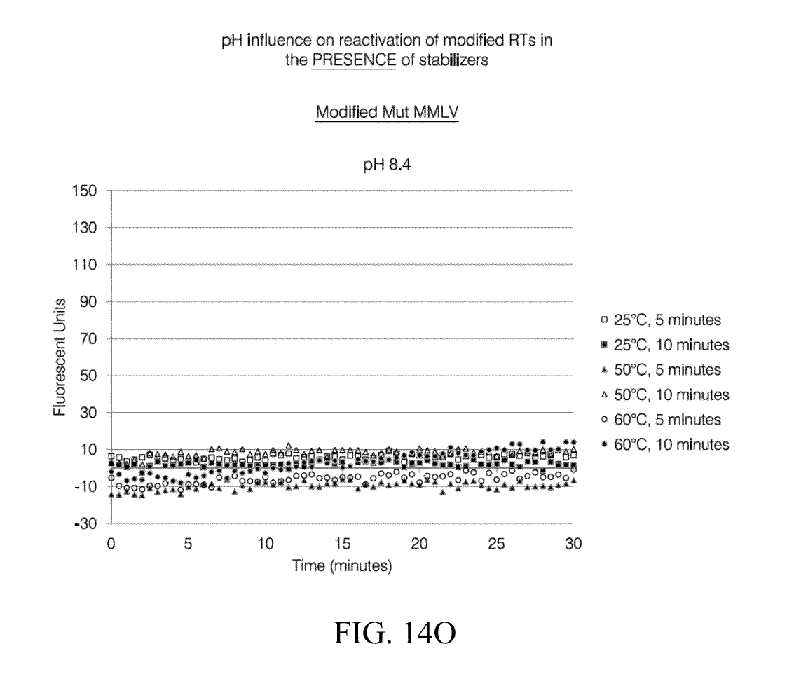

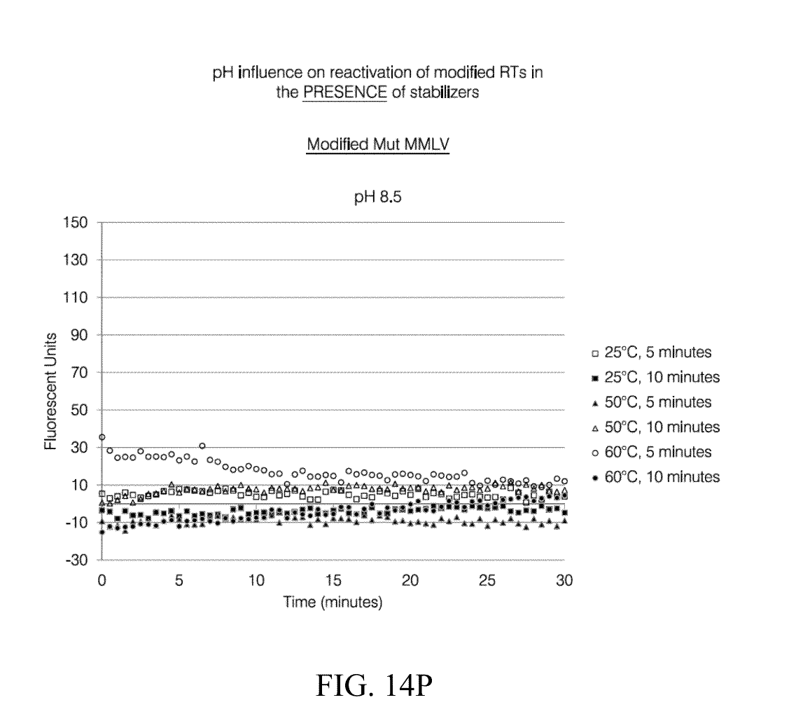

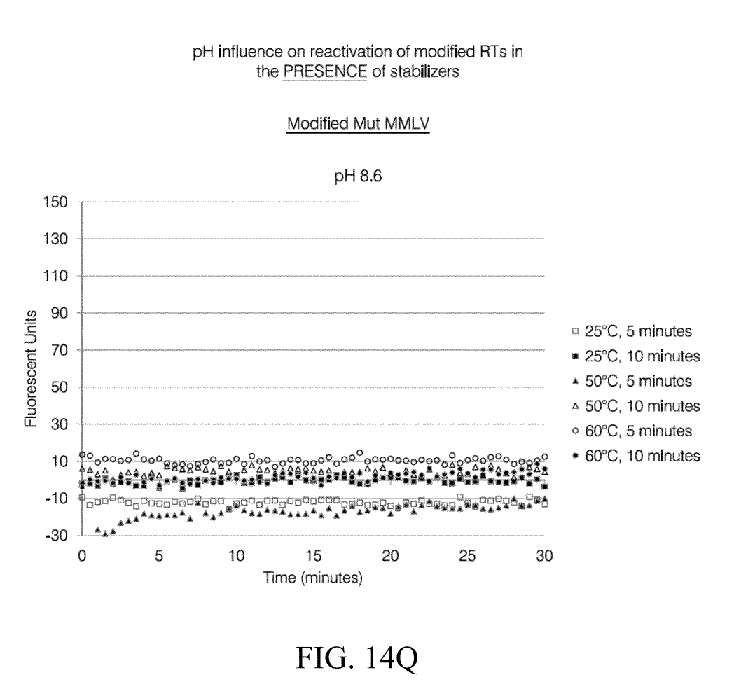

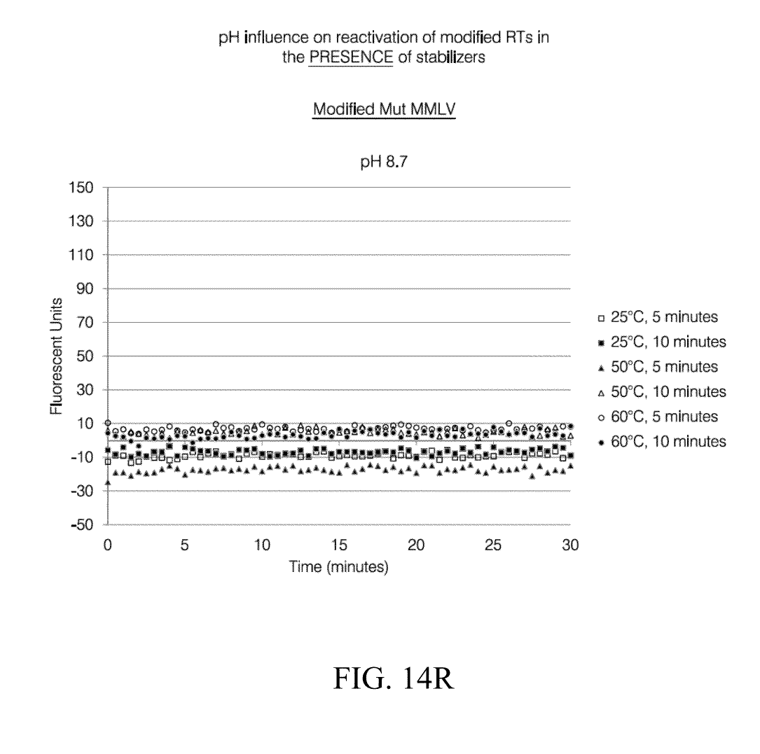

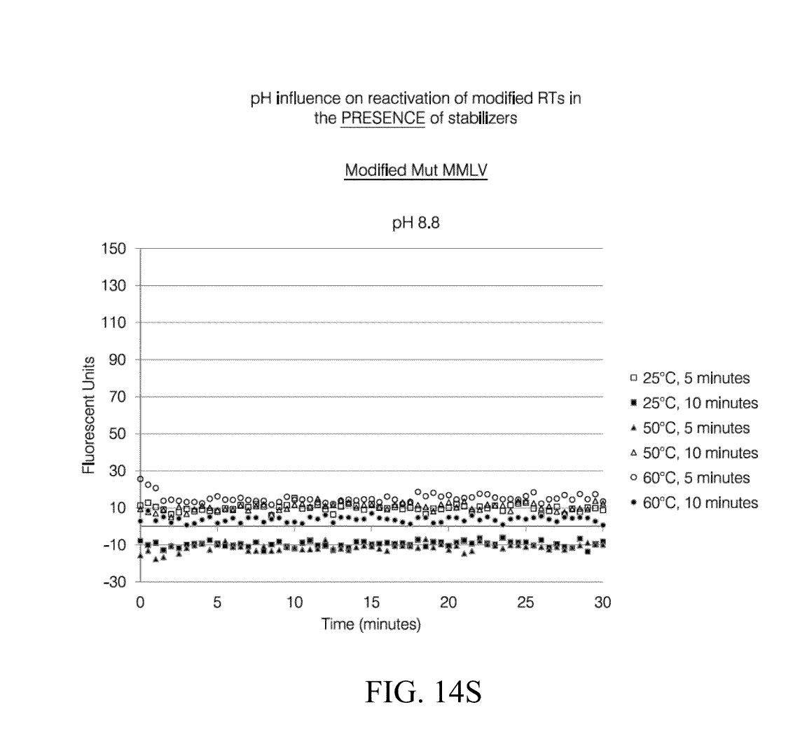

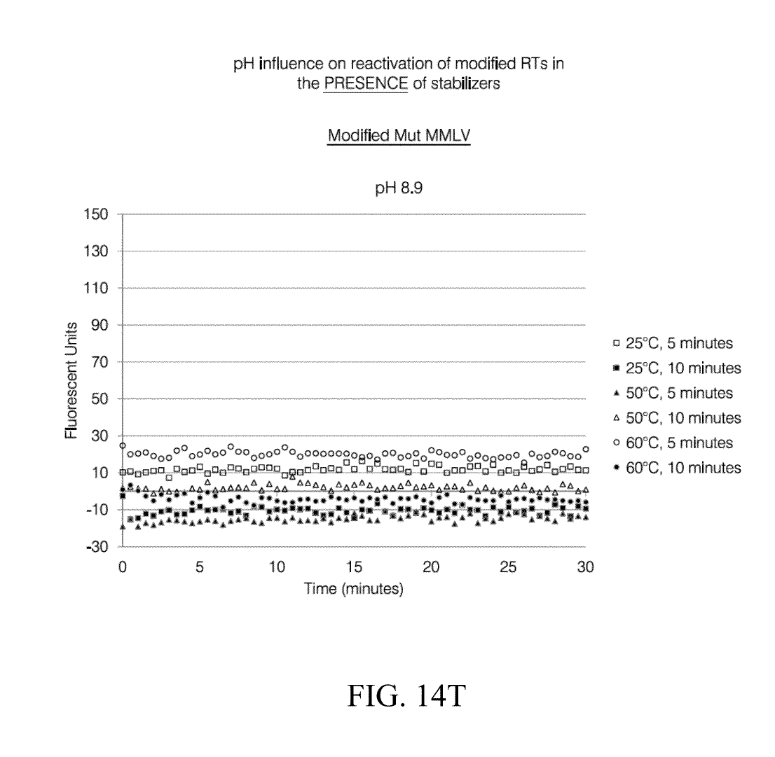

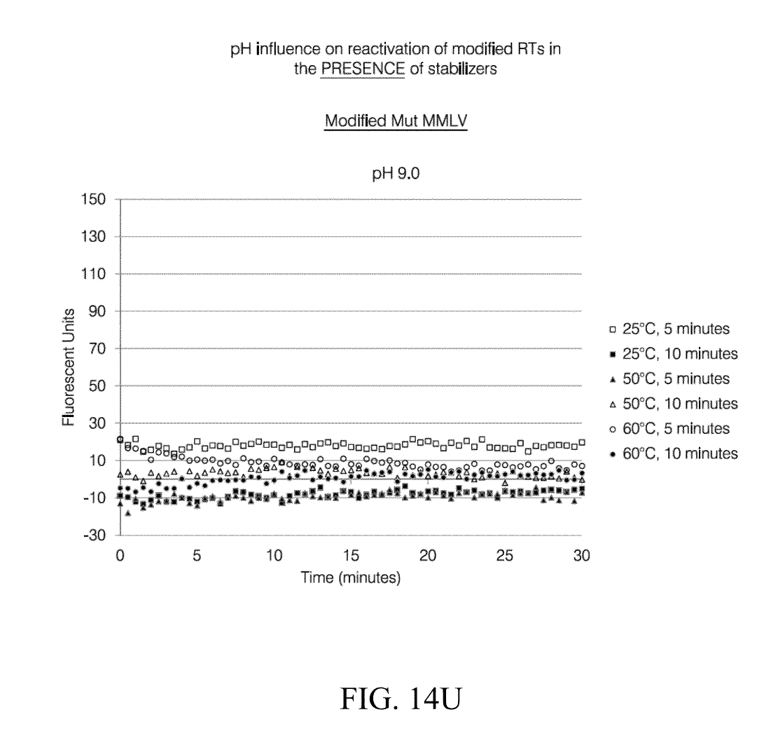

To further delineate the pH range for optimal reactivation of modified enzymes, fluorescent assays as described in Example 1 were performed using reaction buffers ranging from pH 7 to 9 in 0.1 increments and in the presence of 10% glycerol and 0.3 M trehalose additives. The modified enzyme mixtures were preincubated at 25° C., 50° C., or 60° C. for 5 or 10 minutes. Preincubation was stopped by incubating the mixture on ice and then transferring to an assay plate containing 150 nM RNA221 substrate and 0.5 mM dNTPs. According to the data presented in FIGS. 13A through 13U and FIGS. 14A through 14U, the general trend for reactivation of modified thermostable reverse transcriptases is that activation occurs faster as pH decreases. In these assays, minimum or no activation of enzymes occurs above pH 8.3.

### Example 11

### Performance of Chemically Modified Reverse Transcriptases in the Presence of Protein Additives

The following protein additives were tested for their effect on the performance of the chemically modified RTs: Taq mutant M6D, SpermineCassein, β-lactalbumin, α-lactalbumin, apotransferrin and acetylated BSA. The following example of improved modified RT functionality is shown using bovine apotransferrin and acetylated BSA in cDNA extension from an RNA ladder template.

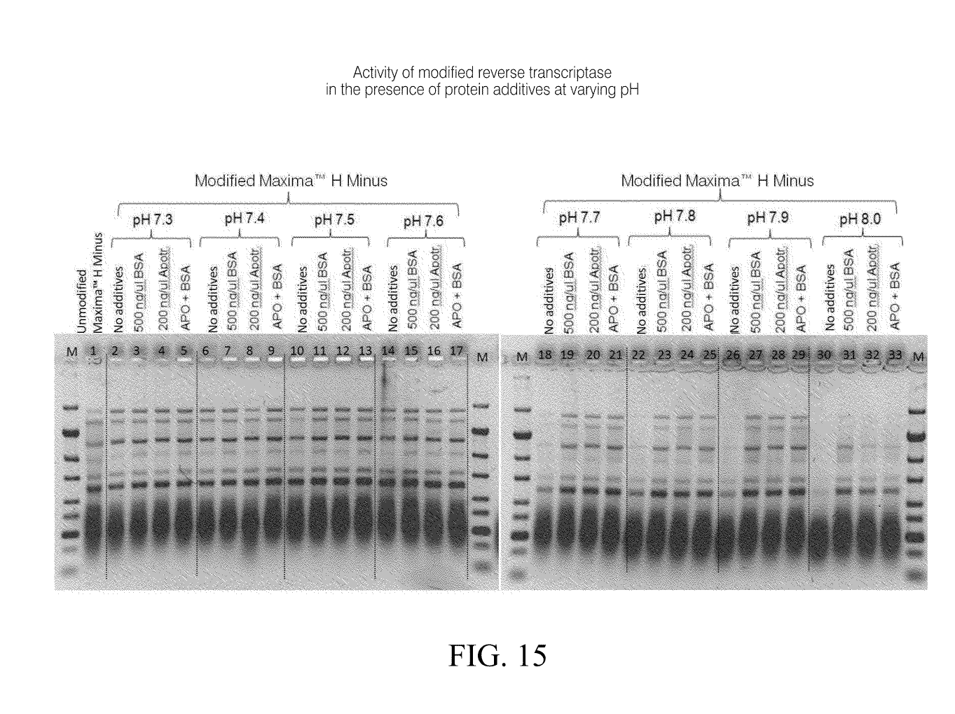

A commercially available RNA ladder (0.5-10 kb, Thermo Fisher Scientific 15623-200) was used as a substrate in 10 ul annealing mixture containing 1 μg RNA ladder, 0.5 mM dNTP, 2.5 μM oligo (dT)₂₅ (SEQ ID NO: 6) in water. The reaction was incubated for 5 min at 65° C. and cooled 4° C. for at least 1 minute. For the RNA extension reaction, 1× buffer (50 mM Tris-HCl, 75 mM KCl and 3 mM MgCl 2 at pH 7.3-pH 8.0), 5mM DTT, 40 U RNase inhibitor, 2 U Modified Maxima™ H Minus (Modified Maxima) enzyme (modified with 400× DMMA) or 2 U unmodified Maxima™ H Minus (Maxima) was prepared in 10 μl to volume with water and where indicated bovine apotransferrin (200 ng/ul) or BSA (500 ng/ul). The annealing and reaction mixes were combined and reactions were incubated at 55° C. for 60 minutes at varying pH as indicated in FIG. 15 (Modified Maxima™ H Minus RT) or at 50° C. for 60 minutes at pH 8.3 (Unmodified Maxima™ H Minus RT). Reactions were then inactivated at 80° C. for 10 minutes. Samples were taken and analyzed on alkaline agarose gels.

While activity of Modified Maxima decreases when pH rises above 7.6 without additions, apotransferrin and BSA partially rescues the activity compared to unmodified Maxima (FIG. 15). In this example, both apotransferrin and BSA stabilize the modified Maxima during reactivation conditions at a pH <8.0.

### Example 12

### Reverse Transcription Primers Compatible with Hot Start Reverse Transcription

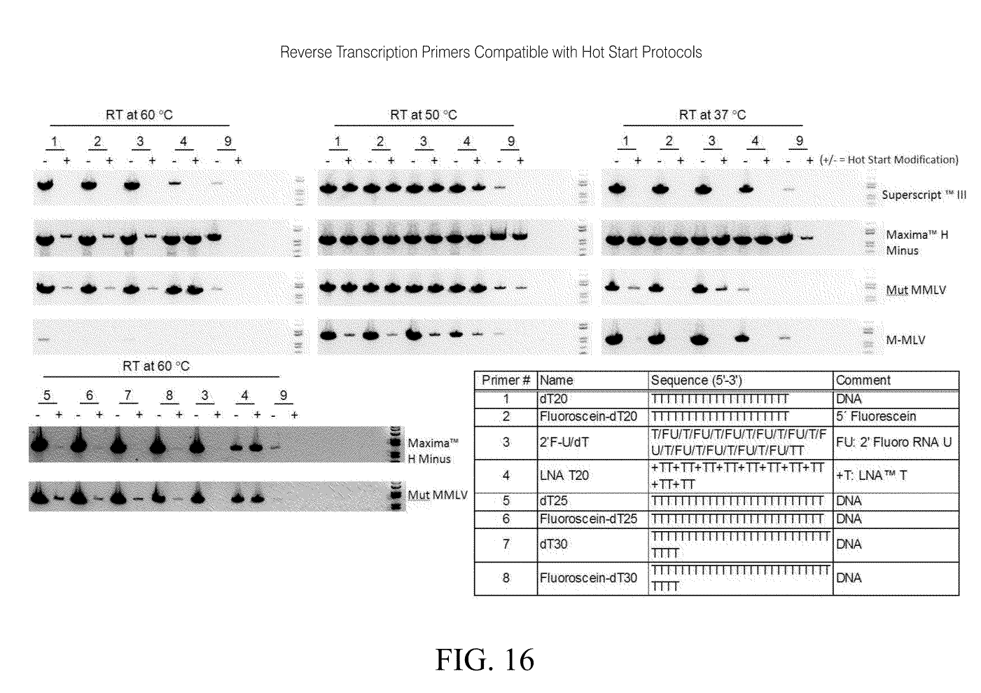

To evaluate the types of reverse transcriptase primers compatible with the hot start enzymes, RT-PCR of human EF1α (955 by product) was performed. 50 ng/μL (final reaction concentration) DNA-free HeLa Total RNA (Ambion®, Catalog #AM7852) and 2.5 μM (final reaction concentration) of indicated primers were initially heated to 65° C. for 5 minutes and incubated on ice for at least 1 minute. Primer sequences were as follows: human EF1α forward primer: 5′-GAAGCTGGTATCTCCAAGAATG-3′ (SEQ ID NO: 3) and human EF1α reverse primer: 5′-CTGCTTGATGACACCCACCGCAACT-3′ (SEQ ID NO: 4). Annealed RNA and primers were then added to 50 mM Tris-HCl (pH 7.3 at room temperature), 75 mM KCl, 3 mM MgCl₂, 5 mM DTT, 0.5 mM dNTPs, 0.05 U/μL RNaseOUT Recombinant RNase Inhibitor, 10% glycerol, 0.3 M trehalose, and 10 U/uL unmodified (−) or modified (+) reverse transcriptase enzymes assembled at room temperature. Reactions were incubated at 37° C., 50° C., and 60° C. for 1 hour followed by inactivation of reverse transcriptases by heating at 70° C. for 15 minutes and digestion of RNA with 0.5 U/μL Ribonuclease H (Invitrogen™, Catalog #18021-071) and 0.02 μg/μL RNase A Ambion® Catalog #AM2272 at 37° C. for 30 minutes. 1 μL out of a 20 μL reverse transcription reaction was added to a 24 μL PCR reaction mix resulting in a 25 μL PCR reaction with 1× High Fidelity PCR Buffer, 2 mM MgSO4, 0.2 mM each dNTP, 0.2 μM human EF1α forward primer, 0.2 μM EF1α reverse primer, and 0.05 U/μL Platinum® Taq DNA Polymerase High Fidelity (Invitrogen™, Catalog #11304-011). Cycling parameters were 94° C. for 5 minutes, followed by 25 cycles of 94° C. for 30 seconds, 60° C. for 30 seconds, 68° C. for 1 minute, a final extension of 68° C. for 5 minutes, and then holding at 4° C. 10 μL of the PCR reaction were resolved for 20 minutes using E-Gel® 48 Agarose Gels, 1% (Invitrogen™) and visualized by E-Gel® Imager System with UV Light Base (Invitrogen™). Unmodified enzymes (− lanes) have residual activity at room temperature and below and should result in PCR product regardless of the primer used as long as the reverse transcription temperature does not immediately deactivate the enzymes. As shown in FIG. 16, all unmodified lanes at all temperatures tested with the exception of M-MLV at 60° C., PCR product is observed. Even in the presence of stabilizers, such as glycerol and trehalose, M-MLV is not active at 60° C. (also shown in Example 5). All primers tested result in stable PCR product when reverse transcription is performed at 50° C., suggesting these primers remain annealed to template RNA at this temperature. Unreliable amplification of cDNA for M-MLV at 50° C. is most likely due to borderline thermostability and thermoreactivity. Only LNA T20 primer (SEQ ID NO: 5) (lanes labeled 4) result in strong PCR product when reverse transcription is performed at 60° C. with modified Maxima™ H MINUS and MUT MMLV (+lanes). Since the modified enzymes do not have residual activity at room temperature and below, only LNA T20 (SEQ ID NO: 5) remains efficiently hybridized to template RNA at 60° C. Modified (+) Superscript III and wild type M-MLV are not sufficiently thermostable or thermoreactive at 60° C. even in the presence of stabilizers to generate cDNA template for PCR using LNA T20 primer (SEQ ID NO: 5). While all primers can anneal at 37° C., the modified enzymes may not be sufficiently reactivated at this temperature to result in cDNA template. Only modified Maxima™ H Minus is sufficiently reactivated to give RT-PCR product.

### Example 13

### Hot Start Reverse Transcriptase Enables Primer-Specific cDNA Synthesis

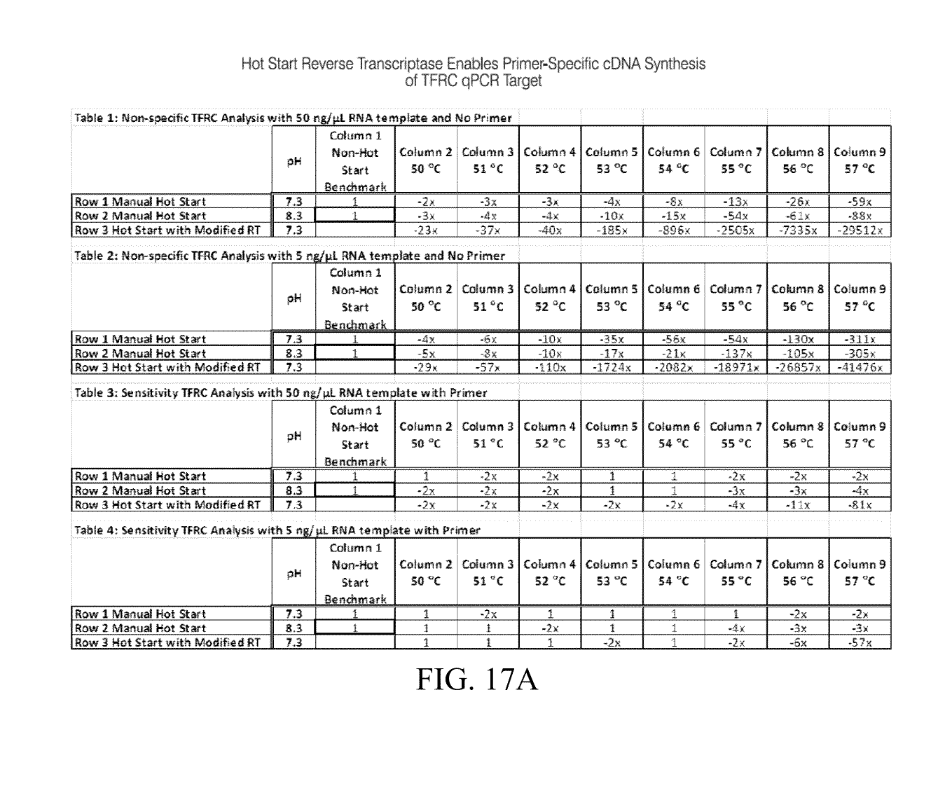

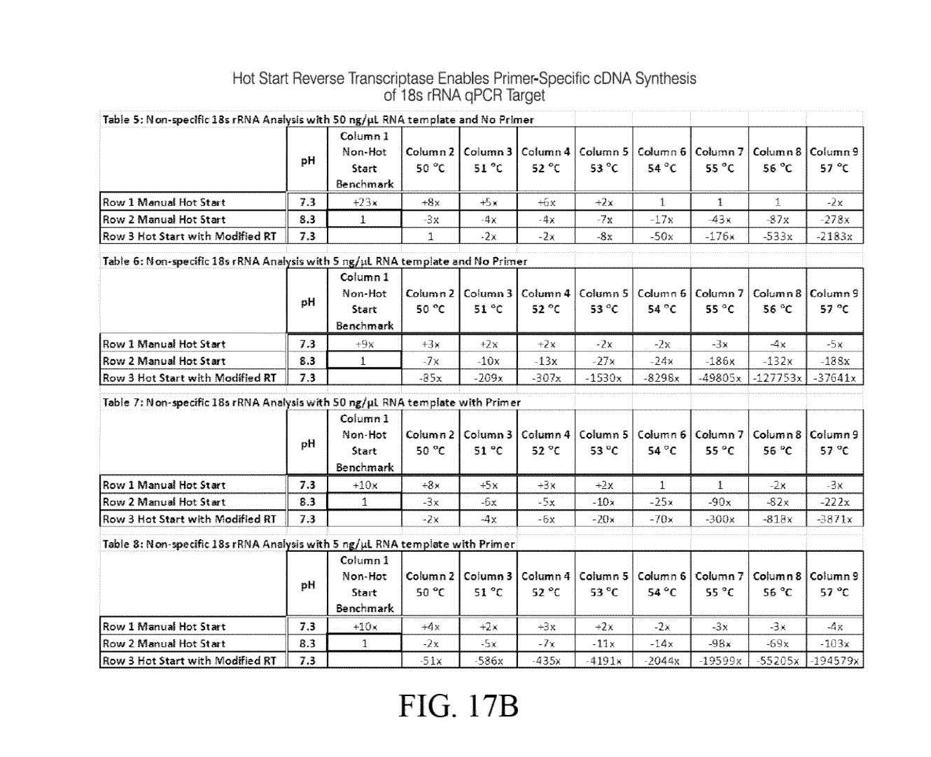

Unmodified reverse transcriptases possess residual polymerization activity at room temperature (−25° C.) and below (ice). This property results in non-specific cDNA generation because RNA secondary structure at these low temperatures results in RNA-RNA priming and non-specific hybridization of DNA primer to RNA. A hot start reverse transcription protocol using modified reverse transcriptases would alleviate these problems. 5 or 50 ng/μL DNA-free HeLa Total RNA (Ambion®, Catalog #AM7852) (final concentration) and 2.5 /μM of oligo(dT)₂₅ (SEQ ID NO: 6) (final concentration) or water (no primer reverse transcriptase reactions) were initially heated to 65° C. for 5 minutes and incubated on ice for at least 1 minute. Annealed RNA and primer were then added to 50 mM Tris-HCl (pH 8.3 or pH 7.3 at room temperature), 75 mM KCl, 3 mM MgCl₂, 5 mM DTT, 0.5 mM dNTPs, 0.05 U/μL RNaseOUT Recombinant RNase Inhibitor, and 10 U/μL modified Maxima™ H Minus reverse transcriptase assembled at room temperature. Reverse transcriptase reactions were incubated at 50-57° C. for 1 hour followed by inactivation of reverse transcriptases by heating at 70° C. for 15 minutes and digestion of RNA with 0.5 U/μL Ribonuclease H (Invitrogen™, Catalog #18021-071) and 0.02 μg/μL (RNase A Ambion® Catalog #AM2272) at 37° C. for 30 minutes. For gene-specific analysis (FIG. 17A: Tables 1-4), 1 μL of a 20 μL reverse transcriptase reaction was added to a qPCR reaction composed of 5 μL EXPRESS qPCR SuperMix Universal (Invitrogen™ 11785-01K), 0.5 μM ROX, 1× Human Transferrin Receptor (TFRC) (187 bp) Primer/Probe set according to the manufacturer's instructions (Applied Biosystems®Catalog #4333770F), and water up to 10 μL. Ribosomal RNA analysis (FIG. 17B: Tables 5-8) was performed similar to gene-specific analysis except 1 μL of a 1000-fold dilution of the reverse transcription reaction and 1× Eukaryotic 18 s rRNA primer/probe set according to the manufacturer's instructions (Applied Biosystems® Catalog #4352930E) were used instead. Thermal cycling was performed using ViiA™ 7 Real-Time PCR System (Applied Biosystems®) with cycling parameters, 50° C. for 2 minutes, 95° C. for 2 minutes, and 40 cycles of 95° C. for 15 seconds and 60° C. for 1 minute. For manual hot start reactions with unmodified Maxima™ H Minus, reactions without enzymes were first heated to designated temperature (50-57° C.) and then reverse transcriptase was added to 10 U/μL. For actual hot start reactions with modified Maxima™ H Minus, all reaction components were added at room temperature prior to heating to designated temperatures. Non-hot start conditions using reaction buffer at pH 8.3 and pH 7.3 where all reactions components were added at room temperature prior to incubating at 50° C. were also performed as positive controls. The non-hot start, pH 8.3 condition was used as a benchmarking control to evaluate pH 7.3 buffer and hot start protocols. Fold change in Transferrin Receptor (TFRC) and 18 s rRNA cDNA synthesis for unmodified enzyme at pH 7.3 non-hot start conditions, manual hot start conditions using unmodified enzyme, and actual hot start conditions using modified enzyme compared to unmodified enzyme at pH 8.3 non-hot start condition (current industry protocol) (bold outline box) was determined by the equation −[2̂(Ct−Ct_{control})]. Negative values indicate a fold reduction and positive values indicate fold increase compared to benchmark control. In the case for 18 s rRNA cDNA analysis, the 1000-fold template dilution was taken into account by the subtraction of ΔCt=9.97.

For Transferrin Receptor (TFRC) cDNA generation, pH 7.3 buffer did not result in any fold change compared to pH 8.3 buffer whether or not DNA primer was added for non-hot start protocol (FIG. 17A: Tables 1-4, Column 1). However for 18 s rRNA non-specific cDNA synthesis (includes both no primer and primer added reactions), pH 7.3 buffer results in increased cDNA generated over pH 8.3 buffer using non-hot start protocol (FIG. 17B: Tables 5-8, Column 1). This indicates that pH 7.3 buffer did not affect Maxima™ H Minus, but has a sequence-dependent effect on non-specific priming off template RNA.

Manual hot start reverse transcription using unmodified enzymes results in a 2-300-fold reduction in the amount of non-specific Transferrin Receptor (TFRC) cDNA generated (when primer is not added to reverse transcription reaction) compared to non-hot start controls in both pH 7.3 and 8.3 buffers. Non-specific Transferrin Receptor (TFRC) cDNA generation is reduced as RNA template concentration decreases and reverse transcription temperature increases (FIG. 17A: Table 1 and 2, row 1 and row 2). Actual hot start reactions with modified Maxima™ H Minus show the same trend except fold reduction ranges from 20-40000-fold (FIGS. 17A and 17B: Table 1 and 2, row 3). For non-specific 18 s ribosomal cDNA generated, reactions occurring at pH 7.3 result in an increase or modest decrease in non-specific priming using unmodified enzyme (FIG. 17B: Tables 5-8, row 1 compared to boxed control). Manual hot-start reactions at pH 8.3 result in a consistent fold reduction in the amount of non-specific cDNA generated that is temperature dependent (FIG. 17B: Tables 5-8, row 2). Bona fide hot start using modified enzymes result in greater fold reduction as temperature is increased and template RNA decreased (FIG. 17B: Tables 5-8, row 3). The benefit of actual hot start is therefore exemplified by the greater decrease in non-specific cDNA generated with and without reverse transcription primer compared to manual hot start and non-hot start. Using modified enzymes for actual hot start is of even greater importance for decreasing or eliminating cDNA from very high copy, non-polyadenylated RNAs, such as 18 s rRNA, a major contaminant in any RNA preparation.

While hot start reverse transcription using modified enzyme leads up to >1000-fold reduction in non-specificity, this same protocol sustains a high degree of sensitivity. Analysis of Transferrin Receptor (TFRC) cDNA when reverse transcription primer is present results in no change or only about a 2-fold reduction compared to non-hot start control when reaction temperature does not exceed 55° C. Sensitivity using manual hot start is very similar to bona fide hot start, but actual hot start with modified reverse transcriptase far exceeds manual hot start in preventing non-specificity.

It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated documents defines a term that contradicts that term's definition in this application, this application controls.

All references cited herein, including patents, patent applications, papers, text books, and the like, and the references cited therein, to the extent that they are not already, are hereby incorporated by reference in their entirety. In the event that one or more of the incorporated literature and similar materials differs.

1. A thermostable and/or thermoreactive reverse transcriptase enzyme comprising a chemical modification, wherein the modified reverse transcriptase is thermostable and/or thermoreactive at a temperature exceeding about 42° C. at a pH between 6.0 to 9.0, and wherein the chemical modification results in at least a 50% decrease in reverse transcriptase activity as compared to a corresponding unmodified reverse transcriptase when activity is measured under non-activating conditions.

2. The modified reverse transcriptase of claim 1, wherein the modified reverse transcriptase is thermostable and/or thermoreactive at a temperature exceeding about 50° C. at a pH between 6.0 to 9.0.

3. The modified reverse transcriptase of claim 1, wherein the modified reverse transcriptase exhibits an increase in reverse transcriptase activity in a reaction mixture under activating conditions compared to the reverse transcriptase activity observed under non-activating conditions.

4. The modified reverse transcriptase of claim 1, wherein the non-activating conditions comprise a pH of at least 8.0 and/or a temperature less than about 37° C.

5. The modified reverse transcriptase of claim 3, wherein the activating conditions comprise a pH less than about 8.0 and/or a temperature greater than about 37° C.

6. The modified reverse transcriptase of claim 1, wherein the at least 50% decrease in reverse transcriptase activity is maintained for at least about 30 minutes under non-activating conditions.

7. The modified reverse transcriptase of claim 3, wherein the increase in reverse transcriptase activity is at least two-fold.

8. The modified reverse transcriptase of claim 3, wherein the increase in reverse transcriptase activity occurs in less than about 30 minutes under activating conditions.

9. The modified reverse transcriptase of claim 1, wherein the chemical modification is made by a modifier reagent selected from the group consisting of maleic anhydride; exo-cis-3,6-endoxo-Δ⁴-tetrahydropthalic anhydride; citraconic anhydride; 3,4,5,6-tetrahydrophthalic anhydride; cis-aconitic anhydride; 2,3-dimethylmaleic anhydride; and dicarboxylic acid imide.

10. (canceled)

11. The modified reverse transcriptase of claim 1, wherein the chemical modification results in at least a 90% decrease in reverse transcriptase activity.

12. (canceled)

13. The modified reverse transcriptase of claim 1, wherein the thermostable and/or thermoreactive reverse transcriptase is a mutant Moloney Murine Leukemia Virus (M-MLV) reverse transcriptase.

14. A composition comprising a thermostable and/or thermoreactive reverse transcriptase enzyme comprising a chemical modification, wherein the modified reverse transcriptase is thermostable and/or thermoreactive at a temperature exceeding about 42° C. at a pH between 6.0 to 9.0, and wherein the chemical modification results in at least a 50% decrease in reverse transcriptase activity as compared to a corresponding unmodified reverse transcriptase when activity is measure under non-activating conditions.

15. The composition of claim 14, further comprising an RNA template.

16. The composition of claim 14, further comprising at least one stabilizing component.

17. The composition of claim 16, wherein the at least one stabilizing component is selected from the group consisting of glycerol, trehalose, lactose, maltose, galactose, glucose, sucrose, dimethyl sulfoxide (DMSO), polyethylene glycol, and sorbitol.

18. The composition of claim 17, wherein when the at least one stabilizing component is glycerol and/or trehalose, the glycerol concentration is between 1% and 15% and the trehalose concentration is between 1% and 18%.

19. The composition of claim 14, further comprising a primer.

20. The composition of claim 19, wherein the primer is an oligo(dT)ₙ primer.

21. (canceled)

22. The composition of claim 14, wherein the composition is at a pH of 8.0 or less.

23. The composition of claim 14, further comprising at least one protein additive.

24.-26. (canceled)

27. A method for reactivation of a reversibly inactivated reverse transcriptase in a reaction mixture comprising reactivating the reversibly inactivated reverse transcriptase at a pH of 8.3 or less.

28.-41. (canceled)

42. A method for the reverse transcription of a target nucleic acid contained in a sample comprising the steps of:
(a) contacting the sample with a reverse transcription reaction mixture containing a primer and a reversibly inactivated thermostable and/or thermoreactive reverse transcriptase, wherein the reverse transcriptase is thermostable and/or thermoreactive at a temperature above 42° C. at a pH between 6.0 to 9.0, and wherein the reverse transcriptase exhibits essentially no reverse transcriptase activity in reaction mixture under non-activating conditions; and
(b) incubating the resulting mixture of step (a) under activating conditions for an amount of time sufficient to reactivate the reversibly inactivated reverse transcriptase and allow formation of primer extension products.

43.-51. (canceled)
